(19) United States
(12) Patent Application Publication
(10) Pub. No.: US 2024/0141384 A1
(43) Pub. Date: May 2, 2024

(54) METHODS AND COMPOSITIONS TO CONFER REGULATION TO GENE THERAPY CARGOES BY HETEROLOGOUS USE OF ALTERNATIVE SPLICING CASSETTES

(71) Applicant: University of Florida Research Foundation, Incoporated, Gainesville, FL (US)

(72) Inventors: Eric Tzy-Shi Wang, Gainesville, FL (US); Keril K. Poukalov, Gainesville, FL (US)

(73) Assignee: University of Florida Research Foundation, Incoporated, Gainesville, FL (US)

(21) Appl. No.: 18/277,969

(22) PCT Filed: Feb. 18, 2022
(86) PCT No.: PCT/US2022/017015

§ 371 Date: Aug. 18, 2023

Related U.S. Application Data
(60) Provisional application No. 63/151,402, filed on Feb. 19, 2021.

(51) Int. Cl. C12N 15/861; A61K 48/00

(52) U.S. Cl. CPC C12N 15/861; A61K 48/0058; C12N 2750/14143; C12N 2830/008; C12N 2840/44

(57) ABSTRACT

Provided herein, in some embodiments, are nucleic acid constructs encoding therapeutic proteins of interest comprising one or more alternatively-spliced exons that regulate the expression of therapeutic proteins of interest. Such constructs may in some embodiments be useful for delivery in a recombinant viral vector.

### RELATED APPLICATIONS

This application claims the benefit under 35 U.S.C. § 119(e) of the filing date of U.S. Provisional Application Ser. No. 63/151,402, filed Feb. 19, 2021, entitled “METHODS AND COMPOSITIONS TO CONFER REGULATION TO GENE THERAPY CARGOES BY HETEROLOGOUS USE OF ALTERNATIVE SPLICING CASSETTES”, the entire content of which is incorporated herein by reference in its entirety.

### SEQUENCE LISTING

In accordance with 37 C.F.R. 1.52(e)(5), the present specification makes reference to a Sequence Listing (submitted electronically as a .txt file named “U119670085WO00-SEQ-KSB”). The .txt file was generated on Feb. 15, 2022 and is 1,016,464 bytes in size. The Sequence Listing is herein incorporated by reference in its entirety.

### BACKGROUND

Recombinant viruses (e.g., recombinant adeno-associated viruses (AAV) and recombinant lentiviruses, etc.) can be used to express therapeutic proteins (i.e., therapeutic cargoes) in patients as a form of genetic therapy. Such therapies seeking to deliver a protein cargo commonly package a recombinant virus genome comprising a coding region of interest along with a 5′ untranslated region, 3′ untranslated region, a promoter that will drive the gene of interest, and, sometimes, a constitutive intron to enhance nuclear export and RNA stability. However, most promoter elements are not able to deliver the therapeutic cargo consistently and reliably in conditions of interest (e.g., a specific tissue, a specific cellular environment, etc.).

New approaches relating to the use of recombinant viruses for delivering therapeutic cargo consistently and reliably in conditions of interest would be an advance in the art.

### SUMMARY OF THE INVENTION

The present disclosure relates to the observation that alternatively-spliced exons may be used in the context of viral vectors (e.g., AAV viral vectors or lentivirus viral vectors) to effectively regulate the expression of a coding region of interest (e.g., a coding region of a transgene that encodes a therapeutic protein). In certain embodiments, the alternatively-spliced exons regulate a coding region of interest in a condition-sensitive manner. As used herein, “condition-sensitive manner” means that the alternatively-spliced exon regulates the expression of a coding region of interest in a manner that is controlled or influenced by one or more conditions, including, but not limited to, environmental conditions, intracellular conditions, extracellular conditions, type of cell (e.g., liver versus kidney cell), gene expression pattern, or disease state. Accordingly, the present disclosure relates to a new approach for regulating expression of a coding region of interest (e.g., a coding region of a transgene that encodes a therapeutic protein) from recombinant viral vectors, optionally in a condition-sensitive manner, by coupling the expression of a coding region of interest with an alternatively-spliced exon. The present disclosure describes a variety of exemplary configurations and methods of coupling the expression of a coding region of interest (or multiple portions of coding regions) with an alternatively-spliced exon, but any suitable arrangement or configuration is contemplated so long as the expression of the coding region of interest (e.g., a coding region of a transgene that encodes a therapeutic protein) is configured to come under regulatory control of an alternatively-spliced exon.

The present disclosure further relates to the following embodiments.

Aspects of the invention relate to a recombinant viral genome capable of delivering (e.g., expressing) a transgene or coding region thereof in a subject, wherein said recombinant viral genome comprises at least one alternatively-spliced exon and a coding region of the transgene. In various aspects, the alternatively-spliced exon undergoes differential splicing in a condition-sensitive manner to result in different spliced transcripts (e.g., mRNA isoforms), whereby the alternatively-spliced exon has been either retained (“spliced in”) or not retained (“spliced-out”) in the resulting spliced transcripts. For example, in a healthy cell environment, the alternatively-spliced exon may be spliced-out of the resulting transcript; however, in a cancer cell, the alternatively-spliced exon may be spliced-in the resulting transcript. And, depending upon the regulatory sequences present in the alternatively-spliced exon, and whether those regulatory sequences impart a positive or negative regulatory control on the expression of the coding region of interest, the alternatively-spliced exon regulates the expression of the coding region of interest by virtue of being either present (spliced-in) or not present (spliced-out) in the resulting mRNA transcript isoform.

In some embodiments, the alternatively-spliced exon may be provided in the form of a transgene comprising the alternatively-spliced exon, one or more introns (or portion(s) thereof), and one or more additional exons (e.g., constitutive exons). Such transgenes comprising an alternatively-spliced exon may be referred to herein as comprising an “alternatively-spliced exon cassettes.” The configuration of the alternatively-spliced exon cassettes and transgenes is not limited in any way, and examples of such configurations are provided in the Figures.

In some embodiments, the transgene comprises an alternatively-spliced exon, one or more introns (or portion(s) thereof) and one or more exons. In various embodiments, the one or more exons can be constitutive exons (i.e., those that are retained in all mRNA isoforms resulting from splicing). In certain embodiments, the transgene or the alternatively-spliced exon cassette comprises one intron (or portion thereof). In some embodiments, the intron (or portion thereof) is located 3′ or 5′ to an alternatively-spliced exon. In other embodiments, the transgene or the alternatively-spliced exon cassette comprises two introns (or portion(s) thereof) (e.g., whereby the one or more introns are flanking introns, i.e., introns that are immediately upstream or downstream of the alternatively-spliced exon).

In some embodiments, an alternative exon cassette comprises a polynucleotide having at least 70%, at least 75%, at least 80%, at least 90%, at least 92%, at least 95%, at least 98%, or at least 99% sequence identity, relative to a nucleic acid sequence as set forth in any one of SEQ ID NOs: 107-778. In some embodiments, an alternative exon cassette comprises a polynucleotide having a nucleic acid sequence as set forth in any one of SEQ ID NOs: 107-778.

In some embodiments, the alternatively-spliced exon comprises at least one modification, relative to a naturally occurring alternatively-spliced exon. In some embodiments, the alternatively-spliced exon comprises at its 3′ end a heterologous start codon or part of a heterologous start codon. In some embodiments, all native start codons located 5′ to the heterologous start codon are disrupted or deleted.

In some embodiments, the alternatively-spliced exon is located 5′ to the coding region of the transgene. In some embodiments, the alternatively-spliced exon cassette comprises two alternatively-spliced exons, each with flanking introns. In some embodiments, the two alternatively-spliced exons are adjacent. In some embodiments, the constitutive exon is located 5′ to the two alternatively-spliced exons.

In some embodiments, each alternatively-spliced exon comprises at its 3′ end a heterologous start codon or part of a heterologous start codon. In some embodiments, all native start codons located 5′ to the heterologous start codon of the 5′-most alternatively-spliced exon are disrupted or deleted.

In some embodiments, only one of the two alternatively-spliced exons is retained in the spliced transcript. In some embodiments, the 5′-most alternatively-spliced exon is retained in the spliced transcript. In some embodiments, the 3′-most alternatively-spliced exon is retained in the spliced transcript.

In some embodiments, the alternatively-spliced exon(s) and flanking intron(s) are located within the coding region of the transgene.

In some embodiments, the alternatively-spliced exon comprises a heterologous, in-frame stop codon. In some embodiments, the heterologous, in-frame stop codon is at least 50 nucleotides upstream of the next 5′ splice junction. In some embodiments, the heterologous stop codon elicits nonsense-mediated decay.

In various embodiments, the alternatively-spliced exon is spliced-in or retained in the presence of one or more conditions (i.e., in a condition-sensitive manner) to result in an mRNA isoform comprising the alternatively-spliced exon and a coding region of interest. In some embodiments, the one or more conditions comprise the conditions that define one cell type from another. In other embodiments, the one or more conditions comprise the intracellular conditions that define a healthy cell state from a diseased cell state. In some embodiments, the one or more conditions comprise the presence or absence of activated T cells and/or the presence or absence of a state of inflammation. In still other embodiments, the one or more conditions comprise one or more signs or symptoms of a disease state, and/or the presence or absence of one or more disease markers. In still other embodiments, the one or more conditions comprise the expression level and/or activity of the endogenous protein that corresponds to the protein encoded by the coding region of interest in the alternatively-spliced exon cassette of the recombinant virus genome. For example, in one embodiment, if the endogenous protein has a low level of expression and/or activity (e.g., due to a defective naturally occurring gene encoding the endogenous protein), the alternatively-spliced exon may be spliced-in, and the coding region of interest may be upregulated (e.g., if the alternatively-spliced exon comprises a positive regulatory sequence). In another embodiment, if the endogenous protein has a low level of expression and/or activity (e.g., due to a defective naturally occurring gene encoding the endogenous protein), the alternatively-spliced exon may be spliced-in, and the coding region of interest may be downregulated (e.g., if the alternatively-spliced exon comprises a negative regulatory sequence). In still other embodiments, if the endogenous protein has a low level of expression and/or activity (e.g., due to a defective naturally occurring gene encoding the endogenous protein), the alternatively-spliced exon may be spliced-out, and the coding region of interest may be upregulated (e.g., if the alternatively-spliced exon comprises a negative regulatory sequence that is removed by the splicing-out of the exon). In another embodiment, if the endogenous protein has a low level of expression and/or activity (e.g., due to a defective naturally occurring gene encoding the endogenous protein), the alternatively-spliced exon may be spliced-out, and the coding region of interest may be downregulated (e.g., if the alternatively-spliced exon comprises a positive regulatory sequence that is removed by the splicing-out of the exon).

In various embodiments, the one or more conditions (e.g., environmental, intracellular, disease state, cell type, expression pattern, etc.) may result in the splicing-in or splicing-out of the alternatively-spliced exon. For example, the one or more conditions may cause the alternatively-spliced exon to be spliced-in, and the coding region of interest may be upregulated (e.g., if the alternatively-spliced exon comprises a positive regulatory sequence). In another embodiment, the one or more conditions may cause the alternatively-spliced exon to be spliced-in, and the coding region of interest may be downregulated (e.g., if the alternatively-spliced exon comprises a negative regulatory sequence). In still other embodiments, the one or more conditions may cause the alternatively-spliced exon to be spliced-out, and the coding region of interest may be upregulated (e.g., if the alternatively-spliced exon comprises a negative regulatory sequence that is removed by the splicing-out of the exon). In another embodiment, the one or more conditions may cause the alternatively-spliced exon to be spliced-out, and the coding region of interest may be downregulated (e.g., if the alternatively-spliced exon comprises a positive regulatory sequence that is removed by the splicing-out of the exon).

In some embodiments, the alternatively-spliced exon comprises an alternatively-spliced exon from a gene selected from the group consisting of: ABCC1, AK125149, ASCC2, BAT2D1, BBX, BRD8, BRE, C17orf70, CAMKK2, CBFB, CCAR1, CCDC7CD6, CHTF8, COL4A3BP, COL6A3, CUGBP1, CUGBP2, CXorf45, DENND3, DGUOK, DKFZp762G094, DNAJC7, DNASE1, EIF4A2, EIF4G2, EIF4H, EXOC7, EZH2, FAM120A, FAM136A, FAM36A, FARSB, FBXO38, FGFR1OP2, FIP1L1, FOXRED1, FUBP3, GALT, GATA3, GOLGA2, HIF1A, HMMR, HRB, IKZF1, ILF3, IRAK4, IRF1, KCTD13, LEF1, LUC7L, LYRM1, MALT1 e7, MAP2K7, MAP3K7, MAP4K2, MBNL2, MFF, NAE1, NCSTN, NR4A3, NRF1, NUP98, PARP6, PCM1, PLAUR, PLSCR3, PPIL5, PPP5C, PTPRC-E4, PTPRC-E6, PTS, RABL5, RAPH1, SEC16A, SFRS3, SFRS7, SLMAP, SNRNP70, STAT6, TBC1D1, TIMM8B, TIR8, TRA2A, TROVE2, UGCGL1, VAP-B, VAV1, ZNF384, ZNF496, CAMK2B, PKP2, LGMN, NRAP, VPS39, KSR1, PDLIM3, BIN1, ARFGAP2, KIF13A, and/or PICALM. In some embodiments, the alternatively-spliced exon comprises an alternatively-spliced exon from or derived from an alternatively-spliced exon of a gene selected from the group consisting of CAMK2B, PKP2, LGMN, NRAP, VPS39, KSR1, PDLIM3, BIN1, ARFGAP2, KIF13A, and/or PICALM. In some embodiments, the alternatively-spliced exon is or is derived from an alternatively-spliced exon of CAMK2B. In some embodiments, the alternatively-spliced exon is or is derived from an alternatively-spliced exon of PKP2. In some embodiments, the alternatively-spliced exon is or is derived from an alternatively-spliced exon of LGMN. In some embodiments, the alternatively-spliced exon is or is derived from an alternatively-spliced exon of NRAP. In some embodiments, the alternatively-spliced exon is or is derived from an alternatively-spliced exon of VPS39. In some embodiments, the alternatively-spliced exon is or is derived from an alternatively-spliced exon of KSR1. In some embodiments, the alternatively-spliced exon is or is derived from an alternatively-spliced exon of PDLIM3. In some embodiments, the alternatively-spliced exon is or is derived from an alternatively-spliced exon of BIN1. In some embodiments, the alternatively-spliced exon is or is derived from an alternatively-spliced exon of ARFGAP2. In some embodiments, the alternatively-spliced exon is or is derived from an alternatively-spliced exon of KIF13A. In some embodiments, the alternatively-spliced exon is or is derived from an alternatively-spliced exon of PICALM.

In some embodiments, the alternatively-spliced exon is or is derived from exon 11 of BIN1. In some embodiments, the alternatively-spliced exon which is or is derived from exon 11 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 37. In some embodiments, the alternatively-spliced exon which is or is derived from exon 11 of BIN1 comprises a polynucleotide having a nucleic acid sequence as set forth in SEQ ID NO: 37. In some embodiments, the alternatively-spliced exon which is or is derived from exon 11 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 38. In some embodiments, the alternatively-spliced exon which is or is derived from exon 11 of BIN1 comprises a polynucleotide having a nucleic acid sequence as set forth in SEQ ID NO: 38.

In some embodiments, a component (e.g., an alternative exon; an intronic sequence) which is “derived from” a gene (e.g., BIN1, SMN1) may be derived from the gene in that the component is taken from its wild-type or natural context and put into a non-natural context (e.g., inserted into the nucleic acid sequence of a transgene), but may comprise the wild-type or natural nucleic acid sequence of said component. In some embodiments, a component (e.g., an alternative exon; an intronic sequence) which is “derived from” a gene (e.g., BIN1, SMN1) may be derived from the gene in that the component is taken from its wild-type or natural context and put into a non-natural context (e.g., inserted into the nucleic acid sequence of a transgene), and may also be derived from the gene in that the nucleic acid sequence of the component is modified, relative to the wild-type or natural nucleic acid sequence of said component. Modifications to the various components (e.g., introns, exons, etc.) are described elsewhere herein.

In some embodiments, the alternatively-spliced exon comprises an alternatively-spliced exon comprising a polynucleotide sequence as set forth in any one of SEQ ID NOs: 23-44.

In some embodiments, the flanking intron(s) (or portion(s) thereof) is a native flanking intron(s) (or portion(s) thereof) of the alternatively-spliced exon(s). In some embodiments, the flanking intron(s) (or portion(s) thereof) comprises at its 5′ end a 5′ splice donor site. In some embodiments, the flanking intron(s) (or portion(s) thereof) comprises at its 3′ end a 3′ splice donor site. In some embodiments, the flanking intron(s) (or portion(s) thereof) comprises no modifications, relative to a naturally occurring intron (or portion thereof). In some embodiments, the flanking intron(s) (or portion(s) thereof) comprises at least one modification, relative to a naturally occurring intron (or portion thereof). In some embodiments, the modification is a substitution or deletion of one or more nucleotides. In some embodiments, the flanking intron(s) (or portion(s) thereof) is a regulated intron (or portion thereof).

In some embodiments, the flanking intron(s) is or is derived from an intron of a gene selected from the group consisting of ABCC1, AK125149, ASCC2, BAT2D1, BBX, BRD8, BRE, C17orf70, CAMKK2, CBFB, CCAR1, CCDC7CD6, CHTF8, COL4A3BP, COL6A3, CUGBP1, CUGBP2, CXorf45, DENND3, DGUOK, DKFZp762G094, DNAJC7, DNASE1, EIF4A2, EIF4G2, EIF4H, EXOC7, EZH2, FAM120A, FAM136A, FAM36A, FARSB, FBXO38, FGFR1OP2, FIP1L1, FOXRED1, FUBP3, GALT, GATA3, GOLGA2, HIF1A, HMMR, HRB, IKZF1, ILF3, IRAK4, IRF1, KCTD13, LEF1, LUC7L, LYRM1, MALT1 e7, MAP2K7, MAP3K7, MAP4K2, MBNL2, MFF, NAE1, NCSTN, NR4A3, NRF1, NUP98, PARP6, PCM1, PLAUR, PLSCR3, PPIL5, PPP5C, PTPRC-E4, PTPRC-E6, PTS, RABL5, RAPH1, SEC16A, SFRS3, SFRS7, SLMAP, SMN1, SNRNP70, STAT6, TBC1D1, TIMM8B, TIR8, TRA2A, TROVE2, UGCGL1, VAP-B, VAV1, ZNF384, ZNF496, CAMK2B, PKP2, LGMN, NRAP, VPS39, KSR1, PDLIM3, BIN1, ARFGAP2, KIF13A, and/or PICALM.

In some embodiments, the flanking intron(s) is or is derived from an intron of SMN1. In some embodiments, the flanking intron(s) which is or is derived from an intron of SMN1 flanks a constitutive exon. In some embodiments, the flanking intron(s) is or is derived from intron 6 and/or intron 7 of SMN1. In some embodiments, the flanking intron which is derived from SMN1 intron 6 is a fragment of (e.g., is truncated relative to) the wild-type or naturally occurring sequence of SMN1 intron 6. In some embodiments, the flanking intron which is derived from SMN1 intron 6 comprises a polynucleotide having a nucleic acid sequence as set forth in SEQ ID NO: 103. In some embodiments, the flanking intron which is derived from SMN1 intron 6 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 103. In some embodiments, the flanking intron which is derived from SMN1 intron 7 is a fragment of (e.g., is truncated relative to) the wild-type or naturally occurring sequence of SMN1 intron 7. In some embodiments, the flanking intron which is derived from SMN1 intron 7 comprises a polynucleotide having a nucleic acid sequence as set forth in SEQ ID NO: 104. In some embodiments, the flanking intron which is derived from SMN1 intron 7 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 104.

In some embodiments, the flanking intron(s) is or is derived from an intron of BIN1. In some embodiments, the flanking intron(s) which is or is derived from an intron of BIN1 flanks an alternative exon. In some embodiments, the flanking intron(s) is or is derived from intron 10 and/or intron 11 of BIN1. In some embodiments, the flanking intron(s) which is or is derived from intron 10 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 15. In some embodiments, the flanking intron(s) which is or is derived from intron 10 of BIN1 comprises a polynucleotide having a nucleic acid sequence as set forth in SEQ ID NO: 15. In some embodiments, the flanking intron(s) which is or is derived from intron 11 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 16. In some embodiments, the flanking intron(s) which is or is derived from intron 11 of BIN1 comprises a polynucleotide having a nucleic acid sequence as set forth in SEQ ID NO: 16.

In some embodiments, the flanking intron(s) comprises an intron comprising a polynucleotide sequence as set forth in any one of SEQ ID NOs: 1-22, 103, and 104.

In some embodiments, the constitutive exon is an exon which is natively associated with the coding region of the transgene. In some embodiments, the constitutive exon is not a exon which is natively associated with the coding region of the transgene. In some embodiments, the constitutive exon is or is derived from the same gene as the alternatively-spliced exon(s). In some embodiments, the gene is the gene from which the coding region of the transgene is also derived. In some embodiments, the constitutive exon is not from or derived from the same gene as the alternatively-spliced exon(s).

In some embodiments, the coding region of the transgene is or is derived from a coding region of a gene selected from the group consisting of MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, MTMR2, LAMP2, KIF5A, a microdystrophin-encoding gene, C9ORF72, HTT, DNM2, BIN1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, a cytochrome b oxidase-encoding gene, a cytochrome c oxidase-encoding gene, CLCN1, SCN4A, DMPK, CNBP, MYOT, LMNA, CAV3, DNAJB6, DES, TNPO3, HNRPDL, CAPN3, DYSF, an alpha-sarcoglycan-encoding gene, a beta-sarcoglycan-encoding gene, a gamma-sarcoglycan-encoding gene, a delta-sarcoglycan-encoding gene, TCAP, TRIM32, FKRP, FXN, POMT1, FKTN, POMT2, POMGnT1, DAG1, ANO5, PLEC1, TRAPPC11, GMPPB, ISPD, LIMS2, POPDC1, TOR1AIP1, POGLUT2, LAMA2, COL6A1, POMT1, POMT2, DUX4, EMD, PAX7, PMP22, MPZ, MFN2, SMCHD1, SMN, Lamin A/C (LAMN), GJB1, ABCC1, AK125149, ASCC2, BAT2D1, BBX, BRD8, BRE, C17orf70, CAMKK2, CBFB, CCAR1, CCDC7CD6, CHTF8, COL4A3BP, COL6A3, CUGBP1, CUGBP2, CXorf45, DENND3, DGUOK, DKFZp762G094, DNAJC7, DNASE1, EIF4A2, EIF4G2, EIF4H, EXOC7, EZH2, FAM120A, FAM136A, FAM36A, FARSB, FBXO38, FGFR1OP2, FIP1L1, FOXRED1, FUBP3, GALT, GATA3, GOLGA2, HIF1A, HMMR, HRB, IKZF1, ILF3, IRAK4, IRF1, KCTD13, LEF1, LUC7L, LYRM1, MALT1 e7, MAP2K7, MAP3K7, MAP4K2, MBNL2, MFF, NAE1, NCSTN, NR4A3, NRF1, NUP98, PARP6, PCM1, PLAUR, PLSCR3, PPIL5, PPP5C, PTPRC-E4, PTPRC-E6, PTS, RABL5, RAPH1, SEC16A, SFRS3, SFRS7, SLMAP, SNRNP70, STAT6, TBC1D1, TIMM8B, TIR8, TRA2A, TROVE2, UGCGL1, VAP-B, VAV1, ZNF384, ZNF496, CAMK2B, PKP2, LGMN, NRAP, VPS39, KSR1, PDLIM3, BIN1, ARFGAP2, KIF13A, and/or PICALM. In some embodiments, the coding region of the transgene is or is derived from MTM1, CAPN3, or FXN. In some embodiments, the coding region of the transgene is or is derived from FXN.

In some embodiments, the coding region of the transgene is or is derived from MTM1. In some embodiments, the coding region of the transgene which is or is derived from MTM1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1881. In some embodiments, the coding region of the transgene which is or is derived from MTM1 comprises a polynucleotide having a nucleic acid sequence as set forth in SEQ ID NO: 1881.

In some embodiments, the coding region of the transgene is or is derived from CAPN3. In some embodiments, the coding region of the transgene which is or is derived from CAPN3 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1882. In some embodiments, the coding region of the transgene which is or is derived from CAPN3 comprises a polynucleotide having a nucleic acid sequence as set forth in SEQ ID NO: 1882.

In some embodiments, a recombinant viral genome of the present disclosure further comprises a promoter. In some embodiments, the promoter is a native promoter of the coding region of the transgene. In some embodiments, the promoter is not a native promoter of the coding region of the transgene. In some embodiments, the promoter is constitutive. In some embodiments, the promoter is inducible. In some embodiments, the promoter is a cell-specific promoter. In some embodiments, the promoter is a tissue-specific promoter. In some embodiments, the promoter is selected from the group consisting of an EF1 alpha promoter, beta actin promoter, CMV, muscle creatine kinase promoter, C5-12 muscle promoter, MHCK7, CBh, synapsin, MECP2, enolase, GFAP, Desmin, and CAG promoter.

In some embodiments, the promoter is an MHCK7 promoter. In some embodiments, an MHCK7 promoter comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1880. In some embodiments, an MHCK7 promoter comprises a polynucleotide having a nucleic acid sequence as set forth in SEQ ID NO: 1880.

In some embodiments, the promoter drives expression of the transgene (e.g., expression of the product encoded by the coding region of interest). In some embodiments, the promoter is a ubiquitous promoter. In some embodiments, a ubiquitous promoter is a promoter selected from the group consisting of: an EF1 alpha promoter, a beta actin promoter, CMV, CBh, and CAG promoter. In some embodiments, the promoter is a tissue-specific promoter, such as a muscle- or heart-biased promoter. In some embodiments, a tissue-specific promoter, such as a muscle- or heart-biased promoter, is a promoter selected from the group consisting of: a muscle creatine kinase promoter, a C5-12 muscle promoter, MHCK7, and Desmin. In some embodiments, the promoter is a neuronal-biased promoter. In some embodiments, a neuronal-biased promoter is a promoter selected from the group consisting of: synapsin and MECP2. In some embodiments, the promoter is an astrocyte-biased promoter. In some embodiments, an astrocyte-biased promoter is a GFAP promoter.

In some embodiments, the coding region of the transgene comprises at least one modification, relative to a coding region of a naturally occurring gene. In some embodiments, the modification is an addition, substitution or deletion of at least one nucleotide. In some embodiments, the coding region of the transgene comprises a deletion of a native start codon, or a portion thereof. In some embodiments, the coding region of the transgene comprises an addition of a non-native stop codon, or a portion thereof. In some embodiments, the transgene comprises one or more recombinant introns (e.g., a 3′ UTR intron). In some embodiments, the one or more recombinant introns (e.g., a 3′ UTR intron), when translated, elicits nonsense mediated decay (NMD).

In some embodiments, the naturally occurring gene is a gene selected from the group consisting of MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, MTMR2, LAMP2, KIF5A, a microdystrophin-encoding gene, C9ORF72, HTT, DNM2, BIN1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, a cytochrome b oxidase-encoding gene, a cytochrome c oxidase-encoding gene, CLCN1, SCN4A, DMPK, CNBP, MYOT, LMNA, CAV3, DNAJB6, DES, TNPO3, HNRPDL, CAPN3, DYSF, an alpha-sarcoglycan-encoding gene, a beta-sarcoglycan-encoding gene, a gamma-sarcoglycan-encoding gene, a delta-sarcoglycan-encoding gene, TCAP, TRIM32, FKRP, FXN, POMT1, FKTN, POMT2, POMGnT1, DAG1, ANO5, PLEC1, TRAPPC11, GMPPB, ISPD, LIMS2, POPDC1, TOR1AIP1, POGLUT2, LAMA2, COL6A1, POMT1, POMT2, DUX4, EMD, PAX7, PMP22, MPZ, MFN2, SMCHD1, SMN, Lamin A/C (LAMN), and/or GJB1. In some embodiments, the naturally occurring gene is MTM1, CAPN3, or FXN. In some embodiments, the naturally occurring gene is MTM1. In some embodiments, the naturally occurring gene is CAPN3. In some embodiments, the naturally occurring gene is FXN.

In some embodiments, the coding region of the transgene comprises a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 1881 or SEQ ID NO: 1882. In some embodiments, the coding region of the transgene comprises a polynucleotide having a nucleic acid sequence as set forth in either SEQ ID NO: 1881 or SEQ ID NO: 1882.

In some embodiments, the recombinant viral genome is a recombinant genome from an adeno-associated virus (rAAV), lentivirus, retrovirus, or foamyvirus. In some embodiments, the recombinant viral genome is from an AAV. In some embodiments, the transgene is flanked by AAV inverted terminal repeat (ITR) sequences. In some embodiments, the ITR sequences comprise AAV1, AAV2, AAV5, AAV7, AAV8, or AAV9 ITR sequences. In some embodiments, the recombinant viral genome is from a lentivirus. In some embodiments, the alternatively-spliced exon cassette is located on the minus strand of the lentivirus genome.

In some embodiments, a recombinant viral genome of the present disclosure further comprises a 3′ untranslated region (UTR) that is endogenous or exogenous to the transgene. In some embodiments, the exogenous 3′ UTR is the 3′ UTR from bovine growth hormone, SV40, EBV, or Myc.

In some embodiments, the exogenous 3′ UTR is SV40. In some embodiments, the SV40 3′ UTR comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1883. In some embodiments, the SV40 3′ UTR comprises a polynucleotide having a nucleic acid sequence as set forth in SEQ ID NO: 1883.

In some embodiments, the exogenous 3′ UTR comprises a polyadenylation (pA) signal. In some embodiments, the pA signal is an SV40 pA signal.

Aspects of the invention contemplate a viral particle comprising a viral genome according to any embodiment of the present disclosure. In some embodiments, the viral particle is an rAAV particle. In some embodiments, the rAAV particle comprises an AAV serotype selected from the group consisting of 1, 2, 3, 4, 5, 6, 7, 8, 9, or 10. In some embodiments, the rAAV particle comprises AAV serotype 9. In some embodiments, the rAAV particle comprises an AAV derivative or pseudotype selected from the group consisting of an AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y→F), AAV8 (Y733F), AAV2.15, AAV2.4, AAVM41, and AAVr3.45.

In some embodiments, the viral particle further comprises at least one helper plasmid. In some embodiments, the helper plasmid comprises a rep gene and a cap gene. In some embodiments, the rep gene encodes Rep78, Rep68, Rep52, or Rep40. In some embodiments, the cap gene encodes a VP1, VP2, and/or VP3 region of the viral capsid protein. In some embodiments, the viral particle comprises two helper plasmids. In some embodiments, the first helper plasmid comprises a rep gene and a cap gene and the second helper plasmid comprises a E1a gene, a E1b gene, a E4 gene, a E2a gene, and a VA gene.

In some embodiments, the viral particle is a recombinant lentivirus particle. In some embodiments, the lentivirus is a human immunodeficiency virus (HIV1 or HIV2), a feline immunodeficiency virus (FIV), a bovine immunodeficiency virus (BIV), a caprine arthritis encephalitis virus, an equine infectious anemia virus, a jembrana disease virus, a puma lentivirus, aimian immunodeficiency virus, or a visna-maedi virus. In some embodiments, the viral particle further comprises a viral envelope.

Aspects of the invention relate to a method of treating a disease or condition in a subject comprising administering a recombinant viral genome or a viral particle according to any embodiment of the present disclosure to the subject. In some embodiments, the subject is a mammal. In some embodiments, the mammal is a human. In some embodiments, the recombinant viral genome or viral particle is administered to the subject at least one time. In some embodiments, the recombinant viral genome or viral particle is administered to the subject 2, 3, 4, 5, 6, 7, 8, 9, or 10 times. In some embodiments, the recombinant viral genome or viral particle is administered to the subject parenterally, subcutaneously, intraocularly, intravitreally, subretinally, intravenously (IV), intracerebro-ventricularly, intramuscularly, intrathecally (IT), intracisternally, intraperitoneally, enterally, via inhalation, topically, or by direct injection to one or more cells, tissues, or organs. In some embodiments, the recombinant viral genome or viral particle is administered to the subject by intravenous injection, intramuscular injection, intrathecal injection, or intravitreal injection.

In some embodiments, the disease or condition is a disease or condition selected from the group consisting of Dentatorubral-pallido-luysian atrophy (DRPLA), myotonic dystrophy type 1 (DM1), myotonic dystrophy type 2 (DM2), Fragile X syndrome of mental retardation (FMR1), Fragile X tremor ataxia syndrome (FXTAS), FRAXE mental retardation (FMR2), Friedreichs ataxia (FRDA), Huntington disease (HD), Huntington disease-like 2 (HDL2), Oculopharyngeal muscular dystrophy (OPMD), Myoclonic epilepsy type 1, Alzheimer's disease, ALS/FTD, spinocerebellar ataxia type 1 (SCA1), spinocerebellar ataxia type 2 (SCA2), spinocerebellar ataxia type 3 (SCA3), spinocerebellar ataxia type 6 (SCA6), spinocerebellar ataxia type 7 (SCAT), spinocerebellar ataxia type 8 (SCA8), spinocerebellar ataxia type 10 (SCA10), spinocerebellar ataxia type 12 (SCA12), spinocerebellar ataxia type 17 (SCA17), Syndromic/non-syndromic X-linked mental retardation, Emery-Dreifuss muscular dystrophy type 2, familial partial lipodystrophy, limb girdle muscular dystrophy type 1B, dilated cardiomyopathy, familial partial lipodystrophy, Charcot-Marie-Tooth disorder type 2B1, mandibuloacral dysplasia, childhood progeria syndrome (Hutchinson-Gilford syndrome), Werner syndrome, Dilated cardiomyopathy (DCM), Hypertrophic cardiomyopathy (HCM), Restrictive cardiomyopathy (RCM), Left Ventricular Non-compaction (LVNC), Arrhythmogenic Right Ventricular Dysplasia (ARVD), takotsubo cardiomyopathy, Duchenne muscular dystrophy, Becker muscular dystrophy, Limb-girdle muscular dystrophy, Facioscapulohumeral muscular dystrophy, Congenital muscular dystrophy, Oculopharyngeal muscular dystrophy, Distal muscular dystrophy, Emery-Dreifuss muscular dystrophy, dementia, Parkinson's disease (PD), a PD-related disorder, Prion disease, a motor neuron disease (MND), Progressive bulbar palsy (PBP), Progressive muscular atrophy (PMA), Primary lateral sclerosis (PLS), Spinal muscular atrophy (SMA), a bladder cancer, a breast cancer, a colorectal cancer, a kidney cancer, a lung cancer, a lymphoma, a melanoma, an oral cancer, an ovarian cancer, an oropharyngeal cancer, a pancreatic cancer, a prostate cancer, a thyroid cancer, a uterine cancer, Down syndrome, Prader-Willi Syndrome (PWS), Bloom Syndrome, Cockayne Syndrome Type I-216400, Cockayne Syndrome Type III, Cockayne Syndrome Type I, Hutchinson-Gilford Progeria Syndrome, Mandibuloacral Dysplasia with Type A Lipodystrophy, Progeria, Adult Onset Progeroid Syndrome, Neonatal Rothmund-Thomson Syndrome, Seip Syndrome, Werner Syndrome, Replication Focus-Forming Activity 1, myotubular myopathy, Danon Disease, and/or centronuclear myopathy.

Aspects of the invention relate to a method of regulating transgene expression (e.g., comprising a coding region of interest which encodes a protein of interest, such as a therapeutic protein) using a viral vector comprising a recombinant viral genome as described herein, wherein the transgene, or coding region of the transgene, are under the regulatory control of an alternatively-spliced exon. In some embodiments, the method comprises inserting into the recombinant viral genome at least one alternatively-spliced exon and at least one coding region of interest (e.g., which encodes a therapeutic protein), wherein the expression of the at least one coding region of interest is regulated by the alternative-spliced exon. In turn, how the regulation of the coding region of interest is imparted depends on (a) the presence or absence of positive or negative regulatory control sequences in the alternatively-spliced exon, and (b) whether the alternatively-splice exon is spliced-in (i.e., retained) or spliced-out (i.e., removed) from the final mRNA transcript isoform. The recombinant viral genome may be configured with one or more additional introns, exons, and/or regulatory sequences (e.g., promoters, enhancers, and the like that control transcription from the recombinant viral genome). In addition, the alternatively-splice exon may be comprised on a cassette (which may be referred to as an alternatively-spliced exon cassette), comprising the alternatively-spliced exon(s) and one or more introns, which may be inserted into the recombinant viral genome in a manner that couples it to the coding region of interest, such that the expression of the coding region of interest comes under regulatory control of the alternatively-spliced exon of the cassette.

In other embodiments, the transgene comprises an alternatively-spliced exon, optionally one or more introns (or portion(s) thereof), optionally one or more constitutive exons, and a coding region of interest.

Aspects of the invention relate to a method of regulating transgene (e.g., comprising a coding region of interest which encodes a protein of interest, such as a therapeutic protein) expression using a viral vector comprising a recombinant viral genome as described herein. In some embodiments, the method comprises: (a) inserting into the recombinant viral genome at least one transgene, wherein the transgene comprises a constitutive exon, at least one alternatively-spliced exon, at least one flanking intron (or portion thereof), and a coding region of a transgene; (b) introducing a heterologous start codon or part of a heterologous start codon at the 3′ end of the alternatively-spliced exon; (c) disrupting or deleting all native start codons located 5′ to the heterologous start codon; and (d) deleting or disrupting one or more native start codons, or a portion(s) thereof, from the coding region of the transgene. In some embodiments, the method comprises: (a) inserting into the recombinant viral genome at least one transgene, wherein the transgene comprises a constitutive exon, at least one alternatively-spliced exon, at least one flanking intron (or portion thereof), and a coding region of a transgene; (b) introducing a heterologous start codon or part of a heterologous start codon at the 3′ end of the alternatively-spliced exon; (c) disrupting or deleting all native start codons located 5′ to the heterologous start codon; and (d) adding a heterologous 3′ UTR, or a portion thereof, to the coding region of the transgene. In some embodiments, translation of the heterologous 3′ UTR elicits nonsense mediated decay. In some embodiments, (a) inserting into the recombinant viral genome at least one alternatively-spliced exon cassette, wherein the alternatively-spliced exon cassette comprises a constitutive exon, at least one alternatively-spliced exon, at least one flanking intron (or portion thereof), and a coding region of a transgene; (b) introducing a heterologous start codon or part of a heterologous start codon at the 3′ end of the alternatively-spliced exon; (c) disrupting or deleting all native start codons located 5′ to the heterologous start codon; (d) deleting or disrupting one or more native start codons, or a portion(s) thereof, from the coding region of the transgene; and (e) adding a heterologous 3′ UTR, or a portion thereof, to the coding region of the transgene. In some embodiments, translation of the heterologous 3′ UTR elicits nonsense mediated decay. In some embodiments, the constitutive exon, alternatively-spliced exon, and flanking intron (or portion thereof) are each located 5′ to the coding region of the transgene.

Aspects of the invention relate to a method of regulating transgene (e.g., comprising a coding region of interest which encodes a protein of interest, such as a therapeutic protein) expression using a viral vector comprising a recombinant viral genome as described herein. In some embodiments, the method comprises: (a) inserting into the recombinant viral genome at least one transgene, wherein the transgene comprises an alternatively-spliced exon and at least one flanking intron (or portion thereof) within the coding region of the transgene; and (b) introducing into the alternatively-spliced exon a heterologous, in-frame stop codon upstream of the next 5′ splice junction. In some embodiments, the heterologous, in-frame stop codon elicits nonsense-mediated decay. In certain embodiments, the in-frame stop codon is inserted at least 100 nucleotides, at least 95 nucleotides, at least 90 nucleotides, at least 85 nucleotides, at least 80 nucleotides, at least 75 nucleotides, at least 70 nucleotides, at least 65 nucleotides, at least 60 nucleotides, at least 55 nucleotides, at least 50 nucleotides, at least 45 nucleotides, at least 40 nucleotides, at least 35 nucleotides, at least 30 nucleotides, at least 25 nucleotides, at least 20 nucleotides, at least 15 nucleotides, at least 10 nucleotides, or at least 5 nucleotides, or between 1 to 5 nucleotides upstream of the next 5′ splice junction.

Aspects of the invention relate to a transgene comprising, in the 5′ to 3′ direction: (i) a nucleotide sequence comprising a first exonic sequence having a 5′ to 3′ orientation, wherein the first exonic sequence comprises a constitutive exon; (ii) a nucleotide sequence comprising a first intronic sequence having a 5′ to 3′ orientation, wherein the first intronic sequence comprises at its 5′ end a 5′ splice donor site and at its 3′ end a 3′ splice acceptor site; (iii) a nucleotide sequence comprising a second exonic sequence having a 5′ to 3′ orientation, wherein the second exonic sequence comprises an alternatively-spliced exon comprising at its 3′ end a heterologous ATG start codon; (iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; and (v) a nucleotide sequence comprising a coding region of the transgene having a 5′ to 3′ orientation, wherein the coding region of the transgene comprises at its 5′ end a modification comprising the removal of a native ATG start codon. In some embodiments, all native ATG start codons located upstream of the heterologous ATG start codon are mutated or deleted.

Other aspects of the invention relate to a transgene comprising, in the 5′ to 3′ direction: (i) a nucleotide sequence comprising a first portion of a coding region of the transgene having a 5′ to 3′ orientation; (ii) a nucleotide sequence comprising a first intronic sequence having a 5′ to 3′ orientation, wherein the first intronic sequence comprises at its 5′ end a 5′ splice donor site and at its 3′ end a 3′ splice acceptor site; (iii) a nucleotide sequence comprising an exonic sequence having a 5′ to 3′ orientation, wherein the exonic sequence comprises an alternatively-spliced exon comprising at its 3′ end a heterologous stop codon; (iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; and (v) a nucleotide sequence comprising a second portion of a coding region of the transgene having a 5′ to 3′ orientation.

Aspects of the invention relate to a transgene comprising, in the 5′ to 3′ direction: (i) a nucleotide sequence comprising a coding region of the transgene having a 5′ to 3′ orientation; (ii) a nucleotide sequence comprising a first intronic sequence having a 5′ to 3′ orientation, wherein the first intronic sequence comprises at its 5′ end a 5′ splice donor site and at its 3′ end a 3′ splice acceptor site; (iii) a nucleotide sequence comprising a first exonic sequence having a 5′ to 3′ orientation, wherein the first exonic sequence comprises an alternatively-spliced exon comprising a positive or negative cis-acting element; (iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; and (v) a nucleotide sequence comprising a second exonic sequence having a 5′ to 3′ orientation, wherein the exonic sequence comprises a constitutive exon.

Aspects of the invention relate to a transgene comprising, in the 5′ to 3′ direction: (i) a nucleotide sequence comprising a first exonic sequence having a 5′ to 3′ orientation, wherein the first exonic sequence comprises a constitutive exon; (ii) a nucleotide sequence comprising a second exonic sequence having a 5′ to 3′ orientation, wherein the second exonic sequence comprises an alternatively-spliced exon comprising at its 3′ end a heterologous ATG start codon; and (iii) a nucleotide sequence comprising a coding region of the transgene having a 5′ to 3′ orientation, wherein the coding region of the transgene comprises at its 5′ end a modification comprising the removal of a native ATG start codon. In some embodiments, all native ATG start codons located upstream of the heterologous ATG start codon are mutated or deleted.

Aspects of the invention relate to a transgene comprising, in the 5′ to 3′ direction: (i) a nucleotide sequence comprising a first portion of a coding region of the transgene having a 5′ to 3′ orientation; (ii) a nucleotide sequence comprising a first exonic sequence having a 5′ to 3′ orientation, wherein the first exonic sequence comprises an alternatively-spliced exon comprising at its 3′ end a heterologous stop codon; (iii) a nucleotide sequence comprising a second portion of a coding region of the transgene having a 5′ to 3′ orientation; (iv) a nucleotide sequence comprising an intronic sequence having a 5′ to 3′ orientation, wherein the intronic sequence comprises at its 5′ end a 5′ splice donor site and at its 3′ end a 3′ splice acceptor site; and (v) a nucleotide sequence comprising a second exonic sequence having a 5′ to 3′ orientation, wherein the second exonic sequence comprises a constitutive exon.

Aspects of the invention relate to a transgene comprising, in the 5′ to 3′ direction: (i) a nucleotide sequence comprising a coding region of the transgene having a 5′ to 3′ orientation; (ii) a nucleotide sequence comprising a first exonic sequence having a 5′ to 3′ orientation, wherein the first exonic sequence comprises an alternatively-spliced exon comprising a positive or negative cis-acting element; and (iii) a nucleotide sequence comprising a second exonic sequence having a 5′ to 3′ orientation, wherein the second exonic sequence comprises a constitutive exon.

Aspects of the invention relate to a transgene comprising, in the 5′ to 3′ direction: (i) a nucleotide sequence comprising a first exonic sequence having a 5′ to 3′ orientation, wherein the first exonic sequence comprises a constitutive exon; (ii) a nucleotide sequence comprising a second exonic sequence having a 5′ to 3′ orientation, wherein the second exonic sequence comprises an alternatively-spliced exon comprising at its 3′ end a heterologous ATG start codon; (iii) a nucleotide sequence comprising an intronic sequence having a 5′ to 3′ orientation, wherein the intronic sequence comprises at its 5′ end a 5′ splice donor site and at its 3′ end a 3′ splice acceptor site; and (iv) a nucleotide sequence comprising a coding region of the transgene having a 5′ to 3′ orientation, wherein the coding region of the transgene comprises at its 5′ end a modification comprising the removal of a native ATG start codon. In some embodiments, all native ATG start codons located upstream of the heterologous ATG start codon are mutated or deleted.

Aspects of the invention relate to a transgene comprising, in the 5′ to 3′ direction: (i) a nucleotide sequence comprising a first portion of a coding region of the transgene having a 5′ to 3′ orientation; (ii) a nucleotide sequence comprising an exonic sequence having a 5′ to 3′ orientation, wherein the exonic sequence comprises an alternatively-spliced exon comprising at its 3′ end a heterologous stop codon; (iii) a nucleotide sequence comprising an intronic sequence having a 5′ to 3′ orientation, wherein the intronic sequence comprises at its 5′ end a 5′ splice donor site and at its 3′ end a 3′ splice acceptor site; and (iv) a nucleotide sequence comprising a second portion of a coding region of the transgene having a 5′ to 3′ orientation.

Aspects of the invention relate to a transgene comprising, in the 5′ to 3′ direction: (i) a nucleotide sequence comprising a coding region of the transgene having a 5′ to 3′ orientation; (ii) a nucleotide sequence comprising a first exonic sequence having a 5′ to 3′ orientation, wherein the exonic sequence comprises an alternatively-spliced exon comprising a positive or negative cis-acting element; (iii) a nucleotide sequence comprising an intronic sequence having a 5′ to 3′ orientation, wherein the intronic sequence comprises at its 5′ end a 5′ splice donor site and at its 3′ end a 3′ splice acceptor site; and (iv) a nucleotide sequence comprising a second exonic sequence having a 5′ to 3′ orientation, wherein the exonic sequence comprises a constitutive exon.

Aspects of the invention relate to a transgene comprising, in the 5′ to 3′ direction: (i) a nucleotide sequence comprising a first exonic sequence having a 5′ to 3′ orientation, wherein the first exonic sequence comprises a constitutive exon; (ii) a nucleotide sequence comprising an intronic sequence having a 5′ to 3′ orientation, wherein the intronic sequence comprises at its 5′ end a 5′ splice donor site and at its 3′ end a 3′ splice acceptor site; (iii) a nucleotide sequence comprising a second exonic sequence having a 5′ to 3′ orientation, wherein the second exonic sequence comprises an alternatively-spliced exon comprising at its 3′ end a heterologous ATG start codon; and (iv) a nucleotide sequence comprising a coding region of the transgene having a 5′ to 3′ orientation, wherein the coding region of the transgene comprises at its 5′ end a modification comprising the removal of a native ATG start codon. In some embodiments, all native ATG start codons located upstream of the heterologous ATG start codon are mutated or deleted.

Aspects of the invention relate to a transgene comprising, in the 5′ to 3′ direction: (i) a nucleotide sequence comprising a first portion of a coding region of the transgene having a 5′ to 3′ orientation; (ii) a nucleotide sequence comprising a first intronic sequence having a 5′ to 3′ orientation, wherein the first intronic sequence comprises at its 5′ end a 5′ splice donor site and at its 3′ end a 3′ splice acceptor site; (iii) a nucleotide sequence comprising a first exonic sequence having a 5′ to 3′ orientation, wherein the first exonic sequence comprises an alternatively-spliced exon comprising at its 3′ end a heterologous stop codon; (iv) a nucleotide sequence comprising a second portion of a coding region of the transgene having a 5′ to 3′ orientation; (v) a nucleotide sequence comprising a second intronic sequence having a 5′ to 3′ orientation, wherein the second intronic sequence comprises at its 5′ end a 5′ splice donor site and at its 3′ end a 3′ splice acceptor site; and (vi) a nucleotide sequence comprising a second exonic sequence having a 5′ to 3′ orientation, wherein the second exonic sequence comprises a constitutive exon.

Aspects of the invention relate to a transgene comprising, in the 5′ to 3′ direction: (i) a nucleotide sequence comprising a coding region of the transgene having a 5′ to 3′ orientation; (ii) a nucleotide sequence comprising an intronic sequence having a 5′ to 3′ orientation, wherein the intronic sequence comprises at its 5′ end a 5′ splice donor site and at its 3′ end a 3′ splice acceptor site; (iii) a nucleotide sequence comprising an exonic sequence having a 5′ to 3′ orientation, wherein the exonic sequence comprises an alternatively-spliced exon comprising a positive or negative cis-acting element; and (iv) a nucleotide sequence comprising a second exonic sequence having a 5′ to 3′ orientation, wherein the exonic sequence comprises a constitutive exon.

Aspects of the invention relate to a transgene comprising, in the 5′ to 3′ direction: (i) a nucleotide sequence comprising a first exonic sequence having a 5′ to 3′ orientation, wherein the first exonic sequence comprises a constitutive exon; (ii) a nucleotide sequence comprising a first intronic sequence having a 5′ to 3′ orientation, wherein the first intronic sequence comprises at its 5′ end a 5′ splice donor site and at its 3′ end a 3′ splice acceptor site; (iii) a nucleotide sequence comprising a second exonic sequence having a 5′ to 3′ orientation, wherein the second exonic sequence comprises an alternatively-spliced exon comprising at its 3′ end a heterologous ATG start codon; (iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; (v) a nucleotide sequence comprising a third exonic sequence having a 5′ to 3′ orientation, wherein the third exonic sequence comprises an alternatively-spliced exon; (vi) a nucleotide sequence comprising a third intronic sequence having a 5′ to 3′ orientation, wherein the third intronic sequence comprises at its 5′ end a 5′ splice donor site and at its 3′ end a 3′ splice acceptor site; and (vii) a nucleotide sequence comprising a coding region of the transgene having a 5′ to 3′ orientation, wherein the coding region of the transgene comprises at its 5′ end a modification comprising the removal of a native ATG start codon. In some embodiments, all native ATG start codons located upstream of the heterologous ATG start codon are mutated or deleted.

Aspects of the invention relate to a transgene comprising, in the 5′ to 3′ direction: (i) a nucleotide sequence comprising a first portion of a coding region of the transgene having a 5′ to 3′ orientation; (ii) a nucleotide sequence comprising a first intronic sequence having a 5′ to 3′ orientation, wherein the first intronic sequence comprises at its 5′ end a 5′ splice donor site and at its 3′ end a 3′ splice acceptor site; (iii) a nucleotide sequence comprising a first exonic sequence having a 5′ to 3′ orientation, wherein the first exonic sequence comprises an alternatively-spliced exon comprising at its 3′ end a heterologous stop codon; (iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; (v) a nucleotide sequence comprising a second exonic sequence having a 5′ to 3′ orientation, wherein the second exonic sequence comprises an alternatively-spliced exon; (vi) a nucleotide sequence comprising a third intronic sequence having a 5′ to 3′ orientation, wherein the third intronic sequence comprises at its 5′ end a 5′ splice donor site and at its 3′ end a 3′ splice acceptor site (m); and (vii) a nucleotide sequence comprising a second portion of a coding region of the transgene having a 5′ to 3′ orientation.

Aspects of the invention relate to a transgene comprising, in the 5′ to 3′ direction: (i) a nucleotide sequence comprising a coding region of the transgene having a 5′ to 3′ orientation; (ii) a nucleotide sequence comprising a first intronic sequence having a 5′ to 3′ orientation, wherein the first intronic sequence comprises at its 5′ end a 5′ splice donor site and at its 3′ end a 3′ splice acceptor site; (iii) a nucleotide sequence comprising a first exonic sequence having a 5′ to 3′ orientation (e), wherein the first exonic sequence comprises a first alternatively-spliced exon comprising a positive or negative cis-acting element; (iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; (v) a nucleotide sequence comprising a second exonic sequence having a 5′ to 3′ orientation, wherein the second exonic sequence comprises a second alternatively-spliced exon; (vi) a nucleotide sequence comprising a third intronic sequence having a 5′ to 3′ orientation, wherein the third intronic sequence comprises at its 5′ end a 5′ splice donor site and at its 3′ end a 3′ splice acceptor site; and (vii) a nucleotide sequence comprising a third exonic sequence having a 5′ to 3′ orientation, wherein the third exonic sequence comprises a constitutive exon.

Aspects of the invention relate to a transgene comprising, in the 5′ to 3′ direction: (i) a nucleotide sequence comprising a first exonic sequence having a 5′ to 3′ orientation, wherein the first exonic sequence comprises a constitutive exon; (ii) a nucleotide sequence comprising a first intronic sequence having a 5′ to 3′ orientation, wherein the first intronic sequence comprises at its 5′ end a 5′ splice donor site and at its 3′ end a 3′ splice acceptor site; (iii) a nucleotide sequence comprising a coding region of the transgene having a 5′ to 3′ orientation; (iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; and (v) a nucleotide sequence comprising a second exonic sequence having a 5′ to 3′ orientation, wherein the second exonic sequence comprises an alternatively-spliced exon.

Aspects of the invention relate to a transgene comprising, in the 5′ to 3′ direction: (i) a nucleotide sequence comprising a first portion of a coding region of the transgene having a 5′ to 3′ orientation; (ii) a nucleotide sequence comprising a first intronic sequence having a 5′ to 3′ orientation, wherein the first intronic sequence comprises at its 5′ end a 5′ splice donor site and at its 3′ end a 3′ splice acceptor site; (iii) a nucleotide sequence comprising a first exonic sequence having a 5′ to 3′ orientation, wherein the first exonic sequence comprises an alternatively-spliced exon comprising at its 3′ end a heterologous stop codon; (iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; (v) a nucleotide sequence comprising a second exonic sequence having a 5′ to 3′ orientation, wherein the second exonic sequence comprises a constitutive exon; (vi) a nucleotide sequence comprising a third intronic sequence having a 5′ to 3′ orientation, wherein the third intronic sequence comprises at its 5′ end a 5′ splice donor site and at its 3′ end a 3′ splice acceptor site; and (vii) a nucleotide sequence comprising a second portion of a coding region of the transgene having a 5′ to 3′ orientation.

Aspects of the invention relate to a transgene comprising, in the 5′ to 3′ direction: (i) a nucleotide sequence comprising a coding region of the transgene having a 5′ to 3′ orientation; (ii) a nucleotide sequence comprising a first intronic sequence having a 5′ to 3′ orientation, wherein the first intronic sequence comprises at its 5′ end a 5′ splice donor site and at its 3′ end a 3′ splice acceptor site; (iii) a nucleotide sequence comprising a first exonic sequence having a 5′ to 3′ orientation, wherein the first exonic sequence comprises an alternatively-spliced exon comprising a positive or negative cis-acting element; (iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; and (v) a nucleotide sequence comprising a second exonic sequence having a 5′ to 3′ orientation, wherein the second exonic sequence comprises a constitutive exon.

Aspects of the disclosure relate to a transgene comprising: (i) a constitutive exon and one or more intronic sequences, each from a first gene; (ii) an alternatively-spliced exon cassette, and (iii) a coding region of interest from a third gene. In some embodiments, the alternatively-spliced exon cassette comprises: (a) an alternatively-spliced exon, and (b) flanking intronic sequences. In some embodiments, each of (a) and (b) are from a second gene. In some embodiments, the alternatively-spliced exon comprises an ATG start codon at its 3′ end.

In some embodiments, the first and second gene are the same gene; the first and third gene are the same gene; or all of the first, second, and third genes are the same gene.

In some embodiments, the first gene is survival motor neuron 1 (SMN1).

In some embodiments, the constitutive exon comprises exon 6 of SMN1, or a portion thereof. In some embodiments, the constitutive exon comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 102. In some embodiments, the constitutive exon comprises a polynucleotide having a nucleic acid sequence as set forth in SEQ ID NO: 102.

In some embodiments, the one or more intronic sequences of (i) are or are derived from intron 6 and/or intron 7 of SMN1. In some embodiments, the one or more intronic sequences of (i) comprise(s) a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 103 and/or SEQ ID NO: 104. In some embodiments, the one or more intronic sequences of (i) comprise(s) a polynucleotide having a nucleic acid sequence as set forth in SEQ ID NO: 103 and/or SEQ ID NO: 104.

In some embodiments, the second gene is a gene selected from the group consisting of: CAMK2B, PKP2, LGMN, NRAP, VPS39, KSR1, PDLIM3, BIN1, ARFGAP2, KIF13A, and/or PICALM. In some embodiments, the second gene is bridging integrator 1 (BIN1).

In some embodiments, the alternatively-spliced exon comprises exon 11 of BIN1. In some embodiments, the alternatively-spliced exon comprises a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 37 or SEQ ID NO: 38. In some embodiments, the alternatively-spliced exon comprises a polynucleotide having a nucleic acid sequence as set forth in either SEQ ID NO: 37 or SEQ ID NO: 38.

In some embodiments, the flanking intronic sequences of (ii) are or are derived from intron 10 and/or intron 11 of BIN1. In some embodiments, the flanking intronic sequences of (ii) each comprise a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 15 or SEQ ID NO: 16. In some embodiments, the flanking intronic sequences of (ii) each comprise a polynucleotide having a nucleic acid sequence as set forth in either SEQ ID NO: 15 or SEQ ID NO: 16.

In some embodiments, the alternatively-spliced exon cassette comprises a polynucleotide having at least 70%, at least 75%, at least 80%, at least 90%, at least 92%, at least 95%, at least 98%, or at least 99% sequence identity, relative to a nucleic acid sequence as set forth in any one of SEQ ID NOs: 107-778. In some embodiments, the alternatively-spliced exon cassette comprises a polynucleotide having a nucleic acid sequence as set forth in any one of SEQ ID NOs: 107-778.

In some embodiments, the third gene is myotubularin 1 (MTM1) or calpain 3 (CAPN3). In some embodiments, the coding region of interest comprises a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 1881 or SEQ ID NO: 1882. In some embodiments, the coding region of interest comprises a polynucleotide having a nucleic acid sequence as set forth in either SEQ ID NO: 1881 or SEQ ID NO: 1882.

In some embodiments, if the wild-type alternatively-spliced exon does not comprise an ATG start codon, the alternatively-spliced exon comprises 1-3 nucleic acid substitutions, relative to the wild-type alternatively-spliced exon, to form the ATG start codon within the alternatively-spliced exon. In some embodiments, the ATG start codon is formed in the alternatively-spliced exon by 1 nucleic acid substitution. In some embodiments, the ATG start codon is formed in the alternatively-spliced exon by 2 nucleic acid substitutions. In some embodiments, the ATG start codon is formed in the alternatively-spliced exon by 3 nucleic acid substitutions.

In some embodiments, the alternatively-spliced exon is retained in the spliced transcript. In some embodiments, all native start codons located 5′ to the ATG start codon located within the alternatively-spliced exon are disrupted or deleted.

In some embodiments, the alternatively-spliced exon cassette is located 5′, relative to the coding region of interest. In some embodiments, the constitutive exon is located 5′, relative to the alternatively-spliced exon cassette. In some embodiments, the one or more intronic sequences of (i) flank the alternatively-spliced exon cassette.

In some embodiments, the alternatively-spliced exon comprises a heterologous, in-frame stop codon. In some embodiments, the heterologous, in-frame stop codon is at least 50 nucleotides upstream of the next 5′ splice junction. In some embodiments, the heterologous, in-frame stop codon elicits nonsense-mediated decay.

In some embodiments, the alternatively-spliced exon is retained in the spliced transcript in distinct tissues. In some embodiments, the alternatively-spliced exon is retained in the spliced transcript in skeletal muscle. In some embodiments, the alternatively-spliced exon is not retained in the spliced transcript in heart and/or liver tissue.

In some embodiments, the flanking intronic sequences of (ii)(b) are or are derived from native flanking introns of the alternatively-spliced exon. In some embodiments, the flanking intronic sequences of (ii)(b) each comprise at least one modification, relative to a naturally occurring intronic sequence. In some embodiments, the modification is a substitution or deletion of one or more nucleic acids.

In some embodiments, the ATG start codon is located at the 3′ end of the alternatively-spliced exon. In some embodiments, the ATG start codon is in the same reading frame as the coding region of interest. In some embodiments, the ATG start codon is within up to 5, 10, 20, or 30 nucleotides upstream of the 3′ end of the alternative-spliced exon. In some embodiments, the ATG start codon is within up to 5, 10, 20, or 30 nucleotides upstream of the 3′ end of the alternative-spliced exon and is in the same reading frame as the coding region of interest.

In some embodiments, if the wild-type alternatively-spliced exon does not comprise an ATG start codon at its 3′ end, the first 10 nucleotides of the flanking intronic sequence which is immediately 3′ to the alternatively-spliced exon comprise 1-5 nucleotide substitutions, relative to the wild-type flanking intronic sequence which is immediately 3′ to the wild-type alternatively-spliced exon.

In some embodiments, the one or more intronic sequences of (i) each comprise at least one modification, relative to a naturally occurring intronic sequence. In some embodiments, the modification is a substitution or deletion of one or more nucleic acids.

In some embodiments, the coding region of interest comprises at least one modification, relative to a naturally occurring coding region of the third gene. In some embodiments, the modification is a substitution or deletion of one or more nucleic acids. In some embodiments, the coding region of interest comprises a deletion or disruption of a native start codon. In some embodiments, the coding region of interest comprises at least one heterologous stop codon. In some embodiments, the at least one heterologous stop codon is at least 50 nucleotides upstream of the next 5′ splice junction. In some embodiments, the at least one heterologous stop codon elicits nonsense-mediated decay.

In some embodiments, a transgene as described in any embodiment of the disclosure further comprises a 3′ untranslated region (UTR). In some embodiments, the 3′ UTR is SV40. In some embodiments, the SV40 3′ UTR comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1883. In some embodiments, the SV40 3′ UTR comprises a polynucleotide having a nucleic acid sequence as set forth in SEQ ID NO: 1883. In some embodiments, the 3′ UTR comprises a polyadenylation (pA) site and a cleavage site. In some embodiments, the polyadenylation site is an SV40 pA site.

In some embodiments, a transgene as described in any embodiment of the disclosure further comprises a promoter, wherein the promoter is located 5′, relative to all of (i), (ii), and (iii). In some embodiments, the promoter is a tissue-specific promoter. In some embodiments, the tissue-specific promoter is an MHCK7 promoter. In some embodiments, an MHCK7 promoter comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1880. In some embodiments, an MHCK7 promoter comprises a polynucleotide having a nucleic acid sequence as set forth in SEQ ID NO: 1880.

In some embodiments, the alternatively-spliced exon cassette comprises a nucleic acid sequence which is 450 to 650 nucleotides in length.

Aspects of the disclosure relate to a recombinant viral genome comprising a transgene as described in any embodiment of the disclosure. In some embodiments, the recombinant viral genome is a genome from a recombinant adeno-associated virus (rAAV). In some embodiments, the transgene is flanked by AAV inverted terminal repeat (ITR) sequences. In some embodiments, the AAV ITR sequences are AAV2 ITR sequences. In some embodiments, an AAV2 ITR comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1879. In some embodiments, an AAV2 ITR comprises a polynucleotide having a nucleic acid sequence as set forth in SEQ ID NO: 1879.

In some embodiments, the recombinant viral genome comprises a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 105 or SEQ ID NO: 106. In some embodiments, the recombinant viral genome comprises a polynucleotide having a nucleic acid sequence as set forth in either SEQ ID NO: 105 or SEQ ID NO: 106.

Aspects of the disclosure relate to an rAAV particle comprising a recombinant viral genome as described in any embodiment of the disclosure. In some embodiments, the rAAV particle comprises AAV serotype 1, 2, 3, 4, 5, 6, 7, 8, 9, or 10, or AAV derivative or pseudotype AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y→F), AAV8 (Y733F), AAV2.15, AAV2.4, AAVM41, and AAVr3.45. In some embodiments, the rAAV particle further comprises at least one helper plasmid. In some embodiments, the helper plasmid comprises a rep gene and a cap gene. In some embodiments, the rep gene encodes Rep78, Rep68, Rep52, or Rep40, and/or wherein the cap gene encodes a VP1, VP2, and/or VP3 region of the viral capsid protein. In some embodiments, the rAAV particle comprises two helper plasmids. In some embodiments, the first helper plasmid comprises a rep gene and a cap gene and the second helper plasmid comprises a E1a gene, a E1b gene, a E4 gene, a E2a gene, and a VA gene.

Aspects of the disclosure relate to a recombinant viral genome comprising a transgene. In some embodiments, the transgene comprises: (i) a constitutive exon and one or more intronic sequences; (ii) an alternative exon cassette; and (iii) a coding region of interest. In some embodiments, the alternative exon cassette comprises: (a) an alternatively-spliced exon; (b) at least a portion of the intron immediately upstream of the alternatively-spliced exon; and (c) at least a portion of the intron immediately downstream of the alternatively-spliced exon. In some embodiments, if the wild-type alternatively-spliced exon does not comprise an ATG start codon at its 3′ end: (1) the 3′ end of the alternatively-spliced exon comprises 1-3 nucleic acid substitutions relative to the wild-type alternatively-spliced exon to form an ATG start codon, and (2) the first 10 nucleotides of the intron immediately downstream of the alternatively-spliced exon comprise 1-5 nucleic acid substitutions relative to the wild-type intron immediately downstream of the wild-type alternatively-spliced exon.

In some embodiments, the 1-5 nucleic acid substitutions of (2) increase splice site strength. In some embodiments, any wild-type start codons within the alternatively-spliced exon located upstream of the ATG start codon at the 3′ end of the alternatively-spliced exon are disrupted or deleted. In some embodiments, the recombinant viral genome further comprises a tissue-specific promoter upstream of the alternative exon cassette. In some embodiments, the coding region of interest is or is derived from a naturally occurring coding region of MTM1 or CAPN3. In some embodiments, the tissue-specific promoter is an MHCK7 promoter. In some embodiments, the alternative exon is exon 11 of the BIN1 gene. In some embodiments, the constitutive exon is exon 6 of the SMN1 gene. In some embodiments, the alternative exon cassette promotes skeletal muscle expression of the coding region of interest and reduces cardiac muscle expression of the coding region of interest. In some embodiments, the alternative exon cassette is approximately 600 nucleotides in length.

Aspects of the disclosure relate to a method of treating a disease or condition in a subject comprising administering a recombinant viral genome or an rAAV particle according to any embodiment of the present disclosure to the subject. In some embodiments, the subject is a mammal. In some embodiments, the mammal is a human. In some embodiments, the recombinant viral genome or rAAV particle is administered to the subject at least one time. In some embodiments, the viral genome or rAAV particle is administered to the subject 2, 3, 4, 5, 6, 7, 8, 9, or 10 times. In some embodiments, the viral genome or rAAV particle is administered to the subject parenterally, subcutaneously, intraocularly, intravitreally, subretinally, intravenously (IV), intracerebro-ventricularly, intramuscularly, intrathecally (IT), intracisternally, intraperitoneally, enterally, via inhalation, topically, or by direct injection to one or more cells, tissues, or organs. In some embodiments, the viral genome or viral particle is administered to the subject by intravenous injection, intramuscular injection, intrathecal injection, or intravitreal injection. In some embodiments, the disease or condition is a disease or condition selected from the group consisting of Dentatorubral-pallido-luysian atrophy (DRPLA), myotonic dystrophy type 1 (DM1), myotonic dystrophy type 2 (DM2), Fragile X syndrome of mental retardation (FMR1), Fragile X tremor ataxia syndrome (FXTAS), FRAXE mental retardation (FMR2), Friedreichs ataxia (FRDA), Huntington disease (HD), Huntington disease-like 2 (HDL2), Oculopharyngeal muscular dystrophy (OPMD), Myoclonic epilepsy type 1, Alzheimer's disease, ALS/FTD, spinocerebellar ataxia type 1 (SCA1), spinocerebellar ataxia type 2 (SCA2), spinocerebellar ataxia type 3 (SCA3), spinocerebellar ataxia type 6 (SCA6), spinocerebellar ataxia type 7 (SCAT), spinocerebellar ataxia type 8 (SCA8), spinocerebellar ataxia type 10 (SCA10), spinocerebellar ataxia type 12 (SCA12), spinocerebellar ataxia type 17 (SCA17), Syndromic/non-syndromic X-linked mental retardation, Emery-Dreifuss muscular dystrophy type 2, familial partial lipodystrophy, limb girdle muscular dystrophy type 1B, dilated cardiomyopathy, familial partial lipodystrophy, Charcot-Marie-Tooth disorder type 2B1, mandibuloacral dysplasia, childhood progeria syndrome (Hutchinson-Gilford syndrome), Werner syndrome, Dilated cardiomyopathy (DCM), Hypertrophic cardiomyopathy (HCM), Restrictive cardiomyopathy (RCM), Left Ventricular Non-compaction (LVNC), Arrhythmogenic Right Ventricular Dysplasia (ARVD), takotsubo cardiomyopathy, Duchenne muscular dystrophy, Becker muscular dystrophy, Limb-girdle muscular dystrophy, Facioscapulohumeral muscular dystrophy, Congenital muscular dystrophy, Oculopharyngeal muscular dystrophy, Distal muscular dystrophy, Emery-Dreifuss muscular dystrophy, dementia, Parkinson's disease (PD), a PD-related disorder, Prion disease, a motor neuron disease (MND), Progressive bulbar palsy (PBP), Progressive muscular atrophy (PMA), Primary lateral sclerosis (PLS), Spinal muscular atrophy (SMA), a bladder cancer, a breast cancer, a colorectal cancer, a kidney cancer, a lung cancer, a lymphoma, a melanoma, an oral cancer, an ovarian cancer, an oropharyngeal cancer, a pancreatic cancer, a prostate cancer, a thyroid cancer, a uterine cancer, Down syndrome, Prader-Willi Syndrome (PWS), Bloom Syndrome, Cockayne Syndrome Type I-216400, Cockayne Syndrome Type III, Cockayne Syndrome Type I, Hutchinson-Gilford Progeria Syndrome, Mandibuloacral Dysplasia with Type A Lipodystrophy, Progeria, Adult Onset Progeroid Syndrome, Neonatal Rothmund-Thomson Syndrome, Seip Syndrome, Werner Syndrome, Replication Focus-Forming Activity 1, myotubular myopathy, Danon Disease, and/or centronuclear myopathy.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a schematic illustrating the concept of a recombinant viral genome (e.g., rAAV or lentivirus) modified to include a transgene comprising a coding region of interest (e.g., encoding a therapeutic protein) under regulatory control by an alternatively-spliced exon (or an alternatively-spliced exon cassette). Step (b) shows the formation of a pre-mRNA which includes the coding region of interest and the alternatively-spliced exon. Step (c) shows the splicing-out or splicing-in of the alternatively-spliced exon based on one or more conditions (e.g., cell type, disease state, or other intracellular environmental signal). The splicing-out of the alternatively-spliced exon results in mRNA isoform 1 in (d), whereas the splicing-in of the alternatively-spliced exon (ASE) results in mRNA isoform 2 in (e). As shown in (g), the absence of the alternatively-spliced exon removes a positive or negative regulatory cis-element. The removal of a positive regulatory cis-element, such as a translation start signal, will result in the downregulation or decreased expression of the transgene, i.e., the reduced expression of the product encoded by the coding region of interest. However, the removal of a negative regulatory cis-element, such as mRNA degradation element, may lead to the upregulation or increased expression of the transgene, i.e., the increased expression of the product encoded by the coding region of interest. As shown in (h), the presence of the alternatively-spliced exon splices-in a positive or negative regulatory cis-element associated with the alternatively-spliced exon. The maintenance of a positive regulatory cis-element, such as a translation start signal, will result in the upregulation or increased expression of the transgene, i.e., the increased expression of the product encoded by the coding region of the transgene. However, the maintenance of a negative regulatory cis-element, such as mRNA degradation element, may lead to the downregulation or decreased expression of the transgene, i.e., the decreased expression of the product encoded by the coding region of the transgene.

FIG. 2 shows different models of alternative splicing which could be utilized in the nucleic acid vectors of the present disclosure. From top to bottom: a skipped exon model of alternative splicing, a retained intron model of alternative splicing, an alternative 5′ splice site model of alternative splicing, an alternative 3′ splice site model of alternative splicing, a mutually exclusive exon model of alternative splicing, and an alternative last exon model of alternative splicing. White regions represent constitutive exons throughout. Gray regions represent alternatively-spliced exons. One or more of the constitutive exons may be modified to contain a coding region of interest, e.g., a coding region of a transgene that encodes a therapeutic protein.

FIGS. 3A-3B show two schematics representing exemplary recombinant viral genomes. FIG. 3A shows a typical recombinant adeno-associated virus (rAAV) genome design. Two AAV inverted terminal repeats (ITRs) flank the transgene. The transgene may comprise a coding region of interest (e.g., encoding a therapeutic protein) under regulatory control of an alternatively-spliced exon (or cassette comprising an alternatively-spliced exon). In various embodiments, the cassettes (e.g., in the context of a transgene) may take on the architectures shown in any of FIG. 2 or 3-8, or any other suitable arrangement of elements so long as the alternatively-spliced exon is configured to regulate the expression of the coding region of interest of the transgene. FIG. 3B shows a typical recombinant lentivirus genome design. The 5′ and 3′ sequences of the lentivirus genome flank the packaging signal (PSI), rev response elements (RRE), and transgene. The transgene may comprise a coding region of interest (e.g., encoding a therapeutic protein) under regulatory control of an alternatively-spliced exon (or cassette comprising an alternatively-spliced exon). When transgenes are introduced using a lentivirus vector genome, the promoter and nucleotide sequence comprising the transgene sequence must be encoded on the minus strand of the lentivirus genome to prevent splicing during virus production and packaging. In various embodiments, the cassettes (e.g., in the context of a transgene) may take on the architectures shown in any of FIG. 2 or 3-8, or any other suitable arrangement of elements so long as the alternatively-spliced exon is configured to regulate the expression of the coding region of interest of the transgene.

FIGS. 4A-4C show three embodiments contemplated for the structural configuration of the cassettes (e.g., in the context of a transgene) that may inserted into a recombinant viral vector genome and which comprise at least (i) an alternatively-spliced exon and (ii) a coding region of interest (e.g., encoding a therapeutic protein) (or an exon comprising the coding region of interest), and wherein the alternatively-spliced exon comprises at least one positive or negative regulatory cis-element. Non-limiting examples of positive or negative regulatory cis-elements located within the alternatively-spliced exons can include, without limitation, a translation start codon, a translation stop codon, a binding site for an RNA binding protein that serves to positively regulate mRNA translation, a binding site for an RNA binding protein that serves to negatively regulate mRNA translation, a binding site for a nucleic acid molecule (e.g., an miRNA) that serves to positively regulate mRNA translation, a binding site for a nucleic acid molecule (e.g., an siRNA) that serves to negatively regulate mRNA stability or degradation, a binding site for an RNA binding protein that serves to positively regulate mRNA stability or degradation, a binding site for an RNA binding protein that serves to negatively regulate mRNA stability or degradation, a binding site for a nucleic acid molecule (e.g., an miRNA) that serves to positively regulate mRNA stability or degradation, or a binding site for a nucleic acid molecule (e.g., an siRNA) that serves to negatively regulate mRNA stability or degradation. This list of examples is not intended to place any limitation on the scope and meaning of the positive and negative cis-elements and the disclosure embraces any genetic element or region positioned within, or at least associated with, an alternatively-spliced exon which exerts a positive or negative control on the overall expression of a transgene (e.g., encoding a therapeutic protein). In some cases, the cis-element is within the alternatively-spliced exon, but in other cases, the cis-element is separate from, but at least associated with, the alternatively-spliced exon, such that it becomes spliced-in or spliced-out at the same time as the alternatively-spliced exon. In various embodiments, the cassettes (e.g., in the context of a transgene) may include one or more additional components, including one or more other constitutive exons, and one or more introns. In FIGS. 4A-4C, the constitutive exons not comprising the coding region of interest are represented by narrow rectangles, introns are represented as dashed lines, and the alternatively-spliced exons are represented as shaded narrow rectangles. The exon or exons comprising the coding region (or portions thereof in the case of where the coding region is split into separate exons) are indicated as solid thick white rectangles.

FIG. 4A is a schematic of a cassette (e.g., in the context of a transgene) embodiment whereby the alternatively-spliced exon is upstream of the exon encoding the coding region of interest. Said another way, in this embodiment, the alternatively-spliced exon is to the 5′ of the exon encoding the coding region of interest.

FIG. 4B is a schematic of a cassette (e.g., in the context of a transgene) embodiment whereby the alternatively-spliced exon is downstream of the exon encoding the coding region of interest. Said another way, in this embodiment, the alternatively-spliced exon is to the 3′ of the exon encoding the coding region of interest.

FIG. 4C is a schematic of a cassette (e.g., in the context of a transgene) embodiment whereby the alternatively-spliced exon is positioned between two separate exons encoding portions of the coding region of interest. Said another way, in this embodiment, the alternatively-spliced exon is between the exons encoding the portions of the coding region of interest.

FIGS. 5A-5G depict various embodiments of the general model of the cassettes (e.g., in the context of a transgene) of FIG. 4A. FIG. 5A depicts an embodiment of the “skipped exon model.” FIG. 5B depicts an embodiment of the “retained intron model.” FIG. 5C depicts an embodiment of the “alternative 5′ splice site model.” FIG. 5D depicts an embodiment of the “alternative 3′ splice site model.” FIG. 5E depicts an embodiment of the “mutually exclusive exon model.” FIG. 5F depicts an exemplary alternatively spliced transcript. FIG. 5G depicts an exemplary constitutively spliced transcript.

FIGS. 6A-6G depict various embodiments of the general model of the cassettes (e.g., in the context of a transgene) of FIG. 4B. FIG. 6A depicts an embodiment of the “alternative last exon model.” FIG. 6B depicts an embodiment of the “skipped exon model.” FIG. 6C depicts an embodiment of the “retained intron model.” FIG. 6D depicts an embodiment of the “alternative 5′ splice site model.” FIG. 6E depicts an embodiment of the “alternative 3′ splice site model.” FIG. 6F depicts an embodiment of the “mutually exclusive exon model.” FIG. 6G depicts an embodiment of the “alternative last exon model.”

FIGS. 7A-7F depict various embodiments of the general model of the cassettes (e.g., in the context of a transgene) of FIG. 4C. FIG. 7A depicts the “skipped exon model.” FIG. 7B depicts the “retained intron model.” FIG. 7C depicts “alternative 5′ splice site model.” FIG. 7D depicts the “alternative 3′ splice site model.” FIG. 7E depicts the “mutually exclusive exon model.” FIG. 7F depicts the “alternative last exon model.”

FIGS. 8A-8B show embodiments of the general model of the cassettes (e.g., in the context of a transgene). FIG. 8A shows an embodiment of the general model of the cassettes (e.g., in the context of a transgene) of FIG. 4A. In the approach shown, the cassette (e.g., in the context of a transgene) comprises a constitutive exon at the left, an alternatively-spliced exon comprising an ATG (an example of a positive regulatory cis-element) in the middle, and a constitutive exon comprising a coding region of interest (shown with the natural ATG start codon removed to eliminate translation of that exon without further positive control by the alternatively-spliced exon). Black lines indicate intronic sequences (e.g., the flanking introns of the alternatively-spliced exon). Alternative reading frames within the exon comprising the coding sequence may in some embodiments be removed, as appropriate. Under alternative splicing conditions, which are specific to the nature of the chosen alternatively-spliced exon, the alternatively-spliced exon will be included, and productive translation of the coding sequence will result. To the contrary, under homeostatic conditions (normal splicing conditions), only the constitutive exon will be included, the presence of the ATG start codon in the alternatively-spliced exon will be eliminated, and the coding sequence will not be translated. The upper dotted lines show the splicing pattern leading to a splicing-in of the alternatively-spliced exon (expression of the coding region). The lower dotted lines show the splicing pattern leading to a splicing-out of the alternative-spliced exon (no or reduced expression of the coding region). FIG. 8B shows an embodiment of the general model of the cassettes (e.g., in the context of a transgene) of FIG. 4C. In the approach shown, the cassette (e.g., in the context of a transgene) comprises an alternatively-spliced exon (shown in gray) positioned between two separate constitutive exons each comprising a portion of the desired coding region. The exon to the left comprises the 5′ end of the coding sequence and the exon to the right comprises the 3′ end of the coding region. An in-frame stop codon is inserted into the alternatively-spliced exon at a location which is >50 nucleotides upstream of the next downstream splice site. Under alternative splicing conditions, which are specific to the nature of the chosen alternatively-spliced exon, the alternatively-spliced exon will be included, and NMD (nonsense-mediated mRNA decay) will result. Under homeostatic conditions (normal splicing conditions), only the constitutive exon will be included, and the 5′ and 3′ ends of the coding sequence will be joined resulting in productive translation of the coding sequence. The upper dotted lines show the splicing pattern leading to a splicing-in of the alternatively-spliced exon (no or reduced expression of the coding region due to active NMD). The lower dotted lines show the splicing pattern leading to a splicing-out of the alternative-spliced exon (expression of the coding region).

FIG. 9 shows a configuration of a gene therapy cargo whose translation can be regulated by alternative splicing. Inclusion of an alternative exon that ends in “ATG” can lead to translation of the downstream coding sequence. Exclusion will prevent appropriate protein translation of the downstream coding sequence.

FIG. 10 shows a construct design for the screening of alternative exon cassettes with regulatory activity. The construct used the SMN1 exon 6 and intron 6/7 context. Test alternative exon cassettes were inserted between portions of SMN1 intron 6 and 7. An MHCK7 was used. The coding sequence was derived from the human MTM1 gene. The 3′ UTR contained an SV40 polyadenylation and cleavage site. AAV2 ITRs flanked the construct. Splice site scores of the flanking constitutive exons are listed.

FIG. 11 shows a strategy to prevent undesired translation of peptides from alternative reading frames of MTM1. Amino acids generated in the MTM1 reading frame are listed (e.g., GCT encodes Alanine); only the 5′ end of MTM1 sequence is shown. Substitutions that preserve MTM1 reading frame but terminate alternative reading frames are shown. Arrows denote point mutations made to generate stop codons that would terminate open reading frames in the +1 and +2 reading frames. Nucleic acid substitutions are denoted by lower-case letters.

FIG. 12 shows a strategy to preserve splice site strength following mutation of bases to introduce ATG to the ends of alternative exons by altering 5′ splice site sequences. Because the addition of ATG to the end of each alternative exon may change the splice site strength, intronic bases to were altered to maintain splice site strength and preserve splicing activity. All upstream ATGs were also removed from alternative exons. Splice site strengths were scored by MaxEntScan and are shown. Splice sites are listed for the endogenous sequence (top), the endogenous sequence altered such that ATG is introduced (middle), and a “compensated” splice site sequence (bottom). Nucleic acid substitutions are denoted by lower-case letters.

FIG. 13 shows a construct barcoding strategy. For the first round of screening, a barcode strategy was used in which synonymous mutations were made and used to identify each candidate alternative exon uniquely. Barcodes were ˜350 NT away from the splice site with the intent of not affecting splicing. Barcodes were comprised of 5 wobble positions and generated by randomly cloning in: AAY CTN AGA TTY GCN (SEQ ID NO: 101) (2*4*2*4=64 possibilities). Barcode sequences (each 5 nucleotides in length) are shown at the end of each row in parentheses.

FIGS. 14A-14C show percent spliced in (psi) values for each tested cassette exon in various tissues. Psi values were plotted in heart (H), tibialis anterior (TA), and liver (L). Data for tibialis anterior was obtained from animals injected intramuscularly, and data from the other tissues was obtained from animals injected intravenously. FIG. 14A shows data obtained from the following tested cassette exons (from left to right): ARFGAP2, BIN1, CAMK2B, and KIF13A. FIG. 14B shows data obtained from the following tested cassette exons (from left to right): KSR1, LGMN, NRAP, and PDLIM3. FIG. 14C shows data obtained from the following tested cassette exons (from left to right): PICALM, PKP2, and VPS39.

FIGS. 15A-15B show percent spliced in (psi) values for each tested exon in tibialis anterior at various times following injection. Psi values were plotted for each sample versus every other sample. The number following the dash indicates the replicate number for that particular week. FIG. 15A shows a first comparison of psi values obtained at different time points following injection. FIG. 15B shows a second comparison of psi values obtained at different time points following injection.

FIGS. 16A-16B show the ratios of RNA binding protein (RBP) RNA expression in heart vs. skeletal muscle, or vice-versa. RNA expression values for RNA binding proteins were obtained from publicly available databases. The ratio of expression in heart versus skeletal muscle was computed; the RBPs showing the strongest bias in either direction were plotted. FIG. 16A shows the RBPs which were found to be enriched in muscle tissue, relative to heart tissue. FIG. 16B shows the RBPs which were found to be depleted in muscle tissue, relative to heart tissue.

FIG. 17 shows that the intronic sequence upstream of BIN1 exon 11 is enriched for CAC motifs. Top: ˜250 nucleotides upstream of BIN1 exon 11 are shown. Every CAC motif is shown in bold text. Bottom: the last 34 bases of the intron are shown from human, rhesus macaque, mouse, dog, and elephant. Every species shown has 2 CAC motifs within this region except for dog.

FIG. 18 shows percent spliced in (psi) values for BIN1 exon 11 in human, rhesus macaque, and dog. Psi values for BIN1 exon 11 for these species were obtained from publicly available datasets and plotted. The dog data includes data from animals modeling XLMTM1, including those also being treated with AAV-MTM1. AAV low, mid, and high denotes AAV-MTM1 treatment in XLMTM1 dogs from Dupont et al. (2020).

FIG. 19 shows splice site variants which were considered in the high throughput screen to optimize the BIN1 exon 11 cassette. The endogenous BIN1 3′ splice site is listed (top), along with the endogenous BIN1 5′ splice site (second row from top), the endogenous BIN1 5′ splice site sequence altered such that ATG is introduced (third row from top), and the “compensated” version characterized in the first screen (bottom). Additional splice sites tested are listed below. Nucleic acid substitutions are denoted by lower-case letters.

FIG. 20 shows intronic variants which were considered in the high throughput screen to optimize the BIN1 exon 11 cassette. Sequence from the downstream intron of BIN1 exon 11 is shown (top). Putative MBNL binding sites (YGCY motifs) are bolded. Putative RBFOX binding sites (TGCATG) are underlined. Sequence that includes 4 possible alterations is shown (bottom). The alterations, denoted with lower-case letters, either generate additional MBNL binding sites (the first, second, and third alterations, from 5′ to 3′) or an additional RBFOX site (the fourth alteration). Consideration of 0, 1, 2, 3, or 4 alterations in all combinations yields 16 possible sequences to test.

FIG. 21 shows a strategy to use PCR amplicons to read the association between barcodes and variants (the codebook). Given short read Illumina sequencing (˜75 nucleotides), a PCR strategy was used to associate the downstream barcode with upstream sequence variants.

FIG. 22 shows the number of barcodes encoding each variant. A histogram of the number of barcodes encoding each variant is shown for the plasmid library. On average, ˜8 barcodes encode each variant.

FIGS. 23A-23C show scatters of percent spliced in (psi) values for each variant in different tissues. Each point represents the mean psi for each variant across all barcodes representing that variant. Data from selected tissues is shown. FIG. 23A shows scatter between 2 heart samples, which lies along the diagonal (indicating reproducibility). FIG. 23B shows scatter between 2 gastrocnemius samples, which also lies along the diagonal (indicating reproducibility). FIG. 23C shows scatter between heart and skeletal muscle samples, which lies above the diagonal. This is because psi for most variants is higher in skeletal muscle than in heart.

FIGS. 24A-24B show scatters of mean percent spliced in (psi) as computed across multiple animals. Each point represents the mean psi for each variant across multiple animals (n=4 for all tissues). FIG. 24A shows data obtained from tibialis anterior (y-axis) versus heart (x-axis) tissue. FIG. 24B shows data obtained from gastrocnemius (y-axis) versus heart (x-axis) tissue.

FIGS. 25A-25D show percent spliced in (psi) values as a function of splice site strength for selected samples. Psi values for each variant were grouped by 3′ or 5′ splice site strength; data is shown only for heart sample 1 and gastrocnemius sample 1. There is a trend such that strong splice sites tend to yield higher inclusion levels. FIG. 25A shows the 3′ splice site strength relative to the psi in heart tissue for heart sample 1. FIG. 25B shows the 5′ splice site strength relative to the psi in heart tissue for heart sample 1. FIG. 25C shows the 3′ splice site strength relative to the psi in gastrocnemius tissue for gastrocnemius sample 1. FIG. 25D shows the 5′ splice site strength relative to the psi in gastrocnemius tissue for gastrocnemius sample 1.

FIGS. 26A-26B show scatters of mean percent spliced in (psi) for each variant as computed across multiple animals when linked to a CAPN3 cargo. Each point represents the mean psi for each variant across multiple animals (n=4 for all tissues). FIG. 26A shows data obtained from tibialis anterior (y-axis) versus heart (x-axis) tissue. FIG. 26B shows data obtained from gastrocnemius (y-axis) versus heart (x-axis) tissue.

FIGS. 27A-27B show scatters of mean percent spliced in (psi) for each variant when linked to an MTM1 cargo versus a CAPN3 cargo. Each point represents the mean psi for each variant across multiple animals (n=4 for all tissues). The psi value for variants linked to the MTM1 cargo is shown on the x-axis and the psi value for the same variants linked to the CAPN3 cargo is shown on the y-axis. FIG. 27A shows data for heart tissue. FIG. 27B shows data for gastrocnemius tissue.

### DETAILED DESCRIPTION

The present disclosure relates to the observation that alternatively-spliced exons may be used in the context of viral vectors (e.g., AAV viral vectors or lentivirus viral vectors) to effectively regulate the expression of a coding region of interest (e.g., a coding region of a transgene that encodes a therapeutic protein). In certain aspects, the alternatively-spliced exons regulate the expression of a coding region of interest in a condition-sensitive manner (e.g., expression in one type of cell but not another, expression in a diseased condition, or expression in the presence of certain intracellular conditions). Accordingly, the present disclosure relates to a new approach for regulating expression of a transgene (or a coding region thereof) from a recombinant viral vector that couples alternatively-spliced exons with the expression of a coding region of interest (e.g., a coding region of a transgene encoding a therapeutic protein). The present disclosure describes a variety of exemplary configurations as to how to combine or otherwise pair the expression of a coding region of interest (or multiple portions of coding regions) with an alternatively-spliced exon, but any suitable arrangement or configuration is contemplated so long as the expression of the coding region of interest (or portions thereof) is configured to come under regulatory control of the alternatively-spliced exon.

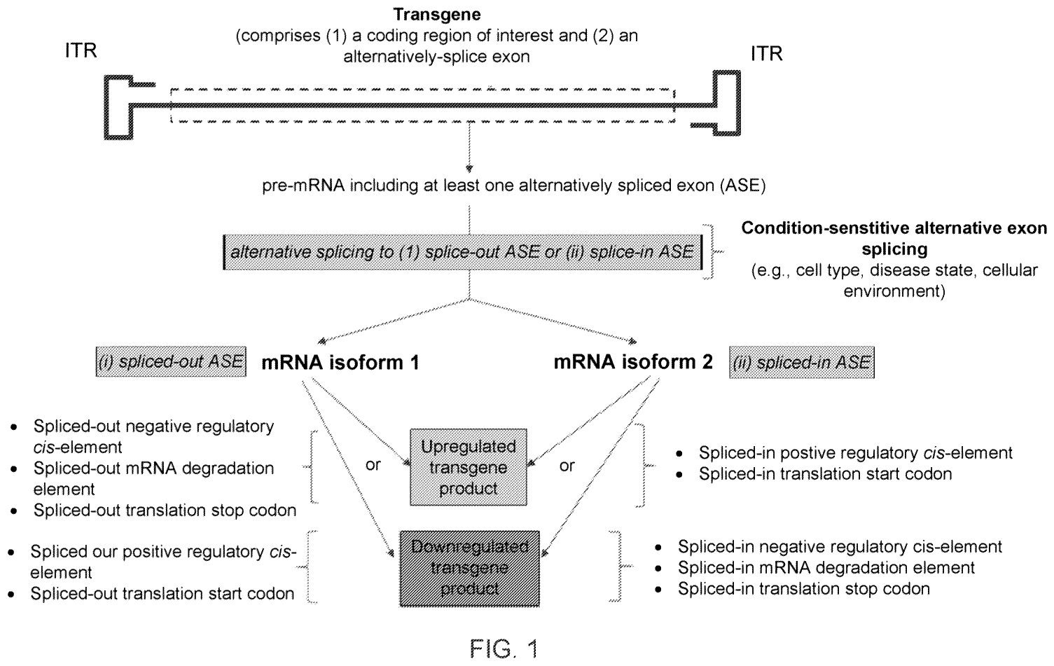

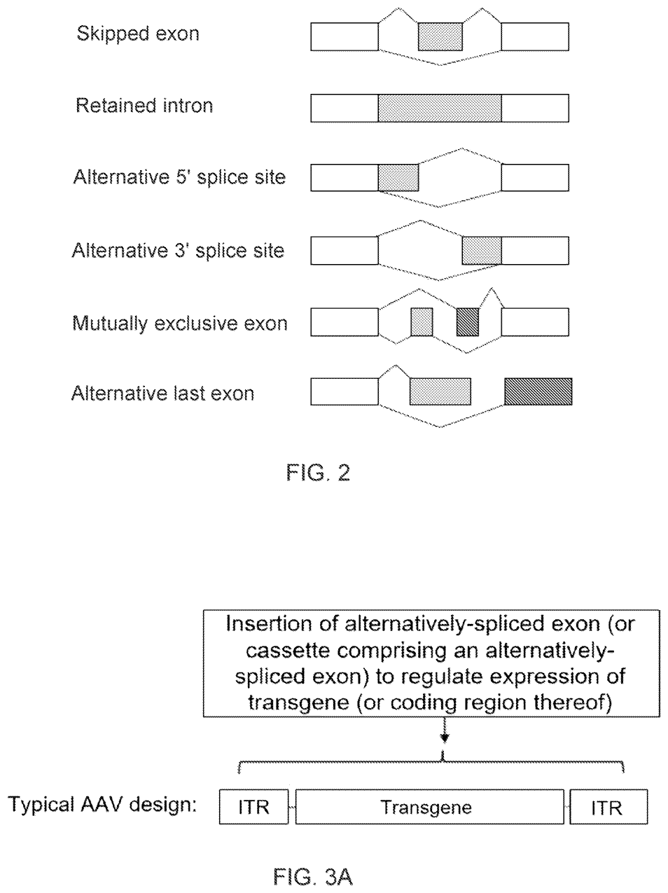

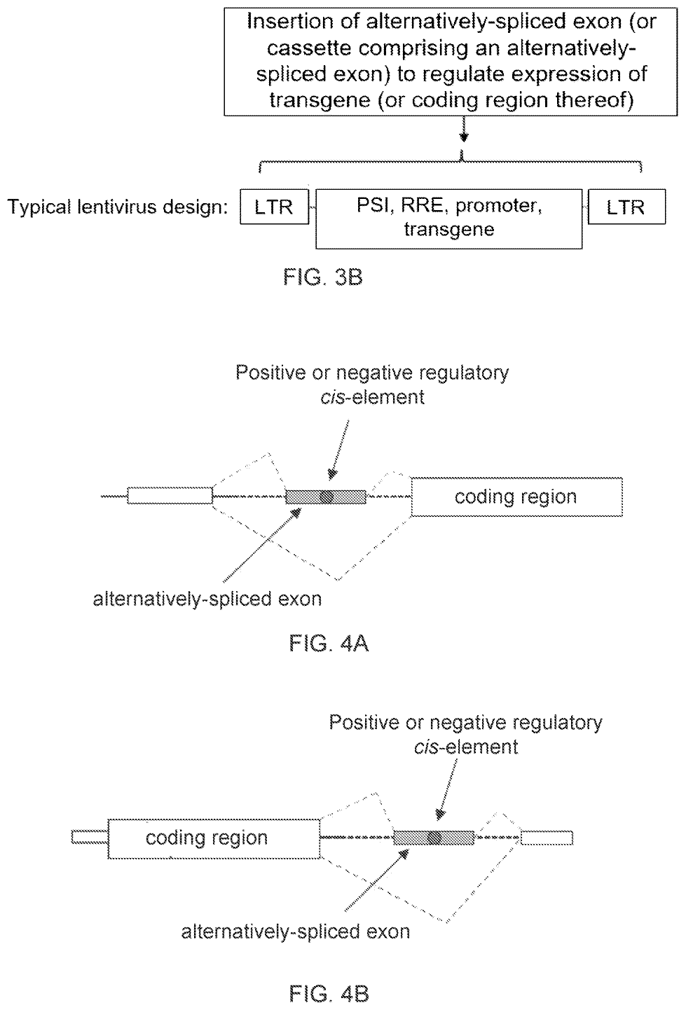

A schematic representing the disclosed new approach for regulating expression of a transgene (or a coding region of a transgene, e.g., a transgene encoding a therapeutic protein) in a recombinant viral genome using alternatively-spliced exons is provided in FIG. 1. As shown in FIG. 1, a viral genome may be configured to include a transgene that comprises a coding region of interest (e.g., encoding a therapeutic protein) and an alternatively-spliced exon (or a cassette comprising an alternatively-spliced exon) which regulates the expression of the coding region of the transgene. In addition, a number of exemplary embodiments of recombinant nucleic acid molecule constructs that comprise an alternatively-spliced exon and a coding region of interest (e.g., encoding a therapeutic protein) are shown in FIG. 2. FIG. 3 depicts, in general, typical AAV and lentivirus vector constructs comprising a coding region of interest whose expression is driven by a promoter, and which further include the insertion (at any suitable location) of a nucleotide sequence comprising an alternatively-spliced exon (or a cassette comprising an alternatively-spliced exon) to further regulate the expression of the coding region (e.g., by controlling translation or mRNA homeostasis, e.g., mRNA levels). In some embodiments, the nucleotide sequence comprising an alternatively-spliced exon may be in the form of a “cassette.” Examples of this are provided in FIGS. 2 and 4-7.

Such constructs represent embodiments that enable the disclosed new approach for regulating transgene expression (e.g., the expression of a therapeutic protein) from recombinant viral vectors in a condition-sensitive manner, whereby the condition-sensitive expression is controlled by alternatively-spliced exons which are included in the recombinant genome of the expression vector in such a manner that imparts a level of control on the expression of a coding region of interest (e.g., encoding a therapeutic protein). It will be understood that alternatively-spliced exons are spliced-in or spliced-out in a manner that can be dependent on one or more environmental conditions, e.g., intracellular conditions, such as a disease state (e.g., cancer) or even a type of cell (e.g., a liver cell versus a neuron, each of which have different intracellular conditions), or the presence of an external factor (such as, for example, an administered agent). Thus, whether the alternatively-spliced exon is spliced-in or spliced-out can be dependent upon the condition of the cell in which the splicing machinery operates.

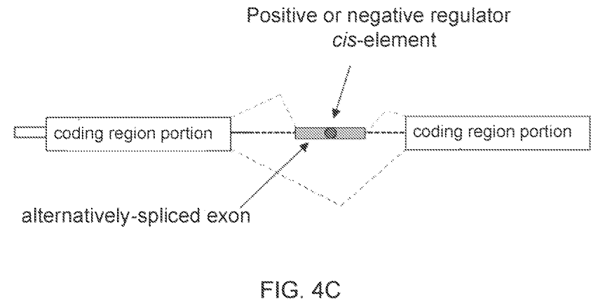

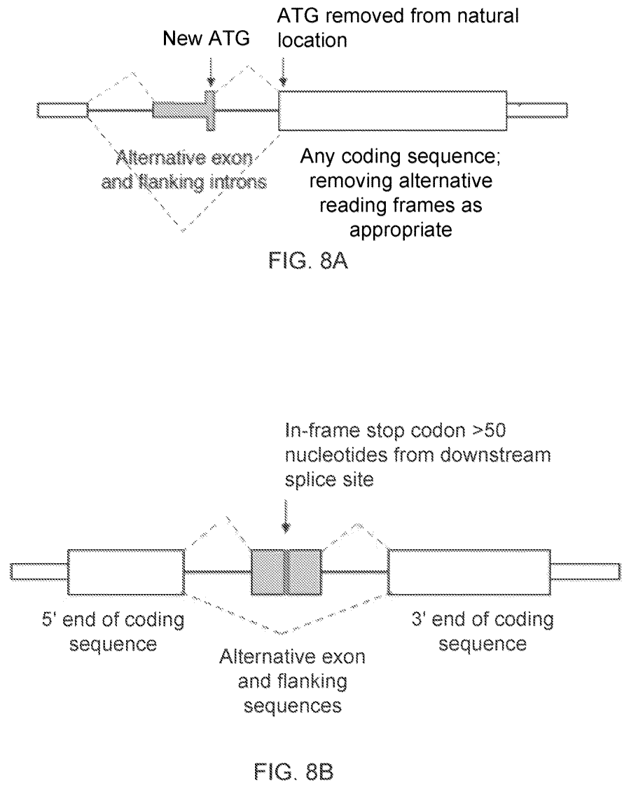

Turning to FIG. 1, a generalized schematic of a recombinant AAV is provided in (a) which comprises a transgene located between the left and right ITRs. The transgene is indicated as comprising a coding region of interest (e.g., which encodes a therapeutic protein) and an alternatively-spliced exon that regulates the expression of the transgene (or the product encoded by the coding region of interest). While the drawing depicts a recombinant AAV genome, other recombinant viral vector genomes may be used, such as recombinant lentivirus genomes. The recombinant viral genomes may be delivered or administered to subjects packaged in a viral vector, which refers to an infectious viral particle comprising a recombinant viral genome within a viral capsid, and in addition which may further include a lipid/protein envelope layer for enveloped viruses. In various embodiments, such as those provided in FIG. 2, or FIGS. 4-8, the coding region (or exon comprising the coding region) may be combined or arranged with the alternatively-spliced exon in the form of a transgene comprising any suitable arrangement of additional components, including one or more constitutive exons (i.e., those exons present in all spliced mRNA isoforms that result from the initial pre-mRNA transcript) and one or more introns. In other embodiments, an alternative exon cassette (comprising the alternatively-spliced exon) may be linked with or coupled to any coding region of interest to impart regulatory control on that coding region of interest.

The alternatively-spliced exon may be any naturally-occurring alternatively-spliced exon or any recombinant alternatively-spliced exon. A variety of configurations are contemplated, and no limitation is implied by FIG. 1 as to the possible configurations that may be employed. For instance, the alternatively-spliced exon may be located between two exons that each separately comprise a portion of the coding region of interest. In other instances, the alternatively-spliced exon is located outside of the exon comprising the coding region of interest. In such embodiments, the alternatively-spliced exon may be located downstream of the exon encoding the coding region of interest. In other such embodiments, the alternatively-spliced exon may be located upstream of the exon encoding the coding region of interest. The general descriptions of the configuration of the cassettes comprising the alternatively-spliced exon and the coding region of interest (or the exon comprising the coding region of interest) embrace any suitable configuration, including those embodiments described in FIGS. 2 and 4-8.

In FIG. 1, step (b) shows the formation of a pre-mRNA (i.e., a primary transcription product which has not yet been processed by splicing) which includes the coding region of interest and the alternatively-spliced exon. Step (c) shows the splicing-out or splicing-in of the alternatively-spliced exon based on one or more conditions (e.g., cell type, disease state, or other intracellular environmental signal). The splicing-out of the alternatively-spliced exon results in mRNA isoform 1 in (d), whereas the splicing-in of the alternatively-spliced exon results in mRNA isoform 2 in (e). As shown in (g), the absence of the alternatively-spliced exon removes a positive or negative regulatory cis-element. The removal of a positive regulatory cis-element, such as a translation start signal, will result in the downregulation or down-expression of the transgene, i.e., the reduced expression of the product encoded by the coding region of interest. However, the removal of a negative regulatory cis-element, such as mRNA degradation element, may lead to the upregulation or up-expression of the transgene, i.e., the increased expression of the product encoded by the coding region of interest. As shown in (h), the presence of the alternatively-spliced exon splices-in a positive or negative regulatory cis-element associated with the alternatively-spliced exon. The maintenance of a positive regulatory cis-element, such as a translation start signal, will result in the upregulation or up-expression of the transgene, i.e., the increased expression of the product encoded by the coding region of the transgene. However, the maintenance of a negative regulatory cis-element, such as mRNA degradation element, may lead to the downregulation or down-expression of the transgene, i.e., the decreased expression of the product encoded by the coding region of the transgene. Other configurations are also possible and contemplated herein and exemplified below in various embodiments provided in FIGS. 2-8.

In certain aspects, the disclosure provides methods and compositions for regulating gene expression using viral vectors comprising a recombinant viral genome described herein. Viral vectors can be used to deliver one or more transgenes (comprising a coding region of interest which encodes a protein of interest, such as a therapeutic protein) for therapeutic, diagnostic, or other purposes. In some aspects, expression of a transgene in a recombinant viral genome can be regulated using alternative splicing of an RNA expressed from the viral genome.

Thus, aspects of the disclosure relate to methods and compositions for regulating expression of a transgene (comprising a coding region of interest which encodes a protein of interest, such as a therapeutic protein) using viral vectors comprising a recombinant viral genome described herein. A recombinant viral genome can be engineered to include one or more exons (e.g., one or more of a constitutive exon, an alternatively-spliced exon, and/or engineered versions thereof) that (a) can be either spliced-in or spliced-out of a pre-mRNA encoded by the genome, and (b) include one or more positive or negative regulatory cis-elements that affect protein expression (e.g., mRNA stability and/or translation of the coding region of interest).

Different intron and exon configurations can be used to provide for alternatively-spliced exon splicing, as discussed in greater detail herein, and shown in FIG. 2 and FIGS. 4-8 as examples. Non-limiting examples include the following models of alternative splicing: skipped exons, retained introns, alternative 5′ splice sites, alternative 3′ splice sites, mutually exclusive exons, and alterative last exons as illustrated in FIGS. 2 and 4-8. Each of these different intron/exon configurations can be used to leverage alternatively-spliced exons which may, in some embodiments, include one or more positive or negative regulatory cis-elements that promote or limit expression of the coding region of interest. For example, such sequences may promote translation and/or stability, or inhibit or terminate RNA translation and/or promote RNA degradation. Such cis-acting elements may in some embodiments be sequences that form secondary structures (e.g., that slow translation), bind to one or more regulatory RNAs (e.g., siRNAs), and/or be targeted by one or more intracellular enzymes (e.g., nucleases).

It will be appreciated that different types of splice sites exist which may result in splicing under specific conditions. Such splice sites can be chosen for their ability to regulate splicing under conditions of interest. Alternatively or additionally, splice sites may be chosen based upon their relative strength, as calculated using a variety of published methods (see, e.g., Yeo & Burge (2004), Maximum entropy modeling of short sequence motifs with applications to RNA splicing signals, J. Comput. Biol., 11(2-3):377-94). Such relative strength may in some embodiments reflect the efficiency of recognition by the core spliceosomal machinery (e.g., U1 and U2 snRNPs). In some embodiments, splice sites may be altered to enhance or diminish recognition by the core spliceosomal machinery. Such alterations may be performed, in some embodiments, to achieve the desired regulatory behavior in conditions of interest. For example, splice sites may be used to make splicing responsive to certain endogenous or exogenous factors such that the alternative splicing of the DNA is specific to, such as, for example, certain tissues, certain diseases, certain intracellular conditions, etc. In some embodiments, splicing may be additionally or alternatively responsive to an exogenous agent (e.g., a small molecule, antibody, or other compound) which regulates splicing of the pre-mRNA.

Alternatively-spliced exons as described herein may in some embodiments be contained within an alternatively-spliced exon cassette, as shown in the various embodiments of FIGS. 2 and 4-8.

Thus, in some embodiments a recombinant viral genome of the present disclosure comprises a transgene comprising at least one alternatively-spliced exon (or “regulatory”) cassette. In some embodiments, a transgene comprising an alternatively-spliced exon cassette comprises at least one alternatively-spliced exon, intronic sequences flanking the alternatively-spliced exon, and an exon comprising a coding region of interest. However, a transgene comprising a regulatory cassette may in some embodiments also contain additional components, such as a constitutive exon, additional intronic sequences, or both. Accordingly, in some embodiments, a transgene comprising an alternatively-spliced exon cassette comprises any one or more of the following components: an alternatively-spliced exon, a flanking intron, an exon comprising a coding region of interest, and/or a constitutive exon.

In some aspects, alternative splicing regulation can be used to help control the expression of a coding region of interest encoded by a recombinant viral genome (e.g., an rAAV recombinant genome, a lentivirus recombinant genome). Thus, aspects of the invention relate to a method of regulating expression of a coding region of interest using a viral vector comprising a recombinant viral genome described herein. In some embodiments, the method comprises: (i) inserting into the recombinant viral genome at least one transgene comprising an alternatively-spliced exon cassette (e.g., such as any of those shown in FIGS. 2 and 4-8); (ii) introducing a heterologous start codon or part of a heterologous start codon at the 3′ end of the alternatively-spliced exon; (iii) disrupting or deleting all native start codons located 5′ to the heterologous start codon; and (iv) deleting a native start codon, or a portion thereof, from, and/or introducing heterologous stop codons into, the exon comprising a coding region of interest. In some embodiments, the constitutive exon, alternatively-spliced exon, and flanking intron are each located 5′ to the coding region of interest. In some embodiments, the method comprises: (i) inserting into the recombinant viral genome at least one transgene comprising an alternatively-spliced exon cassette; and (ii) introducing into the alternatively-spliced exon a heterologous, in-frame stop codon at least 50 nucleotides upstream of the next 5′ splice junction.

In some embodiments, the heterologous, in-frame stop codon elicits nonsense-mediated decay. In some embodiments, a transgene comprising an alternatively-spliced exon cassette comprises any one or more of the following components: an alternatively-spliced exon, a flanking intron, a coding region of interest, and/or a constitutive exon.

Accordingly, compositions and methods described herein can be useful to regulate expression of therapeutic transcripts in the context of viral vector-based treatments for diseases or disorders. Abnormal cellular regulation (e.g., abnormal regulation of intron splicing of one or more genes) can lead to changes in gene regulation and subsequent protein expression associated with a disease state. Some aspects of the invention therefore concern a method of treating a disease or condition in a subject comprising administering a viral vector of the disclosure to a subject, wherein the viral vector comprises a recombinant viral genome described herein. In some aspects, the present application provides compositions and methods that are useful for delivering genes that retain or restore therapeutically effective levels of regulation (e.g., therapeutically effective regulation of intron splicing).

In some aspects, a viral vector (e.g., an rAAV vector; a lentivirus vector, etc.) comprises a recombinant viral genome that includes a nucleic acid that encodes an RNA (e.g., an mRNA) comprising one or more introns. In some embodiments, splicing of at least one intron is regulated by one or more intracellular factor(s). Regulation of intron splicing can control the expression level of the RNA and/or of the type of RNA (e.g., of an RNA splice alternative) inside a cell.

### A. Definitions

Unless otherwise defined herein, all scientific and technical terms used in connection with the present disclosure shall have the meanings that are commonly understood by those of ordinary skill in the art. The meaning and scope of the terms are clear; however, in the event of any latent ambiguity, definitions provided herein take precedent over any dictionary or extrinsic definition. Further, unless otherwise required by context, singular terms shall include pluralities and plural terms shall include the singular. In this disclosure, the use of “or” means “and/or” unless stated otherwise. Furthermore, the use of the term “including,” as well as other forms, such as “includes” and “included,” is not limiting. Things described as “including” or “comprising” can also be configured as “consisting of” or similar language. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of subjects.

That the present disclosure may be more readily understood, select terms are defined below.

### (i) Transgene

As used herein, the term “transgene” refers to any recombinant gene or a segment thereof that includes a non-naturally occurring sequence. The non-naturally occurring sequence may in some embodiments be from a different organism, but it need not be. For example, in some embodiments a transgene is a recombinant gene, or segment thereof, from one organism or infectious agent (e.g., a virus) that is introduced into the genome of another organism or infectious agent. By contrast, in some embodiments, the transgene may contain segments of DNA taken from the same organism, but the segments are arranged in a non-natural configuration. In some embodiments, the non-naturally occurring sequence is an engineered non-naturally occurring sequence. As used herein, a transgene may comprise any combination of naturally-occurring and engineered DNA sequences. In some embodiments, the transgene comprises at least one coding region that encodes a polypeptide of interest (e.g., a therapeutic protein) or fragment thereof. The coding region that encodes a polypeptide of interest (e.g., a therapeutic protein) or fragment thereof may be alternately referred to herein as the “coding region of the transgene.”

A transgene may be introduced into the genome of another organism or infectious agent using recombinant DNA techniques. A transgene may include one or more coding regions of interest that encode a polypeptide of interest, e.g., a therapeutic protein. A transgene may include or may be modified to include one or more regulatory sequences, including, but not limited to, transcription regulatory sequences (e.g., promoter, enhancer, silencer, transcription factor binding sequence, 5′ UTR, or 3′ UTR), post-transcriptional regulatory sequences (e.g., acceptor/donor splicing sites and splicing regulatory sequences), and/or translation regulatory sequences (e.g., translation initiation signals, translation termination signals, mRNA degradation or decay signals, polyadenylation signals). In some embodiments, wherein a transgene is introduced into the genome of another organism using a recombinant adeno associated virus (AAV), the transgene comprises all components (e.g., exons, introns, regulatory sequences, etc.) which are located between the AAV inverted terminal repeat sequences (see, e.g., FIG. 3A).

In some embodiments, a transgene may be modified to comprise an alternatively-spliced exon, defined below, such that the regulation of the expression of the transgene—or of the product encoded by the coding region of the transgene—comes under control of the alternatively-spliced exon. The alternatively-spliced exon may be configured as a “cassette,” defined below.

### (ii) Regulatory Sequence

As used herein, a “regulatory sequence” or, equivalently, a “regulatory element,” may refer to a nucleotide sequence that regulates, directly or indirectly, any aspect of the expression of a gene or transgene, including regulatory sequences that effect transcription of a gene or transgene into one or more mRNAs, the processing of mRNA (e.g., the splicing of a pre-mRNA comprising exons and introns to produce one or more mRNA isoforms), and/or the translation of a coding region in a mRNA to form a polypeptide product.

Where a regulatory sequence or element is near, within, or otherwise proximal to a gene or transgene (or coding sequence thereof), the regulatory sequence may be referred to as a cis-acting regulatory sequence. This is in contrast to a trans acting regulatory sequence, which would be a regulatory sequence which is distal from a gene or transgene being regulated on the same or different nucleic acid molecule comprising a gene or transgene being regulated. Such cis-acting regulatory sequences may be referred to a “positive or negative regulatory cis-elements,” and, in certain embodiments, are located within an “alternatively-spliced exon.”

Non-limiting examples of positive or negative regulatory cis-elements can include, for instance, (1) a nucleotide sequence element that regulates, modulates, or otherwise controls the amount, stability, and/or degradation of an mRNA encoding a coding region of interest (or portions thereof); and/or (2) a nucleotide sequence element that regulates, modulates, or otherwise controls the translation of a coding region of interest (or portions thereof) encoded by an mRNA. Where positive or negative regulatory cis-elements are located within an alternatively-spliced exon, the splicing-in or splicing-out of the alternatively-spliced exons either retains or removes the positive or negative regulatory cis-element from a resulting post-spliced mRNA encoding the coding region of interest. Depending upon whether the alternatively-spliced exon is spliced-in or spliced-out, and then depending upon which one or more positive or negative regulatory cis-elements are associated with the alternatively-spliced exon, there will be a corresponding effect on the overall regulation of the expression of the transgene or a coding region of interest therein. Such effect may in some embodiments be that the expression level is upregulated or downregulated, or, for example, “turned-off” completely.

(iii) Alternatively-Spliced Exon

As will be understood, an “alternatively-spliced exon” or an “alternatively-regulated exon” or a “cassette exon” refers to certain exons which are either retained (e.g., spliced-in) or excluded (e.g., spliced-out) during post-transcriptional splicing of a pre-mRNA. Whether an alternatively-spliced exon is spliced-in or spliced-out may depend of a number of different factors, including, but not limited to one or more cellular conditions, such as the presence or absence of a disease state (e.g., cancer), type of cell (e.g., liver cell versus skeletal cell), other intracellular conditions, or an external engineered factor (e.g., the administration of an agent).

The differential splicing events result in different spliced transcripts (e.g., mRNA isoforms) that either retain or exclude the alternatively-spliced exon. Further, as disclosed herein, the alternatively-spliced exons may comprise one or more positive or negative regulatory cis-elements that exert a positive or negative regulatory control on the expression of a coding region of interest (or portions thereof). Alternatively-spliced exons may be found in nature in a naturally-occurring gene, or may be modified by changing or altering the sequence thereof, including adding or changing the splice site, and/or adding or changing a positive or negative regulatory cis-element. Such altered exons may be referred to as “recombinant” or “synthetic” exons. “Recombinant” or “synthetic” may in some embodiments include naturally occurring exons that have been placed into a heterologous gene (e.g., an unmodified exon placed into a non-natural context). In some embodiments, the cis-elements mediate localization to a specific cellular compartment, such as, for example, an organelle, the cytoskeleton, plasma membrane, the endoplasmic reticulum, the mitochondria, the nucleus, etc.

### (iv) Cassette

As used herein, the term “cassette” refers to any set of introns and/or exons (including an alternatively-spliced exon) capable of exhibiting a splicing pattern to produce different spliced transcript (e.g., mRNA isoforms).

In some embodiments, when the cassette comprises an alternatively-spliced exon and, in some embodiments, the intronic sequences (or portions thereof) flanking the alternatively-spliced exon, the cassette may be referred to as an “alternative splicing cassette” or equivalently, “alternatively-spliced exon cassette” or “alternative exon cassette.” When situated in an alternatively-spliced exon cassette, an alternative-spliced exon may be alternatively referred to as a “cassette exon.” For purposes of clarity, a “cassette,” and in particular, an “alternatively-spliced exon cassette,” may exclude a coding region of interest, but also may be configured to be operatively linked to any coding region of interest such that the alternatively-spliced exon cassette regulates the expression of the coding region of interest.

### (v) Additional Terms

As used herein, an “engineered intron” is an intron which comprises at least one modification, relative to a native intron. For example, an engineered intron may comprise one or more nucleotide deletions, and thus be truncated, relative to a native intron.

As used herein, an “engineered exon” is an exon which comprises at least one modification, relative to a native exon. For example, an engineered exon may comprise one or more nucleotide deletions, and thus be truncated, relative to a native exon.

As used herein, a “flanking” component (e.g., a flanking intron) refers to a component which is located upstream (e.g., 5′) or downstream (e.g., 3′) of a central component (e.g., an exon). A flanking component may in some embodiments be immediately adjacent to the central component, but that is not required by the methods and compositions of the present disclosure. For example, a central alternatively-spliced exon may, in some embodiments, be flanked by two introns, wherein such introns are immediately adjacent to the central alternatively-spliced exon. The same central alternatively-spliced exon may also be flanked by two additional exons, which are located upstream and downstream of the central alternatively-spliced exon, respectively, but which are not immediately adjacent to the central alternatively-spliced exon.

As used herein, a “constitutive exon” is an exon that is present in all spliced transcripts (e.g., mRNA isoforms) formed as a result of splicing a pre-mRNA transcript that is transcribed from a gene. A constitutive exon is therefore common to different mRNA isoforms of a gene.

Additional terms are defined throughout the disclosure.

### B. Alternative Splicing and Models Used Herein

Through alternative splicing of pre-mRNAs, individual mammalian genes often produce multiple mRNAs (i.e., mRNA isoforms) and resultant protein isoforms that may have related, distinct or even opposing functions. The mRNA and protein isoforms produced by alternative splicing (or equivalently, alternative processing) of primary RNA transcripts may differ in structure, function, localization or other properties. Alternative splicing in particular is known to affect more than half of all human genes, and has been proposed as a primary driver of the evolution of phenotypic complexity in mammals. The number of variants of a gene ranges from two to potentially thousands. The resulting proteins may exhibit different and sometimes antagonistic functional and structural properties, and may inhabit the same cell with the resulting phenotype representing a balance between their expression levels. Defects in splicing have been implicated in human diseases, including cancer.

Aspects of the invention utilize alternative splicing mechanisms as a method of regulating the expression of a transgene (e.g., encoding a therapeutic protein). However, unlike naturally occurring alternatively-spliced exons, the alternatively-spliced exons of the application do not necessarily result in alternative sequence isoforms of the encoded protein. In many embodiments, an alternatively-spliced exon impacts the level of protein expression without impacting the sequence of the protein that is expressed. That is, the alternatively-spliced exon is utilized as a means of regulation of the expression of the protein of interest. In some embodiments, retention of the alternatively-spliced exon in the spliced transcript results in the productive translation of a coding region of interest. In some embodiments, exclusion of the alternatively-spliced exon from the spliced transcript results in the coding region of interest not being translated (e.g., the alternatively-spliced exon is spliced out). In some embodiments, retention of the alternatively-spliced exon in the spliced transcript results in nonsense mediated decay. In some embodiments, exclusion of the alternatively-spliced exon from the spliced transcript results in the productive translation of the coding region of interest.

Thus, by manipulating the composition and arrangement of an alternatively-spliced exon cassette, a recombinant viral genome of the present disclosure comprising the alternatively-spliced exon cassette may behave in a predictable manner, and the transgene and/or coding region of interest may be expressed in specific conditions which are therapeutically beneficial (e.g., in a specific cell type, a specific tissue, a disease state, and/or upon an inflammatory response). Transgenes comprising alternatively-spliced exon cassettes may be designed according to any one of several non-limiting models of alternative splicing (shown in FIG. 2 or 4-8), each of which is specifically contemplated herein, in addition to other models of alternative splicing. Thus, aspects of the invention contemplate alternatively-spliced exon cassettes for regulating the expression of coding regions of interest (e.g., encoding therapeutic proteins).

In various aspects, the alternatively-spliced exons are spliced-in or spliced-out in a manner that is dependent upon one or more environmental cues, e.g., cell or tissue type, disease state, or intracellular conditions. The alternatively-spliced exons can be sourced from a naturally occurring gene or may be recombinant, for example, in order to add one or more genetic regulatory elements for influencing expression levels of the transgene and/or coding region of the transgene. Examples of alternatively-spliced exons are disclosed herein.

In various embodiments, the alternatively-spliced exons may comprise one or more regulatory sequences that modulate the expression of a coding sequence of interest. Such regulatory sequences may be referred to a cis-elements. Further, cis-elements that impart a positive regulatory control on a coding sequence of interest may be referred to as a positive regulatory cis-element. To the contrary, cis-elements that impart a negative regulatory control on a coding sequence of interest may be referred to as a negative regulatory cis-element.

Alternatively-spliced exons may be found in nature in a naturally-occurring genes, or may be modified by changing or altering the sequence thereof (e.g., derived from a naturally-occurring gene), including adding or changing the splice site, and/or adding or changing a positive or negative regulatory cis-element. The one or more positive or negative regulatory cis-elements may be located within an alternatively-spliced exon, and may influence the level of expression of a coding region of interest through positive and/or negative controls, and may include any regulatory sequence which exerts—as a consequence being spliced-in or spliced-out of the final mRNA—either a positive or negative regulation on the expression of the coding region.

FIG. 4 shows three non-limiting embodiments contemplated for the structural configuration of a cassette (e.g., comprised within a transgene) for use with a recombinant virus genome, wherein the cassette (e.g., comprised within a transgene) comprises an alternatively-spliced exon and a coding region, wherein the alternatively-spliced exon further comprises at least one positive or negative regulatory cis-element. Non-limiting examples of positive or negative regulatory cis-elements can include, for instance, (1) a nucleotide sequence element that regulates, modulates, or otherwise affects the stability and/or degradation of a mRNA; and (2) a nucleotide sequence element that regulates, modulates, or otherwise affects the translation of a mRNA into one or more encoded polypeptide products (e.g., a therapeutic product). Without limitation, positive or negative regulatory cis-elements may include, but are not limited to, a translation start codon, a translation stop codon, a binding site for an RNA binding protein that serves to positively regulate transgene expression, a binding site for an RNA binding protein that serves to negatively regulate transgene expression, a binding site for a nucleic acid molecule (e.g., an miRNA) that serves to positively regulate transgene expression, or a binding site for a nucleic acid molecule (e.g., an siRNA) that serves to negatively regulate transgene expression. This list of examples is not intended to place any limitation on the scope or meaning of the positive and negative regulatory cis-elements and the disclosure embraces any genetic element or region positioned within or at least associated with an alternatively-spliced exon which exerts a positive or negative control on the overall expression of a transgene (e.g., encoding a therapeutic protein).

In some embodiments, the one or more cis-elements can include, but are not limited to, a translation start codon, a translation stop codon, an siRNA binding site, a miRNA binding site, a sequence forming a stem-loop structure, a sequence forming an RNA dimerization motif, a sequence forming a hairpin structure, a sequence forming an RNA quadruplex, polypurine tract, a sequence forming a pair of kissing loops, and a sequence forming a tetraloop/tetraloop receptor pair. In some embodiments, cis-elements include binding sites recognized by regulatory elements, such as, for example, RNA binding proteins. In some embodiments, an RNA binding protein capable of exerting regulatory control once bound is an RNA binding protein described in Van Nostrand, et al. (2020), A large-scale binding and functional map of human RNA-binding proteins, Nature, 583: 711-719, which is herein incorporated by reference with respect to its description of RNA binding proteins.

In various embodiments, the cassettes (e.g., comprised within a transgene) may include one or more additional components, including one or more other constitutive exons, and one or more introns. In FIGS. 4A-4C, the constitutive exons not comprising the coding region of interest are represented by narrow rectangles, introns are represented as dashed lines, and the alternatively-spliced exons are represented as shaded narrow rectangles. In some embodiments, the exon or exons comprising the coding region (or portions thereof, in embodiments wherein the coding region is split into separate exons) are indicated as solid thick white rectangles. In other embodiments, the alternatively-spliced exon may contain portions of a coding region of interest.

FIG. 4A is a schematic of an embodiment wherein the alternatively-spliced exon is upstream of the exon encoding the coding region of interest. Said another way, in this embodiment, the alternatively-spliced exon is to the 5′ of the exon encoding the coding region of interest.

FIG. 4B is a schematic of an embodiment wherein the alternatively-spliced exon is downstream of the exon encoding the coding region of interest. Said another way, in this embodiment, the alternatively-spliced exon is to the 3′ of the exon encoding the coding region of interest.

FIG. 4C is a schematic of an embodiment wherein the alternatively-spliced exon is positioned between two separate exons encoding portions of the coding region of interest. Said another way, in this embodiment, the alternatively-spliced exon is between the exons encoding the portions of the coding region of interest.

Various specific embodiments of these general groups of configurations are further shown in FIGS. 3-8, and further described as follows.

In some embodiments, a transgene comprising an alternatively-spliced exon cassette comprises a polynucleotide sequence as set forth in any one of SEQ ID NOs: 45-55. In some embodiments, a transgene comprising an alternatively-spliced exon cassette comprises a polynucleotide sequence that is 90%, 91%, 92%, 93%, 94%, 95%, 96%, 97%, 98%, or 99% identical to any one of SEQ ID NOs: 45-55.

### (i) Skipped Exon Model of Alternative Splicing

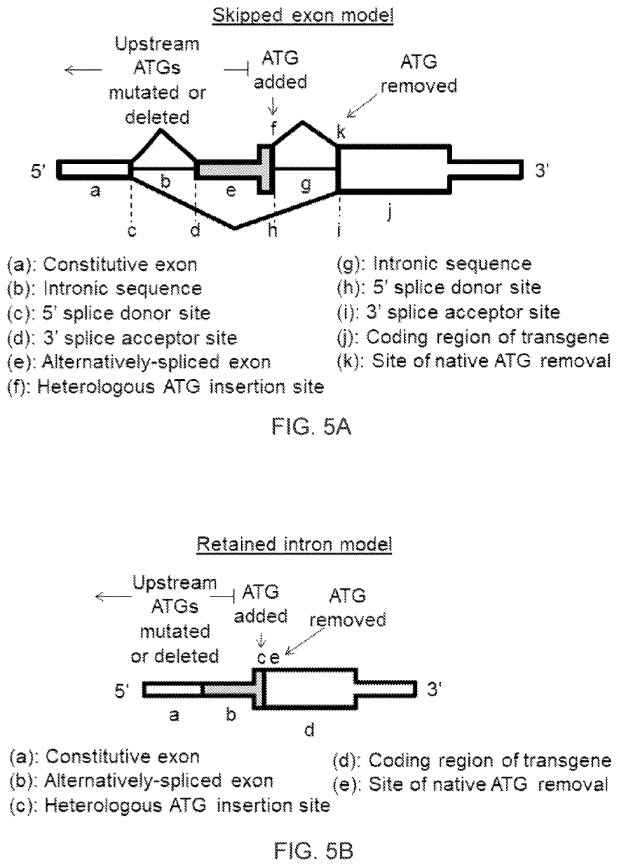

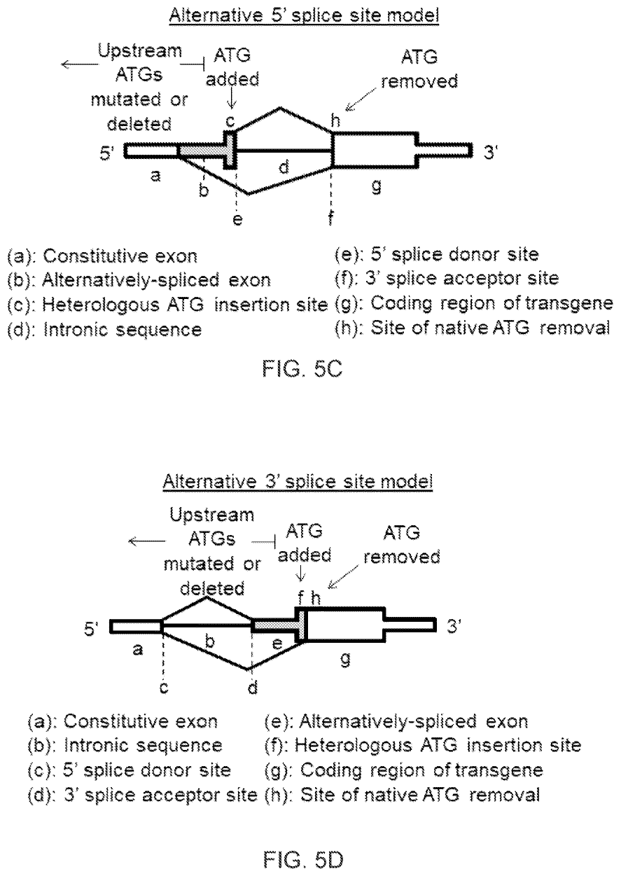

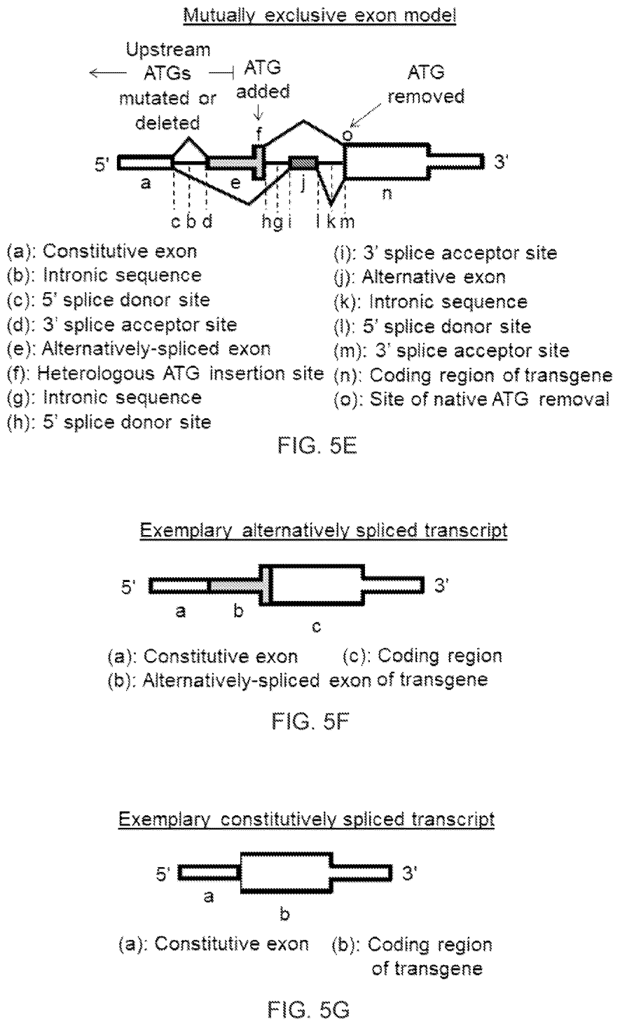

In some embodiments, the nucleic acid vectors of the present invention comprise a transgene comprising an alternatively-spliced exon cassette comprising components which, when alternatively spliced, comprise a skipped exon model of alternative splicing (see, e.g., FIGS. 5A, 6B, and 7A).

Referencing the components as labeled in FIG. 5A, in some embodiments the transgene comprising an alternatively-spliced exon cassette comprises, in the 5′ to 3′ direction:
a nucleotide sequence comprising a first exonic sequence having a 5′ to 3′ orientation (a), wherein the first exonic sequence comprises a constitutive exon;
a nucleotide sequence comprising a first intronic sequence having a 5′ to 3′ orientation (b), wherein the first intronic sequence comprises at its 5′ end a 5′ splice donor site (c) and at its 3′ end a 3′ splice acceptor site (d);
a nucleotide sequence comprising a second exonic sequence having a 5′ to 3′ orientation (e), wherein the second exonic sequence comprises an alternatively-spliced exon comprising at its 3′ end a heterologous ATG start codon (f);
a nucleotide sequence comprising a second intronic sequence having a 5′ to 3′ orientation (g), wherein the second intronic sequence comprises at its 5′ end a 5′ splice donor site (h) and at its 3′ end a 3′ splice acceptor site (i); and
a nucleotide sequence comprising a coding region of interest having a 5′ to 3′ orientation (j), wherein the coding region of interest comprises at its 5′ end a modification comprising the removal of a native ATG start codon (k), and
wherein all native ATG start codons located upstream (e.g., 5′) of the heterologous ATG start codon (f) are mutated or deleted.

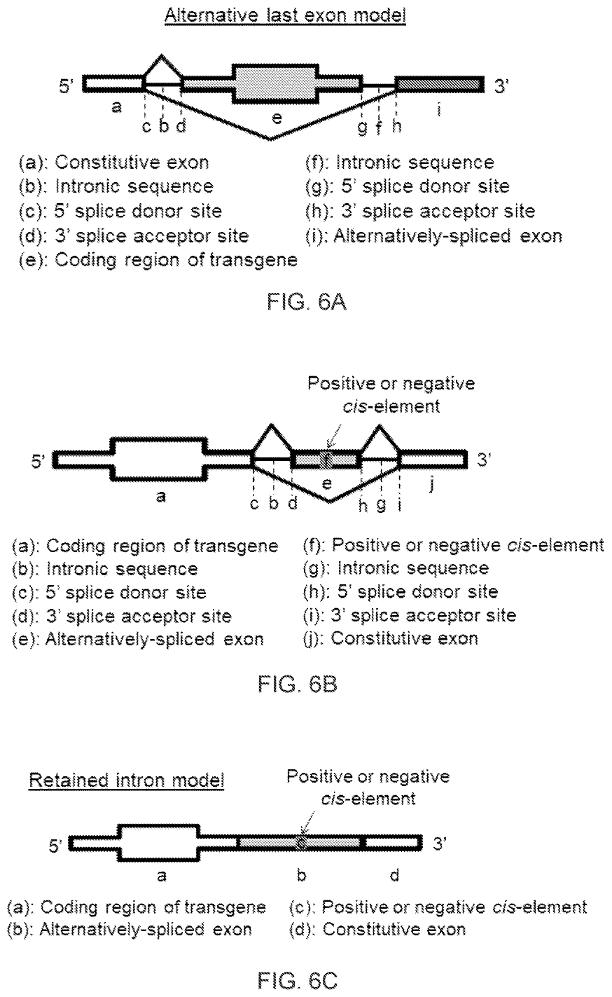

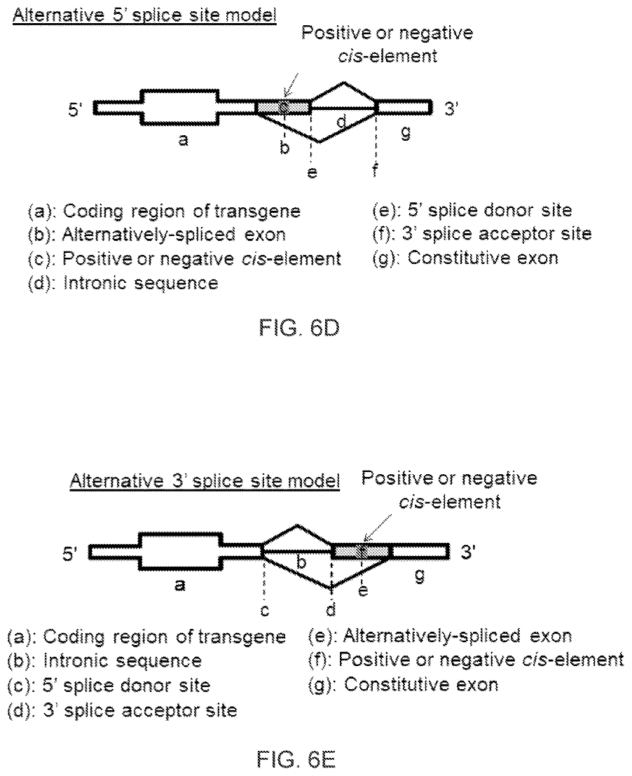

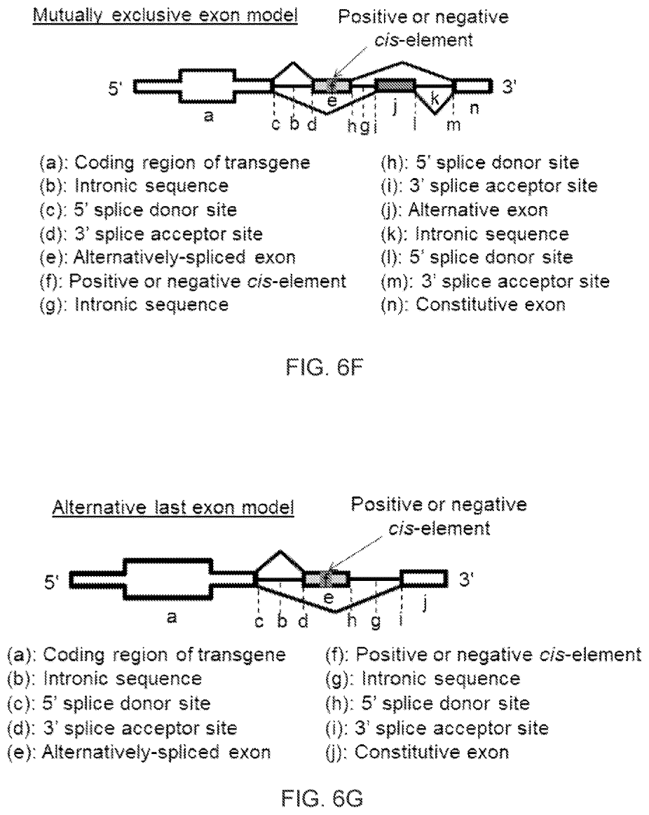

Referencing the components as labeled in FIG. 6B, in some embodiments the transgene comprising an alternatively-spliced exon cassette comprises, in the 5′ to 3′ direction:
a nucleotide sequence comprising a coding region of interest having a 5′ to 3′ orientation (a);
a nucleotide sequence comprising a first intronic sequence having a 5′ to 3′ orientation (b), wherein the first intronic sequence comprises at its 5′ end a 5′ splice donor site (c) and at its 3′ end a 3′ splice acceptor site (d);
a nucleotide sequence comprising a first exonic sequence having a 5′ to 3′ orientation (e), wherein the first exonic sequence comprises an alternatively-spliced exon comprising a positive or negative cis-acting element (f);
a nucleotide sequence comprising a second intronic sequence having a 5′ to 3′ orientation (g), wherein the second intronic sequence comprises at its 5′ end a 5′ splice donor site (h) and at its 3′ end a 3′ splice acceptor site (i); and
a nucleotide sequence comprising a second exonic sequence having a 5′ to 3′ orientation (j), wherein the exonic sequence comprises a constitutive exon.

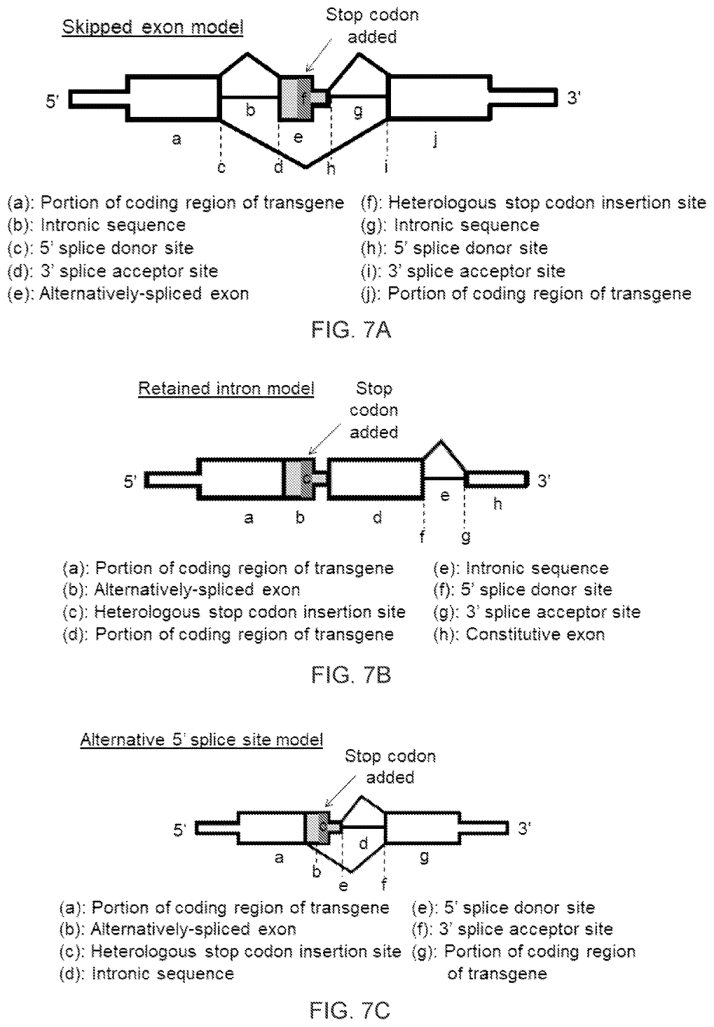

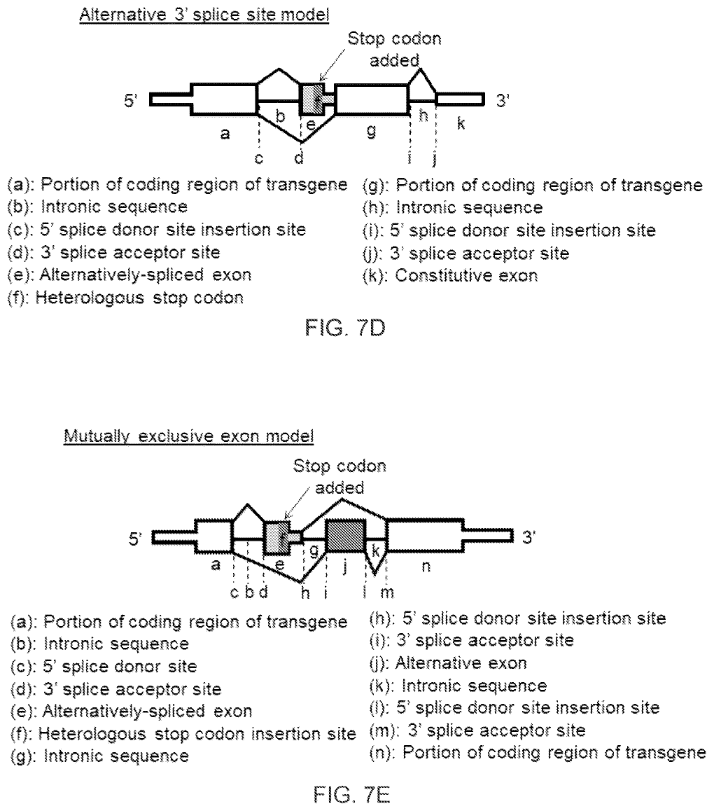

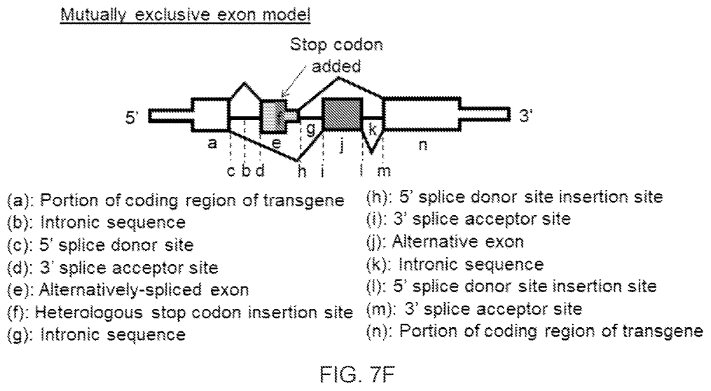

Referencing the components as labeled in FIG. 7A, in some embodiments the transgene comprising an alternatively-spliced exon cassette comprises, in the 5′ to 3′ direction:
a nucleotide sequence comprising a first portion of a coding region of interest having a 5′ to 3′ orientation (a);
a nucleotide sequence comprising a first intronic sequence having a 5′ to 3′ orientation (b), wherein the first intronic sequence comprises at its 5′ end a 5′ splice donor site (c) and at its 3′ end a 3′ splice acceptor site (d);
a nucleotide sequence comprising an exonic sequence having a 5′ to 3′ orientation (e), wherein the exonic sequence comprises an alternatively-spliced exon comprising at its 3′ end a heterologous stop codon (f);
a nucleotide sequence comprising a second intronic sequence having a 5′ to 3′ orientation (g), wherein the second intronic sequence comprises at its 5′ end a 5′ splice donor site (h) and at its 3′ end a 3′ splice acceptor site (i); and
a nucleotide sequence comprising a second portion of a coding region of interest having a 5′ to 3′ orientation (j).

In some embodiments, retention of the alternatively-spliced exon in the spliced transcript results in expression of the coding region of interest. In some embodiments, retention of the alternatively-spliced exon in the spliced transcript does not result in expression of the coding region of interest. In some embodiments, retention of the alternatively-spliced exon in the spliced transcript results in nonsense-mediated decay. In some embodiments, retention of the alternatively-spliced exon in the spliced transcript does not result in nonsense-mediated decay. In some embodiments, retention of the alternatively-spliced exon in the spliced transcript results in expression of the coding region of interest, wherein expression of the coding region of interest is regulated by a positive or negative cis-acting element. In some embodiments, retention of the alternatively-spliced exon in the spliced transcript results in expression of the coding region of interest, wherein expression of the coding region of interest is not regulated by a positive or negative cis-acting element.

### (ii) Retained Intron Model of Alternative Splicing

In some embodiments, the nucleic acid vectors of the present invention comprise a transgene comprising an alternatively-spliced exon cassette comprising components which, when alternatively spliced, comprise a retained intron model of alternative splicing (see, e.g., FIGS. 5B, 6C, and 7B).

Referencing the components as labeled in FIG. 5B, in some embodiments the transgene comprising an alternatively-spliced exon cassette comprises, in the 5′ to 3′ direction:
a nucleotide sequence comprising a first exonic sequence having a 5′ to 3′ orientation (a), wherein the first exonic sequence comprises a constitutive exon;
a nucleotide sequence comprising a second exonic sequence having a 5′ to 3′ orientation (b), wherein the second exonic sequence comprises an alternatively-spliced exon comprising at its 3′ end a heterologous ATG start codon (c); and
a nucleotide sequence comprising a coding region of interest having a 5′ to 3′ orientation (d), wherein the coding region of interest comprises at its 5′ end a modification comprising the removal of a native ATG start codon (e), and
wherein all native ATG start codons located upstream (e.g., 5′) of the heterologous ATG start codon (c) are mutated or deleted.

Referencing the components as labeled in FIG. 6C, in some embodiments, the transgene comprising an alternatively-spliced exon cassette comprises, in the 5′ to 3′ direction:
a nucleotide sequence comprising a coding region of interest having a 5′ to 3′ orientation (a);
a nucleotide sequence comprising a first exonic sequence having a 5′ to 3′ orientation (b), wherein the first exonic sequence comprises an alternatively-spliced exon comprising a positive or negative cis-acting element (c); and
a nucleotide sequence comprising a second exonic sequence having a 5′ to 3′ orientation (d), wherein the second exonic sequence comprises a constitutive exon.

Referencing the components as labeled in FIG. 7B, in some embodiments the transgene comprising an alternatively-spliced exon cassette comprises, in the 5′ to 3′ direction:
a nucleotide sequence comprising a first portion of a coding region of interest having a 5′ to 3′ orientation (a);
a nucleotide sequence comprising a first exonic sequence having a 5′ to 3′ orientation (b), wherein the first exonic sequence comprises an alternatively-spliced exon comprising at its 3′ end a heterologous stop codon (c);
a nucleotide sequence comprising a second portion of a coding region of interest having a 5′ to 3′ orientation (d);
a nucleotide sequence comprising an intronic sequence having a 5′ to 3′ orientation (e), wherein the intronic sequence comprises at its 5′ end a 5′ splice donor site (f) and at its 3′ end a 3′ splice acceptor site (g); and
a nucleotide sequence comprising a second exonic sequence having a 5′ to 3′ orientation (h), wherein the second exonic sequence comprises a constitutive exon.

In some embodiments, retention of the alternative exon in the spliced transcript results in expression of the coding region of interest. In some embodiments, retention of the alternatively-spliced exon in the spliced transcript does not result in expression of the coding region of interest. In some embodiments, retention of the alternatively-spliced exon in the spliced transcript results in nonsense-mediated decay. In some embodiments, retention of the alternatively-spliced exon in the spliced transcript does not result in nonsense-mediated decay. In some embodiments, retention of the alternatively-spliced exon in the spliced transcript results in expression of the coding region of interest, wherein expression of the coding region of interest is regulated by a positive or negative cis-acting element. In some embodiments, retention of the alternatively-spliced exon in the spliced transcript results in expression of the coding region of interest, wherein expression of the coding region of interest is not regulated by a positive or negative cis-acting element.

(iii) Alternative 5′ Splice Site Model of Alternative Splicing

In some embodiments, the nucleic acid vectors of the present invention comprise a transgene comprising an alternatively-spliced exon cassette comprising components which, when alternatively spliced, comprise an alternative 5′ donor site model of alternative splicing (see, e.g., FIGS. 5C, 6D, and 7C).

Referencing the components as labeled in FIG. 5C, in some embodiments the transgene comprising an alternatively-spliced exon cassette comprises, in the 5′ to 3′ direction:
a nucleotide sequence comprising a first exonic sequence having a 5′ to 3′ orientation (a), wherein the first exonic sequence comprises a constitutive exon;
a nucleotide sequence comprising a second exonic sequence having a 5′ to 3′ orientation (b), wherein the second exonic sequence comprises an alternatively-spliced exon comprising at its 3′ end a heterologous ATG start codon (c);
a nucleotide sequence comprising an intronic sequence having a 5′ to 3′ orientation (d), wherein the intronic sequence comprises at its 5′ end a 5′ splice donor site (e) and at its 3′ end a 3′ splice acceptor site (f); and
a nucleotide sequence comprising a coding region of interest having a 5′ to 3′ orientation (g), wherein the coding region of interest comprises at its 5′ end a modification comprising the removal of a native ATG start codon (h), and
wherein all native ATG start codons located upstream (e.g., 5′) of the heterologous ATG start codon (c) are mutated or deleted.

Referencing the components as labeled in FIG. 6D, in some embodiments the transgene comprising an alternatively-spliced exon cassette comprises, in the 5′ to 3′ direction:
a nucleotide sequence comprising a coding region of interest having a 5′ to 3′ orientation (a);
a nucleotide sequence comprising a first exonic sequence having a 5′ to 3′ orientation (b), wherein the exonic sequence comprises an alternatively-spliced exon comprising a positive or negative cis-acting element (c);
a nucleotide sequence comprising an intronic sequence having a 5′ to 3′ orientation (d), wherein the intronic sequence comprises at its 5′ end a 5′ splice donor site (e) and at its 3′ end a 3′ splice acceptor site (f); and
a nucleotide sequence comprising a second exonic sequence having a 5′ to 3′ orientation (g), wherein the exonic sequence comprises a constitutive exon.

Referencing the components as labeled in FIG. 7C, in some embodiments the transgene comprising an alternatively-spliced exon cassette comprises, in the 5′ to 3′ direction:
a nucleotide sequence comprising a first portion of a transgene having a 5′ to 3′ orientation (a);
a nucleotide sequence comprising an exonic sequence having a 5′ to 3′ orientation (b), wherein the exonic sequence comprises an alternatively-spliced exon comprising at its 3′ end a heterologous stop codon (c);
a nucleotide sequence comprising an intronic sequence having a 5′ to 3′ orientation (d), wherein the intronic sequence comprises at its 5′ end a 5′ splice donor site (e) and at its 3′ end a 3′ splice acceptor site (f); and
a nucleotide sequence comprising a second portion of a transgene having a 5′ to 3′ orientation (g).

In some embodiments, retention of the alternatively-spliced exon in the spliced transcript results in expression of the coding region of interest. In some embodiments, retention of the alternatively-spliced exon in the spliced transcript does not result in expression of the coding region of interest. In some embodiments, retention of the alternatively-spliced exon in the spliced transcript results in nonsense-mediated decay. In some embodiments, retention of the alternatively-spliced exon in the spliced transcript does not result in nonsense-mediated decay. In some embodiments, retention of the alternatively-spliced exon in the spliced transcript results in expression of the coding region of interest, wherein expression of the coding region of interest is regulated by a positive or negative cis-acting element. In some embodiments, retention of the alternatively-spliced exon in the spliced transcript results in expression of the coding region of interest, wherein expression of the coding region of interest is not regulated by a positive or negative cis-acting element.

### (iv) Alternative 3′ Splice Site Model of Alternative Splicing

In some embodiments, the nucleic acid vectors of the present invention comprise a transgene comprising an alternatively-spliced exon cassette comprising components which, when alternatively spliced, comprise an alternative 3′ donor site model of alternative splicing (see, e.g., FIGS. 5D, 6E, and 7D).

Referencing the components as labeled in FIG. 5D, in some embodiments the transgene comprising an alternatively-spliced exon cassette comprises, in the 5′ to 3′ direction:
a nucleotide sequence comprising a first exonic sequence having a 5′ to 3′ orientation (a), wherein the first exonic sequence comprises a constitutive exon;
a nucleotide sequence comprising an intronic sequence having a 5′ to 3′ orientation (b), wherein the intronic sequence comprises at its 5′ end a 5′ splice donor site (c) and at its 3′ end a 3′ splice acceptor site (d);
a nucleotide sequence comprising a second exonic sequence having a 5′ to 3′ orientation (e), wherein the second exonic sequence comprises an alternatively-spliced exon comprising at its 3′ end a heterologous ATG start codon (f); and
a nucleotide sequence comprising a coding region of interest having a 5′ to 3′ orientation (g), wherein the coding region of interest comprises at its 5′ end a modification comprising the removal of a native ATG start codon (h),
wherein all native ATG start codons located upstream (e.g., 5′) of the heterologous ATG start codon (f) are mutated or deleted.

Referencing the components as labeled in FIG. 6E, in some embodiments the transgene comprising an alternatively-spliced exon cassette comprises, in the 5′ to 3′ direction:
a nucleotide sequence comprising a coding region of interest having a 5′ to 3′ orientation (a);
a nucleotide sequence comprising an intronic sequence having a 5′ to 3′ orientation (b), wherein the intronic sequence comprises at its 5′ end a 5′ splice donor site (c) and at its 3′ end a 3′ splice acceptor site (d);
a nucleotide sequence comprising an exonic sequence having a 5′ to 3′ orientation (e), wherein the exonic sequence comprises an alternatively-spliced exon comprising a positive or negative cis-acting element (f); and
a nucleotide sequence comprising a second exonic sequence having a 5′ to 3′ orientation (g), wherein the exonic sequence comprises a constitutive exon.

Referencing the components as labeled in FIG. 7D, in some embodiments the transgene comprising an alternatively-spliced exon cassette comprises, in the 5′ to 3′ direction:
a nucleotide sequence comprising a first portion of a coding region of interest having a 5′ to 3′ orientation (a);
a nucleotide sequence comprising a first intronic sequence having a 5′ to 3′ orientation (b), wherein the first intronic sequence comprises at its 5′ end a 5′ splice donor site (c) and at its 3′ end a 3′ splice acceptor site (d);
a nucleotide sequence comprising a first exonic sequence having a 5′ to 3′ orientation (e), wherein the first exonic sequence comprises an alternatively-spliced exon comprising at its 3′ end a heterologous stop codon (f);
a nucleotide sequence comprising a second portion of a coding region of interest having a 5′ to 3′ orientation (g);
a nucleotide sequence comprising a second intronic sequence having a 5′ to 3′ orientation (h), wherein the second intronic sequence comprises at its 5′ end a 5′ splice donor site (i) and at its 3′ end a 3′ splice acceptor site (j); and
a nucleotide sequence comprising a second exonic sequence having a 5′ to 3′ orientation (k), wherein the second exonic sequence comprises a constitutive exon.

In some embodiments, retention of the alternatively-spliced exon in the spliced transcript results in expression of the coding region of interest. In some embodiments, retention of the alternatively-spliced exon in the spliced transcript does not result in expression of the coding region of interest. In some embodiments, retention of the alternatively-spliced exon in the spliced transcript results in nonsense-mediated decay. In some embodiments, retention of the alternatively-spliced exon in the spliced transcript does not result in nonsense-mediated decay. In some embodiments, retention of the alternatively-spliced exon in the spliced transcript results in expression of the coding region of interest, wherein expression of the coding region of interest is regulated by a positive or negative cis-acting element. In some embodiments, retention of the alternatively-spliced exon in the spliced transcript results in expression of the coding region of interest, wherein expression of the coding region of interest is not regulated by a positive or negative cis-acting element.

### (v) Mutually Exclusive Exon Model of Alternative Splicing

In some embodiments, the nucleic acid vectors of the present invention comprise a transgene comprising an alternatively-spliced exon cassette comprising components which, when alternatively spliced, comprise a mutually exclusive exon model of alternative splicing (see, e.g., FIGS. 5E, 6F, and 7E).

Referencing the components as labeled in FIG. 5E, in some embodiments the transgene comprising an alternatively-spliced exon cassette comprises, in the 5′ to 3′ direction:
a nucleotide sequence comprising a first exonic sequence having a 5′ to 3′ orientation (a), wherein the first exonic sequence comprises a constitutive exon;
a nucleotide sequence comprising a first intronic sequence having a 5′ to 3′ orientation (b), wherein the first intronic sequence comprises at its 5′ end a 5′ splice donor site (c) and at its 3′ end a 3′ splice acceptor site (d);
a nucleotide sequence comprising a second exonic sequence having a 5′ to 3′ orientation (e), wherein the second exonic sequence comprises an alternatively-spliced exon comprising at its 3′ end a heterologous ATG start codon (f);
a nucleotide sequence comprising a second intronic sequence having a 5′ to 3′ orientation (g), wherein the second intronic sequence comprises at its 5′ end a 5′ splice donor site (h) and at its 3′ end a 3′ splice acceptor site (i);
a nucleotide sequence comprising a third exonic sequence having a 5′ to 3′ orientation (j), wherein the third exonic sequence comprises an alternatively-spliced exon;
a nucleotide sequence comprising a third intronic sequence having a 5′ to 3′ orientation (k), wherein the third intronic sequence comprises at its 5′ end a 5′ splice donor site (1) and at its 3′ end a 3′ splice acceptor site (m); and
a nucleotide sequence comprising a coding region of interest having a 5′ to 3′ orientation (n), wherein the coding region of interest comprises at its 5′ end a modification comprising the removal of a native ATG start codon (o),
wherein all native ATG start codons located upstream (e.g., 5′) of the heterologous ATG start codon (f) are mutated or deleted.

Referencing the components as labeled in FIG. 6F, in some embodiments, the transgene comprising an alternatively-spliced exon cassette comprises, in the 5′ to 3′ direction:
a nucleotide sequence comprising a coding region of interest having a 5′ to 3′ orientation (a);
a nucleotide sequence comprising a first intronic sequence having a 5′ to 3′ orientation (b), wherein the first intronic sequence comprises at its 5′ end a 5′ splice donor site (c) and at its 3′ end a 3′ splice acceptor site (d);
a nucleotide sequence comprising a first exonic sequence having a 5′ to 3′ orientation (e), wherein the first exonic sequence comprises a first alternatively-spliced exon comprising a positive or negative cis-acting element (f);
a nucleotide sequence comprising a second intronic sequence having a 5′ to 3′ orientation (g), wherein the second intronic sequence comprises at its 5′ end a 5′ splice donor site (h) and at its 3′ end a 3′ splice acceptor site (i);
a nucleotide sequence comprising a second exonic sequence having a 5′ to 3′ orientation (j), wherein the second exonic sequence comprises a second alternatively-spliced exon;
a nucleotide sequence comprising a third intronic sequence having a 5′ to 3′ orientation (k), wherein the third intronic sequence comprises at its 5′ end a 5′ splice donor site (1) and at its 3′ end a 3′ splice acceptor site (m); and a nucleotide sequence comprising a third exonic sequence having a 5′ to 3′ orientation (n), wherein the third exonic sequence comprises a constitutive exon.

Referencing the components as labeled in FIG. 7E, in some embodiments the transgene comprising an alternatively-spliced exon cassette comprises, in the 5′ to 3′ direction:
a nucleotide sequence comprising a first portion of a coding region of interest having a 5′ to 3′ orientation (a);
a nucleotide sequence comprising a first intronic sequence having a 5′ to 3′ orientation (b), wherein the first intronic sequence comprises at its 5′ end a 5′ splice donor site (c) and at its 3′ end a 3′ splice acceptor site (d);
a nucleotide sequence comprising a first exonic sequence having a 5′ to 3′ orientation (e), wherein the first exonic sequence comprises an alternatively-spliced exon comprising at its 3′ end a heterologous stop codon (f);
a nucleotide sequence comprising a second intronic sequence having a 5′ to 3′ orientation (g), wherein the second intronic sequence comprises at its 5′ end a 5′ splice donor site (h) and at its 3′ end a 3′ splice acceptor site (i);
a nucleotide sequence comprising a second exonic sequence having a 5′ to 3′ orientation (j), wherein the second exonic sequence comprises an alternatively-spliced exon;
a nucleotide sequence comprising a third intronic sequence having a 5′ to 3′ orientation (k), wherein the third intronic sequence comprises at its 5′ end a 5′ splice donor site (1) and at its 3′ end a 3′ splice acceptor site (m); and
a nucleotide sequence comprising a second portion of a coding region of interest having a 5′ to 3′ orientation (n).

In some embodiments, retention of the alternatively-spliced exon in the spliced transcript results in expression of the coding region of interest. In some embodiments, retention of the alternatively-spliced exon in the spliced transcript does not result in expression of the coding region of interest. In some embodiments, retention of the alternatively-spliced exon in the spliced transcript results in nonsense-mediated decay. In some embodiments, retention of the alternatively-spliced exon in the spliced transcript does not result in nonsense-mediated decay. In some embodiments, retention of the alternatively-spliced exon in the spliced transcript results in expression of the coding region of interest, wherein expression of the coding region of interest is regulated by a positive or negative cis-acting element. In some embodiments, retention of the alternatively-spliced exon in the spliced transcript results in expression of the coding region of interest, wherein expression of the coding region of interest is not regulated by a positive or negative cis-acting element.

### (vi) Alternative Last Exon Model of Alternative Splicing

In some embodiments, the nucleic acid vectors of the present invention comprise a transgene comprising an alternatively-spliced exon cassette comprising components which, when alternatively spliced, comprise an alternative last exon model of alternative splicing (see, e.g., FIGS. 6A, 6G, and 7F).

Referencing the components as labeled in FIG. 6A, in some embodiments the transgene comprising an alternatively-spliced exon cassette comprises, in the 5′ to 3′ direction:
a nucleotide sequence comprising a first exonic sequence having a 5′ to 3′ orientation (a), wherein the first exonic sequence comprises a constitutive exon;
a nucleotide sequence comprising a first intronic sequence having a 5′ to 3′ orientation (b), wherein the first intronic sequence comprises at its 5′ end a 5′ splice donor site (c) and at its 3′ end a 3′ splice acceptor site (d);
a nucleotide sequence comprising a coding region of interest having a 5′ to 3′ orientation (e);
a nucleotide sequence comprising a second intronic sequence having a 5′ to 3′ orientation (f), wherein the second intronic sequence comprises at its 5′ end a 5′ splice donor site (g) and at its 3′ end a 3′ splice acceptor site (h); and
a nucleotide sequence comprising a second exonic sequence having a 5′ to 3′ orientation (i), wherein the second exonic sequence comprises an alternatively-spliced exon.

Referencing the components as labeled in FIG. 6G, in some embodiments the transgene comprising an alternatively-spliced exon cassette comprises, in the 5′ to 3′ direction:
a nucleotide sequence comprising a coding region of interest having a 5′ to 3′ orientation (a);
a nucleotide sequence comprising a first intronic sequence having a 5′ to 3′ orientation (b), wherein the first intronic sequence comprises at its 5′ end a 5′ splice donor site (c) and at its 3′ end a 3′ splice acceptor site (d);
a nucleotide sequence comprising a first exonic sequence having a 5′ to 3′ orientation (e), wherein the first exonic sequence comprises an alternatively-spliced exon comprising a positive or negative cis-acting element (f);
a nucleotide sequence comprising a second intronic sequence having a 5′ to 3′ orientation (g), wherein the second intronic sequence comprises at its 5′ end a 5′ splice donor site (h) and at its 3′ end a 3′ splice acceptor site (i);
a nucleotide sequence comprising a second exonic sequence having a 5′ to 3′ orientation (j), wherein the second exonic sequence comprises a constitutive exon.

Referencing the components as labeled in FIG. 7F, in some embodiments the transgene comprising an alternatively-spliced exon cassette comprises, in the 5′ to 3′ direction:
a nucleotide sequence comprising a first portion of a transgene having a 5′ to 3′ orientation (a);
a nucleotide sequence comprising a first intronic sequence having a 5′ to 3′ orientation (b), wherein the first intronic sequence comprises at its 5′ end a 5′ splice donor site (c) and at its 3′ end a 3′ splice acceptor site (d);
a nucleotide sequence comprising a first exonic sequence having a 5′ to 3′ orientation (e), wherein the first exonic sequence comprises an alternatively-spliced exon comprising at its 3′ end a heterologous stop codon (f);
a nucleotide sequence comprising a second intronic sequence having a 5′ to 3′ orientation (g), wherein the second intronic sequence comprises at its 5′ end a 5′ splice donor site (h) and at its 3′ end a 3′ splice acceptor site (i);
a nucleotide sequence comprising a second exonic sequence having a 5′ to 3′ orientation (j), wherein the second exonic sequence comprises a constitutive exon;
a nucleotide sequence comprising a third intronic sequence having a 5′ to 3′ orientation (k), wherein the third intronic sequence comprises at its 5′ end a 5′ splice donor site (1) and at its 3′ end a 3′ splice acceptor site (m); and
a nucleotide sequence comprising a second portion of a coding region of interest having a 5′ to 3′ orientation (n).

In some embodiments, retention of the alternatively-spliced exon in the spliced transcript results in expression of the coding region of interest. In some embodiments, retention of the alternatively-spliced exon in the spliced transcript does not result in expression of the coding region of interest. In some embodiments, retention of the alternatively-spliced exon in the spliced transcript results in nonsense-mediated decay. In some embodiments, retention of the alternatively-spliced exon in the spliced transcript does not result in nonsense-mediated decay. In some embodiments, retention of the alternatively-spliced exon in the spliced transcript results in expression of the coding region of interest, wherein expression of the coding region of interest is regulated by a positive or negative cis-acting element. In some embodiments, retention of the alternatively-spliced exon in the spliced transcript results in expression of the coding region of interest, wherein expression of the coding region of interest is not regulated by a positive or negative cis-acting element.

### C. Components of the Recombinant Vector Genomes

In some embodiments, a nucleic acid vector (e.g., a viral vector) of the present invention comprises a transgene comprising at least one alternatively-spliced exon cassette as described herein. Nucleic acid vectors or transgenes may have one alternatively-spliced exon cassette, or multiple such cassettes. In some embodiments, a nucleic acid vector or transgene comprises 1, 2, 3, 4, 5, 6, 7, 8, 9, 10, 11, 12, 13, 14, or 15, or more alternatively-spliced exon cassettes. A transgene comprising an alternatively-spliced exon cassette may, in some embodiments, comprise any one or more of the following components: an alternatively-spliced exon, an intron (e.g., a flanking intron), an exon comprising a coding region of interest, and/or a constitutive exon. In some embodiments, transgene comprising an alternatively-spliced exon cassette comprises an alternatively-spliced exon, a flanking intron, and an exon comprising a coding region of interest (wherein, in some embodiments, the coding region of interest may be split into portions across two or more exons).

### (i) Alternatively-Spliced Exons

In some embodiments, a nucleic acid vector or transgene comprises an alternatively-spliced exon cassette, wherein the alternatively-spliced exon cassette comprises among other components at least one alternatively-spliced exon. In some embodiments, the alternatively-spliced exon cassette comprises 1, 2, 3, or 4 alternatively-spliced exons. In some other embodiments, the alternatively-spliced exon cassette comprises 1, 2, 3, 4, 5, 6, 7, 8, 9, 10, 11, 12, 13, 14, 15, 16, 17, 18, 19, 20, 21, 22, 23, 24, or 25 alternatively-spliced exons. In some embodiments, wherein the alternatively-spliced exon cassette comprises more than one alternatively-spliced exon, the alternatively-spliced exons are adjacent. In some embodiments, wherein the alternatively-spliced exon cassette comprises more than one alternatively-spliced exon, the alternatively-spliced exons are not adjacent.

In some embodiments, the alternatively-spliced exon is synthetic or recombinant. In some embodiments, the alternatively-spliced exon is considered to be synthetic or recombinant because it undergoes one or more nucleic acid modifications, relative to the wild-type alternatively-spliced exon. A nucleic acid modification may be a substitution or deletion of one or more nucleotides that form the nucleic acid sequence of the alternatively-spliced exon.

In some embodiments, an alternative exon comprises an ATG start codon at its 3′ end. In some embodiments, the “3′ end” comprises the 1, 2, or 3 nucleic acids lying at the 3′ end of the alternative exon. As will be understood, in some embodiments a wild-type or naturally occurring alternative exon may comprise an ATG start codon at its 3′ end. In such embodiments, the alternative exon may comprise nucleic acid modifications unrelated to the insertion of a heterologous start codon at the 3′ end of the alternative exon. However, it will be further understood that in some embodiments a wild-type or naturally occurring alternative exon may not comprise an ATG start codon at its 3′ end. In such embodiments, modifications are made to the 3′ end of the alternative exon to introduce a heterologous start codon, such that when the alternative exon is spliced-in or retained in the spliced transcript, the downstream coding sequence is translated as a full-length protein. As will be understood, in some embodiments 1, 2, or 3 nucleic acid substitutions may be necessary in order to introduce the heterologous ATG start codon to the 3′ end of the alternative exon, depending on the sequence which is present at the 3′ end of the wild-type or naturally occurring alternative exon. In such embodiments, the 3′ end of the alternatively-spliced exon comprises 1 nucleotide substitution, relative to the wild-type alternatively-spliced exon, to form the ATG start codon. In such embodiments, the 3′ end of the alternatively-spliced exon comprises 2 nucleotide substitutions, relative to the wild-type alternatively-spliced exon, to form the ATG start codon. In such embodiments, the 3′ end of the alternatively-spliced exon comprises 3 nucleotide substitutions, relative to the wild-type alternatively-spliced exon, to form the ATG start codon.

In some embodiments, the modification comprises the insertion of a heterologous start codon or part of a heterologous start codon at the 3′ end of the alternatively-spliced exon (e.g., 1-3 nucleic acids are added to the 3′ end of the alternatively-spliced exon, rather than substituted, to form an ATG start codon).

In some embodiments, an alternative exon comprises part of an ATG start codon at its 3′ end. In some embodiments, an alternative exon may comprise, for example, “A” as the last nucleic acid, or “AT” as the last two nucleic acids, which formulate the 3′ end of the alternative exon. In such embodiments, the remainder of the ATG start codon may lie at the 5′ end of an exon lying immediately downstream of the alternative exon. For example, in some embodiments the alternative exon may comprise “A” as the last nucleic acid which formulates the 3′ end of the alternative exon, and the exon lying immediately downstream of the alternative exon may comprise “TG” as the first two nucleic acids which formulate the 5′ end of the downstream exon. In some embodiments, the alternative exon may comprise “AT” as the last two nucleic acids which formulate the 3′ end of the alternative exon, and the exon lying immediately downstream of the alternative exon may comprise “G” as the first nucleic acid which formulates the 5′ end of the downstream exon. In some embodiments, the ATG formed as a result of the splicing together of the alternative exon and the exon lying immediately downstream of the alternative exon initiates translation of the exon lying immediately downstream of the alternative exon. In some embodiments, the exon lying immediately downstream of the alternative exon may be, for example, the coding region of the transgene (e.g., an MTM1 coding region).

In some embodiments, an alternative exon comprises an ATG start codon, or part of an ATG start codon, within the nucleic acid sequence of the alternative exon (e.g., not at the 3′ end of the alternative exon). In some embodiments, the ATG start codon is in the same reading frame as the coding region of interest. In some embodiments, the ATG start codon is within up to 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the 3′ end of the alternative-spliced exon. In some embodiments, the ATG start codon is within 4-6, 5-7, 6-8, 7-9, 8-10, 9-11, 10-12, 13-15, 14-16, 15-17, 16-18, 17-19, 18-20, 19-21, 20-22, 21-23, 22-24, 23-25, 24-26, 25-27, 26-28, 27-29, or 28-30 nucleotides upstream of the 3′ end of the alternative-spliced exon. In some embodiments, the ATG start codon is within 4-12, 8-16, 12-20, 16-24, or 20-30 nucleotides upstream of the 3′ end of the alternative-spliced exon. In some embodiments, the ATG start codon is within up to 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the 3′ end of the alternative-spliced exon and is in the same reading frame as the coding region of interest. In some embodiments, the ATG start codon is within 4-6, 5-7, 6-8, 7-9, 8-10, 9-11, 10-12, 13-15, 14-16, 15-17, 16-18, 17-19, 18-20, 19-21, 20-22, 21-23, 22-24, 23-25, 24-26, 25-27, 26-28, 27-29, or 28-30 nucleotides upstream of the 3′ end of the alternative-spliced exon and is in the same reading frame as the coding region of interest. In some embodiments, the ATG start codon is within 4-12, 8-16, 12-20, 16-24, or 20-30 nucleotides upstream of the 3′ end of the alternative-spliced exon and is in the same reading frame as the coding region of interest.

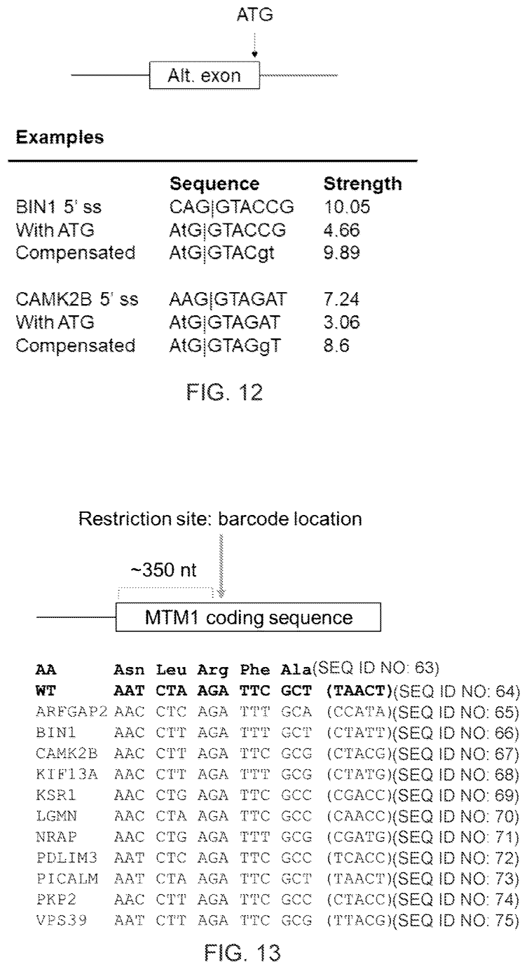

In some embodiments, wherein the alternative exon comprises 1, 2, or 3 nucleic acid substitutions at the 3′ end to result in a heterologous ATG start codon (e.g., if the wild-type alternatively-spliced exon does not comprise an ATG start codon at its 3′ end), the strength of the 5′ splice site of the alternative exon may be diminished, relative to the strength of the 5′ splice site strength of the wild-type or naturally occurring alternative exon. In such embodiments, one or more additional modifications made be made to the intronic sequence located immediately downstream of the sequence comprising the 3′ end of the alternative exon (see FIG. 12). In some embodiments, the first 10 nucleotides of the intronic sequence located immediately downstream of the alternatively-spliced exon comprise 1-5 nucleotide substitutions, relative to the naturally occurring or wild-type intronic sequence located immediately downstream of naturally occurring or wild-type alternative exon. In some embodiments, the first 10 nucleotides of the intronic sequence located immediately downstream of the alternatively-spliced exon comprise 1 nucleotide substitution, relative to the naturally occurring or wild-type intronic sequence located immediately downstream of naturally occurring or wild-type alternative exon. In some embodiments, the first 10 nucleotides of the intronic sequence located immediately downstream of the alternatively-spliced exon comprise 2 nucleotide substitutions, relative to the naturally occurring or wild-type intronic sequence located immediately downstream of naturally occurring or wild-type alternative exon. In some embodiments, the first 10 nucleotides of the intronic sequence located immediately downstream of the alternatively-spliced exon comprise 3 nucleotide substitutions, relative to the naturally occurring or wild-type intronic sequence located immediately downstream of naturally occurring or wild-type alternative exon. In some embodiments, the first 10 nucleotides of the intronic sequence located immediately downstream of the alternatively-spliced exon comprise 4 nucleotide substitutions, relative to the naturally occurring or wild-type intronic sequence located immediately downstream of naturally occurring or wild-type alternative exon. In some embodiments, the first 10 nucleotides of the intronic sequence located immediately downstream of the alternatively-spliced exon comprise 5 nucleotide substitutions, relative to the naturally occurring or wild-type intronic sequence located immediately downstream of naturally occurring or wild-type alternative exon. In some embodiments, the 1-5 nucleotide substitutions restore or partially restore the strength of the 5′ splice site of the alternative exon, relative to the strength of the 5′ splice site of the naturally occurring or wild-type alternative exon.

Additionally or alternatively, in some embodiments the modification comprises disrupting or deleting all native start codons located 5′ to the heterologous start codon. In some embodiments, wherein the alternatively-spliced exon cassette comprises more than one alternatively-spliced exon, all native start codons located 5′ to the heterologous start codon of the 5′-most alternatively-spliced exon are disrupted or deleted. Additionally or alternatively, in some embodiments the modification comprises introducing into the alternatively-spliced exon a heterologous, in-frame stop codon at least 50 nucleotides upstream of the next 5′ splice junction. In some embodiments, the alternatively-spliced exon is a nonsense-mediated decay (NMD) exon. In some embodiments, the NMD exon comprises an in-frame stop codon that is at least 50 nucleotides upstream of the next 5′ splice junction.

In some embodiments, the alternatively-spliced exon is considered to be synthetic when it is situated non-naturally (e.g., is linked to a coding sequence to which it would not be linked in wild-type or naturally-occurring conditions), relative to the wild-type alternatively-spliced exon (e.g., is heterologous). In some embodiments, the alternatively-spliced exon is considered to be synthetic when it (i) undergoes one or more nucleic acid modifications, and (ii) is situated non-naturally, relative to the wild-type alternatively-spliced exon.

In some embodiments, the alternatively-spliced exon is a regulatory exon. In some embodiments, the regulatory exon is an alternatively regulated exon (e.g., an exon known to be subject to alternative splicing mechanisms). It will be appreciated that alternative splicing is a process by which exons or portions of exons or noncoding regions within a pre-mRNA transcript are differentially joined or skipped, resulting in multiple protein isoforms being encoded by a single gene. The regulation of alternative splicing is complex.

Briefly, alternative splicing is known to be regulated by the functional coupling between transcription and splicing. Additional molecular features, such as chromatin structure, RNA structure and alternative transcription initiation or alternative transcription termination, collaborate with these basic components to produce the multiple isoforms that result from alternative splicing (see, e.g., Wang, et al., Biomed Rep. 2015 March; 3(2): 152-158). In certain embodiments, the compositions and methods of the present disclosure utilize the naturally-occurring mechanisms which regulate alternative splicing to express coding regions of interest (e.g., what would be alternatively spliced isoforms in the natural context) in specific biological conditions. In other embodiments, additional genetic elements may be incorporated into the DNA. In some embodiments, such additional genetic elements may become incorporated into the corresponding pre-mRNA, and may consequently influence, control, or otherwise regulate the splicing of the pre-mRNA to form one or more mRNA isoforms.

In some aspects, an alternatively-spliced exon—for which splicing may be regulated—is an exon for which splicing levels differ by at least 5%, for example at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% under two different conditions (e.g., in different tissues, in response to intracellular T cell levels, in response to intracellular levels of one or more RNA binding proteins, in the context of an autoregulated gene, etc). By “splicing levels differ by 5%”, it is meant that the splicing levels for an exon of interest are measured in two different conditions, and the splicing level is compared between the conditions and expressed as a percentage change. For example, if the splicing level in condition A is 80%, and the splicing level in condition B is 85%, the splicing levels between conditions A and B differ by 5%. Likewise, if the splicing level in condition A is 80%, and the splicing level in condition B is 75%, the splicing levels between conditions A and B also differ by 5%.

In some embodiments, the step of calculating a difference in expression of certain isoforms of certain genes in certain conditions as described herein is performed by calculating a percent spliced-in (psi) score. A psi (Ψ) score is a value between 0 to 1 (e.g., 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0, or any value included therein such as e.g. 0.001, 0.0001, 0.0001, etc.) that quantifies alternative splicing occurrences present within a sample, or under certain conditions of interest.

In some embodiments, the Ψ score is calculated (e.g., calculated from RNAseq reads) by dividing the number of inclusion reads (e.g., the number of alternative splicing events for a gene of interest) by the total number of inclusion reads and exclusion reads (e.g., the number of normal (e.g., non-alternative) splicing events for the gene of interest). Therefore, in some embodiments the Ψ score is calculated according to the following formula for the gene of interest:

$$Ψ score = \frac{inclusion reads}{inclusion reads + exclusion reads}$$

In some embodiments, the calculating comprises performing a mixture of isoforms (MISO) analysis. MISO analysis provides an estimate of isoform expression levels within a sample (e.g., a sample comprising a tissue of interest) based on a statistical model and assesses confidence in those estimates. In some embodiments, MISO analysis is performed using MISO software (see, e.g., Katz, Y., E. T. Wang, et al. (2010), Analysis and design of RNA sequencing experiments for identifying isoform regulation, Nat Methods 7(12): 1009-1015).

In some embodiments, a Ψ score higher than (>) 0.50 (for example 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0, or any value included therein such as e.g. 0.5001, 0.50001, etc.) indicates that a greater number of alternative splicing events for the gene of interest are present in the tested sample than the number of regular splicing events. Conversely, in some embodiments a Ψ score lower than (<) 0.50 (for example 0, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or any value included therein such as e.g. 0.499, 0.4999, etc.) indicates that a lower number of alternative splicing events for the gene of interest are present in the tested sample than the number of regular splicing events.

As used herein, delta psi (AN) score is used to refer to the calculation of the difference between two Ψ scores for a single gene of interest (e.g., in different tissues, in different intracellular conditions, etc.). The difference between the two calculated Ψ scores is the AN score. It will be understood that, because a Ψ score may be any value between 0 and 1, as described herein, a AN score (that is, the difference between the two calculated Ψ scores) may also be any value between 0 and 1 (e.g., 0, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0, or any value included therein such as e.g. 0.001, 0.0001, 0.0001, etc.) or any value between 0 and −1 (e.g., 0, −0.01, −0.02, −0.03, −0.04, −0.05, −0.06, −0.07, −0.08, −0.09, −0.10, −0.11, −0.12, −0.13, −0.14, −0.15, −0.16, −0.17, −0.18, −0.19, −0.20, −0.21, −0.22, −0.23, −0.24, −0.25, −0.26, −0.27, −0.28, −0.29, −0.30, −0.31, −0.32, −0.33, −0.34, −0.35, −0.36, −0.37, −0.38, −0.39, −0.40, −0.41, −0.42, −0.43, −0.44, −0.45, −0.46, −0.47, −0.48, −0.49, −0.50, −0.51, −0.52, −0.53, −0.54, −0.55, −0.56, −0.57, −0.58, −0.59, −0.60, −0.61, −0.62, −0.63, −0.64, −0.65, −0.66, −0.67, −0.68, −0.69, −0.70, −0.71, −0.72, −0.73, −0.74, −0.75, −0.76, −0.77, −0.78, −0.79, −0.80, −0.81, −0.82, −0.83, −0.84, −0.85, −0.86, −0.87, −0.88, −0.89, −0.90, −0.91, −0.92, −0.93, −0.94, −0.95, −0.96, −0.97, −0.98, −0.99, or −1.0, or any value included therein such as e.g. −0.001, −0.0001, −0.0001, etc.). In some embodiments, a AN score may be expressed as an absolute value where the absolute value of e.g. −0.1 is 0.1.

In some embodiments, the alternatively-spliced exon is a tissue-specific alternatively-spliced exon. In some embodiments, one or more tissue-specific alternatively-spliced exons are included in a recombinant nucleic acid (e.g., in a rAAV). Non-limiting examples of tissue-specific alternatively-spliced exons are described in Supplemental Table S5 from Wang, E. T., et al., (2008), Nature, 456, 470-76, incorporated herein by reference. Other tissue-specific exons can be identified from transcriptome data. Non-limiting examples of RNA sequence motifs that can exhibit tissue-specific activity, thereby controlling the inclusion or exclusion of tissue-specific exons, are described in Badr, E., et al., (2016), PLOS One, 11(11): e0166978, incorporated herein by reference. In some embodiments, alternative splicing of the tissue-specific exon results in the expression of the transgene (e.g., of the product encoded by the coding region of interest) in heart tissue, but not in skeletal tissue. In some embodiments, alternative splicing of the tissue-specific exon results in the expression of the transgene (e.g., of the product encoded by the coding region of interest) in skeletal tissue, but not in heart tissue. In some embodiments, a tissue-specific alternatively-spliced exon comprises an alternatively-spliced exon from any one or more of: CAMK2B, PKP2, LGMN, NRAP, VPS39, KSR1, PDLIM3, BIN1, ARFGAP2, KIF13A, and/or PICALM.

In some embodiments, the tissue-specific alternatively-spliced exon is or is derived from exon 11 of BIN1. In some embodiments, the tissue-specific alternatively-spliced exon which is or is derived from exon 11 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 37. In some embodiments, the tissue-specific alternatively-spliced exon which is or is derived from exon 11 of BIN1 comprises a polynucleotide having a nucleic acid sequence as set forth in SEQ ID NO: 37. In some embodiments, the tissue-specific alternatively-spliced exon which is or is derived from exon 11 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 38. In some embodiments, the tissue-specific alternatively-spliced exon which is or is derived from exon 11 of BIN1 comprises a polynucleotide having a nucleic acid sequence as set forth in SEQ ID NO: 38.

In some embodiments, an alternatively-spliced exon is an immunoresponsive alternatively-spliced exon (e.g., undergoes alternative splicing in the presence of an enhanced immune response, such as an increased T cell presence). In some embodiments, the immunoresponsive alternatively-spliced exon is alternatively spliced in states of cellular inflammation. In some embodiments, the immunoresponsive alternatively-spliced exon is alternatively spliced when an abnormally elevated quantity of T cells is present in the intracellular environment (e.g., more T cells are present than under homeostatic conditions). In some embodiments, an immunorepressive alternatively-spliced exon comprises an alternatively-spliced exon from any one of ABCC1, AK125149, ASCC2, BAT2D1, BBX, BRD8, BRE, C17orf70, CAMKK2, CBFB, CCAR1, CCDC7CD6, CHTF8, COL4A3BP, COL6A3, CUGBP1, CUGBP2, CXorf45, DENND3, DGUOK, DKFZp762G094, DNAJC7, DNASE1, EIF4A2, EIF4G2, EIF4H, EXOC7, EZH2, FAM120A, FAM136A, FAM36A, FARSB, FBXO38, FGFR1OP2, FIP1L1, FOXRED1, FUBP3, GALT, GATA3, GOLGA2, HIF1A, HMMR, HRB, IKZF1, ILF3, IRAK4, IRF1, KCTD13, LEF1, LUC7L, LYRM1, MALT1 e7, MAP2K7, MAP3K7, MAP4K2, MBNL2, MFF, NAE1, NCSTN, NR4A3, NRF1, NUP98, PARP6, PCM1, PLAUR, PLSCR3, PPIL5, PPP5C, PTPRC-E4, PTPRC-E6, PTS, RABL5, RAPH1, SEC16A, SFRS3, SFRS7, SLMAP, SNRNP70, STAT6, TBC1D1, TIMM8B, TIR8, TRA2A, TROVE2, UGCGL1, VAP-B, VAV1, ZNF384, and ZNF496.

In some embodiments, an alternatively-spliced exon is a cell type-specific alternatively-spliced exon (e.g., undergoes alternative splicing only when located in certain cell types). In some embodiments, a cell type-specific alternatively-spliced exon comprises an alternatively-spliced exon as described in Joglekar, et al. (2021), A spatially resolved brain region- and cell type-specific isoform atlas of the postnatal mouse brain, Nature Comm., 12(463), which is incorporated herein by reference with respect to its description of cell type-specific alternative exons.

In some embodiments, an alternatively-spliced exon is alternatively spliced in cells which exhibit high levels of expression of a particular protein. In some embodiments, an alternatively-spliced exon is alternatively spliced in cells which exhibit low levels of expression of a particular protein. High or low expression of a particular protein may in some embodiments be indicative of a disease state. For example, in some forms of frontotemporal dementia, MAPT exon 10 is aberrantly included, leading to increased levels of the 4R vs. 3R isoform. Increased 4R isoform is associated with neurodegeneration.

Accordingly, in some embodiments an alternatively-spliced exon is alternatively spliced in cells which exhibit disease (e.g., severe disease). In some embodiments, such disease comprises Dentatorubral-pallido-luysian atrophy (DRPLA), myotonic dystrophy type 1 (DM1), myotonic dystrophy type 2 (DM2), Fragile X syndrome of mental retardation (FMR1), Fragile X tremor ataxia syndrome (FXTAS), FRAXE mental retardation (FMR2), Friedreichs ataxia (FRDA), Huntington disease (HD), Huntington disease-like 2 (HDL2), Oculopharyngeal muscular dystrophy (OPMD), Myoclonic epilepsy type 1, Alzheimer's disease, ALS/FTD, spinocerebellar ataxia type 1 (SCA1), spinocerebellar ataxia type 2 (SCA2), spinocerebellar ataxia type 3 (SCA3), spinocerebellar ataxia type 6 (SCA6), spinocerebellar ataxia type 7 (SCAT), spinocerebellar ataxia type 8 (SCA8), spinocerebellar ataxia type 10 (SCA10), spinocerebellar ataxia type 12 (SCA12), spinocerebellar ataxia type 17 (SCA17), Syndromic/non-syndromic X-linked mental retardation, Emery-Dreifuss muscular dystrophy type 2, familial partial lipodystrophy, limb girdle muscular dystrophy type 1B, dilated cardiomyopathy, familial partial lipodystrophy, Charcot-Marie-Tooth disorder type 2B1, mandibuloacral dysplasia, childhood progeria syndrome (Hutchinson-Gilford syndrome), Werner syndrome, Dilated cardiomyopathy (DCM), Hypertrophic cardiomyopathy (HCM), Restrictive cardiomyopathy (RCM), Left Ventricular Non-compaction (LVNC), Arrhythmogenic Right Ventricular Dysplasia (ARVD), takotsubo cardiomyopathy, Duchenne muscular dystrophy, Becker muscular dystrophy, Limb-girdle muscular dystrophy, Facioscapulohumeral muscular dystrophy, Congenital muscular dystrophy, Oculopharyngeal muscular dystrophy, Distal muscular dystrophy, Emery-Dreifuss muscular dystrophy, dementia, Parkinson's disease (PD), a PD-related disorder, Prion disease, a motor neuron disease (MND), Progressive bulbar palsy (PBP), Progressive muscular atrophy (PMA), Primary lateral sclerosis (PLS), Spinal muscular atrophy (SMA), a bladder cancer, a breast cancer, a colorectal cancer, a kidney cancer, a lung cancer, a lymphoma, a melanoma, an oral cancer, an ovarian cancer, an oropharyngeal cancer, a pancreatic cancer, a prostate cancer, a thyroid cancer, a uterine cancer, Down syndrome, Prader-Willi Syndrome (PWS), Bloom Syndrome, Cockayne Syndrome Type I-216400, Cockayne Syndrome Type III, Cockayne Syndrome Type I, Hutchinson-Gilford Progeria Syndrome, Mandibuloacral Dysplasia with Type A Lipodystrophy, Progeria, Adult Onset Progeroid Syndrome, Neonatal Rothmund-Thomson Syndrome, Seip Syndrome, Werner Syndrome, Replication Focus-Forming Activity 1, myotubular myopathy, Danon Disease, and/or centronuclear myopathy.

In some embodiments, an alternatively-spliced exon comprises an exon which may be differentially spliced depending on the intracellular level of the protein encoded by the coding region associated with the alternatively-spliced exon.

In some embodiments, an alternatively-spliced exon comprises an alternatively-spliced exon comprising a polynucleotide sequence as set forth in any one of SEQ ID NOs: 23-44. In some embodiments, an alternatively-spliced exon comprises a polynucleotide sequence that is 90%, 91%, 92%, 93%, 94%, 95%, 96%, 97%, 98%, or 99% identical to any one of SEQ ID NOs: 23-44.

In some embodiments, the alternatively-spliced exon is retained in the spliced transcript. Retention of the alternatively-spliced exon in the spliced transcript occurs under the alternative splicing conditions specific to said alternatively-spliced exon as described herein. In some embodiments, wherein the alternatively-spliced exon cassette comprises more than one alternatively-spliced exon, the 5′-most alternatively-spliced exon is retained in the spliced transcript. In some embodiments, wherein the alternatively-spliced exon cassette comprises more than one alternatively-spliced exon, the 3′-most alternatively-spliced exon is included in the spliced transcript. In some embodiments, wherein the alternatively-spliced exon cassette comprises more than one alternatively-spliced exon, all alternatively-spliced exons are included in the spliced transcript.

In some embodiments, retention of the alternatively-spliced exon in the spliced transcript results in the productive expression of the transgene (e.g., productive translation of the protein). Expression of the product (e.g., therapeutic protein) encoded by the coding region of interest may in some embodiments be desirable. For example, in myotubular myopathy, expression of myotubularin 1 is depleted in skeletal muscle, and therefore restoration of myotubularin 1 in skeletal muscle is desirable. However, in some embodiments, expression of the product (e.g., therapeutic protein) encoded by the coding region of interest may be undesirable. For example, in myotubular myopathy, expression of myotubularin 1 in the heart may be undesirable. Accordingly, in some embodiments retention of the alternatively-spliced exon in the spliced transcript does not result in the productive expression of the transgene (e.g., no productive translation of the protein).

In some embodiments, the alternatively-spliced exon is located 5′ to the coding region of the transgene. In some embodiments, the alternatively-spliced exon is located 3′ to the coding region of the transgene. In some embodiments, the alternatively-spliced exon is located within the coding region of the transgene. In some embodiments, the alternatively-spliced exon is not located within the coding region of the transgene. In some embodiments, the alternatively-spliced exon is located 3′ to a constitutive exon. In some embodiments, the alternatively-spliced exon is located 5′ to a constitutive exon.

### (ii) Constitutive Exons

In some embodiments, the recombinant viral genomes of the present disclosure comprise one or more constitutive exons. In various embodiments, the alternatively-spliced exon and the one or more constitutive exons may be configured as a cassette (e.g., comprised within a transgene. In some embodiments, the transgene comprising an alternatively-spliced exon cassette comprises 1, 2, 3, 4, 5, 6, 7, 8, 9, 10, 11, 12, 13, 14, 15, 16, 17, 18, 19, or 20 constitutive exons. In various embodiments, one or more constitutive exons may comprise a coding region of interest, or a portion thereof. In some embodiments, the constitutive exon is considered to be constitutive when it is present in all isoforms of spliced mRNAs resulting from the splicing of a pre-mRNA transcript.

A constitutive exon may in some embodiments be synthetic, but it need not be. A constitutive exon may be considered synthetic because it undergoes one or more nucleic acid modifications, relative to the wild-type constitutive exon. A nucleic acid modification may be a substitution or deletion of one or more nucleotides that form the nucleic acid sequence of the constitutive exon. In some embodiments, the modification comprises disrupting or deleting all native start codons located within the constitutive exon.

In some embodiments, the constitutive exon is considered to be synthetic when it is situated non-naturally (e.g., is linked to a coding sequence to which it would not be linked in wild-type or naturally-occurring conditions), relative to the wild-type constitutive exon (e.g., is heterologous). In some embodiments, the constitutive exon is considered to be synthetic when it (i) undergoes one or more nucleic acid modifications, and (ii) is situated non-naturally, relative to the wild-type constitutive exon.

In some embodiments, the constitutive exon is naturally occurring (e.g., does not comprise any nucleic acid modifications, relative to the wild-type constitutive exon). In some embodiments, the constitutive exon is a native exon associated with the coding region of the transgene. In some embodiments, the constitutive exon is from or is derived from the same gene as the alternatively-spliced exon.

In some embodiments, the constitutive exon is from or is derived from a constitutive exon of a gene selected from the group consisting of: MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, MTMR2, LAMP2, KIF5A, a microdystrophin-encoding gene, C9ORF72, HTT, DNM2, BIN1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, a cytochrome b oxidase-encoding gene, a cytochrome c oxidase-encoding gene, CLCN1, SCN4A, DMPK, CNBP, MYOT, LMNA, CAV3, DNAJB6, DES, TNPO3, HNRPDL, CAPN3, DYSF, an alpha-sarcoglycan-encoding gene, a beta-sarcoglycan-encoding gene, a gamma-sarcoglycan-encoding gene, a delta-sarcoglycan-encoding gene, TCAP, TRIM32, FKRP, FXN, POMT1, FKTN, POMT2, POMGnT1, DAG1, ANO5, PLEC1, TRAPPC11, GMPPB, ISPD, LIMS2, POPDC1, TOR1AIP1, POGLUT2, LAMA2, COL6A1, POMT1, POMT2, DUX4, EMD, PAX7, PMP22, MPZ, MFN2, SMCHD1, SMN, Lamin A/C (LAMN), and/or GJB1.

In some embodiments, the constitutive exon is from or is derived from a constitutive exon of a gene(s) selected from the group consisting of: ABCC1, AK125149, ASCC2, BAT2D1, BBX, BRD8, BRE, C17orf70, CAMKK2, CBFB, CCAR1, CCDC7CD6, CHTF8, COL4A3BP, COL6A3, CUGBP1, CUGBP2, CXorf45, DENND3, DGUOK, DKFZp762G094, DNAJC7, DNASE1, EIF4A2, EIF4G2, EIF4H, EXOC7, EZH2, FAM120A, FAM136A, FAM36A, FARSB, FBXO38, FGFR1OP2, FIP1L1, FOXRED1, FUBP3, GALT, GATA3, GOLGA2, HIF1A, HMMR, HRB, IKZF1, ILF3, IRAK4, IRF1, KCTD13, LEF1, LUC7L, LYRM1, MALT1 e7, MAP2K7, MAP3K7, MAP4K2, MBNL2, MFF, NAE1, NCSTN, NR4A3, NRF1, NUP98, PARP6, PCM1, PLAUR, PLSCR3, PPIL5, PPP5C, PTPRC-E4, PTPRC-E6, PTS, RABL5, RAPH1, SEC16A, SFRS3, SFRS7, SLMAP, SNRNP70, STAT6, TBC1D1, TIMM8B, TIR8, TRA2A, TROVE2, UGCGL1, VAP-B, VAV1, ZNF384, and/or ZNF496.

In some embodiments, the constitutive exon is from or is derived from a constitutive exon of a gene(s) selected from the group consisting of: CAMK2B, PKP2, LGMN, NRAP, VPS39, KSR1, PDLIM3, BIN1, ARFGAP2, KIF13A, and/or PICALM. In some embodiments, the constitutive exon is from or is derived from a constitutive exon of SMN1. In some embodiments, the constitutive exon is from or is derived from exon 6 of SMN1. In some embodiments, the constitutive exon which is derived from SMN1 exon 6 is a fragment of (e.g., is truncated relative to) the wild-type or naturally occurring sequence of SMN1 exon 6. In some embodiments, the constitutive exon which is derived from SMN1 exon 6 comprises a polynucleotide having a nucleic acid sequence as set forth in SEQ ID NO: 102. In some embodiments, the constitutive exon which is derived from SMN1 exon 6 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 102.

In some embodiments, the constitutive exon is not a native exon associated with the coding region of the transgene. In some embodiments, the constitutive exon is not from nor is derived from the same gene as the alternatively-spliced exon.

In some embodiments, a constitutive exon is located 5′ to the alternatively-spliced exon. Additionally or alternatively, in some embodiments a constitutive exon is located 3′ to the alternatively-spliced exon. In some embodiments, a constitutive exon is located 5′ to the coding region of the transgene. Additionally or alternatively, in some embodiments a constitutive exon is located 3′ to coding region of the transgene.

In some embodiments, the constitutive exon is retained in the spliced transcript (e.g., spliced in). In some embodiments, wherein the transgene comprising an alternatively-spliced exon cassette comprises more than one constitutive exon, the 5′-most constitutive exon is retained in the spliced transcript. In some embodiments, wherein the transgene comprising an alternatively-spliced exon cassette comprises more than one constitutive exon, the 3′-most constitutive exon is retained in the spliced transcript. In some embodiments, wherein the transgene comprising an alternatively-spliced exon cassette comprises more than one constitutive exon, all constitutive exons are retained in the spliced transcript. In some embodiments, the constitutive exon is excluded from the spliced transcript (e.g., spliced out).

(iii) Introns

In other embodiments, the recombinant viral genomes of the present disclosure comprise one or more introns. In various embodiments, the alternatively-spliced exon and the one or more introns (or portions thereof) may be configured as a cassette. In some embodiments, a nucleic acid (e.g., a nucleic acid comprising a recombinant viral genome) comprises an alternatively-spliced exon cassette encoding at least one transgene that contains at least one recombinant (e.g., engineered, truncated) intron that supports sufficient splice regulation of the transgene to be therapeutically effective. In some embodiments an alternatively-spliced exon cassette is an RNA molecule (e.g., a pre-mRNA) that contains one or more (e.g., two or more) recombinant (e.g., engineered; e.g., truncated) introns flanking one or more exons. In some embodiments, an alternatively-spliced exon cassette is a DNA molecule that encodes the RNA molecule containing one or more recombinant (e.g., engineered; e.g., truncated) introns. In some embodiments, a transgene comprising an alternatively-spliced exon cassette contains other regulatory sequences (e.g., promoters, 5′ or 3 UTRs, or other regulatory sequences) in addition to the gene coding (e.g., protein coding) sequences and the at least one recombinant (e.g., engineered; e.g., truncated) intron for which splicing can be regulated, as described elsewhere herein.

Accordingly, in some embodiments, a recombinant viral genome of the present disclosure comprises a transgene comprising an alternatively-spliced exon cassette, wherein the alternatively-spliced exon cassette comprises among other components at least one intron (or portion thereof). In some embodiments, the intron is a flanking intron (or portion thereof). In some embodiments, the alternatively-spliced exon cassette comprises 1, 2, 3, 4, 5, 6, 7, or 8 flanking introns (or portion(s) thereof).

In some embodiments, an exon (e.g., an alternatively-spliced exon, or a constitutive exon) is flanked by one or more introns (e.g., flanking introns), or portion(s) thereof. In some embodiments, an alternatively-spliced exon is flanked by one or more introns (or portion(s) thereof). In some embodiments, an alternatively-spliced exon is flanked by one intron (or portion thereof). In some embodiments, wherein the alternatively-spliced exon is flanked by one intron, the flanking intron (or portion thereof) is located 3′ to the alternatively-spliced exon. In some embodiments, wherein the alternatively-spliced exon is flanked by one intron, the flanking intron (or portion thereof) is located 5′ to the alternatively-spliced exon. In some embodiments, an alternatively-spliced exon is flanked by two introns (or portions thereof). In some embodiments, wherein the alternatively-spliced exon cassette comprises more than one alternatively-spliced exon, each alternatively-spliced exon is flanked by at least one, and in some embodiments two, flanking intron(s) (or portion(s) thereof). In some embodiments, an intron is a native flanking intron or native flanking intronic sequence of the alternatively-spliced exon. In some embodiments, an intron is not a native flanking intron or native flanking intronic sequence of the alternatively-spliced exon.

In some embodiments, a constitutive exon is flanked by one or more introns (or portion(s) thereof). In some embodiments, a constitutive exon is flanked by one intron (or portion thereof). In some embodiments, wherein the constitutive exon is flanked by one intron, the flanking intron (or portion thereof) is located 3′ to the constitutive exon. In some embodiments, wherein the constitutive exon is flanked by one intron, the flanking intron (or portion thereof) is located 5′ to the constitutive exon. In some embodiments, a constitutive exon is flanked by two introns (or portions thereof). In some embodiments, wherein the alternatively-spliced exon cassette comprises more than one constitutive exon, each constitutive exon is flanked by at least one, and in some embodiments two, flanking intron(s) (or portion(s) thereof). In some embodiments, an intron is a native flanking intron or native flanking intronic sequence of the constitutive exon. In some embodiments, an intron is not a native flanking intron or native flanking intronic sequence of the constitutive exon.

In some embodiments, an intron is a natural intron, and comprises no modifications, relative to a native intron.

An intron or intronic sequence may in some embodiments be synthetic, but it need not be. A synthetic intron or intronic sequence may be considered synthetic because it undergoes one or more nucleic acid modifications, relative to the wild-type or native intron. A nucleic acid modification may be a substitution or deletion of one or more nucleotides that form the nucleic acid sequence of the intron or intronic sequence.

In some embodiments, an intron or intronic sequence is considered to be synthetic when it is situated non-naturally (e.g., is linked to an exon to which it would not be linked in wild-type or naturally-occurring conditions), relative to the wild-type intron or intronic sequence (e.g., is heterologous). In some embodiments, the intron or intronic sequence is considered to be synthetic when it (i) undergoes one or more nucleic acid modifications, and (ii) is situated non-naturally, relative to the wild-type intron or intronic sequence.

In some embodiments, an intron (e.g., a flanking intron) (or portion thereof) comprising one or more nucleic acid modifications, relative to the wild-type intron, is an engineered intron or intronic sequence. In some embodiments, the engineered intron or intronic sequence comprises a splice donor and splice acceptor site, and a functional branch point to which the splice donor site can be joined in the first trans-esterification reaction of splicing.

In some embodiments, an intron (e.g., a flanking intron) or intronic sequence comprising one or more nucleic acid modifications, relative to the wild-type intron, comprises a truncated version of a natural intron. By “truncated version of a natural intron”, it is meant that the naturally-occurring, full-length intron is shortened (e.g., truncated) via the removal of nucleotides. In some embodiments, an engineered (e.g., recombinant) intron or intronic sequence is a truncated version of a natural intron. However, in some embodiments an engineered intron or intronic sequence can be designed to include functional splice donor and acceptor sites and a functional branch point in addition to one or more regulatory regions that are derived from different introns, or that are non-naturally occurring sequences (e.g., sequence variants of naturally-occurring sequences, consensus sequences, or de novo designed sequences). Accordingly, in some embodiments an engineered intron or intronic sequence is not a truncated version of a naturally occurring intron, but contains one or more sequences from a naturally occurring intron.

In some embodiments, an intron (e.g., a flanking intron) (or portion thereof) comprising one or more nucleic acid modifications, relative to the wild-type intron, is truncated at its 5′ end. In some embodiments, 1-10,000 nucleotides are truncated from the 5′ end (e.g., 1-50, 50-100, 100-500, 500-1,000, 1,000-5,000, 5,000-10,000, 10,000-20,000, 20,000-50,000, or 50,000-100,000 nucleotides are truncated from the 5′ end). In some embodiments, the 5′ splice site is not retained in the truncated intron (or portion thereof). In some embodiments, the 5′ splice site is retained in the truncated intron (or portion thereof). In some embodiments, a different 5′ splice site is included in the truncated intron (or portion thereof).

In some embodiments, an intron (e.g., a flanking intron) (or portion thereof) comprising one or more nucleic acid modifications, relative to the wild-type intron, is truncated at its 3′ end. In some embodiments, 1-10,000 nucleotides are truncated from the 3′ end (e.g., 1-50, 50-100, 100-500, 500-1,000, 1,000-5,000, 5,000-10,000, 10,000-20,000, 20,000-50,000, or 50,000-100,000 nucleotides are truncated from the 3′ end). In some embodiments, the 3′ splice site is not retained in the truncated intron (or portion thereof). In some embodiments, the 3′ splice site is retained in the truncated intron (or portion thereof). In some embodiments, a different 3′ splice site is included in the truncated intron (or portion thereof).

In some embodiments, an intron (e.g., a flanking intron) (or portion thereof) comprising one or more nucleic acid modifications, relative to the wild-type intron, is truncated at one or more internal locations. In some embodiments, 1-10,000 internal nucleotides are removed (e.g., 1-50, 50-100, 100-500, 500-1,000, 1,000-5,000, 5,000-10,000, 10,000-20,000, 20,000-50,000, or 50,000-100,000 internal nucleotides are removed). In some embodiments, the splice regulatory region is not retained in the truncated intron (or portion thereof). In some embodiments, the splice regulatory region is retained in the truncated intron (or portion thereof). In some embodiments, a different splice regulatory region is included in the truncated intron (or portion thereof).

In some embodiments, an intron (e.g., a flanking intron) (or portion thereof) comprising one or more nucleic acid modifications, relative to the wild-type intron, comprises one or more 5′, 3′, and/or internal deletions. It should be understood that the extent of truncation may depend on the size of the intron (or portion thereof) and the size of the gene. A truncation may require removal of sufficient intronic sequence to result in a recombinant gene construct that is small enough to be packaged in a recombinant virus of interest (e.g., in a recombinant AAV or lentivirus).

However, an intron typically includes one or more sequences required for efficient splicing and/or regulated splicing. In some embodiments, an intron or intronic sequence comprises one or more splice junction sites (e.g., a 5′ splice donor site, and/or a 3′ splice acceptor site). In some embodiments, an intron or intronic sequence retains a splice donor site (e.g., towards the 5′ end of the intron or intronic sequence), a branch site (e.g., towards the 3′ end of the intron or intronic sequence), a splice acceptor site (e.g., at the 3′ end of the intron or intronic sequence), and a splice regulatory sequence. In some embodiments, the intron or intronic sequence comprises a 5′ splice donor site. In some embodiments, the 5′ splice donor site is a GU or an AU. In some embodiments, the intron or intronic sequence comprises a 3′ splice acceptor site. In some embodiments, the 3′ splice acceptor site is an AG or an AC. In some embodiments, an intron or intronic sequence comprises at its 5′ end a 5′ splice donor site and at its 3′ end a 3′ splice acceptor site. In some embodiments, a regulatory sequence comprises a response element within an AG exclusion zone of the intron. In some embodiments, the intron or intronic sequence retains sequence motifs bound by the encoded protein (e.g., YGCY motifs for MBNL1, or GCAUG for RBFOX, or YCAY for NOVA, etc.). In some embodiments, an intron or intronic sequence is spliced out, and is not included in the spliced transcript.

In some embodiments, an intron or intronic sequence may include one or more human, non-human primate, and/or other mammalian or non-mammalian intron splice-regulatory sequences. In some embodiments, the regulatory sequences may have 80%-100% (e.g., 80-85%, 85%-90%, greater than 90%, 90%-95%, or 95%-100%) sequence identity, relative to a wild-type regulatory sequence.

In some embodiments, an intron or intronic sequence is approximately 50 to 4000 nucleotides long. In some embodiments, an intron or intronic sequence is approximately 50 to 100, 75-125, 100-150, 125-175, 200-250, 225-275, 300-350, 325-375, 400-450, 425-475, 500-550, 525-575, 600-650, 625-675, 700-750, 725-775, 800-850, 825-875, 900-950, 925-975, 950-1000, 1025-1075, 1050 to 1100, 1075-1125, 1100-1150, 1125-1175, 1200-1250, 1225-1275, 1300-1350, 1325-1375, 1400-1450, 1425-1475, 1500-1550, 1525-1575, 1600-1650, 1625-1675, 1700-1750, 1725-1775, 1800-1850, 1825-1875, 1900-1950, 1925-1975, 1950-2000, 2025-2075, 2050 to 2100, 2075-2125, 2100-2150, 2125-2175, 2200-2250, 2225-2275, 2300-2350, 2325-2375, 2400-2450, 2425-2475, 2500-2550, 2525-2575, 2600-2650, 2625-2675, 2700-2750, 2725-2775, 2800-2850, 2825-2875, 2900-2950, 2925-2975, 2950-3000, 3025-3075, 3050 to 3100, 3075-3125, 3100-3150, 3125-3175, 3200-3250, 3225-3275, 3300-3350, 3325-3375, 3400-3450, 3425-3475, 3500-3550, 3525-3575, 3600-3650, 3625-3675, 3700-3750, 3725-3775, 3800-3850, 3825-3875, 3900-3950, 3925-3975, or 3950-4000 nucleotides long, or any integer contained therein (e.g., 51, 52, 53, 54, 55, etc.). In some embodiments, an intron or intronic sequence is approximately 50-60, 55-65, 60-70, 65-75, 70-80, 75-85, 80-90, 95-105, 100-110, 105-115, 110-120, 115-125, 120-130, 125-135, 130-140, 135-145, 140-150, 145-155, 150-160, 155-165, 160-170, 165-175, 170-180, 175-185, 180-190, 185-195, or 190-200 nucleotides long, or any integer contained therein (e.g., 100, 101, 102, 103, 104, 105, etc.). In some embodiments, an intron or intronic sequence is approximately 50-80, 60-90, 70-100, 80-110, 90-120, 100-130, 110-140, 120-150, 130-160, 140-170, 150-180, 160-190, or 170-200 nucleotides long, or any integer contained therein (e.g., 120, 121, 122, 123, 124, 125, etc.).

In some embodiments, a natural or wild-type intron is truncated or otherwise modified so as to retain only the sequence which regulates the up- or down-stream alternative exon. In some embodiments, said regulatory sequence is located within approximately 100-300 nucleotides upstream or downstream of the exon-intron (or intron-exon) border. In some embodiments, said regulatory sequence is located within approximately 100-110, 105-115, 110-120, 115-125, 120-130, 125-135, 130-140, 135-145, 140-150, 145-155, 150-160, 155-165, 160-170, 165-175, 170-180, 175-185, 180-190, 185-195, 190-200, 205-215, 210-220, 215-225, 220-230, 225-235, 230-240, 235-245, 240-250, 245-255, 250-260, 255-265, 260-270, 265-275, 270-280, 275-285, 280-290, 285-295, or 290-300 nucleotides upstream or downstream of the exon-intron (or intron-exon) border. In some embodiments, said regulatory sequence is located within approximately 100-130, 110-140, 120-150, 130-160, 140-170, 150-180, 160-190, 170-200, 210-240, 220-250, 230-260, 240-270, 250-280, 260-290, or 270-300 nucleotides upstream or downstream of the exon-intron (or intron-exon) border.

In some embodiments, the only intron that is comprised within an alternatively-spliced exon cassette is a truncated regulated intron. A regulated intron may in some embodiments be a regulated intron that flanks the alternative exon in its natural or wild-type context. In some embodiments, two regulated introns flank the alternative exon in its natural or wild-type context. A regulated intron may be located 5′ or 3′ relative to the alternative exon in its natural or wild-type context. In some embodiments, a regulated intron or truncated regulated intron is 5′ relative to the alternative exon within an alternative exon cassette of the disclosure. In some embodiments, a regulated intron or truncated regulated intron is 3′ relative to the alternative exon within an alternative exon cassette of the disclosure. In some embodiments, two or more regulated introns are retained and truncated in an alternatively-spliced exon cassette. In some embodiments, the two or more truncated regulated introns flank the alternative exon within the alternative exon cassette. In some embodiments, all other (e.g., non-regulatory) introns and intronic sequences have been removed. However, in some embodiments, one or more of the other introns (e.g., the introns that are not subject to regulated splicing) or intronic sequences may be retained (and optionally truncated) depending on the size of the nucleic acid and the size limitations of the virus, respectively. In some embodiments, the only introns or intronic sequences in an alternatively-spliced exon cassette are truncated introns or intronic sequences (e.g., only one, 2, 3, 4, 5, 6, 7, 8, 9, 10 truncated introns or intronic sequences). In some embodiments, an alternatively-spliced exon cassette does not contain any full-length introns. In some embodiments, an alternatively-spliced exon cassette does not contain any truncated introns or intronic sequences that are not regulated.

In some embodiments, the intron(s) or intronic sequence(s) flanking an alternative exon(s) comprise an intron or intronic sequence from or derived from a gene selected from the group consisting of: ABCC1, AK125149, ASCC2, BAT2D1, BBX, BRD8, BRE, C17orf70, CAMKK2, CBFB, CCAR1, CCDC7CD6, CHTF8, COL4A3BP, COL6A3, CUGBP1, CUGBP2, CXorf45, DENND3, DGUOK, DKFZp762G094, DNAJC7, DNASE1, EIF4A2, EIF4G2, EIF4H, EXOC7, EZH2, FAM120A, FAM136A, FAM36A, FARSB, FBXO38, FGFR1OP2, FIP1L1, FOXRED1, FUBP3, GALT, GATA3, GOLGA2, HIF1A, HMMR, HRB, IKZF1, ILF3, IRAK4, IRF1, KCTD13, LEF1, LUC7L, LYRM1, MALT1 e7, MAP2K7, MAP3K7, MAP4K2, MBNL2, MFF, NAE1, NCSTN, NR4A3, NRF1, NUP98, PARP6, PCM1, PLAUR, PLSCR3, PPIL5, PPP5C, PTPRC-E4, PTPRC-E6, PTS, RABL5, RAPH1, SEC16A, SFRS3, SFRS7, SLMAP, SNRNP70, STAT6, TBC1D1, TIMM8B, TIR8, TRA2A, TROVE2, UGCGL1, VAP-B, VAV1, ZNF384, and/or ZNF496.

In some embodiments, the intron(s) or intronic sequence(s) flanking an alternative exon(s) comprise an intron or intronic sequence from or derived from a gene selected from the group consisting of: CAMK2B, PKP2, LGMN, NRAP, VPS39, KSR1, PDLIM3, BIN1, ARFGAP2, KIF13A, and/or PICALM.

In some embodiments, the intron(s) or intronic sequence(s) flanking an alternative exon(s) is or is derived from an intron of BIN1. In some embodiments, the intron(s) or intronic sequence(s) flanking an alternative exon(s) is or is derived from intron 10 and/or intron 11 of BIN1. In some embodiments, intron(s) or intronic sequence(s) flanking an alternative exon(s) which is or is derived from intron 10 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 15. In some embodiments, the intron(s) or intronic sequence(s) flanking an alternative exon(s) which is or is derived from intron 10 of BIN1 comprises a polynucleotide having a nucleic acid sequence as set forth in SEQ ID NO: 15. In some embodiments, the intron(s) or intronic sequence(s) flanking an alternative exon(s) which is or is derived from intron 11 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 16. In some embodiments, the intron(s) or intronic sequence(s) flanking an alternative exon(s) which is or is derived from intron 11 of BIN1 comprises a polynucleotide having a nucleic acid sequence as set forth in SEQ ID NO: 16.

In some embodiments, the intron(s) or intronic sequence(s) flanking an alternative exon(s) comprise an intron or intronic sequence comprising a polynucleotide sequence as set forth in any one of SEQ ID NOs: 1-22, 103, and 104. In some embodiments, an intron or intronic sequence comprises a polynucleotide sequence that is 90%, 91%, 92%, 93%, 94%, 95%, 96%, 97%, 98%, or 99% identical to any one of SEQ ID NOs: 1-22, 103, and 104.

In some embodiments, all the introns (or portion(s) thereof) and exons (or portion thereof) of an alternatively-spliced exon cassette are from the same gene. Some embodiments of the present invention contemplate heterologous gene constructs, wherein introns (or portion(s) thereof) and exons (or portion(s) thereof) from different genes are integrated into a single alternatively-spliced exon cassette or transgene. In some embodiments, at least one intron (or portion thereof) and at least one exon (or portion thereof) of the nucleic acid construct are from different genes.

In some embodiments, an intron (or portion thereof) and/or an exon (or portion thereof) is from or derived from a gene(s) which comprises any one or more of: MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, MTMR2, LAMP2, KIF5A, a microdystrophin-encoding gene, C9ORF72, HTT, DNM2, BIN1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, a cytochrome b oxidase-encoding gene, a cytochrome c oxidase-encoding gene, CLCN1, SCN4A, DMPK, CNBP, MYOT, LMNA, CAV3, DNAJB6, DES, TNPO3, HNRPDL, CAPN3, DYSF, an alpha-sarcoglycan-encoding gene, a beta-sarcoglycan-encoding gene, a gamma-sarcoglycan-encoding gene, a delta-sarcoglycan-encoding gene, TCAP, TRIM32, FKRP, FXN, POMT1, FKTN, POMT2, POMGnT1, DAG1, ANO5, PLEC1, TRAPPC11, GMPPB, ISPD, LIMS2, POPDC1, TOR1AIP1, POGLUT2, LAMA2, COL6A1, POMT1, POMT2, DUX4, EMD, PAX7, PMP22, MPZ, MFN2, SMCHD1, SMN1, and/or GJB1.

In some embodiments, an intron (or portion thereof) and/or an exon (or portion thereof) is from or derived from a gene(s) which comprises any one or more of: ABCC1, AK125149, ASCC2, BAT2D1, BBX, BRD8, BRE, C17orf70, CAMKK2, CBFB, CCAR1, CCDC7CD6, CHTF8, COL4A3BP, COL6A3, CUGBP1, CUGBP2, CXorf45, DENND3, DGUOK, DKFZp762G094, DNAJC7, DNASE1, EIF4A2, EIF4G2, EIF4H, EXOC7, EZH2, FAM120A, FAM136A, FAM36A, FARSB, FBXO38, FGFR1OP2, FIP1L1, FOXRED1, FUBP3, GALT, GATA3, GOLGA2, HIF1A, HMMR, HRB, IKZF1, ILF3, IRAK4, IRF1, KCTD13, LEF1, LUC7L, LYRM1, MALT1 e7, MAP2K7, MAP3K7, MAP4K2, MBNL2, MFF, NAE1, NCSTN, NR4A3, NRF1, NUP98, PARP6, PCM1, PLAUR, PLSCR3, PPIL5, PPP5C, PTPRC-E4, PTPRC-E6, PTS, RABL5, RAPH1, SEC16A, SFRS3, SFRS7, SLMAP, SNRNP70, STAT6, TBC1D1, TIMM8B, TIR8, TRA2A, TROVE2, UGCGL1, VAP-B, VAV1, ZNF384, and/or ZNF496.

In some embodiments, an intron (or portion thereof) and/or an exon (or portion thereof) is from or derived from a gene(s) which comprises any one or more of: CAMK2B, PKP2, LGMN, NRAP, VPS39, KSR1, PDLIM3, BIN1, ARFGAP2, KIF13A, and/or PICALM.

In some embodiments, one or more introns (or portions thereof) and/or an exon (or portion thereof) is from or derived from BIN1.

In some embodiments, the one or more introns (or portions thereof) is or is derived from an intron(s) of BIN1. In some embodiments, the one or more introns (or portions thereof) is or is derived from intron 10 and/or intron 11 of BIN1. In some embodiments, the one or more introns (or portions thereof) which is or is derived from intron 10 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 15. In some embodiments, the one or more introns (or portions thereof) which is or is derived from intron 10 of BIN1 comprises a polynucleotide having a nucleic acid sequence as set forth in SEQ ID NO: 15. In some embodiments, the one or more introns (or portions thereof) which is or is derived from intron 11 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 16. In some embodiments, the one or more introns (or portions thereof) which is or is derived from intron 11 of BIN1 comprises a polynucleotide having a nucleic acid sequence as set forth in SEQ ID NO: 16.

In some embodiments, an exon (or portion thereof) is or is derived from exon 11 of BIN1. In some embodiments, the exon (or portion thereof) which is or is derived from exon 11 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 37. In some embodiments, the exon (or portion thereof) which is or is derived from exon 11 of BIN1 comprises a polynucleotide having a nucleic acid sequence as set forth in SEQ ID NO: 37. In some embodiments, the exon (or portion thereof) which is or is derived from exon 11 of BIN1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 38. In some embodiments, the exon (or portion thereof) which is or is derived from exon 11 of BIN1 comprises a polynucleotide having a nucleic acid sequence as set forth in SEQ ID NO: 38.

In some embodiments, the one or more introns (or portions thereof) and/or the exon (or portion thereof) which are from or derived from BIN1 together comprise an alternative exon cassette. In some embodiments, the alternative exon cassette (which comprises the one or more introns (or portions thereof) and/or the exon (or portion thereof) which are from or derived from BIN1) comprises a polynucleotide having at least 70%, at least 75%, at least 80%, at least 90%, at least 92%, at least 95%, at least 98%, or at least 99% sequence identity, relative to a nucleic acid sequence as set forth in any one of SEQ ID NOs: 107-778. In some embodiments, the alternative exon cassette (which comprises the one or more introns (or portions thereof) and/or the exon (or portion thereof) which are from or derived from BIN1) comprises a polynucleotide having a nucleic acid sequence as set forth in any one of SEQ ID NOs: 107-778.

In some embodiments, an alternative exon cassette (e.g., which comprises the one or more introns (or portions thereof) and/or the exon (or portion thereof) which are from or derived from BIN1) is selected for inclusion in a transgene based on the psi values which the alternative exon cassette achieves in a specific tissue of interest (see, e.g., Table 4; Table 5). For example, if the coding region of the transgene encodes a protein which would be therapeutically useful in skeletal tissue (e.g., MTM1), but which would not be desirable to express in heart tissue, the alternative exon cassette selected for inclusion in a transgene would be one wherein a high psi value is observed for skeletal tissue, and wherein a low psi value is observed for heart tissue (e.g., the A psi between skeletal tissue and heart tissue is large). In some embodiments, wherein the coding region of the transgene encodes a protein which would be therapeutically useful in skeletal tissue (e.g., MTM1), but which would not be desirable to express in heart tissue, the alternative exon cassette selected from inclusion in a transgene would be one wherein a high psi value is observed for skeletal tissue. In some embodiments, wherein the coding region of the transgene encodes a protein which would be therapeutically useful in skeletal tissue (e.g., MTM1), but which would not be desirable to express in heart tissue, the alternative exon cassette selected from inclusion in a transgene would be one wherein a low psi value is observed for heart tissue. As will be understood, the alternative exon cassette which is included in a transgene may be selected based on a variety of factors including, but not limited to: the identity of the protein cargo to be encoded by the coding region of interest; the A psi observed between a first tissue (or condition, etc.) which is of interest and a second tissue (or condition, etc.) which is not of interest; the psi observed in a tissue (or condition, etc.) which is of interest; and/or the psi observed in a tissue (or condition, etc.) which is not of interest. However, various other factors may also impact which alternative exon cassette is selected for inclusion in a transgene, as described throughout the disclosure.

In some embodiments, an intron (or portion thereof) and/or an exon (or portion thereof) is from or derived from SMN1.

In some embodiments, an intron(s) is or is derived from intron 6 and/or intron 7 of SMN1. In some embodiments, the intron which is derived from SMN1 intron 6 is a fragment of (e.g., is truncated relative to) the wild-type or naturally occurring sequence of SMN1 intron 6. In some embodiments, the intron which is derived from SMN1 intron 6 comprises a polynucleotide having a nucleic acid sequence as set forth in SEQ ID NO: 103. In some embodiments, the intron which is derived from SMN1 intron 6 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 103. In some embodiments, the intron which is derived from SMN1 intron 7 is a fragment of (e.g., is truncated relative to) the wild-type or naturally occurring sequence of SMN1 intron 7. In some embodiments, the intron which is derived from SMN1 intron 7 comprises a polynucleotide having a nucleic acid sequence as set forth in SEQ ID NO: 104. In some embodiments, the intron which is derived from SMN1 intron 7 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 104.

In some embodiments, an exon is or is derived from exon 6 of SMN1. In some embodiments, the exon which is derived from SMN1 exon 6 is a fragment of (e.g., is truncated relative to) the wild-type or naturally occurring sequence of SMN1 exon 6. In some embodiments, the exon which is derived from SMN1 exon 6 comprises a polynucleotide having a nucleic acid sequence as set forth in SEQ ID NO: 102. In some embodiments, the exon which is derived from SMN1 exon 6 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 102.

### (iv) Positive or Negative Regulatory Cis-Element

In other embodiments, the recombinant viral genomes of the present disclosure comprise one or more regulatory sequences. In some embodiments, the regulatory sequences impart a positive control on the expression of a coding sequence of interest. In other embodiments, the regulatory sequences impart a negative control on the expression of a coding sequence of interest. Regulatory sequences may be present, inserted, or otherwise included in an alternatively-spliced exon. Such sequences may be referred to as positive or negative regulatory control cis-elements or “regulatory cis-elements” or merely as “cis-elements.”

The one or more cis-elements located within an alternatively-spliced exon and which may influence the level of expression of a coding region of interest through positive and/or negative controls may comprehensively include any genetic element which exerts—as a consequence being spliced-in or spliced-out of the final mRNA—either a positive or negative regulation on the expression of the coding region. Non-limiting examples of positive or negative regulatory cis-elements located within the alternatively-spliced exons can include, without limitation, a translation start codon, a translation stop codon, a binding site for an RNA binding protein that serves to positively regulate mRNA translation, a binding site for an RNA binding protein that serves to negatively regulate mRNA translation, a binding site for a nucleic acid molecule (e.g., an miRNA) that serves to positively regulate mRNA translation, or a binding site for a nucleic acid molecule (e.g., an siRNA) that serves to negatively regulate mRNA stability or degradation, a binding site for an RNA binding protein that serves to positively regulate mRNA stability or degradation, a binding site for an RNA binding protein that serves to negatively regulate mRNA stability or degradation, a binding site for a nucleic acid molecule (e.g., an miRNA) that serves to positively regulate mRNA stability or degradation, a binding site for a nucleic acid molecule (e.g., an siRNA) that serves to negatively regulate mRNA stability or degradation, a nuclease recognition site, a sequence that can form a secondary structure that slows down translation (for example a stem loop that delays the ribosome), or a sequence that can form a secondary structure that promotes translation. This list of examples is not intended to place any limitation on the scope and meaning of the positive and negative cis-elements and the disclosure embraces any genetic element or region positioned within or at least associated with an alternatively-spliced exon which exerts a positive or negative control on the overall expression of a coding region of the transgene (e.g., encoding a therapeutic protein).

In some cases, the cis-element is located within the alternatively-spliced exon, but in other cases, the cis-element is separate from, but at least associated with, the alternatively-spliced exon, such that it is spliced-in or spliced-out at the same time as the alternatively-spliced exon. Non-limiting examples of positive or negative regulatory cis-elements can include, for instance, (1) a nucleotide sequence element that regulates, modulates, or otherwise affects the stability and/or degradation of a mRNA; and (2) a nucleotide sequence element that regulates, modulates, or otherwise affects the translation of a mRNA into one or more encoded polypeptide products (e.g., a therapeutic product).

In some embodiments, the one or more cis-elements can include, but are not limited to, a translation start codon, a translation stop codon, an siRNA binding site, a miRNA binding site, a sequence forming a stem-loop structure, a sequence forming an RNA dimerization motif, a sequence forming a hairpin structure, a sequence forming an RNA quadruplex, polypurine tract, a sequence forming a pair of kissing loops, and a sequence forming a tetraloop/tetraloop receptor pair. In some embodiments, cis-elements include binding sites recognized by regulatory elements, such as, for example, RNA binding proteins.

In some embodiments, an RNA binding protein may be involved in binding to one or more positive or negative cis-elements and, as such, may be involved in regulating the expression of the coding region of interest.

In some embodiments, the RNA binding protein is a sequence-specific RNA binding protein. In some embodiments, a useful sequence-specific RNA binding protein binds to a target sequence with a binding affinity (e.g., Kd) of 0.01-1000 nM or less (e.g., 0.01 to 1, 1-10, 10-50, 50-100, 100-500, 500-1,000 nM). In some embodiments, an RNA binding protein has serine/arginine domains that act as splicing enhancers, or glycine-rich domains that act as splicing repressors. In some embodiments, an RNA binding protein acts as an intronic splicing enhancer, intronic splicing silencer, exonic splicing enhancer, or exonic splicing silencer.

Different types of sequence-specific RNA binding proteins can be used. In some embodiments, a sequence-specific RNA binding protein is one that contains zinc fingers, RNA recognition motifs, KH domains, deadbox domains, or dsRBDs. Non-limiting examples of RBPs that contain zinc fingers include: MBNL, TIS11, or TTP. Non-limiting examples of RBPs that contain RNA recognition motifs include hnRNPs and SR proteins, RbFox, PTB, Tra2beta. Non-limiting examples of RNA binding proteins that contain KH domains include Nova, SF1, and FBP. Non-limiting examples of RNA binding proteins that contain deadbox domains are DDX5, DDX6, and DDX17. Non-limiting examples of RNA binding proteins that contain dsRBDs include ADAR, Staufen, and TRBP.

Further examples of these types of RNA binding proteins and their respective sequence specific binding motifs are known in the art, and can be found, for example, in Perez-Perri, J. I., et al., (2018), Nat. Comm., 9:4408; Van Nostrand, E. L., et al., (2020), Nature, 583, 711-19; and Corley, M., et al., (2020), Cell, (20): 30159-3, the contents of which are hereby incorporated by reference with respect to RNA protein binding sites and RNA binding proteins.

### (v) Splicing Factors

In some embodiments, the recombinant viral vector genomes may further comprise one or more regulatory sequences and/or genes encoding factors that regulate splicing, including splicing of the alternatively-spliced exon.

In some embodiments, that regulatory gene encodes a tissue-specific RNA binding protein, an autoregulatory RNA binding protein, or a condition-specific RNA binding protein. In some embodiments, the protein auto-regulates splicing of the mRNA encoded by the recombinant viral genome. In some embodiments, splicing can be regulated by two or more different splice regulatory proteins that bind to splicing regulatory regions. For example, in some embodiments, NRAP exon 12 is highly included in skeletal muscle but absent in heart. In some embodiments, TPM2 exon 2 is low in heart but high in smooth muscle. In some embodiments, SLC25A3 is very high in heart but low in brain. Many other examples can be found in the literature and one example of a list of such “switch-like exons” can be found in Wang, E. T., et al., (2008), Nature, 456(7221):470-6. Such sequences may be included in the recombinant viral genomes to further regulate splicing under certain desired conditions.

In some embodiments, the recombinant viral genome may further encode a splice-regulatory protein, which can include, for instance, MBNL protein, an SR protein (e.g., SRSF1, SRSF2, SRSF3, SRSF4, SRSF5, SRSF6, SRSF7, SRSF8, SRSF9, SRSF10, SRSF11, or SRSF12), an hnRNP protein, an RbFox protein, a CELF protein, a Nova protein, or a PTB protein.

In some embodiments, the viral vectors may also encode a splicing factor in the form of an RNA, which may comprise a regulatory RNA molecule, a short hairpin RNA molecule (shRNA), a microRNA molecule, a transfer RNA molecule (tRNA), or an RNA that comprises a DMPK-targeting shRNA or microRNA. The RNA that regulates splicing may also comprise a repeat-targeting shRNA or microRNA (e.g., a CUG shRNA, CAG shRNA, or GGGGCC shRNA), e.g., which targets an RNA binding protein or other member of a related biological pathway.

In some embodiments, the viral vectors may also encode a splicing factor that comprises a protein-RNA complex, the protein-RNA complex comprises a ribosome, snRNP complex, or other macromolecular complex that can interact with RNA to regulate splicing decisions. In some embodiments, wherein the intracellular factor comprises a protein-RNA complex, a snRNP complex comprises U1 snRNP or U2 snRNP. In some embodiments, wherein the intracellular factor comprises a protein-RNA complex, the RNA comprises a ribozyme that targets one or more CUG repeats. In some embodiments, wherein the intracellular factor comprises a protein-RNA complex, the RNA comprises a ribozyme that targets specific mRNAs.

Non-limiting examples of RNA binding protein motifs and RNA target sequences that can confer or regulate spicing activity are described, for example, in Ray, D., et al., (2014), Nature, 499(7457): 172-77; Lambert, N., et al., (2014), Mol. Cell., 54(5): 887-900; and Van Nostrand, E. L., et al., (2020), Nature, and may be incorporated in the recombinant viral vector genomes described herein to further regulate splicing activity.

### (vi) Nonsense Mediated Decay (NMD) Exons

In some embodiments, the recombinant viral vector genomes may comprise an alternatively-spliced exon cassette configured to regulate expression of a coding region of interest by including a nonsense mediated decay (NMD) exon (e.g., an alternative exon comprising a heterologous stop codon) within the RNA. In certain embodiments, the NMD exon is flanked by introns (or portion(s) thereof) for which alternative splicing is regulated. In some embodiments, an NMD exon is an exon that encodes at least one stop codon that is in frame with a previous exon, wherein the stop codon is upstream (5′) from the 3′ splice site of the exon. In various embodiments, the in-frame stop codon is inserted at least 100 nucleotides, at least 95 nucleotides, at least 90 nucleotides, at least 85 nucleotides, at least 80 nucleotides, at least 75 nucleotides, at least 70 nucleotides, at least 65 nucleotides, at least 60 nucleotides, at least 55 nucleotides, at least 50 nucleotides, at least 45 nucleotides, at least 40 nucleotides, at least 35 nucleotides, at least 30 nucleotides, at least 25 nucleotides, at least 20 nucleotides, at least 15 nucleotides, at least 10 nucleotides, or at least 5 nucleotides, or between 1 to 5 nucleotides upstream of the next 5′ splice junction.

In some embodiments, if the NMD exon is included in the spliced RNA, it causes degradation of the RNA via nonsense-mediated decay. In some embodiments, if the NMD exon is spliced out, the resulting transcript is stable, and in some embodiments encodes a functional (e.g., full-length) protein of interest.

In some embodiments, an alternatively-spliced exon cassette for which splicing is regulated is a construct configured to regulate expression of a protein by including a 5′ exon comprising an amino terminal amino acid encoding sequence (e.g., an ATG or part of the ATG) and/or translation control sequences, wherein the 5′ exon is separated from subsequent exon(s) by an intron for which splicing is regulated. In some embodiments, if the intron is spliced out of the RNA transcript, the recombinant 5′ exon is spliced in frame to the subsequent exon(s) and the resulting spliced transcript encodes a protein that is expressed. In some embodiments, if the intron is not spliced out of the RNA transcript, the recombinant 5′ exon is not spliced to the subsequent exon(s) and as a result a protein is not expressed from the transcript. In some embodiments, an intron (or portion thereof) for which splicing is regulated can be included within a gene that encodes a regulatory RNA (e.g., an siRNA). In some embodiments, an intron(s) (or portion thereof) for which splicing is regulated and that encodes regulatory RNA(s) can be included in an alternatively-spliced exon cassette encoding an RNA transcript.

(vii) Transgenes and Coding Regions Thereof

In various embodiments, the recombinant genomes disclosed herein may comprise one or more transgenes. A transgene may be recombinant (or “synthetic”), and may be modified to comprise an alternatively-spliced exon or an alternatively-spliced exon cassette described herein (e.g., see FIG. 1) such that the expression of the transgene or coding region of interest comes under the regulatory control of alternatively-spliced exon. A transgene (e.g., a coding region of a transgene) may encode any therapeutic agent, including, but not limited to a therapeutic protein, an antibody or fragment thereof, a bispecific antibody or fragment thereof, antigen-binding fragments, a nucleic acid molecule-based therapeutic (e.g., an siRNA, a microRNA, or an oligonucleotide), genome editing components (e.g., CRISPR/Cas9 based proteins and protein fusion and guide RNA molecules), and complexes (e.g., nucleoprotein complexes).

A coding region of a transgene may be naturally-occurring, and may in some embodiments comprise no nucleic acid modifications, relative to the coding region of a wild-type gene. In some embodiments, a coding region of a transgene may be synthetic. The coding region of a transgene may be considered synthetic if it undergoes one or more nucleic acid modifications, relative to the coding region of a wild-type gene. A nucleic acid modification may be a substitution or deletion of one or more nucleotides that form the nucleic acid sequence of the coding region of the transgene. In some embodiments, the modification comprises disrupting or deleting a native start codon located at the 5′ end of the coding region of the transgene. In some embodiments, the modification comprises the insertion of an alternatively-spliced exon into the coding region of the transgene.

In some embodiments, the coding region of the transgene may comprise one or more nucleic acid modifications (e.g., substitutions) such that the coding region comprises a “barcode” sequence. Barcode sequences may be useful in some embodiments to characterize the identity of the transgene (e.g., a transgene comprising a BIN1 alternative exon cassette and MTM1 coding sequence), for example when multiple transgenes are being tested together. In some embodiments, the wobble positions of five codons within the coding region of the transgene are modified to produce a barcode sequence. As will be understood, a “wobble position” is the third nucleic acid of a codon. Nucleic acids lying at wobble positions can be modified without altering the identity of the amino acid encoded by the associated codon (see FIG. 13, SEQ ID NO: 63). Thus, in some embodiments, the third nucleic acid of each of five consecutive codons in the coding region of the transgene is modified (e.g., 5 total substitutions are made; SEQ ID NOs: 65-75). In some embodiments, said modifications result in the formation of a barcode sequence which is 5 nucleic acid sequences in length. In some embodiments, the resultant barcode sequence is unique to the transgene within which it is comprised, and can be used to characterize the identity of said transgene.

In some embodiments, the five codons which are modified are located approximately 350 nucleotides from the 5′ end of the coding region of the transgene. In some embodiments, the five codons which are modified are located approximately 100, 125, 150, 175, 200, 225, 250, 275, 300, 325, 350, 375, 400, 425, 450, 475, 500, 525, or 550 nucleotides from the 5′ end of the coding region of the transgene. In some embodiments, the five codons which are modified are located approximately 100, 125, 150, 175, 200, 225, 250, 275, 300, 325, 350, 375, 400, 425, 450, 475, 500, 525, or 550 nucleotides from the 5′ end of the coding region of the transgene. In some embodiments, the five codons which are modified are located approximately 100-130, 120-150, 140-170, 160-190, 180-210, 200-230, 220-250, 240-270, 260-290, 280-310, 300-330, 320-350, 340-370, 370-400, 390-420, 410-440, 430-460, 450-480, 470-500, 490-520, 510-540, or 530-560 nucleotides from the 5′ end of the coding region of the transgene.

In some embodiments, a coding region of a transgene may naturally comprise one or more internal, out-of-frame ATG start codons. As will be understood, in the splicing condition wherein the alternative exon (comprising an ATG start codon at its 3′ end) is spliced-out, translation of the coding region via an alternate, out-of-frame ATG start codon located within the coding region of the transgene would be undesirable. However, any modification made to the coding region of the transgene must also preserve translation of the full-length protein when the alternative exon is spliced-in. Accordingly, in some embodiments one or more modifications are made to the coding region of the transgene which preserve translation of the full-length protein in the condition wherein the alternative exon is spliced-in, but which disrupt or terminate translation of the full-length protein in the condition wherein the alternative exon is spliced-out. In some embodiments, one or more nucleic acid substitutions are made within the coding region of the transgene to introduce one or more heterologous stop codons located downstream of (e.g., 3′ relative to) one or more of the internal, out-of-frame start codons located within the coding region of the transgene. As will be understood, such substitutions may comprise the substitution of 1, 2, or 3 nucleic acids to produce any of a TAA, TGA, or TAG stop codon, depending on the nucleic acids which are naturally present at the desired location within the coding sequence. Additionally or alternatively, in some embodiments a 3′ UTR intron is included in the transgene which elicits nonsense-mediated decay in the condition wherein the alternative exon is spliced-out (such that translation of the full-length protein is disrupted or terminated), but which preserves translation of the full-length protein in the condition wherein the alternative exon is spliced-in.

In some embodiments, the coding region of the transgene is from or is derived from a coding region from a gene selected from the group consisting of: MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, MTMR2, LAMP2, KIF5A, microdystrophin, C9ORF72, HTT, DNM2, BIN1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, cytochrome b/cytochrome c oxidase, CLCN1, SCN4A, DMPK, CNBP, MYOT, LMNA (Lamin A/C), CAV3, DNAJB6, DES, TNPO3, HNRPDL, CAPN3, DYSF, alpha-sarcoglycan, beta-sarcoglycan, gamma-sarcoglycan, delta-sarcoglycan, TCAP, TRIM32, FKRP, FXN, POMT1, FKTN, POMT2, POMGnT1, DAG1, ANO5, PLEC1, TRAPPC11, GMPPB, ISPD, LIMS2, POPDC1, TOR1AIP1, POGLUT2, LAMA2, COL6A1, POMT1, POMT2, DUX4, EMD, PAX7, PMP22, MPZ, MFN2, SMCHD1, or GJB1. In some embodiments, the coding region of the transgene is from or is derived from a coding region of FXN.

In some embodiments, the coding region of the transgene is from or is derived from a coding region of MTM1. In some embodiments, the coding region of the transgene which is or is derived from MTM1 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1881. In some embodiments, the coding region of the transgene which is or is derived from MTM1 comprises a polynucleotide having a nucleic acid sequence as set forth in SEQ ID NO: 1881.

In some embodiments, the coding region of the transgene is from or is derived from a coding region of CAPN3. In some embodiments, the coding region of the transgene which is or is derived from CAPN3 comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1882. In some embodiments, the coding region of the transgene which is or is derived from CAPN3 comprises a polynucleotide having a nucleic acid sequence as set forth in SEQ ID NO: 1882.

In other embodiments, the transgene may encode one or more therapeutic proteins (e.g., a biologic or biosimilar thereof), including, but not limited to: adalimumab, rituximab, pegfilgrastim, infliximab, bevacizumab, trastuzumab, etanercept, and epoetin.

### D. Packaging Recombinant Viral Genomes into Viral Vectors

Aspects of the present disclosure provide for the packaging of the herein disclosed recombinant viral genomes into viral vectors (i.e., complete viral particles which may infect cells to deliver the recombinant genomes, and the concomitant expression of the transgenes in a manner dependent on the alternatively-splice exons). Thus, in some embodiments a recombinant viral genome comprising an alternatively-spliced exon cassette as described herein is provided in a viral vector (e.g., an rAAV vector; a lentivirus vector). The viral vectors may include rAAV particles, lentivirus particles, or other viral vectors.

In some embodiments, the recombinant viral genomes packaged into the rAAV or lentiviral vectors further comprise a promoter. In some embodiments, the promoter is a constitutive promoter or a regulated promoter. In some embodiments, the regulated promoter is an inducible promoter. In some embodiments, the promoter comprises any one of: CMV, EF1alpha, CBh, synapsin, enolase, MECP2, MHCK7, Desmin, or GFAP.

In some embodiments, an MHCK7 promoter comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1880. In some embodiments, an MHCK7 promoter comprises a polynucleotide having a nucleic acid sequence as set forth in SEQ ID NO: 1880.

In some embodiments, the promoter is a ubiquitous promoter. In some embodiments, a ubiquitous promoter is a promoter selected from the group consisting of: an EF1 alpha promoter, a beta actin promoter, CMV, CBh, and CAG promoter. In some embodiments, the promoter is a tissue-specific promoter, such as a muscle- or heart-biased promoter. In some embodiments, a tissue-specific promoter, such as a muscle- or heart-biased promoter, is a promoter selected from the group consisting of: a muscle creatine kinase promoter, a C5-12 muscle promoter, MHCK7, and Desmin. In some embodiments, the promoter is a neuronal-biased promoter. In some embodiments, a neuronal-biased promoter is a promoter selected from the group consisting of: synapsin and MECP2. In some embodiments, the promoter is an astrocyte-biased promoter. In some embodiments, an astrocyte-biased promoter is a GFAP promoter. Thus, in some embodiments, the nucleic acid comprises a promoter and sequence corresponding to an RNA molecule that is capable of being expressed from the nucleic acid.

In some embodiments, the recombinant viral genome is sufficiently small to be effectively packaged in an AAV viral particle (e.g., the gene construct may be around 0.5-5 kb long, for example around 4.9 kb, 4.8 kb, 4.7 kb, 4.6 kb, 4.5 kb, 4.4 kb, 4.3 kb, 4.2 kb, 4.1 kb, 4 kb, 3.5 kb, or 3 kb long). So as to fit into the AAV viral particle, in some embodiments a nucleic acid comprises one or more truncated and/or recombinant introns, as described elsewhere herein. Accordingly, a recombinant intron for an rAAV vector is typically shorter than 4 kb, but can be between around 20 bases long and around 2,000 bases long to provide space for other components (e.g., exons, regulatory sequences, other introns, viral packaging sequences) in the nucleic acid (e.g., recombinant gene) construct. In some embodiments a recombinant intron is around 50 bases, around 100 bases, around 250 bases, around 500 bases, around 1,000 bases, around 1,500 bases, or around 2,000 bases long. In some embodiments, a recombinant intron is shorter than 4 kb, shorter than 3 kb, shorter than 2 kb, shorter than 1 kb, 100-900 bases long, or shorter than 500 bases long.

In some embodiments, the recombinant viral genome contains sufficient viral sequences for packaging in a viral vector (e.g., an rAAV particle). For example, in some embodiments a recombinant viral genome is flanked by viral sequences (for example, terminal repeat sequences) that are useful to package the recombinant viral genome in a viral particle (e.g., encapsidated by viral capsid proteins and/or an envelope, where appropriate). In some embodiments, the flanking terminal repeat sequences are rAAV inverted terminal repeats (ITRs). In some embodiments, the AAV ITR sequences comprise AAV1, AAV2, AAV5, AAV7, AAV8, or AAV9 ITR sequences.

In some embodiments, the AAV ITR sequences comprise AAV2 ITR sequences. In some embodiments, an AAV2 ITR comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1879. In some embodiments, an AAV2 ITR comprises a polynucleotide having a nucleic acid sequence as set forth in SEQ ID NO: 1879.

In some embodiments, the recombinant viral genome comprises a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 105 or SEQ ID NO: 106. In some embodiments, the recombinant viral genome comprises a polynucleotide having a nucleic acid sequence as set forth in either SEQ ID NO: 105 or SEQ ID NO: 106.

In some embodiments, the recombinant viral genome is a lentivirus genome comprising a DNA molecule, wherein the DNA molecule comprises sequences that encode an RNA molecule.

(i) Manufacture of rAAV Vectors

In some embodiments, the recombinant viral genome is encapsidated by an rAAV particle as described herein. The rAAV particle may be of any AAV serotype (e.g., 1, 2, 3, 4, 5, 6, 7, 8, 9, or 10), including any derivative (including non-naturally occurring variants of a serotype) or pseudotype. In some embodiments, the rAAV particle is an AAV8 particle, which may be pseudotyped with AAV2 ITRs. In some embodiments, an AAV2 ITR comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 1879. In some embodiments, an AAV2 ITR comprises a polynucleotide having a nucleic acid sequence as set forth in SEQ ID NO: 1879.

Non-limiting examples of derivatives and pseudotypes include AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAV5hH10, AAV2 (Y→F), AAV8 (Y733F), AAV2.15, AAV2.4, AAVM41, and AAVr3.45; or a derivative thereof. In some embodiments, the rAAV vector is of serotype AAV8. In some embodiments, the rAAV vector is pseudotyped. Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. 2012 Jan. 24. The AAV vector toolkit: poised at the clinical crossroads. Asokan A1, Schaffer D V, Samulski R J.). In some embodiments, the rAAV particle is a pseudotyped rAAV particle, which comprises (a) a nucleic acid vector comprising ITRs from one serotype (e.g., AAV2) and (b) a capsid comprised of capsid proteins derived from another serotype (e.g., AAV1, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Exemplary rAAV nucleic acid vectors useful according to the disclosure include single-stranded (ss) or self-complementary (sc) AAV nucleic acid vectors, such as single-stranded or self-complementary recombinant viral genomes.

Methods of producing rAAV particles and recombinant viral genomes are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid containing the recombinant viral genome may be combined with one or more helper plasmids, e.g., that contain a rep gene (e.g., encoding Rep78, Rep68, Rep52 and Rep40) and a cap gene (encoding VP1, VP2, and VP3, including a modified VP3 region), and transfected into a producer cell line such that the rAAV particle can be packaged and subsequently purified.

In some embodiments, the one or more helper plasmids includes a first helper plasmid comprising a rep gene and a cap gene and a second helper plasmid comprising a E1a gene, a E1b gene, a E4 gene, a E2a gene, and a VA gene. In some embodiments, the rep gene is a rep gene derived from AAV2 and the cap gene is derived from AAV2 and includes modifications to the gene in order to produce a modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

As used herein, the terms “engineered” and “recombinant” cells are intended to refer to a cell into which an exogenous polynucleotide segment (such as DNA segment that leads to the transcription of a biologically active molecule) has been introduced. Therefore, engineered cells are distinguishable from naturally occurring cells, which do not contain a recombinantly introduced exogenous DNA segment. Engineered cells are, therefore, cells that comprise at least one or more heterologous polynucleotide segments introduced through the hand of man.

To express a therapeutic agent in accordance with the present invention one may prepare a tyrosine capsid-modified rAAV particle containing an expression vector that comprises a therapeutic agent-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded polypeptide. This is the meaning of “recombinant expression” in this context. In some embodiments, the recombinant nucleic acid (e.g., viral) vector constructs are those that comprise an rAAV nucleic acid vector that contains a therapeutic gene of interest operably linked to one or more promoters that is capable of expressing the gene in one or more selected mammalian cells. Such nucleic acid vectors are described in detail herein.

In some embodiments, wherein the recombinant viral genome is an rAAV genome, the transgene comprising an alternatively-spliced exon cassette comprises a polynucleotide sequence as set forth in any one of SEQ ID NOs: 45-55. In some embodiments, wherein the recombinant viral genome is an rAAV genome, the transgene comprising an alternatively-spliced exon cassette comprises a polynucleotide sequence that is 90%, 91%, 92%, 93%, 94%, 95%, 96%, 97%, 98%, or 99% identical to any one of SEQ ID NOs: 45-55.

### (ii) Manufacture of Lentivirus Vectors

In some embodiments, a viral vector of the present disclosure comprises a recombinant lentivirus genome. Lentiviruses are the only type of virus that are diploid; they have two strands of RNA. The lentivirus is a retrovirus, meaning it has a single stranded RNA genome with a reverse transcriptase enzyme, which functions to perform transcription of the viral genetic material upon entering the cell. Lentiviruses also have a viral envelope with protruding glycoproteins that aid in attachment to the outer membrane of a host cell.

Within the lentivirus genome are RNA sequences that code for specific proteins that facilitate the incorporation of the viral sequences into genome of a host cell. The “gag” gene codes for the structural components of the viral nucleocapsid proteins: the matrix (MA/p17), the capsid (CA/p24) and the nucleocapsid (NC/p7) proteins. The “pol” domain codes for the reverse transcriptase and integrase enzymes. Lastly, the “env” domain of the viral genome encodes for the glycoproteins and envelope on the surface of the virus. The ends of the genome are flanked with long terminal repeats (LTRs). LTRs are necessary for integration of the dsDNA into the host chromosome. LTRs also serve as part of the promoter for transcription of the viral genes.

In some embodiments, the env, gag, and/or pol vector(s) forming the particle do not contain a nucleic acid sequence from the lentiviral genome that expresses an envelope protein. In some embodiments, a separate vector containing a nucleic acid sequence encoding an envelope protein operably linked to a promoter is used (e.g., an env vector). In some embodiments, such env vector also does not contain a lentiviral packaging sequence. In some embodiments, the env nucleic acid sequence encodes a lentiviral envelope protein.

The native lentivirus promoter is located in the U3 region of the 3′ LTR. As will be understood by those of skill in the art, the presence of the lentivirus promoter can in some embodiments interfere with heterologous promoters operably linked to a transgene. To minimize such interference and better regulate the expression of transgenes, in some embodiments the lentiviral promoter is deleted. In some embodiments, the lentivirus vector contains a deletion within the viral promoter. After reverse transcription, such a deletion is in some embodiments transferred to the 5′ LTR, yielding a vector/provirus that is incapable of synthesizing vector transcripts from the 5′ LTR in the next round of replication.

In some embodiments, the lentivirus particle is expressed by a vector system encoding the necessary viral proteins to produce a lentivirus particle. In some embodiments, there is at least one vector containing a nucleic acid sequence encoding the lentiviral Pol proteins necessary for reverse transcription and integration, operably linked to a promoter. In some embodiments, the Pol proteins are expressed by multiple vectors. In some embodiments, there is also a vector containing a nucleic acid sequence encoding the lentiviral Gag proteins necessary for forming a viral capsid operably linked to a promoter. In some embodiments, the gag-pol genes are on the same vector. In some embodiments, the gag nucleic acid sequence is on a separate vector than at least some of the pol nucleic acid sequence. In some embodiments, the gag nucleic acid sequence is on a separate vector from all the pol nucleic acid sequences that encode Pol proteins.

In some embodiments, the lentivirus vector does not contain nucleotides from the lentiviral genome that package lentiviral RNA, referred to as the lentiviral packaging sequence.

It will be understood that selective inclusion of envelopes could result in changes in infectivity, such that the lentivirus vector could infect many different types of cells, and could be targeted to specific cell types of interest. Accordingly, in some embodiments, the envelope protein is not from the lentivirus, but from a different virus. The resultant lentivirus particle is referred to as a pseudotyped particle. In some embodiments, env gene that encodes an envelope protein that targets an endocytic compartment such as that of the influenza virus, VSV-G, alpha viruses (Semliki forest virus, Sindbis virus), arenaviruses (lymphocytic choriomeningitis virus), flaviviruses (tick-borne encephalitis virus, Dengue virus), rhabdoviruses (vesicular stomatitis virus, rabies virus), and orthomyxoviruses (influenza virus) is used.

In some embodiments, the lentivirus is a human immunodeficiency virus (HIV1 or HIV2), a feline immunodeficiency virus (FIV), a bovine immunodeficiency virus (BIV), a caprine arthritis encephalitis virus, an equine infectious anemia virus, a jembrana disease virus, a puma lentivirus, aimian immunodeficiency virus, or a visna-maedi virus.

In some embodiments, a nucleic acid sequence encoding a transgene comprising an alternatively-spliced exon cassette of the present invention is inserted into the empty lentiviral particles by use of a plurality of vectors each containing a nucleic acid segment of interest and a lentiviral packaging sequence necessary to package lentiviral RNA into the lentiviral particles (the packaging vector). In some embodiments, the packaging vector contains a 5′ and 3′ lentiviral LTR with the desired nucleic acid segment inserted between them. The nucleic acid segment can be antisense molecules or, in some embodiments, encodes a therapeutic protein. As will be understood, proper orientation of the transgene within the lentiviral genome is necessary to avoid the loss of introns (e.g., the splicing-out of introns) during viral packaging. Accordingly, in some embodiments, the transgene is oriented in the anti-sense orientation within the lentiviral genome. In some embodiments, orienting the transgene in the anti-sense direction within the lentiviral genome avoids the loss of introns (e.g., the splicing-out of introns) during viral packaging.

In some embodiments, the packaging vector contains a selectable marker gene. Such marker genes are well known in the art and include such genes as green fluorescent protein (GFP), blue fluorescent protein (BFP), luciferase, LacZ, nerve growth factor receptor (NGFR), etc.

### E. Methods of Delivering Viral Vectors

Some aspects of the invention contemplate a method of treating a disease or condition in a subject comprising administering a viral vector of the present disclosure to a subject, wherein the viral vectors comprise a recombinant viral genome described herein. Accordingly, provided herein is a method of delivering the disclosed viral (e.g., rAAV; lentivirus) particles. In some embodiments, viral particles are delivered by administering any one of the compositions disclosed herein to a subject. In some embodiments, “administering” or “administration” means providing a material to a subject in a manner that is pharmacologically useful. In some embodiments, viral particles are delivered to one or more tissues and cell types in a subject. In some embodiments, viral particles are delivered to one or more of muscle, heart, CNS, and immune cells. In some embodiments, delivery of a viral particle restores transcriptome homeostasis.

Delivery vehicles, vectors, particles, nanoparticles, formulations and components thereof which are suitable for expression of one or more elements of an engineered AAV capsid system as described herein are as described in, for example, International Patent Application Publication Nos. WO 2021/050974 and WO 2021/077000 and International Application No. PCT/US2021/042812, the contents of each of which are incorporated by reference herein.

In some embodiments, a viral particle is administered to the subject parenterally. In some embodiments, a viral particle is administered to a subject subcutaneously, intraocularly, intravitreally, subretinally, intravenously (IV), intracerebro-ventricularly, intramuscularly, intrathecally (IT), intracisternally, intraperitoneally, enterally, via inhalation, topically, or by direct injection to one or more cells, tissues, or organs. In some embodiments, a viral particle is administered to the subject by injection into the hepatic artery or portal vein.

To “treat” a disease, as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host organ, tissue, or cell. A therapeutically acceptable amount may be an amount that is capable of treating a disease. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, a single composition comprising viral particles as disclosed herein is administered only once. In some embodiments, a subject may need more than 1 administration of a viral composition (e.g., 2, 3, 4, 5, 6, 7, 8, 9, or 10 or more times). For example, a subject may need to be provided a second administration of any one of the viral compositions as disclosed herein 1 day, 1 week, 1 month, 1 year, 2 years, 5 years, or 10 years after the subject was administered a first composition. In some embodiments, a first composition of viral particles is different from the second composition of viral particles.

In some embodiments, the administration of the composition is repeated at least once (e.g., at least once, at least twice, at least thrice, at least four times, at least five times, at least six times, at least 10 times, at least 25 times, or at least 50 times), and wherein the time between a repeated administration and a previous administration is at least 1 month (e.g., at least 1 month, at least 2 months, at least 3 months, at least 4 months, at least 5 months, at least 6 months, or at least 12 months). In some embodiments, the administration of the composition is repeated at least once, and wherein the time between a repeated administration and a previous administration is at least 1 year (e.g., at least 1, at least 2, at least 3, at least 4, at least 5, at least 10, or at least 20 years).

In some embodiments, the administration of the composition is facilitated by AAV capsids such as AAV1-9, e.g., with AAV2 ITRs, or other capsids that sufficiently deliver to affected tissues.

Additional AAV vectors are described in International Patent Application Publication No. WO 2019/2071632, the content of which is incorporated by reference herein.

Further AAV vectors are described in International Patent Application Publication Nos. WO 2020/086881 and WO 2020/235543, the contents of each of which are incorporated by reference herein.

Further AAV vectors are described in International Patent Application Publication Nos. WO 2005/033321; WO 2006/110689; WO 2007/127264; WO 2008/027084; WO 2009/073103; WO 2009/073104; WO 2009/105084; WO 2009/134681; WO 2009/136977; WO 2010/051367; WO 2010/138675; WO 2001/038187; WO 2012/112832; WO 2015/054653; WO 2016/179496; WO 2017/100791; WO 2017/019994; WO 2018/209154; WO 2019/067982; WO 2019/195701; WO 2019/217911; WO 2020/041498; WO 2020/210839; U.S. Pat. Nos. 7,906,111; 9,737,618; 10,265,417; 10,485,883; 10,695,441; 10,722,598; 8,999,678; 10,301,648; 10,626,415; 9,198,984; 10,155,931; 8,524,219; 9,206,238; 8,685,387; 9,359,618; 8,231,880; 8,470,310; 9,597,363; 8,940,290; 9,593,346; 10,501,757; 10,786,568; 10,973,928; 10,519,198; 8,846,031; 9,617,561; 9,884,071; 10,406,173; 9,596,220; 9,719,010; 10,117,125; 10,526,584; 10,881,548; 10,738,087; U.S. Patent Publication No. 2011-023353; U.S. Patent Publication No. 2019-0015527; U.S. Patent Publication No. 2020-155704; U.S. Patent Publication No 2017-0191079; U.S. Patent Publication No. 2019-0218574; U.S. Patent Publication No. 2020-0208176; U.S. Patent Publication No. 2020-0325491; U.S. Patent Publication No. 2019-0055523; U.S. Patent Publication No. 2020-0385689; U.S. Patent Publication No. 2009-0317417; U.S. Patent Publication No. 2016-0051603; U.S. Patent Publication No. 2016-00244783; U.S. Patent Publication No. 2017-0183636; U.S. Patent Publication No. 2020-0263201; U.S. Patent Publication No. 2020-0101099; U.S. Patent Publication No. 2020-0318082; U.S. Patent Publication No. 2018-0369414; U.S. Patent Publication No. 2019-0330278; U.S. Patent Publication No. 2020-0231986, the contents of each of which are incorporated by reference herein.

### F. Subjects

Aspects of the disclosure relate to methods for use with a subject (e.g., a mammal). In some embodiments, a mammalian subject is a human, a non-human primate, or other mammalian subject. In some embodiments, the subject has one or more mutations associated with aberrant intron and/or alternative splicing.

In some embodiments, a subject suffers from or is at risk of developing a disease or condition associated with aberrant splice regulation resulting in one or more symptoms of a disease or condition. Non-limiting examples of these diseases/conditions include instances in which the homeostasis of RNA binding proteins is altered (e.g., other repeat expansion diseases), or diseases/conditions in which there are mutations in RNA binding protein sequences. In some embodiments, the disease or condition is selected from: a repeat expansion disease, a laminopathy, a cardiomyopathy, a muscular dystrophy, a neurodegenerative disease, a cancer, an intellectual disability, and/or premature aging.

In a non-limiting example, compositions of this application are administered to a subject resulting in regulated overexpression of the RNA binding protein exhibiting aberrant activity. In another non-limiting example, compositions of this application are administered to a subject resulting in the regulated addition of additional non-mutated, non-aberrant RNA binding protein(s).

In some embodiments, the disease or condition is selected from the group consisting of: Dentatorubral-pallido-luysian atrophy (DRPLA), myotonic dystrophy type 1 (DM1), myotonic dystrophy type 2 (DM2), Fragile X syndrome of mental retardation (FMR1), Fragile X tremor ataxia syndrome (FXTAS), FRAXE mental retardation (FMR2), Friedreichs ataxia (FRDA), Huntington disease (HD), Huntington disease-like 2 (HDL2), Oculopharyngeal muscular dystrophy (OPMD), Myoclonic epilepsy type 1, Alzheimer's disease, ALS/FTD, spinocerebellar ataxia type 1 (SCA1), spinocerebellar ataxia type 2 (SCA2), spinocerebellar ataxia type 3 (SCA3), spinocerebellar ataxia type 6 (SCA6), spinocerebellar ataxia type 7 (SCAT), spinocerebellar ataxia type 8 (SCA8), spinocerebellar ataxia type 10 (SCA10), spinocerebellar ataxia type 12 (SCA12), spinocerebellar ataxia type 17 (SCA17), Syndromic/non-syndromic X-linked mental retardation, Emery-Dreifuss muscular dystrophy type 2, familial partial lipodystrophy, limb girdle muscular dystrophy type 1B, dilated cardiomyopathy, familial partial lipodystrophy, Charcot-Marie-Tooth disorder type 2B1, mandibuloacral dysplasia, childhood progeria syndrome (Hutchinson-Gilford syndrome), Werner syndrome, Dilated cardiomyopathy (DCM), Hypertrophic cardiomyopathy (HCM), Restrictive cardiomyopathy (RCM), Left Ventricular Non-compaction (LVNC), Arrhythmogenic Right Ventricular Dysplasia (ARVD), takotsubo cardiomyopathy, Duchenne muscular dystrophy, Becker muscular dystrophy, Limb-girdle muscular dystrophy, Facioscapulohumeral muscular dystrophy, Congenital muscular dystrophy, Oculopharyngeal muscular dystrophy, Distal muscular dystrophy, Emery-Dreifuss muscular dystrophy, dementia, Parkinson's disease (PD), a PD-related disorder, Prion disease, a motor neuron disease (MND), Progressive bulbar palsy (PBP), Progressive muscular atrophy (PMA), Primary lateral sclerosis (PLS), Spinal muscular atrophy (SMA), a bladder cancer, a breast cancer, a colorectal cancer, a kidney cancer, a lung cancer, a lymphoma, a melanoma, an oral cancer, an ovarian cancer, an oropharyngeal cancer, a pancreatic cancer, a prostate cancer, a thyroid cancer, a uterine cancer, Down syndrome, Prader-Willi Syndrome (PWS), Bloom Syndrome, Cockayne Syndrome Type I-216400, Cockayne Syndrome Type III, Cockayne Syndrome Type I, Hutchinson-Gilford Progeria Syndrome, Mandibuloacral Dysplasia with Type A Lipodystrophy, Progeria, Adult Onset Progeroid Syndrome, Neonatal Rothmund-Thomson Syndrome, Seip Syndrome, Werner Syndrome, Replication Focus-Forming Activity 1, myotubular myopathy, Danon Disease, and/or centronuclear myopathy.

Non-limiting examples of symptoms of these diseases/conditions include neurodevelopmental, neurofunctional, or neurodegenerative changes (e.g., ALS, FTD, Spinocerebellar Ataxias, FXTAS, or Huntington's Disease symptoms) or abnormal proliferation or migration of cells (e.g., as in cancer). For example, myotonic dystrophy type 1 and type 2 (dystrophia myotonica, DM1 and DM2, respectively) are caused by expanded CTG repeats in the DMPK gene and CCTG repeats in the CNBP gene, respectively. Both diseases are highly multi-systemic with symptoms in skeletal muscles, cardiac tissue, gastrointestinal tract, endocrine system, and central nervous system, among others.

In some aspects, the present disclosure relates to methods and compositions that are useful for treating myotonic dystrophy type 1 and type 2 (dystrophia myotonica, DM1 and DM2, respectively), for example by delivering viral particles comprising viral constructs (e.g., containing one or more alternative spicing cassettes) to cells or tissue in a subject. In addition to the symptoms described above, DM1 can also manifest in a severe form called congenital DM1, in which profound developmental delays occur. A 25% chance of death before the age of 18 months and 50% chance of survival into mid-30s has been reported. Methods and compositions of the application can be useful to treat, alleviate, or otherwise improve one or more symptoms of DM1.

Accordingly, in some embodiments one or more viral constructs can be delivered to a subject having one or more symptoms of myotonic dystrophy. Such symptoms may include, but are not limited to, delayed muscle relaxation, muscle weakness, prolonged involuntary muscle contraction, loss of muscle, abnormal heart rhythm, cataracts, or difficulty swallowing. In some embodiments, a viral composition provided herein is administered to a subject having congenital DM1 or DM2. In some embodiments, the viral constructs treat, alleviate, ameliorate, or otherwise improve one or more symptoms associated with DM1 and/or DM2. In some embodiments, the viral constructs reduce muscle weakness, reduce muscle loss, reduce muscle wasting, reduce prolonged muscle contractions, improve speech, and/or improve swallowing in a subject. In some embodiments, treatment reduces or corrects one or more other symptoms of myotonic dystrophy.

In some embodiments, splicing of a recombinant intron and/or an alternatively-spliced exon is sufficiently regulated to be therapeutically effective.

### G. Enumerated Embodiments

Certain embodiments are set forth in the enumerated clauses below.

Clause 1. A recombinant viral genome for delivering a transgene, wherein said genome comprises at least one alternatively-spliced exon cassette comprising at least one alternatively-spliced exon, at least one flanking intron, and a coding region of the transgene.
Clause 2. The viral genome of clause 1, wherein the alternatively-spliced exon is retained in the spliced transcript.
Clause 3. The viral genome of clause 1 or clause 2, wherein the alternatively-spliced exon cassette further comprises at least one constitutive exon.
Clause 4. The viral genome of any preceding clause, wherein the alternatively-spliced exon cassette comprises one flanking intron.
Clause 5. The viral genome of clause 4, wherein the flanking intron is located 3′ or 5′ to the alternatively-spliced exon.
Clause 6. The viral genome of any one of clauses 1-3, wherein the alternatively-spliced exon cassette comprises two flanking introns.
Clause 7. The viral genome of any preceding clause, wherein the alternatively-spliced exon comprises at least one modification, relative to a naturally occurring alternatively-spliced exon.
Clause 8. The viral genome of any preceding clause, wherein the alternatively-spliced exon comprises at its 3′ end a heterologous start codon or part of a heterologous start codon.
Clause 9. The viral genome of clause 8, wherein all native start codons located 5′ to the heterologous start codon are disrupted or deleted.
Clause 10. The viral genome of any preceding clause, wherein the alternatively-spliced exon is located 5′ to the coding region of the transgene.
Clause 11. The viral genome of any one of clauses 1-7, wherein the alternatively-spliced exon cassette comprises two alternatively-spliced exons, each with flanking introns.
Clause 12. The viral genome of clause 11, wherein the two alternatively-spliced exons are adjacent.
Clause 13. The viral genome of clause 11 or clause 12, wherein the constitutive exon is located 5′ to the two alternatively-spliced exons.
Clause 14. The viral genome of any one of clauses 11-13, wherein each alternatively-spliced exon comprises at its 3′ end a heterologous start codon or part of a heterologous start codon.
Clause 15. The viral genome of clause 14, wherein all native start codons located 5′ to the heterologous start codon of the 5′-most alternatively-spliced exon are disrupted or deleted.
Clause 16. The viral genome of any one of clauses 11-15, wherein only one of the two alternatively-spliced exons is retained in the spliced transcript.
Clause 17. The viral genome of any one of clauses 11-16, wherein the 5′-most alternatively-spliced exon is retained in the spliced transcript.
Clause 18. The viral genome of any one of clauses 11-16, wherein the 3′-most alternatively-spliced exon is retained in the spliced transcript.
Clause 19. The viral genome of any preceding clause, wherein the alternatively-spliced exon(s) and flanking intron(s) are located within the coding region of the transgene.
Clause 20. The viral genome of any preceding clause, wherein the alternatively-spliced exon comprises a heterologous, in-frame stop codon.
Clause 21. The viral genome of clause 20, wherein the heterologous, in-frame stop codon is at least 50 nucleotides upstream of the next 5′ splice junction.
Clause 22. The viral genome of clause 20 or clause 21, wherein the heterologous stop codon elicits nonsense-mediated decay.
Clause 23. The viral genome of any preceding clause, wherein the alternatively-spliced exon is retained in the spliced transcript in distinct tissues or in distinct cell types.
Clause 24. The viral genome of any preceding clause, wherein the alternatively-spliced exon is retained in the spliced transcript in the presence of activated T cells, and/or in states of inflammation.
Clause 25. The viral genome of any preceding clause, wherein the alternatively-spliced exon is retained in the spliced transcript in cells exhibiting one or more signs or symptoms of a disease state, and/or in cells exhibiting non-homeostatic levels of the protein encoded by the natural gene comprising the transgene.
Clause 26. The viral genome of any preceding clause, wherein the alternatively-spliced exon comprises an alternatively-spliced exon from a gene selected from the group consisting of ABCC1, AK125149, ASCC2, BAT2D1, BBX, BRD8, BRE, C17orf70, CAMKK2, CBFB, CCAR1, CCDC7CD6, CHTF8, COL4A3BP, COL6A3, CUGBP1, CUGBP2, CXorf45, DENND3, DGUOK, DKFZp762G094, DNAJC7, DNASE1, EIF4A2, EIF4G2, EIF4H, EXOC7, EZH2, FAM120A, FAM136A, FAM36A, FARSB, FBXO38, FGFR1OP2, FIP1L1, FOXRED1, FUBP3, GALT, GATA3, GOLGA2, HIF1A, HMMR, HRB, IKZF1, ILF3, IRAK4, IRF1, KCTD13, LEF1, LUC7L, LYRM1, MALT1 e7, MAP2K7, MAP3K7, MAP4K2, MBNL2, MFF, NAE1, NCSTN, NR4A3, NRF1, NUP98, PARP6, PCM1, PLAUR, PLSCR3, PPIL5, PPP5C, PTPRC-E4, PTPRC-E6, PTS, RABL5, RAPH1, SEC16A, SFRS3, SFRS7, SLMAP, SNRNP70, STAT6, TBC1D1, TIMM8B, TIR8, TRA2A, TROVE2, UGCGL1, VAP-B, VAV1, ZNF384, ZNF496, CAMK2B, PKP2, LGMN, NRAP, VPS39, KSR1, PDLIM3, BIN1, ARFGAP2, KIF13A, and/or PICALM.
Clause 27. The viral genome of any preceding clause, wherein the alternatively-spliced exon comprises an alternatively-spliced exon comprising a polynucleotide sequence as set forth in any one of SEQ ID NOs: 23-44.
Clause 28. The viral genome of any preceding clause, wherein the flanking intron(s) is a native flanking intron(s) of the alternatively-spliced exon(s).
Clause 29. The viral genome of any preceding clause, wherein the flanking intron(s) comprises at its 5′ end a 5′ splice donor site.
Clause 30. The viral genome of any preceding clause, wherein the flanking intron(s) comprises at its 3′ end a 3′ splice donor site.
Clause 31. The viral genome of any preceding clause, wherein the flanking intron(s) comprises no modifications, relative to a naturally occurring intron.
Clause 32. The viral genome of any one of clauses 1-31, wherein the flanking intron(s) comprises at least one modification, relative to a naturally occurring intron.
Clause 33. The viral genome of clause 32, wherein the modification is a substitution or deletion of one or more nucleotides.
Clause 34. The viral genome of any preceding clause, wherein the flanking intron(s) is a regulated intron.
Clause 35. The viral genome of any preceding clause, wherein the flanking intron(s) comprises an intron from a gene selected from the group consisting of ABCC1, AK125149, ASCC2, BAT2D1, BBX, BRD8, BRE, C17orf70, CAMKK2, CBFB, CCAR1, CCDC7CD6, CHTF8, COL4A3BP, COL6A3, CUGBP1, CUGBP2, CXorf45, DENND3, DGUOK, DKFZp762G094, DNAJC7, DNASE1, EIF4A2, EIF4G2, EIF4H, EXOC7, EZH2, FAM120A, FAM136A, FAM36A, FARSB, FBXO38, FGFR1OP2, FIP1L1, FOXRED1, FUBP3, GALT, GATA3, GOLGA2, HIF1A, HMMR, HRB, IKZF1, ILF3, IRAK4, IRF1, KCTD13, LEF1, LUC7L, LYRM1, MALT1 e7, MAP2K7, MAP3K7, MAP4K2, MBNL2, MFF, NAE1, NCSTN, NR4A3, NRF1, NUP98, PARP6, PCM1, PLAUR, PLSCR3, PPIL5, PPP5C, PTPRC-E4, PTPRC-E6, PTS, RABL5, RAPH1, SEC16A, SFRS3, SFRS7, SLMAP, SNRNP70, STAT6, TBC1D1, TIMM8B, TIR8, TRA2A, TROVE2, UGCGL1, VAP-B, VAV1, ZNF384, ZNF496, CAMK2B, PKP2, LGMN, NRAP, VPS39, KSR1, PDLIM3, BIN1, ARFGAP2, KIF13A, and/or PICALM.
Clause 36. The viral genome of any preceding clause, wherein the flanking intron(s) comprises an intron comprising a polynucleotide sequence as set forth in any one of SEQ ID NOs: 1-22, 103, and 104.
Clause 37. The viral genome of any one of clauses 3-36, wherein the constitutive exon is a native exon of the transgene.
Clause 38. The viral genome of any one of clauses 3-36, wherein the constitutive exon is not a native exon of the transgene.
Clause 39. The viral genome of any one of clauses 3-38, wherein the constitutive exon is from the same gene as the alternatively-spliced exon(s).
Clause 40. The viral genome of clause 39, wherein the gene is the transgene.
Clause 41. The viral genome of any one of clauses 3-38, wherein the constitutive exon is not from the same gene as the alternatively-spliced exon(s).
Clause 42. The viral genome of any one of clauses 39-41, wherein the gene is a gene selected from the group consisting of MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, MTMR2, LAMP2, KIF5A, a microdystrophin-encoding gene, C9ORF72, HTT, DNM2, BIN1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, a cytochrome b oxidase-encoding gene, a cytochrome c oxidase-encoding gene, CLCN1, SCN4A, DMPK, CNBP, MYOT, LMNA, CAV3, DNAJB6, DES, TNPO3, HNRPDL, CAPN3, DYSF, an alpha-sarcoglycan-encoding gene, a beta-sarcoglycan-encoding gene, a gamma-sarcoglycan-encoding gene, a delta-sarcoglycan-encoding gene, TCAP, TRIM32, FKRP, FXN, POMT1, FKTN, POMT2, POMGnT1, DAG1, ANO5, PLEC1, TRAPPC11, GMPPB, ISPD, LIMS2, POPDC1, TOR1AIP1, POGLUT2, LAMA2, COL6A1, POMT1, POMT2, DUX4, EMD, PAX7, PMP22, MPZ, MFN2, SMCHD1, SMN, Lamin A/C (LAMN), GJB1, ABCC1, AK125149, ASCC2, BAT2D1, BBX, BRD8, BRE, C17orf70, CAMKK2, CBFB, CCAR1, CCDC7CD6, CHTF8, COL4A3BP, COL6A3, CUGBP1, CUGBP2, CXorf45, DENND3, DGUOK, DKFZp762G094, DNAJC7, DNASE1, EIF4A2, EIF4G2, EIF4H, EXOC7, EZH2, FAM120A, FAM136A, FAM36A, FARSB, FBXO38, FGFR1OP2, FIP1L1, FOXRED1, FUBP3, GALT, GATA3, GOLGA2, HIF1A, HMMR, HRB, IKZF1, ILF3, IRAK4, IRF1, KCTD13, LEF1, LUC7L, LYRM1, MALT1 e7, MAP2K7, MAP3K7, MAP4K2, MBNL2, MFF, NAE1, NCSTN, NR4A3, NRF1, NUP98, PARP6, PCM1, PLAUR, PLSCR3, PPIL5, PPP5C, PTPRC-E4, PTPRC-E6, PTS, RABL5, RAPH1, SEC16A, SFRS3, SFRS7, SLMAP, SNRNP70, STAT6, TBC1D1, TIMM8B, TIR8, TRA2A, TROVE2, UGCGL1, VAP-B, VAV1, ZNF384, ZNF496, CAMK2B, PKP2, LGMN, NRAP, VPS39, KSR1, PDLIM3, BIN1, ARFGAP2, KIF13A, and/or PICALM.
Clause 43. The viral genome of any preceding clause, further comprising a promoter.
Clause 44. The viral genome of clause 43, wherein the promoter is a native promoter of the transgene.
Clause 45. The viral genome of clause 43, wherein the promoter is not a native promoter of the transgene.
Clause 46. The viral genome of any one of clauses 43-45, wherein the promoter is constitutive.
Clause 47. The viral genome of any one of clauses 43-45, wherein the promoter is inducible.
Clause 48. The viral genome of any one of clauses 43-47, wherein the promoter is a tissue-specific promoter.
Clause 49. The viral genome of any one of clauses 43-48, wherein the promoter is selected from the group consisting of an EF1 alpha promoter, beta actin promoter, CMV, muscle creatine kinase promoter, C5-12 muscle promoter, MHCK7, CBh, synapsin, MECP2, enolase, GFAP, Desmin, and CAG promoter.
Clause 50. The viral genome of any one of clauses 43-49, wherein the promoter drives expression of the transgene.
Clause 51. The viral genome of any one of clauses 1-50, wherein the coding region of the transgene comprises at least one modification, relative to a coding region of a naturally occurring gene.
Clause 52. The viral genome of clause 51, wherein the modification is a substitution or deletion of at least one nucleotide.
Clause 53. The viral genome of clause 51 or clause 52, wherein the coding region of the transgene comprises a deletion of a native start codon, or a portion thereof.
Clause 54. The viral genome of any preceding clause, wherein the transgene comprises one or more recombinant introns.
Clause 55. The viral genome of any one of clauses 51-54, wherein the naturally occurring gene is a gene selected from the group consisting of MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, MTMR2, LAMP2, KIF5A, a microdystrophin-encoding gene, C9ORF72, HTT, DNM2, BIN1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, a cytochrome b oxidase-encoding gene, a cytochrome c oxidase-encoding gene, CLCN1, SCN4A, DMPK, CNBP, MYOT, LMNA, CAV3, DNAJB6, DES, TNPO3, HNRPDL, CAPN3, DYSF, an alpha-sarcoglycan-encoding gene, a beta-sarcoglycan-encoding gene, a gamma-sarcoglycan-encoding gene, a delta-sarcoglycan-encoding gene, TCAP, TRIM32, FKRP, FXN, POMT1, FKTN, POMT2, POMGnT1, DAG1, ANDS, PLEC1, TRAPPC11, GMPPB, ISPD, LIMS2, POPDC1, TOR1AIP1, POGLUT2, LAMA2, COL6A1, POMT1, POMT2, DUX4, EMD, PAX7, PMP22, MPZ, MFN2, SMCHD1, SMN, Lamin A/C (LAMN), and/or GJB1.
Clause 56. The viral genome of any preceding clause, wherein the viral genome is a genome from a recombinant adeno-associated virus (rAAV), lentivirus, retrovirus, or foamyvirus.
Clause 57. The viral genome of clause 56, wherein the viral genome is from an rAAV.
Clause 58. The viral genome of clause 56 or clause 57, wherein the transgene is flanked by AAV inverted terminal repeat (ITR) sequences.
Clause 59. The viral genome of clause 58, wherein the ITR sequences comprise AAV1, AAV2, AAV5, AAV7, AAV8, or AAV9 ITR sequences.
Clause 60. The viral genome of clause 56, wherein the viral genome is from a lentivirus.
Clause 61. The viral genome of clause 60, wherein the alternatively-spliced exon cassette is located on the minus strand of the lentivirus genome.
Clause 62. The viral genome of any preceding clause, further comprising a 3′ untranslated region (UTR) that is endogenous or exogenous to the transgene.
Clause 63. The viral genome of clause 62, wherein the exogenous 3′ UTR is the 3′ UTR from bovine growth hormone, SV40, EBV, or Myc.
Clause 64. A viral particle comprising a viral genome according to any preceding clause. Clause 65. The viral particle of clause 64, wherein the viral particle is an rAAV particle. Clause 66. The viral particle of clause 65, wherein the rAAV particle comprises AAV serotype 1, 2, 3, 4, 5, 6, 7, 8, 9, or 10.
Clause 67. The viral particle of clause 65, wherein the rAAV particle comprises AAV derivative or pseudotype AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAV5hH10, AAV2 (Y→F), AAV8 (Y733F), AAV2.15, AAV2.4, AAVM41, and AAVr3.45.
Clause 68. The viral particle of any one of clauses 64-67, further comprising at least one helper plasmid.
Clause 69. The viral particle of clause 68, wherein the helper plasmid comprises a rep gene and a cap gene.
Clause 70. The viral particle of clause 69, wherein the rep gene encodes Rep78, Rep68, Rep52, or Rep40.
Clause 71. The viral particle of clause 69 or clause 70, wherein the cap gene encodes a VP1, VP2, and/or VP3 region of the viral capsid protein.
Clause 72. The viral particle of any one of clauses 68-71, wherein the viral particle comprises two helper plasmids.
Clause 73. The viral particle of clause 72, wherein the first helper plasmid comprises a rep gene and a cap gene and the second helper plasmid comprises a E1a gene, a E1b gene, a E4 gene, a E2a gene, and a VA gene.
Clause 74. The viral particle of clause 64, wherein the viral particle is a recombinant lentivirus particle.
Clause 75. The viral particle of clause 74, wherein the lentivirus is a human immunodeficiency virus (HIV1 or HIV2), a feline immunodeficiency virus (FIV), a bovine immunodeficiency virus (BIV), a caprine arthritis encephalitis virus, an equine infectious anemia virus, a jembrana disease virus, a puma lentivirus, aimian immunodeficiency virus, or a visna-maedi virus.
Clause 76. The viral particle of clause 74 or clause 75, further comprising a viral envelope.
Clause 77. A method of treating a disease or condition in a subject comprising administering a viral genome according to any one of clauses 1-63 or a viral particle according to any one of clauses 64-76 to the subject.
Clause 78. The method of clause 77, wherein the subject is a mammal.
Clause 79. The method of clause 78, wherein the mammal is a human.
Clause 80. The method of any one of clauses 77-79, wherein the viral genome or viral particle is administered to the subject at least one time.
Clause 81. The method of clause 80, wherein the viral genome or viral particle is administered to the subject 2, 3, 4, 5, 6, 7, 8, 9, or 10 times.
Clause 82. The method of any one of clauses 77-81, wherein the viral genome or viral particle is administered to the subject parenterally, subcutaneously, intraocularly, intravitreally, subretinally, intravenously (IV), intracerebro-ventricularly, intramuscularly, intrathecally (IT), intracisternally, intraperitoneally, enterally, via inhalation, topically, or by direct injection to one or more cells, tissues, or organs.
Clause 83. The method of any one of clauses 77-82, wherein the viral genome or viral particle is administered to the subject by intravenous injection, intramuscular injection, intrathecal injection, or intravitreal injection.
Clause 84. The method of any one of clauses 77-83, wherein the disease or condition is a disease or condition selected from the group consisting of Dentatorubral-pallido-luysian atrophy (DRPLA), myotonic dystrophy type 1 (DM1), myotonic dystrophy type 2 (DM2), Fragile X syndrome of mental retardation (FMR1), Fragile X tremor ataxia syndrome (FXTAS), FRAXE mental retardation (FMR2), Friedreichs ataxia (FRDA), Huntington disease (HD), Huntington disease-like 2 (HDL2), Oculopharyngeal muscular dystrophy (OPMD), Myoclonic epilepsy type 1, Alzheimer's disease, ALS/FTD, spinocerebellar ataxia type 1 (SCA1), spinocerebellar ataxia type 2 (SCA2), spinocerebellar ataxia type 3 (SCA3), spinocerebellar ataxia type 6 (SCA6), spinocerebellar ataxia type 7 (SCAT), spinocerebellar ataxia type 8 (SCA8), spinocerebellar ataxia type 10 (SCA10), spinocerebellar ataxia type 12 (SCA12), spinocerebellar ataxia type 17 (SCA17), Syndromic/non-syndromic X-linked mental retardation, Emery-Dreifuss muscular dystrophy type 2, familial partial lipodystrophy, limb girdle muscular dystrophy type 1B, dilated cardiomyopathy, familial partial lipodystrophy, Charcot-Marie-Tooth disorder type 2B1, mandibuloacral dysplasia, childhood progeria syndrome (Hutchinson-Gilford syndrome), Werner syndrome, Dilated cardiomyopathy (DCM), Hypertrophic cardiomyopathy (HCM), Restrictive cardiomyopathy (RCM), Left Ventricular Non-compaction (LVNC), Arrhythmogenic Right Ventricular Dysplasia (ARVD), takotsubo cardiomyopathy, Duchenne muscular dystrophy, Becker muscular dystrophy, Limb-girdle muscular dystrophy, Facioscapulohumeral muscular dystrophy, Congenital muscular dystrophy, Oculopharyngeal muscular dystrophy, Distal muscular dystrophy, Emery-Dreifuss muscular dystrophy, dementia, Parkinson's disease (PD), a PD-related disorder, Prion disease, a motor neuron disease (MND), Progressive bulbar palsy (PBP), Progressive muscular atrophy (PMA), Primary lateral sclerosis (PLS), Spinal muscular atrophy (SMA), a bladder cancer, a breast cancer, a colorectal cancer, a kidney cancer, a lung cancer, a lymphoma, a melanoma, an oral cancer, an ovarian cancer, an oropharyngeal cancer, a pancreatic cancer, a prostate cancer, a thyroid cancer, a uterine cancer, Down syndrome, Prader-Willi Syndrome (PWS), Bloom Syndrome, Cockayne Syndrome Type I-216400, Cockayne Syndrome Type III, Cockayne Syndrome Type I, Hutchinson-Gilford Progeria Syndrome, Mandibuloacral Dysplasia with Type A Lipodystrophy, Progeria, Adult Onset Progeroid Syndrome, Neonatal Rothmund-Thomson Syndrome, Seip Syndrome, Werner Syndrome, Replication Focus-Forming Activity 1, myotubular myopathy, Danon Disease, and/or centronuclear myopathy.
Clause 85. A method of regulating transgene expression using a viral vector comprising a viral genome, the method comprising:
(a) inserting into the viral genome at least one alternatively-spliced exon cassette, wherein the alternatively-spliced exon cassette comprises a constitutive exon, at least one alternatively-spliced exon, at least one flanking intron, and a coding region of a transgene;
(b) introducing a heterologous start codon or part of a heterologous start codon at the 3′ end of the alternatively-spliced exon;
(c) disrupting or deleting all native start codons located 5′ to the heterologous start codon; and
(d) deleting a native start codon, or a portion thereof, from the coding region of the transgene,
wherein the constitutive exon, alternatively-spliced exon, and flanking intron are each located 5′ to the coding region of the transgene.
   Clause 86. A method of regulating transgene expression using a viral vector comprising a viral genome, the method comprising:
(a) inserting into the viral genome at least one alternatively-spliced exon cassette, wherein the alternatively-spliced exon cassette comprises an alternatively-spliced exon and at least one flanking intron within the coding region of the transgene; and
(b) introducing into the alternatively-spliced exon a heterologous, in-frame stop codon at least 50 nucleotides upstream of the next 5′ splice junction, wherein the heterologous, in-frame stop codon elicits nonsense-mediated decay.
   Clause 87. An alternatively-spliced exon cassette comprising, in the 5′ to 3′ direction:
(i) a nucleotide sequence comprising a first exonic sequence having a 5′ to 3′ orientation, wherein the first exonic sequence comprises a constitutive exon;
(ii) a nucleotide sequence comprising a first intronic sequence having a 5′ to 3′ orientation, wherein the first intronic sequence comprises at its 5′ end a 5′ splice donor site and at its 3′ end a 3′ splice acceptor site;
(iii) a nucleotide sequence comprising a second exonic sequence having a 5′ to 3′ orientation, wherein the second exonic sequence comprises an alternatively-spliced exon comprising at its 3′ end a heterologous ATG start codon;
(iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; and
(v) a nucleotide sequence comprising a coding region of a transgene having a 5′ to 3′ orientation, wherein the coding region of the transgene comprises at its 5′ end a modification comprising the removal of a native ATG start codon, wherein all native ATG start codons located upstream of the heterologous ATG start codon are mutated or deleted.
   Clause 88. An alternatively-spliced exon cassette comprising, in the 5′ to 3′ direction:
(i) a nucleotide sequence comprising a first portion of a coding region of a transgene having a 5′ to 3′ orientation;
(ii) a nucleotide sequence comprising a first intronic sequence having a 5′ to 3′ orientation, wherein the first intronic sequence comprises at its 5′ end a 5′ splice donor site and at its 3′ end a 3′ splice acceptor site;
(iii) a nucleotide sequence comprising an exonic sequence having a 5′ to 3′ orientation, wherein the exonic sequence comprises an alternatively-spliced exon comprising at its 3′ end a heterologous stop codon;
(iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; and
(v) a nucleotide sequence comprising a second portion of the coding region of the transgene having a 5′ to 3′ orientation.
   Clause 89. An alternatively-spliced exon cassette comprising, in the 5′ to 3′ direction:
(i) a nucleotide sequence comprising a coding region of a transgene having a 5′ to 3′ orientation;
(ii) a nucleotide sequence comprising a first intronic sequence having a 5′ to 3′ orientation, wherein the first intronic sequence comprises at its 5′ end a 5′ splice donor site and at its 3′ end a 3′ splice acceptor site;
(iii) a nucleotide sequence comprising a first exonic sequence having a 5′ to 3′ orientation, wherein the first exonic sequence comprises an alternatively-spliced exon comprising a positive or negative cis-acting element;
(iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; and
(v) a nucleotide sequence comprising a second exonic sequence having a 5′ to 3′ orientation, wherein the exonic sequence comprises a constitutive exon.
   Clause 90. An alternatively-spliced exon cassette comprising, in the 5′ to 3′ direction:
(i) a nucleotide sequence comprising a first exonic sequence having a 5′ to 3′ orientation, wherein the first exonic sequence comprises a constitutive exon;
(ii) a nucleotide sequence comprising a second exonic sequence having a 5′ to 3′ orientation, wherein the second exonic sequence comprises an alternatively-spliced exon comprising at its 3′ end a heterologous ATG start codon; and
(iii) a nucleotide sequence comprising a coding region of a transgene having a 5′ to 3′ orientation, wherein the coding region of the transgene comprises at its 5′ end a modification comprising the removal of a native ATG start codon, wherein all native ATG start codons located upstream of the heterologous ATG start codon are mutated or deleted.
   Clause 91. An alternatively-spliced exon cassette comprising, in the 5′ to 3′ direction:
(i) a nucleotide sequence comprising a first portion of a coding region of a transgene having a 5′ to 3′ orientation;
(ii) a nucleotide sequence comprising a first exonic sequence having a 5′ to 3′ orientation, wherein the first exonic sequence comprises an alternatively-spliced exon comprising at its 3′ end a heterologous stop codon;
(iii) a nucleotide sequence comprising a second portion of the coding region of the transgene having a 5′ to 3′ orientation;
(iv) a nucleotide sequence comprising an intronic sequence having a 5′ to 3′ orientation, wherein the intronic sequence comprises at its 5′ end a 5′ splice donor site and at its 3′ end a 3′ splice acceptor site; and
(v) a nucleotide sequence comprising a second exonic sequence having a 5′ to 3′ orientation, wherein the second exonic sequence comprises a constitutive exon.
   Clause 92. An alternatively-spliced exon cassette comprising, in the 5′ to 3′ direction:
(i) a nucleotide sequence comprising a coding region of a transgene having a 5′ to 3′ orientation;
(ii) a nucleotide sequence comprising a first exonic sequence having a 5′ to 3′ orientation, wherein the first exonic sequence comprises an alternatively-spliced exon comprising a positive or negative cis-acting element; and
(iii) a nucleotide sequence comprising a second exonic sequence having a 5′ to 3′ orientation, wherein the second exonic sequence comprises a constitutive exon.
   Clause 93. An alternatively-spliced exon cassette comprising, in the 5′ to 3′ direction:
(i) a nucleotide sequence comprising a first exonic sequence having a 5′ to 3′ orientation, wherein the first exonic sequence comprises a constitutive exon;
(ii) a nucleotide sequence comprising a second exonic sequence having a 5′ to 3′ orientation, wherein the second exonic sequence comprises an alternatively-spliced exon comprising at its 3′ end a heterologous ATG start codon;
(iii) a nucleotide sequence comprising an intronic sequence having a 5′ to 3′ orientation, wherein the intronic sequence comprises at its 5′ end a 5′ splice donor site and at its 3′ end a 3′ splice acceptor site; and
(iv) a nucleotide sequence comprising a coding region of a transgene having a 5′ to 3′ orientation, wherein the coding region of the transgene comprises at its 5′ end a modification comprising the removal of a native ATG start codon, wherein all native ATG start codons located upstream of the heterologous ATG start codon are mutated or deleted.
   Clause 94. An alternatively-spliced exon cassette comprising, in the 5′ to 3′ direction:
(i) a nucleotide sequence comprising a first portion of a coding region of a transgene having a 5′ to 3′ orientation;
(ii) a nucleotide sequence comprising an exonic sequence having a 5′ to 3′ orientation, wherein the exonic sequence comprises an alternatively-spliced exon comprising at its 3′ end a heterologous stop codon;
(iii) a nucleotide sequence comprising an intronic sequence having a 5′ to 3′ orientation, wherein the intronic sequence comprises at its 5′ end a 5′ splice donor site and at its 3′ end a 3′ splice acceptor site; and
(iv) a nucleotide sequence comprising a second portion of the coding region of the transgene having a 5′ to 3′ orientation.
   Clause 95. An alternatively-spliced exon cassette comprising, in the 5′ to 3′ direction:
(i) a nucleotide sequence comprising a coding region of a transgene having a 5′ to 3′ orientation;
(ii) a nucleotide sequence comprising a first exonic sequence having a 5′ to 3′ orientation, wherein the exonic sequence comprises an alternatively-spliced exon comprising a positive or negative cis-acting element;
(iii) a nucleotide sequence comprising an intronic sequence having a 5′ to 3′ orientation, wherein the intronic sequence comprises at its 5′ end a 5′ splice donor site and at its 3′ end a 3′ splice acceptor site; and
(iv) a nucleotide sequence comprising a second exonic sequence having a 5′ to 3′ orientation, wherein the exonic sequence comprises a constitutive exon.
   Clause 96. An alternatively-spliced exon cassette comprising, in the 5′ to 3′ direction:
(i) a nucleotide sequence comprising a first exonic sequence having a 5′ to 3′ orientation, wherein the first exonic sequence comprises a constitutive exon;
(ii) a nucleotide sequence comprising an intronic sequence having a 5′ to 3′ orientation, wherein the intronic sequence comprises at its 5′ end a 5′ splice donor site and at its 3′ end a 3′ splice acceptor site;
(iii) a nucleotide sequence comprising a second exonic sequence having a 5′ to 3′ orientation, wherein the second exonic sequence comprises an alternatively-spliced exon comprising at its 3′ end a heterologous ATG start codon; and
(iv) a nucleotide sequence comprising a coding region of a transgene having a 5′ to 3′ orientation, wherein the coding region of the transgene comprises at its 5′ end a modification comprising the removal of a native ATG start codon, wherein all native ATG start codons located upstream of the heterologous ATG start codon are mutated or deleted.
   Clause 97. An alternatively-spliced exon cassette comprising, in the 5′ to 3′ direction:
(i) a nucleotide sequence comprising a first portion of a coding region of a transgene having a 5′ to 3′ orientation;
(ii) a nucleotide sequence comprising a first intronic sequence having a 5′ to 3′ orientation, wherein the first intronic sequence comprises at its 5′ end a 5′ splice donor site and at its 3′ end a 3′ splice acceptor site;
(iii) a nucleotide sequence comprising a first exonic sequence having a 5′ to 3′ orientation, wherein the first exonic sequence comprises an alternatively-spliced exon comprising at its 3′ end a heterologous stop codon;
(iv) a nucleotide sequence comprising a second portion of the coding region of the transgene having a 5′ to 3′ orientation;
(v) a nucleotide sequence comprising a second intronic sequence having a 5′ to 3′ orientation, wherein the second intronic sequence comprises at its 5′ end a 5′ splice donor site and at its 3′ end a 3′ splice acceptor site; and
(vi) a nucleotide sequence comprising a second exonic sequence having a 5′ to 3′ orientation, wherein the second exonic sequence comprises a constitutive exon.
   Clause 98. An alternatively-spliced exon cassette comprising, in the 5′ to 3′ direction:
(i) a nucleotide sequence comprising a coding region of a transgene having a 5′ to 3′ orientation;
(ii) a nucleotide sequence comprising an intronic sequence having a 5′ to 3′ orientation, wherein the intronic sequence comprises at its 5′ end a 5′ splice donor site and at its 3′ end a 3′ splice acceptor site;
(iii) a nucleotide sequence comprising an exonic sequence having a 5′ to 3′ orientation, wherein the exonic sequence comprises an alternatively-spliced exon comprising a positive or negative cis-acting element; and
(iv) a nucleotide sequence comprising a second exonic sequence having a 5′ to 3′ orientation, wherein the exonic sequence comprises a constitutive exon.
   Clause 99. An alternatively-spliced exon cassette comprising, in the 5′ to 3′ direction:
(i) a nucleotide sequence comprising a first exonic sequence having a 5′ to 3′ orientation, wherein the first exonic sequence comprises a constitutive exon;
(ii) a nucleotide sequence comprising a first intronic sequence having a 5′ to 3′ orientation, wherein the first intronic sequence comprises at its 5′ end a 5′ splice donor site and at its 3′ end a 3′ splice acceptor site;
(iii) a nucleotide sequence comprising a second exonic sequence having a 5′ to 3′ orientation, wherein the second exonic sequence comprises an alternatively-spliced exon comprising at its 3′ end a heterologous ATG start codon;
(iv) a nucleotide sequence comprising a second intronic sequence having a 5′ to 3′ orientation, wherein the second intronic sequence comprises at its 5′ end a 5′ splice donor site and at its 3′ end a 3′ splice acceptor site;
(v) a nucleotide sequence comprising a third exonic sequence having a 5′ to 3′ orientation, wherein the third exonic sequence comprises an alternatively-spliced exon;
(vi) a nucleotide sequence comprising a third intronic sequence having a 5′ to 3′ orientation, wherein the third intronic sequence comprises at its 5′ end a 5′ splice donor site and at its 3′ end a 3′ splice acceptor site; and
(vii) a nucleotide sequence comprising a coding region of a transgene having a 5′ to 3′ orientation, wherein the coding region of the transgene comprises at its 5′ end a modification comprising the removal of a native ATG start codon,
wherein all native ATG start codons located upstream of the heterologous ATG start codon are mutated or deleted.
   Clause 100. An alternatively-spliced exon cassette comprising, in the 5′ to 3′ direction:
(i) a nucleotide sequence comprising a first portion of a coding region of a transgene having a 5′ to 3′ orientation;
(ii) a nucleotide sequence comprising a first intronic sequence having a 5′ to 3′ orientation, wherein the first intronic sequence comprises at its 5′ end a 5′ splice donor site and at its 3′ end a 3′ splice acceptor site;
(iii) a nucleotide sequence comprising a first exonic sequence having a 5′ to 3′ orientation, wherein the first exonic sequence comprises an alternatively-spliced exon comprising at its 3′ end a heterologous stop codon;
(iv) a nucleotide sequence comprising a second intronic sequence having a 5′ to 3′ orientation, wherein the second intronic sequence comprises at its 5′ end a 5′ splice donor site and at its 3′ end a 3′ splice acceptor site;
(v) a nucleotide sequence comprising a second exonic sequence having a 5′ to 3′ orientation, wherein the second exonic sequence comprises an alternatively-spliced exon;
(vi) a nucleotide sequence comprising a third intronic sequence having a 5′ to 3′ orientation, wherein the third intronic sequence comprises at its 5′ end a 5′ splice donor site and at its 3′ end a 3′ splice acceptor site (m); and
(vii) a nucleotide sequence comprising a second portion of the coding region of the transgene having a 5′ to 3′ orientation.
   Clause 101. An alternatively-spliced exon cassette comprising, in the 5′ to 3′ direction:
(i) a nucleotide sequence comprising a coding region of a transgene having a 5′ to 3′ orientation;
(ii) a nucleotide sequence comprising a first intronic sequence having a 5′ to 3′ orientation, wherein the first intronic sequence comprises at its 5′ end a 5′ splice donor site and at its 3′ end a 3′ splice acceptor site;
(iii) a nucleotide sequence comprising a first exonic sequence having a 5′ to 3′ orientation (e), wherein the first exonic sequence comprises a first alternatively-spliced exon comprising a positive or negative cis-acting element;
(iv) a nucleotide sequence comprising a second intronic sequence having a 5′ to 3′ orientation, wherein the second intronic sequence comprises at its 5′ end a 5′ splice donor site and at its 3′ end a 3′ splice acceptor site;
(v) a nucleotide sequence comprising a second exonic sequence having a 5′ to 3′ orientation, wherein the second exonic sequence comprises a second alternatively-spliced exon;
(vi) a nucleotide sequence comprising a third intronic sequence having a 5′ to 3′ orientation, wherein the third intronic sequence comprises at its 5′ end a 5′ splice donor site and at its 3′ end a 3′ splice acceptor site; and
(vii) a nucleotide sequence comprising a third exonic sequence having a 5′ to 3′ orientation, wherein the third exonic sequence comprises a constitutive exon.
   Clause 102. An alternatively-spliced exon cassette comprising, in the 5′ to 3′ direction:
(i) a nucleotide sequence comprising a first exonic sequence having a 5′ to 3′ orientation, wherein the first exonic sequence comprises a constitutive exon;
(ii) a nucleotide sequence comprising a first intronic sequence having a 5′ to 3′ orientation, wherein the first intronic sequence comprises at its 5′ end a 5′ splice donor site and at its 3′ end a 3′ splice acceptor site;
(iii) a nucleotide sequence comprising a coding region of a transgene having a 5′ to 3′ orientation;
(iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; and
(v) a nucleotide sequence comprising a second exonic sequence having a 5′ to 3′ orientation, wherein the second exonic sequence comprises an alternatively-spliced exon.
   Clause 103. An alternatively-spliced exon cassette comprising, in the 5′ to 3′ direction:
(i) a nucleotide sequence comprising a first portion of a coding region of a transgene having a 5′ to 3′ orientation;
(ii) a nucleotide sequence comprising a first intronic sequence having a 5′ to 3′ orientation, wherein the first intronic sequence comprises at its 5′ end a 5′ splice donor site and at its 3′ end a 3′ splice acceptor site;
(iii) a nucleotide sequence comprising a first exonic sequence having a 5′ to 3′ orientation, wherein the first exonic sequence comprises an alternatively-spliced exon comprising at its 3′ end a heterologous stop codon;
(iv) a nucleotide sequence comprising a second intronic sequence having a 5′ to 3′ orientation, wherein the second intronic sequence comprises at its 5′ end a 5′ splice donor site and at its 3′ end a 3′ splice acceptor site;
(v) a nucleotide sequence comprising a second exonic sequence having a 5′ to 3′ orientation, wherein the second exonic sequence comprises a constitutive exon;
(vi) a nucleotide sequence comprising a third intronic sequence having a 5′ to 3′ orientation, wherein the third intronic sequence comprises at its 5′ end a 5′ splice donor site and at its 3′ end a 3′ splice acceptor site; and
(vii) a nucleotide sequence comprising a second portion of the coding region of the transgene having a 5′ to 3′ orientation.
   Clause 104. An alternatively-spliced exon cassette comprising, in the 5′ to 3′ direction:
(i) a nucleotide sequence comprising a coding region of a transgene having a 5′ to 3′ orientation;
(ii) a nucleotide sequence comprising a first intronic sequence having a 5′ to 3′ orientation, wherein the first intronic sequence comprises at its 5′ end a 5′ splice donor site and at its 3′ end a 3′ splice acceptor site;
(iii) a nucleotide sequence comprising a first exonic sequence having a 5′ to 3′ orientation, wherein the first exonic sequence comprises an alternatively-spliced exon comprising a positive or negative cis-acting element;
(iv) a nucleotide sequence comprising a second intronic sequence having a 5′ to 3′ orientation, wherein the second intronic sequence comprises at its 5′ end a 5′ splice donor site and at its 3′ end a 3′ splice acceptor site; and
(v) a nucleotide sequence comprising a second exonic sequence having a 5′ to 3′ orientation, wherein the second exonic sequence comprises a constitutive exon.
   Clause 105. A transgene comprising:
(i) a constitutive exon and one or more intronic sequences, each from a first gene;
(ii) an alternatively-spliced exon cassette, wherein the alternatively-spliced exon cassette comprises:
   (a) an alternatively-spliced exon, and
   (b) flanking intronic sequences,
   wherein each of (a) and (b) are from a second gene; and
(iii) a coding region of interest from a third gene,
wherein the alternatively-spliced exon comprises an ATG start codon.
   Clause 106. The transgene of clause 105, wherein the first and second gene are the same gene; the first and third gene are the same gene; or all of the first, second, and third genes are the same gene.
   Clause 107. The transgene of clause 105 or clause 106, wherein the first gene is survival motor neuron 1 (SMN1).
   Clause 108. The transgene of any one of clauses 105-107, wherein the constitutive exon comprises exon 6 of SMN1, or a portion thereof.
   Clause 109. The transgene of any one of clauses 105-108, wherein the constitutive exon comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 102.
   Clause 110. The transgene of any one of clauses 105-109, wherein the constitutive exon comprises a polynucleotide having a nucleic acid sequence as set forth in SEQ ID NO: 102.
   Clause 111. The transgene of any one of clauses 105-110, wherein the one or more intronic sequences of (i) are or are derived from intron 6 and/or intron 7 of SMN1.
   Clause 112. The transgene of any one of clauses 105-111, wherein the one or more intronic sequences of (i) comprise(s) a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 103 and/or SEQ ID NO: 104.
   Clause 113. The transgene of any one of clauses 105-112, wherein the one or more intronic sequences of (i) comprise(s) a polynucleotide having a nucleic acid sequence as set forth in SEQ ID NO: 103 and/or SEQ ID NO: 104.
   Clause 114. The transgene of any one of clauses 105-113, wherein the second gene is a gene selected from the group consisting of: CAMK2B, PKP2, LGMN, NRAP, VPS39, KSR1, PDLIM3, BIN1, ARFGAP2, KIF13A, and/or PICALM.
   Clause 115. The transgene of any one of clauses 105-114, wherein the second gene is bridging integrator 1 (BIN1).
   Clause 116. The transgene of any one of clauses 105-115, wherein the alternatively-spliced exon comprises exon 11 of BIN1.
   Clause 117. The transgene of any one of clauses 105-116, wherein the alternatively-spliced exon comprises a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 37 or SEQ ID NO: 38.
   Clause 118. The transgene of any one of clauses 105-117, wherein the alternatively-spliced exon comprises a polynucleotide having a nucleic acid sequence as set forth in either SEQ ID NO: 37 or SEQ ID NO: 38.
   Clause 119. The transgene of any one of clauses 105-118, wherein the flanking intronic sequences of (ii) are or are derived from intron 10 and/or intron 11 of BIN1.
   Clause 120. The transgene of any one of clauses 105-119, wherein the flanking intronic sequences of (ii) each comprise a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 15 or SEQ ID NO: 16.
   Clause 121. The transgene of any one of clauses 105-120, wherein the flanking intronic sequences of (ii) each comprise a polynucleotide having a nucleic acid sequence as set forth in either SEQ ID NO: 15 or SEQ ID NO: 16.
   Clause 122. The transgene of any one of clauses 105-121, wherein the alternatively-spliced exon cassette comprises a polynucleotide having at least 70%, at least 75%, at least 80%, at least 90%, at least 92%, at least 95%, at least 98%, or at least 99% sequence identity, relative to a nucleic acid sequence as set forth in any one of SEQ ID NOs: 107-778.
   Clause 123. The transgene of any one of clauses 105-122, wherein the alternatively-spliced exon cassette comprises a polynucleotide having a nucleic acid sequence as set forth in any one of SEQ ID NOs: 107-778.
   Clause 124. The transgene of any one of clauses 105-123, wherein the third gene is myotubularin 1 (MTM1) or calpain 3 (CAPN3).
   Clause 125. The transgene of any one of clauses 105-124, wherein the coding region of interest comprises a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 1881 or SEQ ID NO: 1882.
   Clause 126. The transgene of any one of clauses 105-125, wherein the coding region of interest comprises a polynucleotide having a nucleic acid sequence as set forth in either SEQ ID NO: 1881 or SEQ ID NO: 1882.
   Clause 127. The transgene of any one of clauses 105-126, wherein, if the wild-type alternatively-spliced exon does not comprise an ATG start codon, the alternatively-spliced exon comprises 1-3 nucleic acid substitutions, relative to the wild-type alternatively-spliced exon, to form the ATG start codon within the alternatively-spliced exon.
   Clause 128. The transgene of clause 127, wherein the ATG start codon is formed in the alternatively-spliced exon by 1 nucleic acid substitution.
   Clause 129. The transgene of clause 127, wherein the ATG start codon is formed in the alternatively-spliced exon by 2 nucleic acid substitutions.
   Clause 130. The transgene of clause 127, wherein the ATG start codon is formed in the alternatively-spliced exon by 3 nucleic acid substitutions.
   Clause 131. The transgene of any one of clauses 105-130, wherein the alternatively-spliced exon is retained in the spliced transcript.
   Clause 132. The transgene of any one of clauses 105-131, wherein all native start codons located 5′ to the ATG start codon located within the alternatively-spliced exon are disrupted or deleted.
   Clause 133. The transgene of any one of clauses 105-132, wherein the alternatively-spliced exon cassette is located 5′, relative to the coding region of interest.
   Clause 134. The transgene of any one of clauses 105-133, wherein the constitutive exon is located 5′, relative to the alternatively-spliced exon cassette.
   Clause 135. The transgene of any one of clauses 105-134, wherein the one or more intronic sequences of (i) flank the alternatively-spliced exon cassette.
   Clause 136. The transgene of any one of clauses 105-135, wherein the alternatively-spliced exon comprises a heterologous, in-frame stop codon.
   Clause 137. The transgene of clause 136, wherein the heterologous, in-frame stop codon is at least 50 nucleotides upstream of the next 5′ splice junction.
   Clause 138. The transgene of clause 136, wherein the heterologous, in-frame stop codon elicits nonsense-mediated decay.
   Clause 139. The transgene of any one of clauses 105-138, wherein the alternatively-spliced exon is retained in the spliced transcript in distinct tissues.
   Clause 140. The transgene of clause 139, wherein the alternatively-spliced exon is retained in the spliced transcript in skeletal muscle and/or wherein the alternatively-spliced exon is not retained in the spliced transcript in heart and/or liver tissue.
   Clause 141. The transgene of any one of clauses 105-140, wherein the flanking intronic sequences of (ii)(b) are or are derived from native flanking introns of the alternatively-spliced exon.
   Clause 142. The transgene of any one of clauses 105-141, wherein the flanking intronic sequences of (ii)(b) each comprise at least one modification, relative to a naturally occurring intronic sequence.
   Clause 143. The transgene of clause 142, wherein the modification is a substitution or deletion of one or more nucleic acids.
   Clause 145. The transgene of any one of clauses 105-143, wherein the ATG start codon is located at the 3′ end of the alternatively-spliced exon.
   Clause 145. The transgene of clause 144, wherein, if the wild-type alternatively-spliced exon does not comprise an ATG start codon at its 3′ end, the first 10 nucleotides of the flanking intronic sequence which is immediately 3′ to the alternatively-spliced exon comprise 1-5 nucleotide substitutions, relative to the wild-type flanking intronic sequence which is immediately 3′ to the wild-type alternatively-spliced exon.
   Clause 146. The transgene of any one of clauses 105-145, wherein the one or more intronic sequences of (i) each comprise at least one modification, relative to a naturally occurring intronic sequence.
   Clause 147. The transgene of clause 146, wherein the modification is a substitution or deletion of one or more nucleic acids.
   Clause 148. The transgene of any one of clauses 105-147, wherein the coding region of interest comprises at least one modification, relative to a naturally occurring coding region of the third gene.
   Clause 149. The transgene of clause 148, wherein the modification is a substitution or deletion of one or more nucleic acids.
   Clause 150. The transgene of clause 148, wherein the coding region of interest comprises a deletion or disruption of a native start codon.
   Clause 151. The transgene of clause 148, wherein the coding region of interest comprises at least one heterologous stop codon.
   Clause 152. The transgene of clause 151, wherein the at least one heterologous stop codon is at least 50 nucleotides upstream of the next 5′ splice junction.
   Clause 153. The transgene of clause 151, wherein the at least one heterologous stop codon elicits nonsense-mediated decay.
   Clause 154. The transgene of any one of clauses 105-153, further comprising a 3′ untranslated region (UTR).
   Clause 155. The transgene of clause 154, wherein the 3′ UTR comprises a polyadenylation (pA) site and a cleavage site.
   Clause 156. The transgene of clause 155, wherein the polyadenylation site is an SV40 pA site.
   Clause 157. The transgene of any one of clauses 105-156, further comprising a promoter,
wherein the promoter is located 5′, relative to all of (i), (ii), and (iii).
   Clause 158. The transgene of clause 157, wherein the promoter is a tissue-specific promoter.
   Clause 159. The transgene of clause 158, wherein the tissue-specific promoter is an MHCK7 promoter.
   Clause 160. The transgene of any one of clauses 105-159, wherein the alternatively-spliced exon cassette comprises a nucleic acid sequence which is 450 to 650 nucleotides in length.
   Clause 161. A recombinant viral genome comprising the transgene of any one of clauses 105-160.
   Clause 162. The recombinant viral genome of clause 161, wherein the recombinant viral genome is a genome from a recombinant adeno-associated virus (rAAV).
   Clause 163. The recombinant viral genome of clause 162, wherein the transgene is flanked by AAV inverted terminal repeat (ITR) sequences.
   Clause 164. The recombinant viral genome of clause 163, wherein the AAV ITR sequences are AAV2 ITR sequences.
   Clause 165. The recombinant viral genome of any one of clauses 161-164, wherein the recombinant viral genome comprises a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 105 or SEQ ID NO: 106.
   Clause 166. The recombinant viral genome of any one of clauses 161-165, wherein the recombinant viral genome comprises a polynucleotide having a nucleic acid sequence as set forth in either SEQ ID NO: 105 or SEQ ID NO: 106.
   Clause 167. An rAAV particle comprising a recombinant viral genome according to any one of clauses 161-166.
   Clause 168. The rAAV particle of clause 167, wherein the rAAV particle comprises AAV serotype 1, 2, 3, 4, 5, 6, 7, 8, 9, or 10, or AAV derivative or pseudotype AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAV5hH10, AAV2 (Y→F), AAV8 (Y733F), AAV2.15, AAV2.4, AAVM41, and AAVr3.45.
   Clause 169. The rAAV particle of clause 167 or clause 168, further comprising at least one helper plasmid.
   Clause 170. The rAAV particle of clause 169, wherein the helper plasmid comprises a rep gene and a cap gene.
   Clause 171. The rAAV particle of clause 170, wherein the rep gene encodes Rep78, Rep68, Rep52, or Rep40, and/or wherein the cap gene encodes a VP1, VP2, and/or VP3 region of the viral capsid protein.
   Clause 172. The rAAV particle of clause 169, wherein the rAAV particle comprises two helper plasmids.
   Clause 173. The rAAV particle of clause 172, wherein the first helper plasmid comprises a rep gene and a cap gene and the second helper plasmid comprises a E1a gene, a E1b gene, a E4 gene, a E2a gene, and a VA gene.
   Clause 174. A recombinant viral genome comprising a transgene, wherein the transgene comprises:
(i) a constitutive exon and one or more intronic sequences;
(ii) an alternative exon cassette comprising:
   (a) an alternatively-spliced exon;
   (b) at least a portion of the intron immediately upstream of the alternatively-spliced exon; and
   (c) at least a portion of the intron immediately downstream of the alternatively-spliced exon,
   wherein, if the wild-type alternatively-spliced exon does not comprise an ATG start codon at its 3′ end:
      (1) the 3′ end of the alternatively-spliced exon comprises 1-3 nucleic acid substitutions relative to the wild-type alternatively-spliced exon to form an ATG start codon, and
      (2) the first 10 nucleotides of the intron immediately downstream of the alternatively-spliced exon comprise 1-5 nucleic acid substitutions relative to the wild-type intron immediately downstream of the wild-type alternatively-spliced exon; and
(iii) a coding region of interest.
   Clause 175. The recombinant viral genome of clause 174, wherein the 1-5 nucleic acid substitutions of (2) increase splice site strength.
   Clause 176. The recombinant viral genome of clause 174 or clause 175, wherein any wild-type start codons within the alternatively-spliced exon located upstream of the ATG start codon at the 3′ end of the alternatively-spliced exon are disrupted or deleted.
   Clause 177. The recombinant viral genome of any one of clauses 174-176, further comprising a tissue-specific promoter upstream of the alternative exon cassette.
   Clause 178. The recombinant viral genome of any one of clauses 174-177, wherein the coding region of interest is or is derived from a naturally occurring coding region of MTM1 or CAPN3.
   Clause 179. The recombinant viral genome of any one of clauses 174-178, wherein the tissue-specific promoter is an MHCK7 promoter.
   Clause 180. The recombinant viral genome of any one of clauses 174-179, wherein the alternative exon is exon 11 of the BIN1 gene.
   Clause 181. The recombinant viral genome of any one of clauses 174-180, wherein the constitutive exon is exon 6 of the SMN1 gene.
   Clause 182. The recombinant viral genome of any one of clauses 174-181, wherein the alternative exon cassette promotes skeletal muscle expression of the coding region of interest and reduces cardiac muscle expression of the coding region of interest.
   Clause 183. The recombinant viral genome of any one of clauses 174-182, wherein the alternative exon cassette is approximately 600 nucleotides in length.
   Clause 184. A method of treating a disease or condition in a subject comprising administering a recombinant viral genome according to any one of clauses 163-166 or 174-183, or an rAAV particle according to any one of clauses 167-173, to the subject.
   Clause 185. The method of clause 184, wherein the subject is a mammal.
   Clause 186. The method of clause 185, wherein the mammal is a human.
   Clause 187. The method of any one of clauses 184-186, wherein the recombinant viral genome or rAAV particle is administered to the subject at least one time.
   Clause 188. The method of clause 187, wherein the viral genome or rAAV particle is administered to the subject 2, 3, 4, 5, 6, 7, 8, 9, or 10 times.
   Clause 189. The method of any one of clauses 184-188, wherein the viral genome or rAAV particle is administered to the subject parenterally, subcutaneously, intraocularly, intravitreally, subretinally, intravenously (IV), intracerebro-ventricularly, intramuscularly, intrathecally (IT), intracisternally, intraperitoneally, enterally, via inhalation, topically, or by direct injection to one or more cells, tissues, or organs.
   Clause 190. The method of any one of clauses 184-189, wherein the viral genome or viral particle is administered to the subject by intravenous injection, intramuscular injection, intrathecal injection, or intravitreal injection.
   Clause 191. The method of any one of clauses 184-190, wherein the disease or condition is a disease or condition selected from the group consisting of Dentatorubral-pallido-luysian atrophy (DRPLA), myotonic dystrophy type 1 (DM1), myotonic dystrophy type 2 (DM2), Fragile X syndrome of mental retardation (FMR1), Fragile X tremor ataxia syndrome (FXTAS), FRAXE mental retardation (FMR2), Friedreichs ataxia (FRDA), Huntington disease (HD), Huntington disease-like 2 (HDL2), Oculopharyngeal muscular dystrophy (OPMD), Myoclonic epilepsy type 1, Alzheimer's disease, ALS/FTD, spinocerebellar ataxia type 1 (SCA1), spinocerebellar ataxia type 2 (SCA2), spinocerebellar ataxia type 3 (SCA3), spinocerebellar ataxia type 6 (SCA6), spinocerebellar ataxia type 7 (SCAT), spinocerebellar ataxia type 8 (SCA8), spinocerebellar ataxia type 10 (SCA10), spinocerebellar ataxia type 12 (SCA12), spinocerebellar ataxia type 17 (SCA17), Syndromic/non-syndromic X-linked mental retardation, Emery-Dreifuss muscular dystrophy type 2, familial partial lipodystrophy, limb girdle muscular dystrophy type 1B, dilated cardiomyopathy, familial partial lipodystrophy, Charcot-Marie-Tooth disorder type 2B1, mandibuloacral dysplasia, childhood progeria syndrome (Hutchinson-Gilford syndrome), Werner syndrome, Dilated cardiomyopathy (DCM), Hypertrophic cardiomyopathy (HCM), Restrictive cardiomyopathy (RCM), Left Ventricular Non-compaction (LVNC), Arrhythmogenic Right Ventricular Dysplasia (ARVD), takotsubo cardiomyopathy, Duchenne muscular dystrophy, Becker muscular dystrophy, Limb-girdle muscular dystrophy, Facioscapulohumeral muscular dystrophy, Congenital muscular dystrophy, Oculopharyngeal muscular dystrophy, Distal muscular dystrophy, Emery-Dreifuss muscular dystrophy, dementia, Parkinson's disease (PD), a PD-related disorder, Prion disease, a motor neuron disease (MND), Progressive bulbar palsy (PBP), Progressive muscular atrophy (PMA), Primary lateral sclerosis (PLS), Spinal muscular atrophy (SMA), a bladder cancer, a breast cancer, a colorectal cancer, a kidney cancer, a lung cancer, a lymphoma, a melanoma, an oral cancer, an ovarian cancer, an oropharyngeal cancer, a pancreatic cancer, a prostate cancer, a thyroid cancer, a uterine cancer, Down syndrome, Prader-Willi Syndrome (PWS), Bloom Syndrome, Cockayne Syndrome Type I-216400, Cockayne Syndrome Type III, Cockayne Syndrome Type I, Hutchinson-Gilford Progeria Syndrome, Mandibuloacral Dysplasia with Type A Lipodystrophy, Progeria, Adult Onset Progeroid Syndrome, Neonatal Rothmund-Thomson Syndrome, Seip Syndrome, Werner Syndrome, Replication Focus-Forming Activity 1, myotubular myopathy, Danon Disease, and/or centronuclear myopathy.
   Clause 192. The transgene of any one of clauses 105-160, wherein the ATG start codon is in the same reading frame as the coding region of interest.
   Clause 193. The transgene of any one of clauses 105-160, wherein the ATG start codon is within up to 5, 10, 20, or 30 nucleotides upstream of the 3′ end of the alternative-spliced exon.
   Clause 194. The transgene of any one of clauses 105-160, wherein the ATG start codon is within up to 5, 10, 20, or 30 nucleotides upstream of the 3′ end of the alternative-spliced exon and is in the same reading frame as the coding region of interest.

These and other aspects of the application are illustrated by the following non-limiting examples.

### Examples

Virally-mediated gene therapies that seek to deliver a protein cargo commonly package a coding region of interest along with a 5′ untranslated region, 3′ untranslated region, a promoter that will drive the gene of interest, and, sometimes, a constitutive intron to enhance nuclear export and RNA stability. However, almost all multi-exonic human genes in the human genome (>95%) are alternatively spliced such that multiple isoforms are generated from a single gene locus. These isoforms may exhibit distinct functions or expression patterns in different cellular conditions. Therefore, they comprise an important aspect of gene regulation and allow multiple species to be generated from a single locus.

There are many descriptions of tissue-specific exons in the literature; these types of data have been derived from microarray or RNAseq analyses of human tissues, or other conditions in which a perturbation is made and the transcriptome is profiled. The inclusion level of an exon is commonly described by “percent spliced in” (psi) and describes the percentage of mRNAs transcribed from a locus that are spliced to contain an alternatively-spliced exon of interest. For example, an exon that has a psi of 10% in a given tissue is included in the mature mRNA 10% of the time. Some examples of tissue-specific or tissue-biased exons include TPM1 exon 2 (<5% psi in heart but >95% psi in colon), or SLC25A3 exon 3 (>90% in heart but <5% in brain). Exons with a strong shift in psi between tissues are sometimes referred to as “switch-like” exons. Switchlike exons tend to exhibit greater phylogenetic conservation in their proximal introns, as compared to constitutively spliced exons or alternatively-spliced exons that do not exhibit switch-like behavior.

To date, tissue-specific alternative splicing regulation has not been used to control virally-mediated gene therapies, and there has been no straightforward method for how to do so. Described here are specific sequences that may confer tissue-specific regulation for virally-mediated gene therapies (e.g., AAV; lentivirus). In some embodiments, the virus is an adeno-associated virus (AAV). In embodiments where the virus is an AAV, the orientation of the cargo is invariant. This is because the AAV ITRs are symmetric. In some embodiments, the virus is a lentivirus. In embodiments, where the virus is a lentivirus, a cargo with spliced introns must be placed on the minus strand. This is because lentivirus packaging undergoes an RNA intermediate, and the introns must not be lost. Examples 1-6 describe an AAV-mediated gene therapy, however it should be understood that either an AAV or a lentivirus may be utilized according the methods described in the Examples.

### A. Example 1: Regulation of AAV Cargo Using Skipped Exon Trio

Alternatively-spliced exons and their flanking introns can be incorporated into AAV cargoes by at least two distinct methods to confer similar tissue-specific behavior. Both approaches utilize a skipped exon “trio” where there are two flanking constitutive exons and the middle exon is alternative.

In the first approach, the exon trio is placed at the start of the AAV cargo and an ATG or part of an ATG translation start codon is introduced at the end of the middle (alternative) exon. The downstream (constitutive) exon is omitted, but the transgene cargo of interest sans ATG is inserted in its place, such that inclusion of the alternatively-spliced exon results in joining of the ATG from the alternatively-spliced exon with the rest of the transgene of interest upon splicing. ATGs that lie upstream of the intended start codon are mutated or removed. Thus, this results in translation of the transgene only in settings that include the alternatively-spliced exon.

In the second approach, the alternatively-spliced exon and flanking introns are placed within the coding region of the AAV cargo. A stop codon is introduced within the alternatively-spliced exon such that it follows nonsense-mediated decay (NMD) rules, and thus elicits NMD when included. This results in productive translation of the transgene only in settings that exclude the alternatively-spliced exon. If the exon is too short to elicit NMD, another constitutive intron can be placed downstream in the transgene such that NMD rules (e.g., the stop codon should be >50 nucleotides from the next splice junction) are satisfied.

These two approaches may be applied not only to tissue-specific exons, but also exons that respond to different cellular states or conditions. For example, it may be desirable to confer regulatory behavior that occurs in:
(1) distinct tissues or cell types;
(2) activated T-cells, or states of inflammation;
(3) cells in which the transgene is highly expressed; and/or
(4) cells that exhibit severe disease.

The general approach described herein is advantageous over protein-based regulatory strategies because no additional protein components are necessary to confer regulation; all regulation occurs using endogenous machinery, and no neo-antigens are generated that could be immunogenic. All of the regulation occurs at the RNA level.

In some embodiments, the virus is an adeno-associated virus (AAV). In embodiments where the virus is an AAV, the orientation of the cargo is invariant. This is because the AAV ITRs are symmetric. In some embodiments, the virus is a lentivirus. In embodiments, where the virus is a lentivirus, a cargo with spliced introns must be placed on the minus strand. This is because lentivirus packaging undergoes an RNA intermediate, and the introns must not be lost.

### B. Example 2: Regulated Expression of AAV Cargo in Muscle Versus Heart Tissue

Commonly used methods to regulate tissue-specific expression include tissue-specific promoters and microRNAs. However, these methods are not quite specific enough to provide the level of control needed for certain therapeutic interventions. In contrast, there are exons that show close to 0% psi in heart but >90% psi in skeletal muscle. A regulatory cassette is generated using alternatively-spliced exons that allows an AAV transgene cargo to be expressed in skeletal muscle, but not in the heart. The exons shown in Table 1 will be tested to evaluate differential expression in skeletal versus heart tissue. These exons are good candidates for this type of tissue-specific behavior because they show robust switch-like behavior between heart and muscle. Some of the exons shown in Table 1 are conserved between mouse and human, and, correspondingly, the switch-like behavior is conserved across species. In some embodiments, the intronic sequences that flank the exons shown in Table 1 are also included as part of the regulatory cassette.

These exons were chosen because of their switch-like behavior between heart and muscle, and because they are all <250 nucleotides in length, with reasonably conserved intronic sequences that flank the exons. Additionally, these exons are all amenable to being cloned out of their endogenous context and placed into a minigene to act as regulatory cassettes to control AAV cargo expression. It is expected that incorporation of these exons into an AAV-delivered transgene will enable production of a protein cargo in the skeletal muscle and will result in decreased production of that cargo in the heart.

TABLE 1
Candidate exons compiled from heart and skeletal muscle RNAseq data.

| | | | | | Altered | |
|---|---|---|---|---|---|---|
| | | | | | Sequence | |
| | | | | | to remove | Full |
| | | | | | upstream ATGs | sequence |
| | Coordi- | Sequence of | Endogenous | Sequence of | and add ATG | in |
| | nates | truncated | Sequence | truncated | or part of ATG | AAV |
| Gene | (hg19) | upstream intron | of Exon | downstream intron | at end of exon | context |
| CAMK2B | chr7: | CTGTTACTTTTGCTGTGAT | TGGGCAGACAGACCACC | GTAGgTGTGTCTCGACCA | TGGGCAGACAGACCA | (SEQ |
| | 44279188: | GCTGTAATGCCGGGAACGC | GCTCCGGCCACAATGTC | GCGTCCCGCCCGCTCCCG | CCGCTCCGGCCACAA | ID NO: |
| | 44279262 | GTGCACACGGTCACACCAA | CACCGCGGCCTCCGGCA | CCCGTCCCTCCTGCCAGC | GTCCACCGCGGCCTC | 45) |
| | | CACTAATAGGACTGTCCTG | CCACCATGGGGCTGGTG | ATGCAGCCCCCTGCTGCA | CGGCACCACCAGGGG | |
| | | TCTGCTGTGTGCTCACCAC | GAACAAG (SEQ ID | CGCAGCCGCT (SEQ ID | CTGGTGGAACAAtG | |
| | | ACCCTTTGGGCATGAGAAG | NO: 23) | NO: 2) | (SEQ ID NO: 24) | |
| | | CCCCCACTGGGGTTTTCTA | 5′ splice site of | | | |
| | | AGGAGAAAGGAGGCAAATG | alternatively- | | | |
| | | CTTTTCCGTGTCAATCAGT | spliced exon | | | |
| | | CCAATCTTGTTTTCACTCT | was modified to | | | |
| | | CTTGAGCAAAGGATTCTGG | ATG\|GTAGGT | | | |
| | | AACCATCTGTCACCTAAAC | | | | |
| | | TTTAACTCTAATCTTCTTC | | | | |
| | | TGCTTCCTTTGTCTCTTTT | | | | |
| | | CTTCCCTTACCTCGCCCAC | | | | |
| | | CCCTCGTCTGTGTCCGCCC | | | | |
| | | ACCCCTCCCTTCCCCTCGT | | | | |
| | | CTCTAACCCGGTGCTAACA | | | | |
| | | G (SEQ ID NO: 1) | | | | |
| PKP2 | chr12: | GGAAAATTCCACTCCCTTT | ACCATACAGTCAATTTA | GTGAGACCCCTGTCTCTA | ACCATACAGTCAATT | (SEQ |
| | 32996116: | GTTCCAATTAATCCTCTCT | AGAAGTAGGAATGGCTG | CTCAAAATACAAAAAAAT | TAAGAAGTAGGAAGG | ID NO: |
| | 32996247 | GGTTTTTATTGTAAGGTGT | GCCGGGCGCGGTGGCTC | TAGCCG (SEQ ID NO: | CTGGCCGGGCGCGGT | 46) |
| | | ACTTTTTCTTTGAGTGTCC | ACGCCTGTAATCCCAGC | 4) | GGCTCACGCCTGTAA | |
| | | TGTGTGGTCCTTTTTAAAA | ACTTTGGGAGGCCAAGG | | TCCCAGCACTTTGGG | |
| | | GGAGGAAATGTCATTATGC | CGGGCGGATCACGAGGT | | AGGCCAAGGCGGGCG | |
| | | TTCACATTCTTAAGCTTCT | CAGGAGTTCGAGACCAG | | GATCACGAGGTCAGG | |
| | | GGCAGGCAGAGACTATTAA | CCTGACCAACATG | | AGTTCGAGACCAGCC | |
| | | TTTGTTTGCTTATTTAGGA | (SEQ ID NO: 25) | | TGACCAACATG | |
| | | CTAAAGAAGCTTTTGTTTT | | | (SEQ ID NO: 26) | |
| | | TTTTCTTCATTTCTCTCTT | | | | |
| | | CTTTCTAACTTGCTTTTGT | | | | |
| | | AGCTTAGTAACCAAAACTC | | | | |
| | | AGCCTCAGACTTGTCTTTA | | | | |
| | | AATTGTTTTCAACCACCTT | | | | |
| | | CTGTGCCCTGAGTACCTAT | | | | |
| | | GCTTCCTCTTTCCTTTGTA | | | | |
| | | CAG (SEQ ID NO: 3) | | | | |
| LGMN | chr14: | CCTGGATGTGAACTTGACT | GGTCTTACTCTGTTGCC | GTaTGTGCCACCATACTT | GGTCTTACTCTGTTG | (SEQ |
| | 93207407: | CTGCTACTTAGATGGCCCT | CAGGCTGGAGTGCAGTG | GGCTAATTTTTGTATTTT | CCCAGGCTGGAGTGC | ID NO: |
| | 93207524 | GTGAACTTGACCTTATTAC | GCACAATCTTGGCTCAC | TAGTAGAGATGGGGTTTC | AGTGGCACAATCTTG | 47) |
| | | TTGCATTGTTGGTGATAAT | TGCAACCTCTGCCTCCC | ACCATGTTGGCCAGGCTG | GCTCACTGCAACCTC | |
| | | TTATCTGGAATGGGCACTG | GGGTTCAAGCAATTCTC | GTCTCGAACTCCTGGCGT | TGCCTCCCGGGTTCA | |
| | | CATCCCAAACTTCTCAAAT | CTGCCTCAGCCTCCCGA | CAAGTGATCAACCCGTCT | AGCAATTCTCCTGCC | |
| | | GGATTAAACCTGACTTGAT | GTAGCTGGGATTACAG | CGGCCTCCCAAAATGCTG | TCAGCCTCCCGAGTA | |
| | | GGTACATTTCTTGGATCCA | (SEQ ID NO: 27) | GGATTACAGGCATGAGCC | GCTGGGATTACAtG | |
| | | AGTGAAGTCttttttttCT | 5′ splice site of | ACTGCACCCGGCCCCAAG | (SEQ ID NO: 28) | |
| | | TTTCTTGACAG (SEQ ID | alternatively- | TGAAGTCTTTTAAGTGAA | | |
| | | NO: 5) | spliced exon | TTACTGACCTGGTA | | |
| | | | was modified to | (SEQ ID NO: 6) | | |
| | | | ATG\|GTATGT | | | |
| NRAP | chr10: | ACCAGTTTTGCCATTTTTT | GTGGAGTATAAGAAGGA | GTGAGTTGTTAACGCTAA | GTGGAGTATAAGAAG | (SEQ |
| | 115402693: | GAGTTATGAAATCTTATAT | TCTGGAAAGTAGTAGAG | GCTTTTGTTTGGGCACTG | GATCTGGAAAGTAGT | ID NO: |
| | 115402797 | TATTTGTTCTTGAAAGCGA | GTCACAGTATCAACTAC | TTGGTGAGCTTAGTCTTT | AGAGGTCACAGTATC | 48) |
| | | CTATATGCTTTTAATTTTC | TGTGAAACACCTCAATT | AGGICTCCTAAGAGGGAC | AACTACTGTGAAACA | |
| | | TGAATGAAACCAATTATTT | CAGGAACGTGAGCAAGA | AGTCTATGGGAATGGATC | CCTCAATTCAGGAAC | |
| | | GCTATTTATTATGATTCAT | TCTCAAAATTTACCAGT | TATGTGTCTTGGAGAAGC | GTGAGCAAGATCTCA | |
| | | TTTTATAAAGGAAAATATG | GAT (SEQ ID NO: | ATGGCCTT (SEQ ID | AAATTTACCAGTGAT | |
| | | TCCTGCTAACTTAGCATAT | 29) | NO: 8) | (SEQ ID NO: 30) | |
| | | TCTATGCTTGATATGTTAA | | | | |
| | | AATCTTGGGTTGAAAGTTT | | | | |
| | | TCTAAAAATATCCTAGTCA | | | | |
| | | AGTCCTGGGACATTTTCAA | | | | |
| | | GAGTGACTTCGGATTTGGT | | | | |
| | | TCTATTGTGTGTCTGGTGT | | | | |
| | | TTTGATTTCCAAG (SEQ | | | | |
| | | ID NO: 7) | | | | |
| VPS39 | chr15: | TTTTTAAACCCCGAACCCA | TGCCAGCAGATGTAGCA | GTAAGTGTAGACGCATTG | TGCCAGCAGtTGTAG | (SEQ |
| | 42484264: | GTATGTAGTTATTGCTTCA | TCACCTGAAAGCGGCA | AATTGCTTTAGCTCTGGA | CATCACCTGAAAGCG | ID NO: |
| | 42484296 | TGATAGCTGCTCTGATGAT | (SEQ ID NO: 31) | GATGGGTTTGAGTGTTTG | GCA (SEQ ID NO: | 49) |
| | | GATGATGATAATTATTCTC | | CTCTGTCTCCGTGATACA | 32) | |
| | | TTTTTGACTCATTGGTGAC | | GAACTTAGTTAGAATAGT | | |
| | | TTTTGAGGCTGAATATTTT | | TGCTGGAAATTAGTTGGT | | |
| | | TGTCATCCCCAGCAAGGCT | | CTAGATCTAAGGATGCTT | | |
| | | GAATTCACATGTTTTTATG | | TGGGCTGGGTTTCATGGC | | |
| | | GTATATAACTTTCTTCCCC | | TTGGGCTCTTATATTTGA | | |
| | | TTTTTTGTAACAG (SEQ | | CTGTTACTTTTTACACCC | | |
| | | ID NO: 9) | | TTCCTTGTGGAACACTTT | | |
| | | | | GCCAAGGTACTGATTTTG | | |
| | | | | CAAGATAAGATCTTCAGA | | |
| | | | | CTCACGAGTACCTTGCCG | | |
| | | | | CTTCAGTGAACTTTGTGT | | |
| | | | | GATCCTTTCTGCT | | |
| | | | | (SEQ ID NO: 10) | | |
| KSR1 | chr17: | TCTTTTCCTCCTTCCTGCA | CTGCCTACTTCATTCAT | GTGAGTCCTTTGCATGGT | CTGCCTACTTCATTC | (SEQ |
| | 25928386: | TGGTTTGGCCTGTCCTTGC | CATAGACAGCAGTTTAT | TCCATTAACTGCCCTTTG | ATCATAGACAGCAGT | ID NO: |
| | 25928427 | CAGGTCCATCTGGGGTAGG | CTTTCCAG (SEQ ID | CTGTGACCCCTTCTGTCC | TTATCTTTCCAtG | 50) |
| | | GCTGTCCCAGCCCAGTAAC | NO: 33) | TGCCCACCTGGGCAGGGC | (SEQ ID NO: 34) | |
| | | AAGTCCCTGCCTCCCAAGC | | GCCCTCTCTCTGGGTACC | | |
| | | CGGTACCTGCAATTCCGCT | | TTTGGAGATACTTGAGTC | | |
| | | CCTCCCTCCTGCGTTGGCC | | TCCCTCCACCTGTTCTGA | | |
| | | GTTCTGTGTGTGTGACTCA | | GAGCTGACTGCCCATAGA | | |
| | | CCTCCTCTTCTGTTTAAAG | | GGATGGGAAGAGGGCAGG | | |
| | | (SEQ ID NO: 11) | | GCAAGTGCCCTGGTGGTA | | |
| | | | | GGCCTCTTACGTGAGAAG | | |
| | | | | TTCTTGCTGGGAGGGACC | | |
| | | | | CTGGAATCCATCCCATCC | | |
| | | | | AGCCTCTTGGCACAGAGA | | |
| | | | | CTGCAGGCAACCTTTTCA | | |
| | | | | AGGTCACACTGCTAG | | |
| | | | | (SEQ ID NO: 12) | | |
| PDLIM3 | chr4: | AAATCAAGATTTGGGATGT | TGGATGCAGCACTCCCT | GTAAgATGAGTCTCTTTG | TGGATtGCAGCACTC | (SEQ |
| | 186429453: | AATTCACCTCTCACTTTTG | CCGGGATTGACTGTGGC | AACCATGGGCATATTAAC | CCTCCGGGATTGACT | ID NO: |
| | 186429716 | TCTTCAATTCCTTTTAACC | AGTGGACGCAGCACCCC | TAAACACGTGGGCAATGT | GTGGCAGTGGACGCA | 51) |
| | | TCTCTGTACGTGTTCTCTG | TTCTTCTGTCAGTACTG | CAGCTTTACTGGTGTCTT | GCACCCCTTCTTCTG | |
| | | TGTTCTTCTGTCTGGACTG | TTAGTACCATTTGCCCA | TTAACATTGTCTT | TCAGTACTGTTAGTA | |
| | | TCTGGCTCCCGGTGCCATC | GGTGACTTGAAAGTTGC | (SEQ ID NO: 14) | CCATTTGCCCAGGTG | |
| | | TCTGTGTAACGTGTATCCA | GGCTAAGCTGGCCCCTA | | ACTTGAAAGTTGCGG | |
| | | TTAACACAG (SEQ ID | ACATTCCTTTGGAAATG | | CTAAGCTGGCCCCTA | |
| | | NO: 13) | GAACTTCCTGGTGTGAA | | ACATTCCTTTGGAAG | |
| | | | GATTGTACATGCTCAGT | | GAACTTCCTGGTGTG | |
| | | | TTAATACACCTATGCAG | | AAGATTGTACTGCTC | |
| | | | TTGTACTCAGATGACAA | | AGTTTAATACACCTT | |
| | | | TATTATGGAAACACTCC | | GCAGTTGTACTCAGT | |
| | | | AGGGTCAGGTTTCAACA | | GACAATATTgTGGAA | |
| | | | GCCCTAGGGGAAACACC | | ACACTCCAGGGTCAG | |
| | | | TTTGATGAG (SEQ ID | | GTTTCAACAGCCCTA | |
| | | | NO: 35) | | GGGGAAACACCTTTG | |
| | | | 5′ splice site of | | cTGAtG (SEQ ID | |
| | | | alternatively- | | NO: 36) | |
| | | | spliced exon | | | |
| | | | was modified to | | | |
| | | | ATG\|GTAAGA | | | |
| BIN1 | chr2: | GAGCCTCCTGCCCCTCACC | AAAGAAAAGTAAACTGT | GTACgtGCAGTGAGTGCT | AAAGAAAAGTAAACT | (SEQ |
| | 127818172: | AGGCCCTGTTAGCATCACC | TTTCGCGGCTGCGCAGA | GCGGAGGGGCGCAGAGGC | GTTTTCGCGGCTGCG | ID NO: |
| | 127818216 | TCGGGCACCTGGCCACAGC | AAGAAGAACAG (SEQ | CCGCGCCCTGGCTGGCCC | CAGAAAGAAGAACAt | 52) |
| | | AGGGGCCAGTCAGGGCACC | ID NO: 37) | TGTGCATGCGCCTTGCGC | G (SEQ ID NO: | |
| | | CCGGGATAGCACGCCCAGG | 5′ splice site of | CCTGCTCCCAGGTGCCAC | 38) | |
| | | CCCTGTGCAAGGCCTCTGG | alternatively- | TAACCCGTAATCTGGCTC | | |
| | | CACTTAGGAGAGGCTTTTG | spliced exon | TGTGTGCAGTGCTGCCCG | | |
| | | CCCCTTTGTCCTCTGAGCA | was modified to | GCAGGGCTGTCGTGTGCG | | |
| | | GAAGGGTTGGCAAAGAGGG | ATG\|GTACGT | TGTTGGGTGGGAAGGCGG | | |
| | | AAGGGGACAGGCCAGTTCT | | AGGCGGCGCGGGGGGGGC | | |
| | | GCACCTGGCCTTTCTCCAG | | TGGCCTCTGAGCATCTGG | | |
| | | AATGAAGGCCTCCACCTCC | | CTGCATTTAGCACG | | |
| | | CGTCCGTCCCCACAG | | (SEQ ID NO: 16) | | |
| | | (SEQ ID NO: 15) | | | | |
| ARFGAP2 | chr11: | TTTCTCAGCCAGTACCCCA | AGTCCGTGTATCTGTCA | GTAAgcGACAGCTGCTTG | AGTCCGTGTATCTGT | (SEQ |
| | 47194261: | GCCTGTCCTTTAACTCTTT | GCTAAAGGGGCCCTTCC | GGGAGCCCAGCCCCTGTT | CAGCTAAAGGGGCCC | ID NO: |
| | 47194302 | CCTACATTTTCCTTAGTGT | TGCCAGAG (SEQ ID | CTCAGCTTCCTGCTTTAG | TTCCTGCCAGAtG | 53) |
| | | CTTTCAGACTTGGTTCAGG | NO: 39) | AAGCGGAGCCTGGTGCTC | (SEQ ID NO: 40) | |
| | | GCGAACAGTCCATTTGCTG | 5′ splice site of | ACCATATGCTCCCTGCCT | | |
| | | ACCTCGGTTTGTTCTGTTG | alternatively- | CGCAGTCTTTGTTTTGGT | | |
| | | ACTCTTGGGGGTTGGGGCT | spliced exon | TCAGGGAATTTGGACGTC | | |
| | | GGAACCCAGCAGAGTGACT | was modified to | TCTGGTGCTTCGGACGTC | | |
| | | GTCAGACTGATCCATGAGA | ATG\|GTAAGC | TCTGGTGCTTCAGGTCTA | | |
| | | AGGCTGGCATAGCTTGGTC | | GGTTCCTGTCTC (SEQ | | |
| | | TGAAGCAGGGCTTCATAGA | | ID NO: 18) | | |
| | | GACCGCTGGGCTTGGGAGC | | | | |
| | | TCTGTGGAGGCTCTGGGGT | | | | |
| | | GGGCCTCTTGTTAGCCACA | | | | |
| | | AGTCTGTTTCTCCTCCAG | | | | |
| | | (SEQ ID NO: 17) | | | | |
| KIF13A | chr6: | GACTTCATCACaaaaGAAG | GTGGCTTCCCGATAATA | GTTTgTAACTGATTGGCA | GTGGCTTCCCGATAA | (SEQ |
| | 17822017: | TTATGTTTCCCCCTACCCC | AGCCTGTCTGCCTGGTG | TTGGCATGAGGGAGCTTG | TAAGCCTGTCTGCCT | ID NO: |
| | 17822136 | ATCCTTTACCCAGTTCCAG | GTCATCCCCTTGGTGGT | GCTGGTTTTTAGCAGTGC | GGTGGTCATCCCCTT | 54) |
| | | TGGAGCTTCCCTTTGGGGC | CTTATTTGAAGATCCTC | TGCTGATTGATGCTCATG | GGTGGTCTTATTTGA | |
| | | ATACACACAGTCTCTGATG | TTTCCTCTCATGTGGTT | CTCCATGCTTTGGCAAAA | AGATCCTCTTTCCTC | |
| | | CAAGTGCTGATGTGCCGGT | TCAGACGAAGCTCCTCC | ATCTAACTAACATGATTG | TCAaGTGGTTTCAGA | |
| | | GGCTGGACGGTGTTCACTG | ACAAACACTGGGATAAA | GGAAGAAGCTGGAGTTTT | CGAAGCTCCTCCACA | |
| | | TGATTCCCCTCTACTGCTA | G (SEQ ID NO: 41) | CTGGCTGATGAAGGIGTT | AACACTGGGATAAAt | |
| | | G (SEQ ID NO: 19) | 5′ splice site of | CTGTATTCTTCATTGAGG | G (SEQ ID NO: | |
| | | | alternatively- | AATGTTCCTTTTCAATTA | 42) | |
| | | | spliced exon | TGAACACCCCACCCCCAA | | |
| | | | was modified to | CACACACACAC (SEQ | | |
| | | | ATG\|GTTTGT | ID NO: 20) | | |
| PICALM | chr11: | AGGTATTTTTTGTTTTTGA | ATCCTTTCTCTGCTACT | GTAAAGTACCATTTAACC | ATCCTTTCTCTGCTA | (SEQ |
| | 85701293: | TAATTTAGATATTTAAGAT | GTAGATGCTGTTGATGA | TTTTTTTCCAATCAATGT | CTGTAGAGCTGTTGT | ID NO: |
| | 85701442 | TAATAAACAATAATATAGT | TGCCATTCCAAGCTTAA | TTATACTGCCTAACTATA | GATCCATTCCAAGCT | 55) |
| | | TACTACTTTTCACTTTGCA | ATCCTTTCCTCACAAAA | TTTTACGTGTCCATTAAT | TAAATCCTTTCCTCA | |
| | | TCTTTTGACTAATTACAAT | AGTAGTGGTGATGTTCA | TATTTTAAAAGCACTGCA | CAAAAAGTAGTGGTG | |
| | | ACTAATTTATTTACTTCCT | CCTTTCCATTTCTTCAG | ATAGTTACTTTAGATTTT | AGTTCACCTTTCCAT | |
| | | TTCTTCATTTTGCTATAAT | ATGTATCTACTTTTACT | AATATCAAATTCTAAAAC | TTCTTCAGATtGTAT | |
| | | ACATTTGGACTGCACAG | ACTAGGACACCTACTCA | TAACCAGCCATAATGTAG | CTACTTTTACTACTA | |
| | | (SEQ ID NO: 21) | TGAAATGTTTGTTG | TGAGGTTTTTAAATTGAC | GGACACCTACTCATt | |
| | | | (SEQ ID NO: 43) | ACTGGTATCCCAATTTTA | GAAAGTTTGTaTG | |
| | | | 5′ splice site of | TTTATACAGACTTTTAGG | (SEQ ID NO: 44) | |
| | | | alternatively- | AA (SEQ ID NO: 22) | | |
| | | | spliced exon | | | |
| | | | was modified to | | | |
| | | | ATG\|GTAAAG | | | |

### C. Example 3: Splicing Events that Exhibit Regulation During T-Cell Activation

A regulatory cassette (e.g., an alternatively-spliced exon cassette) is designed that exhibits dynamic behavior during T-cell activation. In some embodiments, such a cassette controls regulators of T-cell biology in the context of lentiviral-based cargoes (e.g., CAR-T approaches). For example, upon T-cell activation, a cargo produced using a regulatory cassette as described herein modulates the outcome of that T-cell. Exons from genes that have been previously shown to exhibit splicing changes upon T-cell activation, as published in the literature and shown in Table 2, will be tested.

In some embodiments, the intronic sequences flanking the exons shown in Table 2, along with the exons, will be introduced into a lentivirus splicing reporter and tested in resting and activated T-cells to assess activity. Sequence cassettes that exhibit behavior that is similar to their endogenous counterparts will be further developed to control heterologous cargoes. Exons from these genes were selected because they have been observed to change in splicing behavior following T-cell activation. It is expected that, when taken out of their endogenous context and placed within an AAV-delivered transgene, some of the exons will recapitulate behavior in activated T-cells.

TABLE 2
Exons from genes previously shown to exhibit splicing changes upon T-cell activation.

| | Evidence in Primary T | Evidence in Jurkat Splicing | |
|---|---|---|---|
| Gene | cells (Yes/No) | Line 1 (Yes/No) | Reference (PMID) |
| ABCC1 | Yes | | 26443849 |
| AK125149 | Yes | | 26443849 |
| ASCC2 | Yes | | 26443849 |
| BAT2D1 | | Yes | 22454538 |
| BBX | Yes | | 26443849 |
| BRD8 | | Yes | 22454538 |
| BRE | Yes | | 26443849 |
| C17orf70 | | Yes | 22454538 |
| CAMKK2 | Yes | | 26443849 |
| CBFB | Yes | | 26443849 |
| CCAR1 | Yes | | 26443849 |
| CCDC7 | Yes | | 26443849 |
| CD6 | Yes | | 24890719 |
| CHTF8 | Yes | | 26443849 |
| COL4A3BP | | Yes | 22454538 |
| COL6A3 | Yes | | 26443849 |
| CUGBP1 | Yes | | 17307815 |
| CUGBP2 | Yes | | 17307815 |
| CXorf45 | Yes | | 26443849 |
| DENND3 | Yes | | 26443849 |
| DGUOK | | Yes | 22454538 |
| DKFZp762G094 | Yes | | 26443849 |
| DNAJC7 | Yes | | 26443849 |
| DNASE1 | Yes | | 30521874 |
| EIF4A2 | Yes | | 26443849 |
| EIF4G2 | Yes | | 17307815 |
| EIF4H | Yes | | 26443849 |
| EXOC7 | Yes | | 26443849 |
| EZH2 | Yes | | 26443849 |
| FAM120A | Yes | | 26443849 |
| FAM136A | | Yes | 22454538 |
| FAM36A | | Yes | 22454538 |
| FARSB | | Yes | 22454538 |
| FBXO38 | Yes | | 26443849 |
| FGFR1OP2 | Yes | | 26443849 |
| FIP1L1 | | Yes | 22454538 |
| FOXRED1 | Yes | | 26443849 |
| FUBP3 | Yes | | 26443849 |
| GALT | | Yes | 22454538 |
| GATA3 | Yes | | 17307815 |
| GOLGA2 | Yes | | 26443849 |
| HIF1A | Yes | | 17307815 |
| HMMR | Yes | | 17307815 |
| HRB | Yes | | 17307815 |
| IKZF1 | Yes | | 26443849 |
| ILF3 | Yes | | 17307815 |
| IRAK4 | Yes | | 26443849 |
| IRF1 | Yes | | 17307815 |
| KCTD13 | Yes | | 26443849 |
| LEF1 | Yes | | 26443849 |
| LUC7L | Yes | | 26443849 |
| LYRM1 | Yes | | 26443849 |
| MALT1 e7 | Yes | | 27068814 |
| MAP2K7 | Yes | | 17307815 |
| MAP3K7 | | Yes | 22454538 |
| MAP4K2 | Yes | | 17307815 |
| MBNL2 | Yes | | 26443849 |
| MFF | Yes | | 26443849 |
| NAE1 | Yes | | 26443849 |
| NCSTN | | Yes | 22454538 |
| NR4A3 | Yes | | 26443849 |
| NRF1 | Yes | | 26443849 |
| NUP98 | Yes | | 26443849 |
| PARP6 | | Yes | 22454538 |
| PCM1 | Yes | | 26443849 |
| PLAUR | Yes | | 26443849 |
| PLSCR3 | | Yes | 22454538 |
| PPIL5 | Yes | | 26443849 |
| PPP5C | Yes | | 26443849 |
| PTPRC-E4 | | Yes | 22454538 |
| PTPRC-E6 | Yes | | 17307815 |
| PTS | Yes | | 26443849 |
| RABL5 | | Yes | 22454538 |
| RAPH1 | Yes | | 26443849 |
| SEC16A | | Yes | 22454538 |
| SFRS3 | Yes | | 26443849 |
| SFRS7 | Yes | | 26443849 |
| SLMAP | Yes | | 26443849 |
| SNRNP70 | Yes | | 26443849 |
| STAT6 | | Yes | 22454538 |
| TBC1D1 | | Yes | 22454538 |
| TIMM8B | Yes | | 26443849 |
| TIR8 | | Yes | 22454538 |
| TRA2A | Yes | | 26443849 |
| TROVE2 | Yes | | 26443849 |
| UGCGL1 | Yes | | 26443849 |
| VAP-B | Yes | | 26443849 |
| VAV1 | Yes | | 17307815 |
| ZNF384 | | Yes | 22454538 |
| ZNF496 | | Yes | 22454538 |

### D. Example 4: Broad Discovery of Tissue-Specific AAV Cassettes

A broad screen was performed to identify tissue-specific exon cassettes that exhibit similar behavior when placed within the context of an AAV cargo. These exons were identified using RNAseq data and exons that are <200 nucleotides long and exhibit high conservation across multiple species were chosen. These alternatively-spliced exons and their proximal introns are packaged into a heterologous context such that their inclusion level can be assessed by RT-PCR or deep sequencing. Nucleotide barcodes are included in the 3′ untranslated region such that the identity of each exon cassette can be determined by deep sequencing the barcode. The exon cassettes are packaged as a pool into an AAV library and administered to mice. Tissues or cell types of interest are harvested, and RNA originating from the AAV transgenes is prepared for deep sequencing such that psi values can be associated with each barcode in each tissue. Exon cassettes that exhibit tissue-specific behaviors of interest are identified using this procedure.

Examples of datasets used to identify tissue specific exons can be found in Wang, et al. (2008), Alternative isoform regulation in human tissue transcriptomes, Nature 456(7221): 470-76; Li, et al. (2017), A Comprehensive Mouse Transcriptomic BodyMap across 17 Tissues by RNA-seq, Sci. Rep. 7(1): 4200; and the GEO dataset entitled “[E-MTAB-513] Illumina Human Body Map 2.0 Project” (Series GSE30611).

### E. Example 5: Research Operating Procedure

A general research operating procedure for how to develop gene therapies that take advantage of alternative regulation is also provided. This approach can be generalized to facilitate the identification of particular sequences that confer regulatory behavior that is desired. In some embodiments, it is desirable to prevent over-dosing or over-expression in a given tissue.

The procedure is as follows:
(1) The cargo of interest is expressed using AAV in the tissue or cell types of interest.
(2) Transcriptome profiling is performed to identify exons that are sensitive to transgene over-expression.
(3) Using the exons identified in (2), alternatively-spliced exon cassettes that allow for control of the transgene are designed. The design may in some embodiments use the two methods described in Example 1 (e.g., place the ATG within the alternatively-spliced exon, or make the alternatively-spliced exon an NMD substrate). In some embodiments, modifications are made to ensure that the cassette responds appropriately to the transgene. In some embodiments, this work is done in vitro. In some embodiments, this work is done in vivo.
(4) A library of mutagenized splice sites or intronic elements is made that uses the alternatively-spliced exon cassette identified in (3) as the starting point. Barcodes are incorporated such that mutations can be linked to distinct barcodes. An AAV library is generated and administered in vivo in all the settings of interest (e.g., transgene over-expression or wild-type animals). Psi values of all variants are read out by deep sequencing and “winners” are chosen.
(5) The winners are individually tested in vivo.

### F. Example 6: Engineering Tissue-Specific Alternative Splicing to Regulate Gene Therapy Cargoes

A major challenge in the gene therapy field is to develop strategies yielding precise cargo expression—in levels, location, and timing. Because functional transduction of many tissues and cell types by viral vectors remains relatively inefficient, existing cargo sequences often incorporate strong promoters and minimal 5′ and 3′ UTR elements that enhance RNA stability and translation efficiency, aiming to maximize gene expression levels. However, over-expression of some cargoes in certain cell types and tissues may lead to toxicity, thus narrowing or eliminating the therapeutic windows available to treat disease. Solutions to achieve cell type-specific expression include use of tissue-specific promoters, incorporation of regulatory elements within mRNA sequence (e.g., microRNA binding sites), and packaging of cargoes into capsid variants exhibiting cell type-specific tropisms. These approaches, however, provide limited control, and fail to incorporate certain basic mechanisms of gene regulation ubiquitously employed by the naturally-occurring genome. One of these mechanisms is alternative splicing, which has been relatively unexplored as a mechanism by which to regulate gene therapy cargo expression.

Alternative splicing occurs in −95% of all multi-exonic human genes, with a major portion of regulated exons showing a tissue or cell type-specific bias (1). The most studied form of alternative splicing is the “skipped exon” or “cassette exon”, in which an alternative exon can be included or excluded between a pair of constitutive exons. The present inventors have identified a subset of “switch-like” cassette exons that show differences in inclusion level between tissues; these exons tend to preserve reading frame more frequently than other cassette exons and display increased phylogenetic conservation in the −200 intronic nucleotides both upstream and downstream of these exons.

Regulation of alternative splicing is controlled by core spliceosomal machinery, along with RNA binding proteins (RBPs); many RBPs themselves show tissue-specific expression profiles (2). Mechanistic studies of alternative splicing regulation are often performed by cloning the cassette exon sequence (e.g., upstream intron, cassette exon, and downstream intron) into a heterologous context in which the flanking constitutive exons are taken from a separate gene (3). For example, beta globin exons 1-3 (4) and SMN1 exons 6-8 (5,6) are commonly employed exon/intron contexts into which cassette exon sequences have been incorporated for further study. In addition, the behavior of alternatively spliced exons can be recapitulated in heterologous contexts and has even been re-purposed to control fluorescent reporter expression (7). Similar concepts have been used to regulate AAV-mediated gene expression in vivo using alternative splicing, wherein expression of a target gene is controlled via exposure to, for example, an aptamer ligand, such as a small molecule (8,9). However, no attempts have been made to use exons displaying cell- or tissue-specific, or endogenous activity-dependent, splicing patterns to regulate gene therapy cargoes.

The current gene therapy landscape is focused on a multitude of disease indications, but several broad areas could benefit from improved cell or tissue type-specific regulation. Firstly, observed toxicities of AAV-delivered therapies in dorsal root ganglia suggest that minimization of heterologous cargo expression in this tissue could be beneficial, even if a major portion of the toxicity is capsid-mediated. Secondly, a great number of gene therapies are being developed for neuromuscular or cardiac indications; however, some cargoes that are therapeutic in one tissue may be toxic when over-expressed in the other, and there are limited approaches available to fully de-target either tissue.

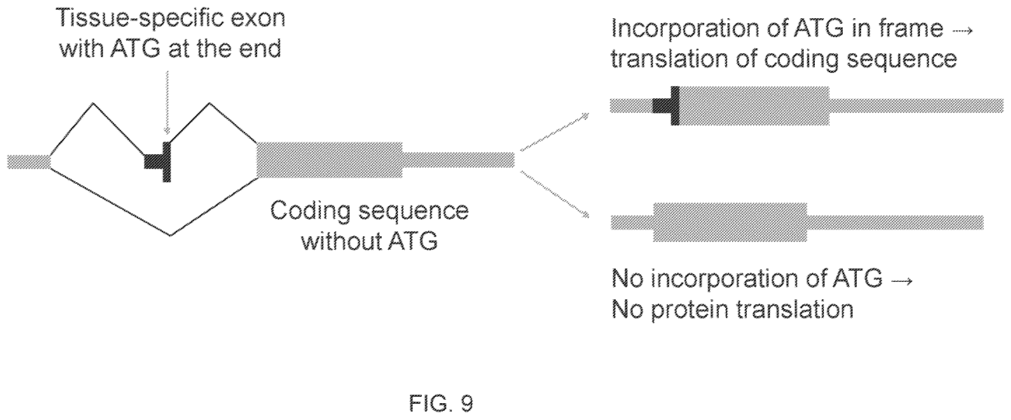

Described herein is a general approach to re-purpose, engineer, and optimize alternative splicing cassettes to de-target specific tissues and cell types. Alternative splicing cassettes were engineered to control protein cargo expression in the context of AAV. These cassettes were designed such that incorporation of the AUG translation initiation codon within the cassette exon would lead to cargo production upon inclusion (FIG. 9), and/or such that incorporation of a premature stop codon within the cassette exon would lead to nonsense-mediated decay of the cargo mRNA upon inclusion. Screens were performed across hundreds of candidates in vivo, and proof of concept is provided herein for how to further optimize sequences that confer switch-like behavior. Individual sequences of interest were tested and both splicing patterns and total protein output were assessed as gold standards for the extent of de-targeting.

The approach described herein, Tissue-specific Alternative splicing to Restrict Globally Expressed Therapeutic (TARGET), is broadly applicable to any set of tissues or cell types and can be applied to any cargo that satisfies viral packaging limit restrictions in any virus that supports packaging of splicing-competent transgenes. Some viruses that undergo splicing during packaging (e.g., lentivirus) would require encoding of the transgene on the minus strand of the viral genome to avoid removal of introns during the packaging process.

### Results

### Identification of Alternative Exon Candidates and Selection of Transgene Context

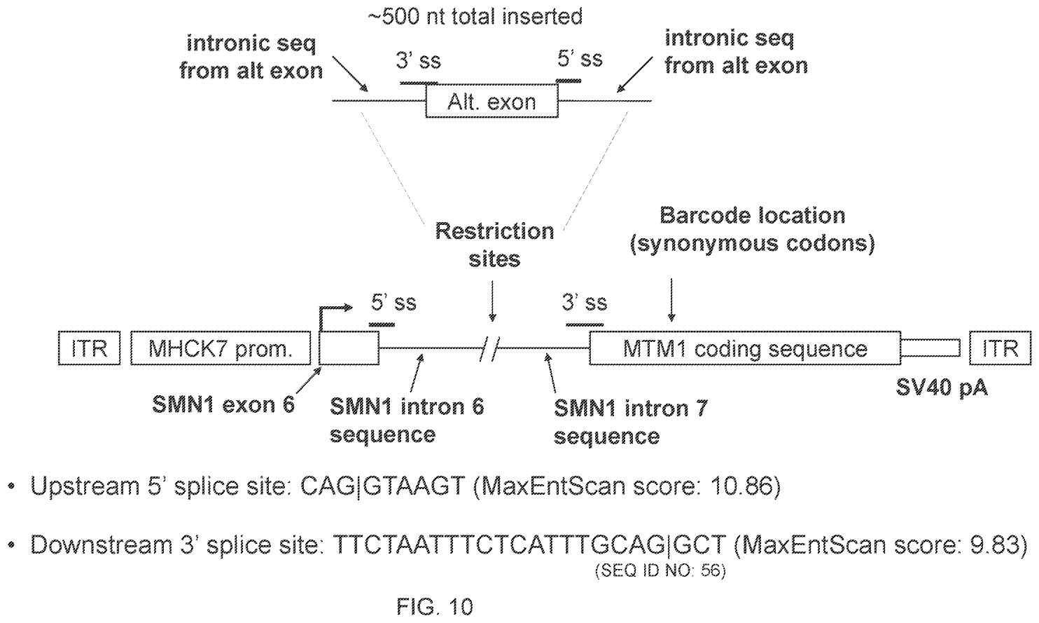

RNAseq datasets were analyzed to identify candidate exons that display extreme “switch-like” behavior between human heart (10) and skeletal muscle (SRA project SRP082676). These candidates were further filtered by those that were also conserved to mouse, and those which displayed similar percent spliced in (psi) values in mouse heart (low psi) and skeletal muscle (high psi). A set of 11 cassette exons were selected and ˜500 nucleotides of total sequence were cloned—including the cassette exon and immediately adjacent flanking introns—into the SMN1 exon 6/intron 7 context, which has been previously used to study alternative splicing regulation (11) (FIG. 10). The MTM1 coding sequence, which expresses the myotubularin protein, a protein that is missing in boys affected by X-linked myotubular myopathy (12) (XLM™), was chosen as the therapeutic cargo. Although MTM1 expression in skeletal muscle is therapeutic, questions have been raised about whether over-expression in heart can lead to toxicity (13), providing motivation to identify cassettes that may preferentially de-target heart but preserve skeletal muscle expression.

### Mutations to Facilitate Translation Initiation at the End of the Alternative Exon and Avoid Spurious Downstream Translation

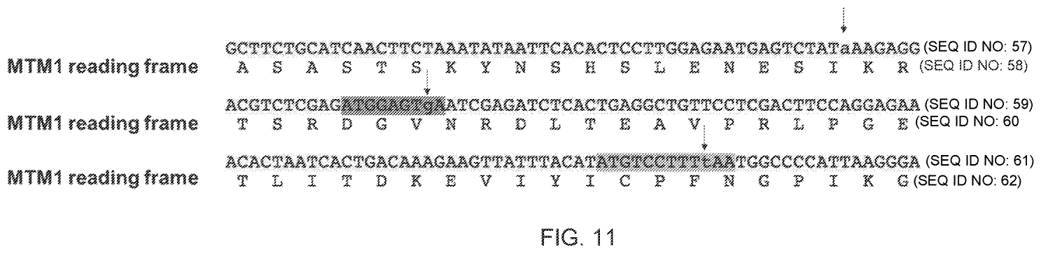

For each of the alternative exon candidates, the final nucleotides of the exon were altered to either be “ATG”, “AT”, or “A” (depending on which nucleotides naturally occurred), such that initiation of translation could be achieved when the exon was included. Additionally, any upstream ATGs within the alternative exon were removed by substitution or deletion, to avoid translation initiation at an earlier location. In the case of exon skipping, downstream ATGs within the MTM1 coding sequence might lead to translation of unwanted protein fragments; thus, stop codons were introduced within 15 nucleotides of each of these ATGs, such that translation would terminate within just a few (<5) amino acids (FIG. 11). These ATGs and stop codons all resided in a reading frame distinct from the normal MTM1 reading frame, and thus mutations required to generate these stop codons could preserve the amino acid composition of MTM1. For other cargoes in which internal methionines are present, new out-of-frame short peptide sequences could be introduced upstream of these methionines such that translation of these short, benign peptides is favored over translation of a N-terminally truncated cargo (re-initiation of translation following a stop codon typically does not occur unless there are additional regulatory elements such as internal ribosomal entry sites).

### Mutations to Preserve Splice Site Strength and Considerations of the Kozak Sequence

Because changes to the end of the alternative exon sequence can affect the strength of the alternative exon's 5′ splice site, both the original and altered 5′ splice sites of the alternative exon were scored using MaxEntScan (14) and compensatory mutations were made to the intronic bases of the alternative exon's 5′ splice site to compensate for any potential weakening of the splice site signal (FIG. 12; Table 3). The bases of the alternative exon which upstream of the ATG initiation sequence were also analyzed for translation initiation potential (15), and almost all sequences in this set showed reasonably strong scores. Additional mutations within the alternative exon could be made to increase similarity to the Kozak consensus sequence.

Barcoding Method to Uniquely Identify Each Alternative Exon within the Pool of Candidates

A unique nucleotide “barcode” sequence was introduced within the MTM1 coding sequence such that it preserved the amino acid composition of MTM1, but also uniquely identified the upstream alternative exon cassette (FIG. 13). This barcode was necessary so that the frequency of alternative exon inclusion could be properly computed; the alternative exon identity is evident when it is included, but the barcode is required for identification when it is skipped. The number of deep sequencing reads that cross the splice site junctions (read 1 of each read pair) thus can be associated with the deep sequencing reads that capture each barcode (read 2 of each read pair), facilitating calculation of percent spliced in (psi, Ψ) for each candidate. This is similar in principle to other published approaches (6,16).

### Viral Packaging, Delivery In Vivo, Library Preparation, and Sequencing

All 11 alternative exon candidates (see Table 3 for exon coordinates, psi values, translational initiation scores, and sequence alterations) were packaged into AAV9 as a pool and administered to mice systemically (retro-orbital injection, 4 C57/BL6 mice and 2 FVB mice at 6 weeks of age, 2e13 vg/kg) and intramuscularly (4 C57/BL6 mice at 6 weeks of age, tibialis anterior, 2e11 vg total into one leg). Mice were sacrificed after 4 weeks; the heart and liver were harvested from the systemically injected animals and the tibialis anterior (TA) was harvested from the intramuscularly injected animals. Reverse transcription and polymerase chain reaction was performed using primers targeting the upstream SMN1 exon 6 and also a region in MTM1 3′ of the barcode. Illumina adapters with unique indexes to identify each sample were incorporated into the final amplicon libraries and then sequenced.

TABLE 3
Alternative exon candidates.

| | | | Coordinates | Last 3 nt | Translation | Kozak | Splice | Alter- |
|---|---|---|---|---|---|---|---|---|
| Gene | Heart PSI | Muscle PSI | (hg19) | of exon | initiation | score | Site | ation? |
| CAMK2B | 0.01, 0.01, | 0.94, 0.95, | chr7: | AtG | GGAACAAtGGC | 92 | AAGGTAGAT | AtGGT |
| | 0.01 | 0.96 | 44279188: | | (SEQ ID | | | AGGT |
| | | | 44279262 | | NO: 90) | | | |
| PKP2 | 0.01, 0.01, | 0.98, 0.90, | chr12: | ATG | ACCAACATGGC | 105 | ATGGTGAGA | |
| | 0.01 | 0.91 | 32996116: | | (SEQ ID | | | |
| | | | 32996247 | | NO: 91) | | | |
| LGMN | 0.03, 0.01, | 0.32, 0.60, | chr14: | AtG | GATTACAtGGC | 90 | CAGGTGTGT | AtGGT |
| | 0.03 | 0.69 | 93207407: | | (SEQ ID | | | aTGT |
| | | | 93207524 | | NO: 92) | | | |
| NRAP | 0.04, 0.03, | 0.96, 0.94, | chr10: | GAT | CCAGTGATGGC | 103 | GATGTGAGT | |
| | 0.01 | 0.81 | 115402693: | | (SEQ ID | | | |
| | | | 115402797 | | NO: 93) | | | |
| VPS39 | 0.02, 0.03, | 0.85, 0.95, | chr15: | GCA | AGCGGCATGGC | 110 | GCAGTAAGT | |
| | 0.04 | 0.93 | 42484264: | | (SEQ ID | | | |
| | | | 42484296 | | NO: 94) | | | |
| KSR1 | 0.20, 0.15, | 0.99, 0.92, | chr17: | AtG | CTTTCCAtGGC | 98 | CAGGTGAGT | AtGGT |
| | 0.06 | 0.99 | 25928386: | | (SEQ ID | | | GAGT |
| | | | 25928427 | | NO: 95) | | | |
| PDLIM3 | 0.13, 0.20, | 0.92, 0.97, | chr4: | AtG | TTGcTGAtGGC | 90 | GAGGTAATA | AtGGT |
| | 0.07 | 0.96 | 186429453: | | (SEQ ID | | | AAgA |
| | | | 186429716 | | NO: 96) | | | |
| BIN1 | 0.08, 0.20, | 0.93, 0.94, | chr2: | AtG | AAGAACAtGGC | 120 | CAGGTACCG | AtGGT |
| | 0.10 | 0.94 | 127818172: | | (SEQ ID | | | ACgt |
| | | | 127818216 | | NO: 97) | | | |
| ARFGAP2 | 0.08, 0.06, | 0.90, 0.87, | chr11: | AtG | TGCCAGAtGGC | 105 | GAGGTAACT | AtGGT |
| | 0.02 | 0.82 | 47194261: | | (SEQ ID | | | Aagc |
| | | | 47194302 | | NO: 98) | | | |
| KIF13A | 0.01, 0.07, | 0.82, 0.78, | chr6: | AtG | GGATAAAtGGC | 122 | AAGGTTTTT | AtGGT |
| | 0.04 | 0.71 | 17822017: | | (SEQ ID | | | TTgT |
| | | | 17822136 | | NO: 99) | | | |
| PICALM | 0.04, 0.03, | 0.68, 0.75, | chr11: | aTG | GTTTGTaTGGC | 65 | TTGGTAAAG | aTGGT |
| | 0.03 | 0.80 | 85701293: | | (SEQ ID | | | AAAG |
| | | | 85701442 | | NO: 100) | | | |

### Analysis of Deep Sequencing Reads

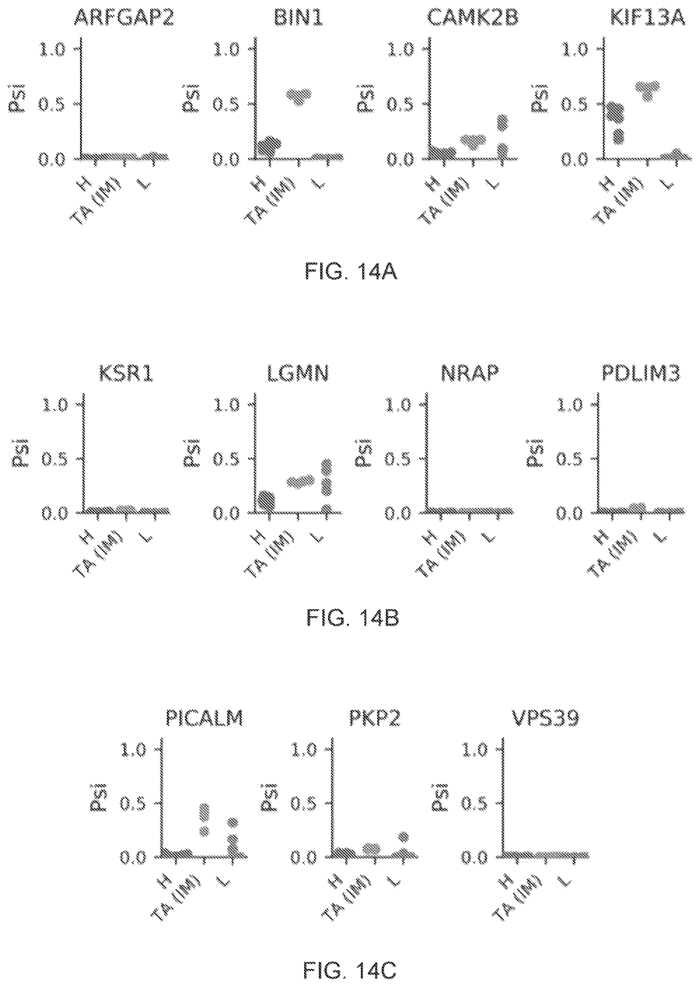

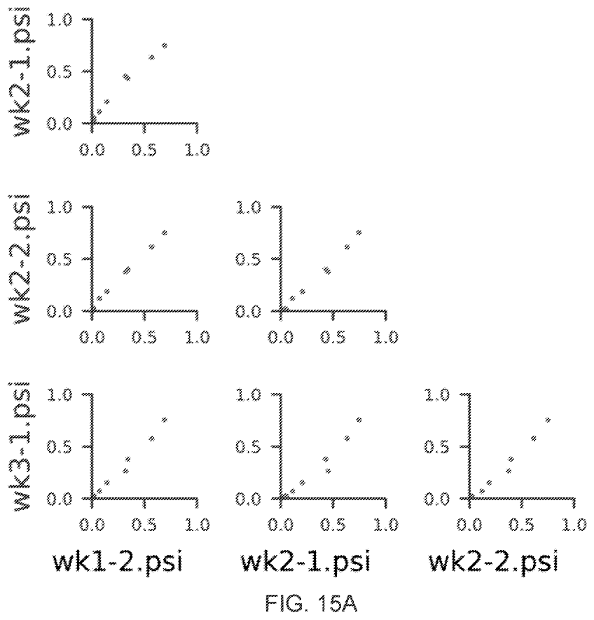

Psi values were computed by associating junction reads to barcodes and computing the frequency of inclusion versus exclusion of each exon (FIGS. 14A-14C). Exons from BIN1, CAMK2B, KIF13A, LGMN, and PICALM showed higher inclusion levels in skeletal muscle (TA) and heart (H), and the BIN1 exon candidate showed the largest dynamic range, with −15% inclusion in heart and −60% in TA. In addition, the BIN1 exon also showed −0% inclusion in liver (L). These results provide proof of concept for the overall screening strategy and identify alternative exon cassettes that would be predicted to minimize translation of MTM1 in heart as compared to skeletal muscle.

### Supplemental Data: Reproducibility of Results as a Function of Time Following Dosing

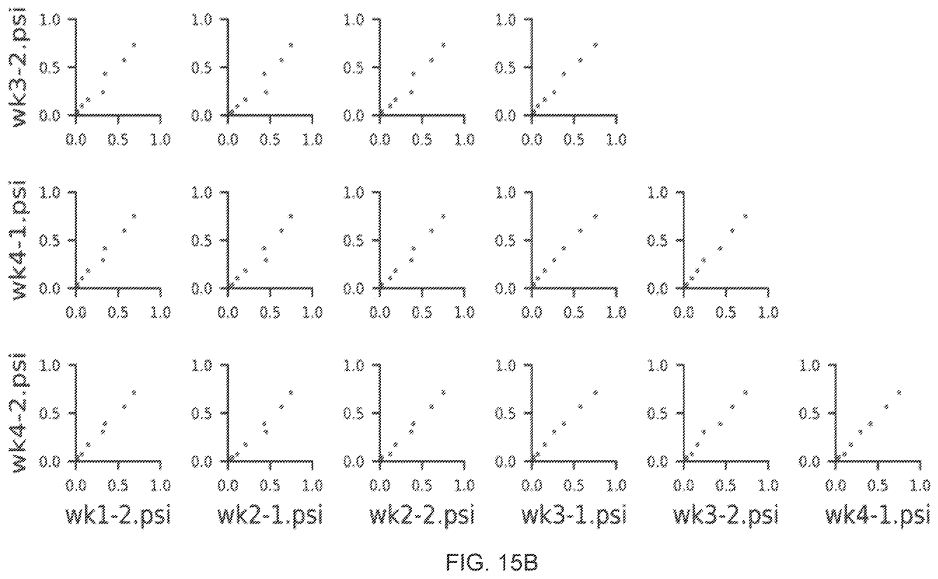

To assess whether the time following intramuscular administration might influence the psi value assessment, the same library was administered into 7 additional mice intramuscularly (2e11 vg total into one tibialis anterior (TA) of each mouse). The TAs were harvested 1, 2, 3, or 4 weeks following dosing. Sequencing libraries were generated and the psi values were correlated for each exon candidate across all samples. The results were strongly concordant, regardless of what time point was analyzed (FIGS. 15A-15B).

Screens to Identify Sequences that Further De-Target Heart but Maximize Expression in Skeletal Muscle

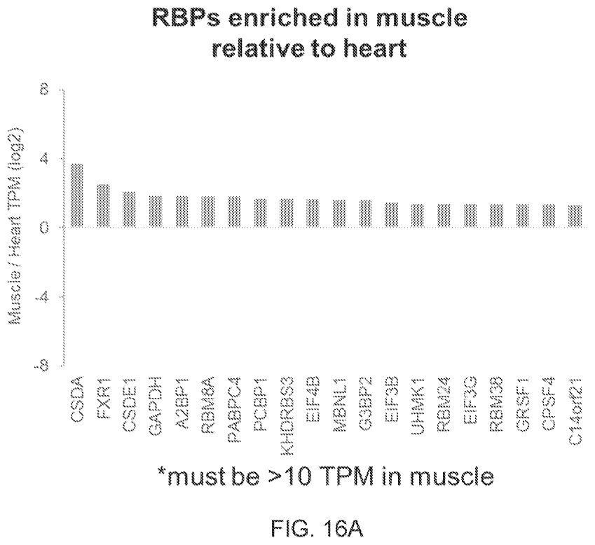

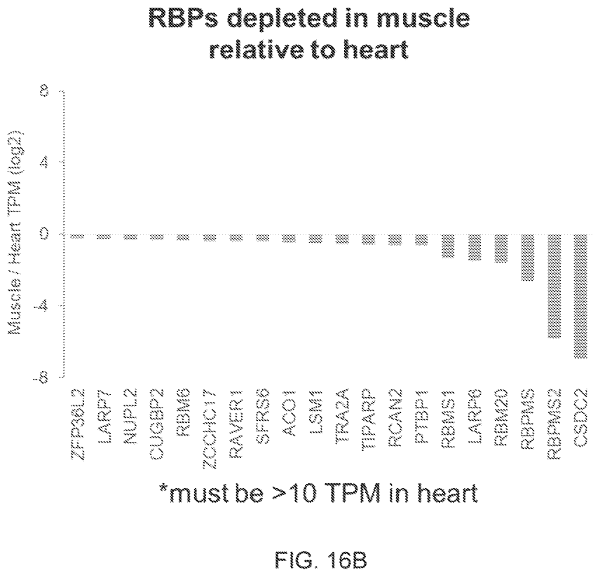

Based upon the initial hits of the first screen, described above, alternative exon cassette sequences were identified which might further enhance the switch-like behavior in heart versus skeletal muscle. A higher throughput approach was taken to simultaneously screen many sequence variants of candidate alternative exon cassettes. Core splice site sequences as well as intronic/exonic sequences play important roles in splicing decisions, by modulating the ability of specific trans-factors to bind a pre-mRNA. The core splicing signals, which include the 3′ splice site, 5′ splice site, and branch point, can all influence the frequency with which an alternatively spliced exon is chosen. These core splicing signals are recognized by the U1, U5, and U2 snRNPs, among other components; but they may also be bound by other RNA binding proteins (RBPs), which play roles in modulating how well the basal splicing machinery can recognize the core signals. Furthermore, RBPs can bind to intronic or exonic sequence in the vicinity of these core splicing signals to affect overall splicing decisions. The abundance of certain RBPs in certain contexts can therefore influence splicing patterns in those contexts. To aid efforts to further optimize sequences that display switch-like alternative splicing in heart versus skeletal muscle, the expression level of RNA binding proteins in these 2 tissues was analyzed (FIGS. 16A-16B). RNA expression levels were obtained from GTEX (17), and RBPs were defined from RBPDB (18). The ratio of expression in heart versus skeletal muscle was computed, and used to identify RBPs showing strongest differential expression between these 2 tissues; these RBPs would be predicted to be trans-factors that might be responsible for influencing splicing decisions of highly heart versus skeletal muscle-specific exons.

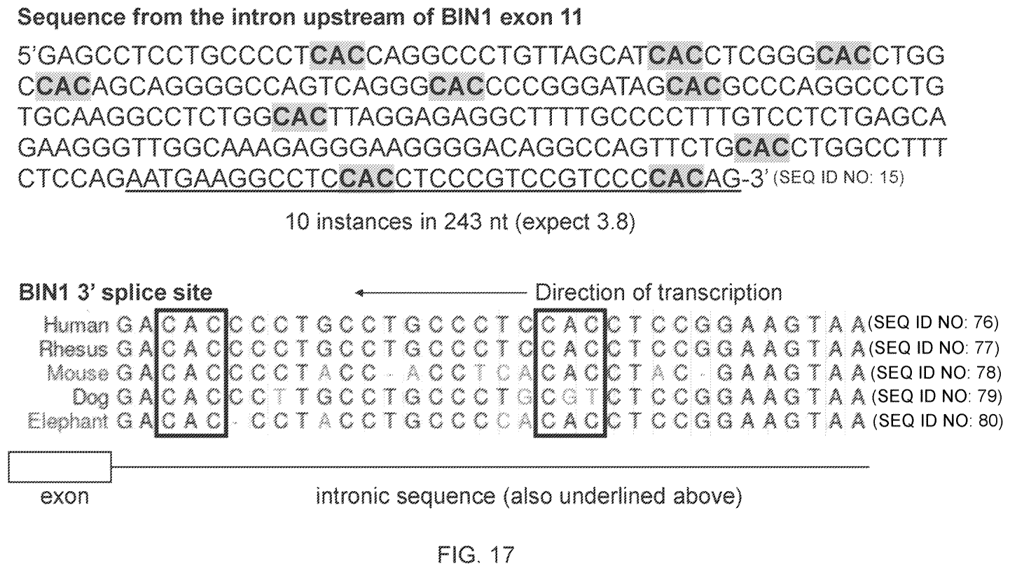

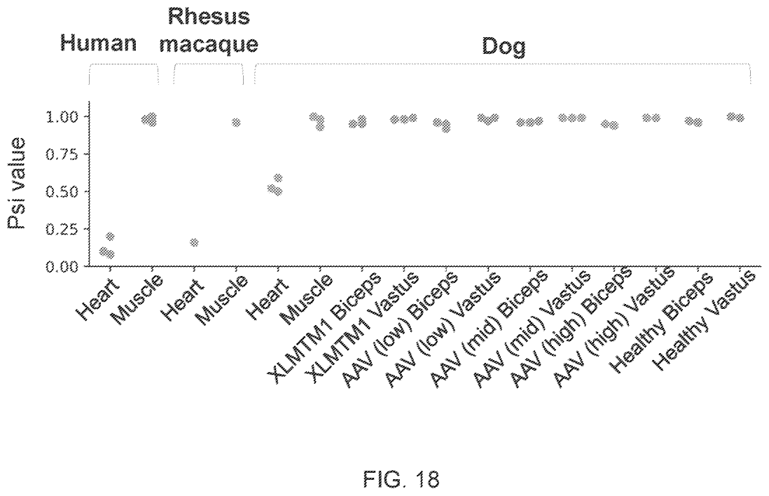

The high throughput screening approach described herein was first applied to BIN1 exon 11 because it showed the largest dynamic range in psi between heart and skeletal muscle (see FIGS. 14A-14C). BIN1 exon 11 has been previously studied and demonstrated to be responsive to RNA binding proteins such as the Muscleblind-like proteins (19) and RBFOX proteins (20); consistent with this, RBFOX1 and MBNL1 are the 5th- and 11th-most enriched RBPs, respectively, in skeletal muscle relative to heart. The upstream intron of BIN1 exon 11 is enriched for CAC motifs (10 instances versus an expectation of 3.8); pairs of CAC motifs separated by a variable spacer are known to bind RBPMS2 (21). RBPMS2 represses exon inclusion when binding to upstream introns (22) and is the 2nd-most enriched RBP in heart as compared to skeletal muscle. Notably, the psi values of BIN1 exon 11 in human and rhesus macaque heart are all close to 0%, but in dog, which contains only 1 instead of 2 CAC motifs in the 3′ splice site of BIN1 exon 11, unlike the other organisms, shows a psi value of −50% for BIN1 exon 11 (23) (FIGS. 17 and 18). Thus, RBPMS2 might be a critical factor that represses BIN1 exon 11 in heart.

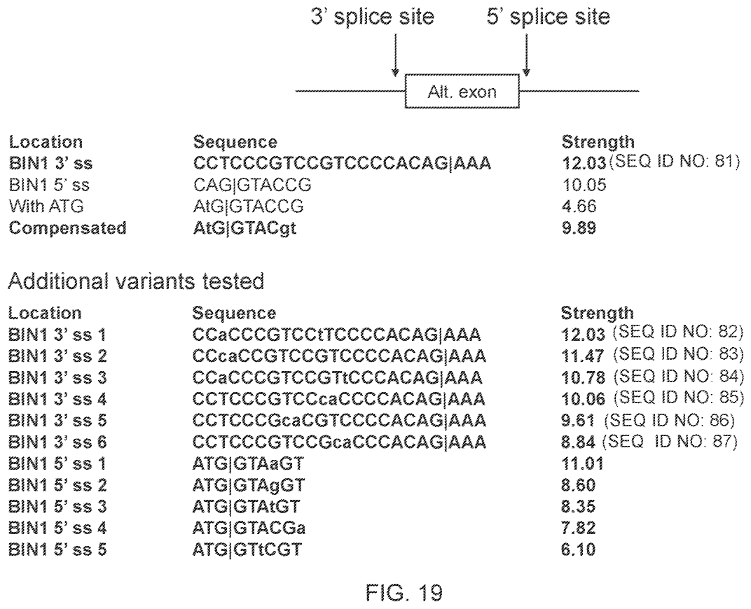

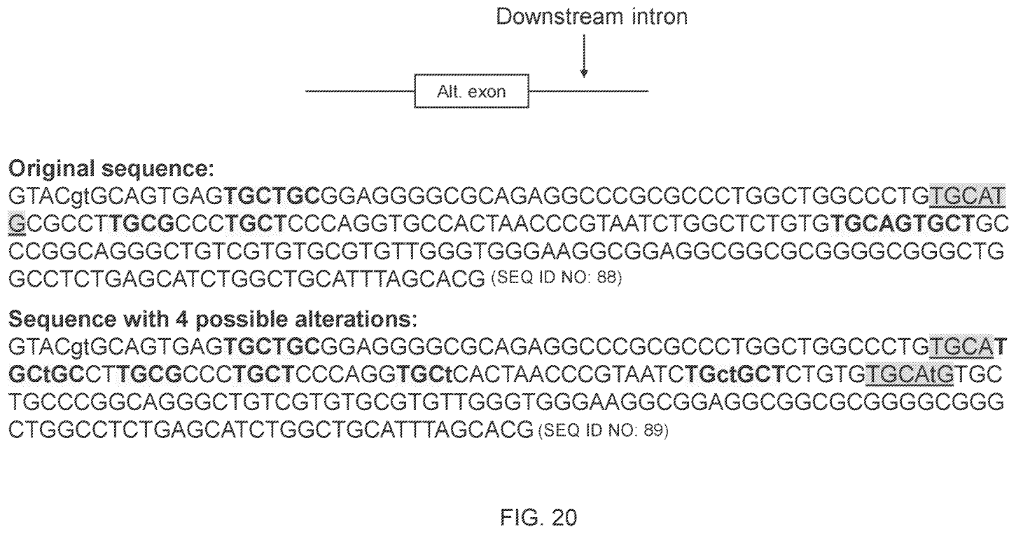

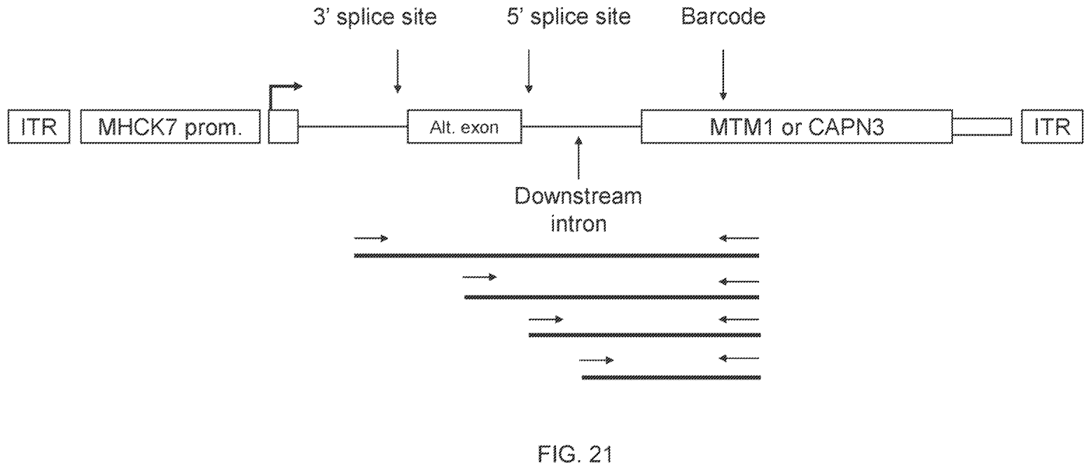

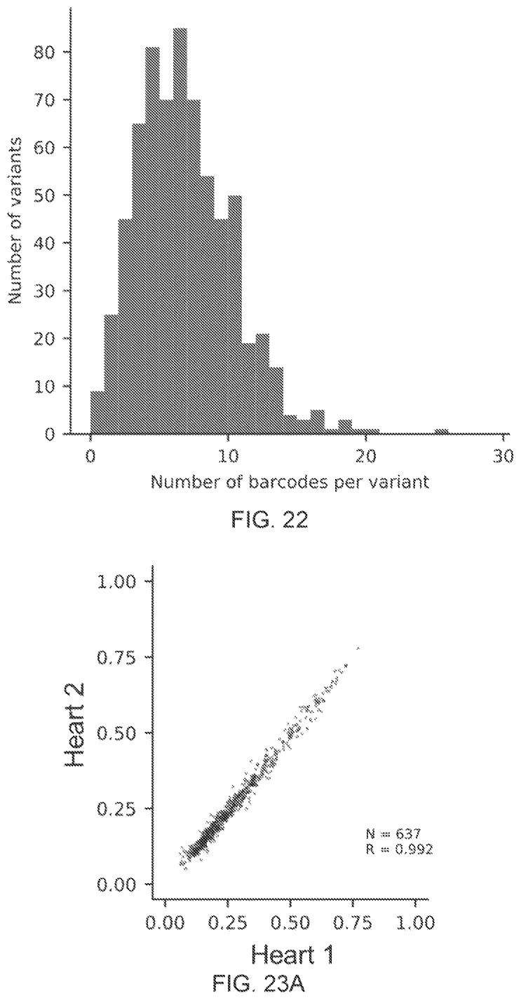

Given all of the above information, the 3′ splice site, 5′ splice site (FIG. 19), and downstream intron (FIG. 20) of BIN1 exon 11 was systematically altered to explore different splice site strengths, different configurations of CAC motifs within the 3′ splice site, and different frequencies of MBNL and RBFOX binding sites within the downstream intron. In total, AAV plasmid libraries were generated that contained 7 possible 3′ splice sites, 6 possible 5′ splice sites, and 16 possible downstream intronic sequences. The splice sites varied in strength, and the intronic sequences varied in the number of predicted MBNL and RBFOX binding sites. A total of 672 sequence variants were possible; each variant was linked to unique 10 nucleotide-long barcodes placed within the downstream coding sequence of MTM1. Each variant could be linked to several unique barcodes, such that multiple barcodes could serve as “replicates” for each sequence variant. Deep sequencing of PCR products amplified from plasmid libraries (FIG. 21) showed the presence of 663/672 variants, with an average of −8 barcodes per variant (FIG. 22).

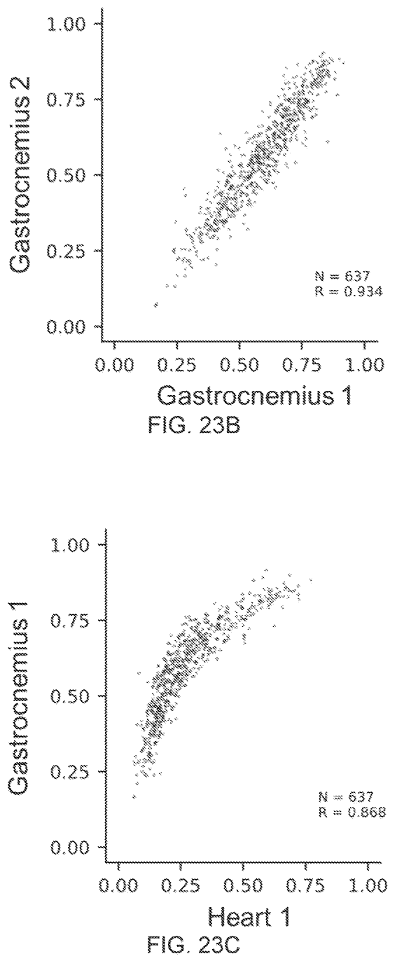

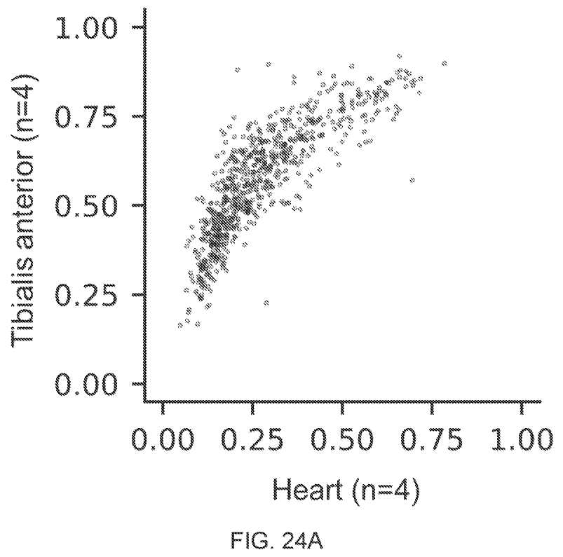

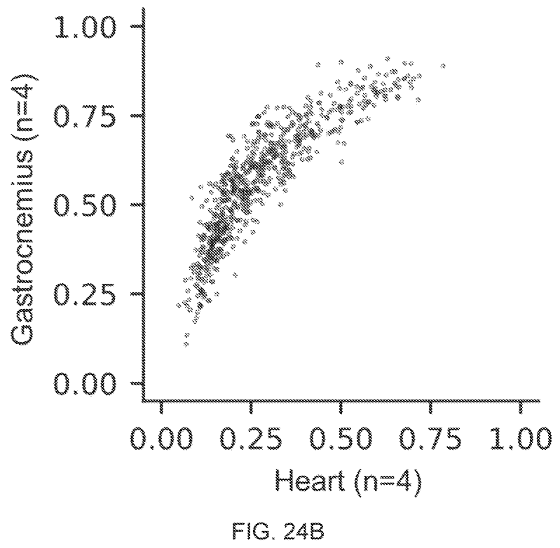

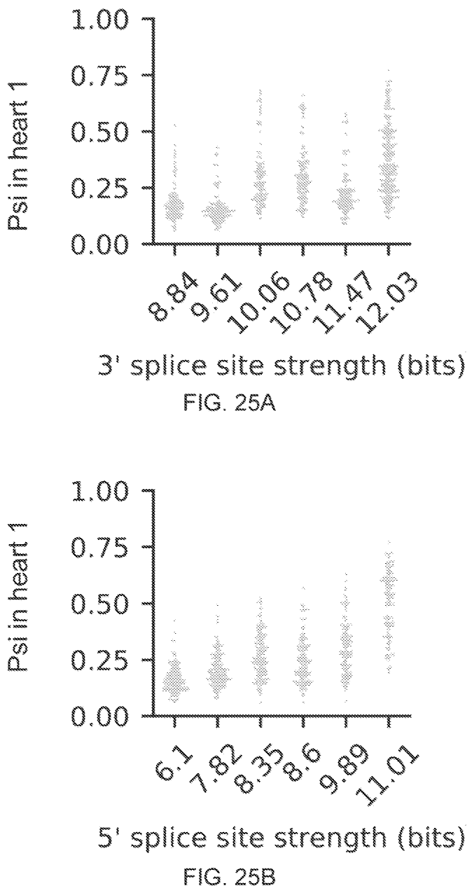

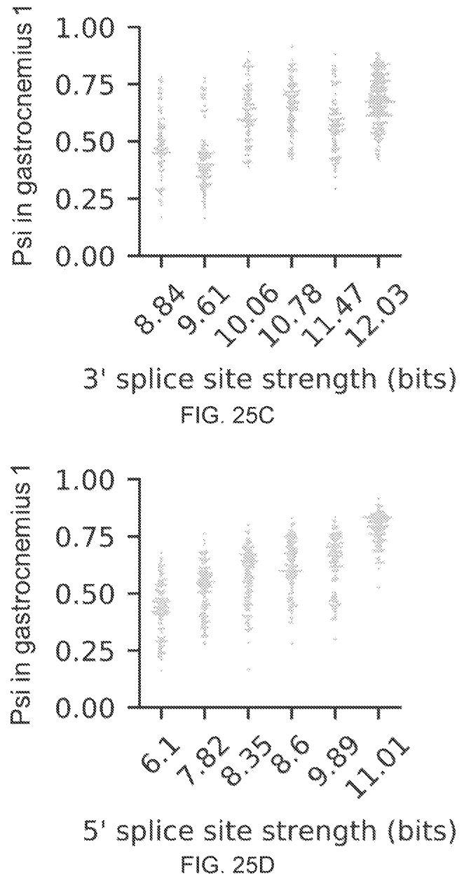

Viruses were generated using the eMyoAAV capsid (24) and administered to mice at a titer of 2.5e13 vg/kg. Heart, tibialis anterior, and triceps muscles were collected from mice sacrificed 3 weeks following administration. Sequencing libraries were prepared by RT-PCR and sequenced by Illumina sequencing. Psi values were computed for each barcode and a psi value for each variant was obtained by averaging the psi across every barcode for each variant. The psi value for each variant is shown for 2 heart samples in a scatter plot (FIG. 23A), and similarly, for 2 gastrocnemius samples (FIG. 23B), or a heart sample versus a gastrocnemius sample (FIG. 23C). The mean psi was computed for each variant across replicate tissues from multiple animals (n=4 animals for each tissue). These mean psi values for each variant were also plotted (FIGS. 24A-24B) and listed (Table 4). Psi values for each variant were also plotted as a function of 3′ splice site strength or 5′ splice site strength in heart and gastrocnemius (FIGS. 25A-25D), and clear dependencies of psi on splice site strength were observed. This relationship is particularly strong between 5′ splice strength and psi, supporting the idea that this screening approach can accurately quantitate relative psi values and identify sequence variants that exhibit specific splicing patterns.

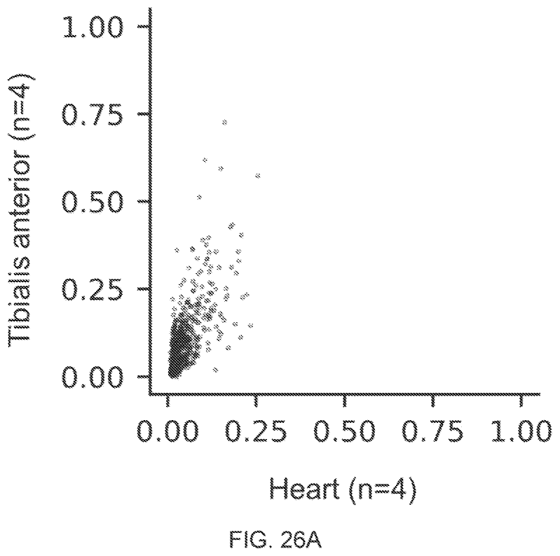

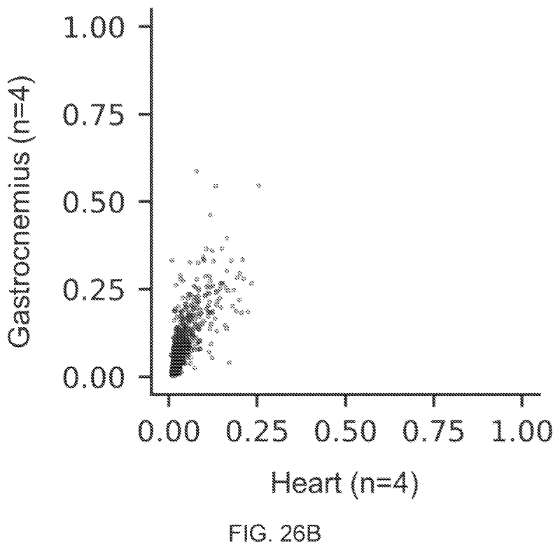

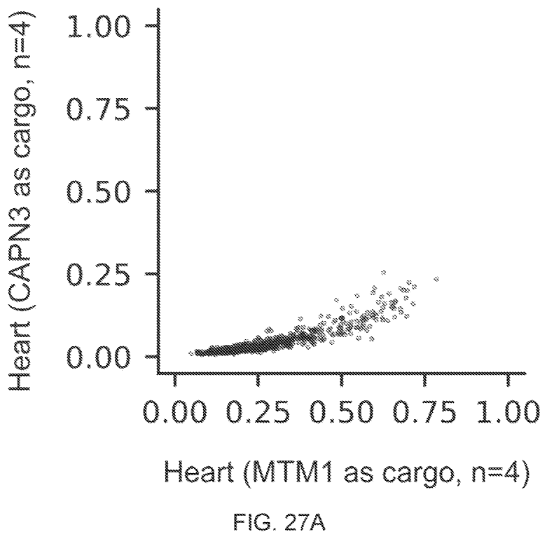

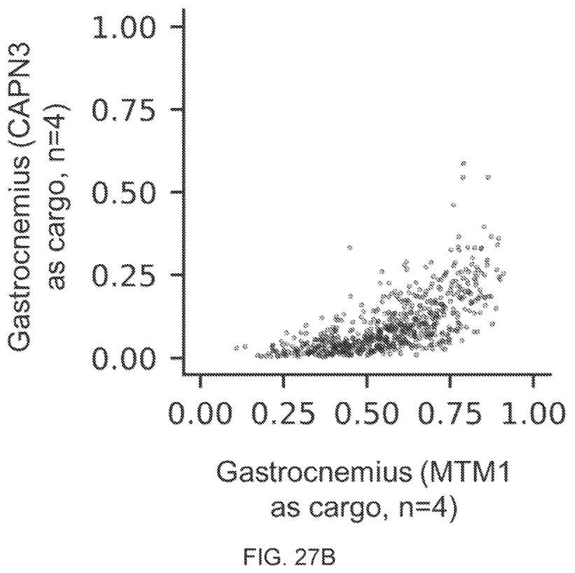

The same BIN1 exon 11 variants were also tested with a different cargo, CAPN3. A separate AAV library was generated in which all 672 BIN1 variants (Table 4) were cloned upstream of the CAPN3 coding sequence, analogously to how they were cloned upstream of the MTM1 coding sequence. Similarly, a 10 nucleotide barcode was embedded within the CAPN3 coding sequence to identify each splice variant. The mean psi values across heart, gastrocnemius, and tibialis anterior tissues from 4 animals were plotted as scatters (FIGS. 26A-26B), showing that some variants show lower inclusion in heart than in skeletal muscles. The overall behavior of each variant is strongly correlated across MTM1 and CAPN3 cargoes, but the baseline inclusion level of the BIN1 cassette is lower when linked to the CAPN3 cargo; this trend is observable when plotting the scatter of psi variants for each cargo in heart and gastrocnemius (FIGS. 27A-27B).

TABLE 4
Table of BIN1 exon 11 variants screened and associated psi values.

| | 3′ splice site | 5′ splice site | Intron | MTM1_Heart | MTM1_Gastroc | MTM1_Tibialis | CAPN3_Heart | CAPN3_Gastroc | CAPN3_Tibialis | SEQ |
|---|---|---|---|---|---|---|---|---|---|---|
| ID | ID | ID | insertions | (psi) | (psi) | (psi) | (psi) | (psi) | (psi) | ID NO: |
| 1 | BIN1 3′ ss | Compensated | None | 0.256 | 0.561 | 0.56975 | 0.034 | 0.09325 | 0.07625 | 107 |
| 2 | BIN1 3′ ss | Compensated | 4 | 0.335 | 0.61925 | 0.63175 | 0.032 | 0.28975 | 0.128 | 108 |
| 3 | BIN1 3′ ss | Compensated | 3 | 0.277 | 0.58325 | 0.607 | 0.03625 | 0.012 | 0.053 | 109 |
| 4 | BIN1 3′ ss | Compensated | 3, 4 | 0.34675 | 0.60175 | 0.5075 | 0.0705 | 0.1965 | 0.06125 | 110 |
| 5 | BIN1 3′ ss | Compensated | 2 | 0.29425 | 0.7735 | 0.89575 | 0.032 | 0.1075 | 0.0895 | 111 |
| 6 | BIN1 3′ ss | Compensated | 2, 4 | 0.33325 | 0.69075 | 0.739 | 0.03575 | 0.03925 | 0.01975 | 112 |
| 7 | BIN1 3′ ss | Compensated | 2, 3 | 0.272 | 0.7025 | 0.6855 | 0.02125 | 0.0845 | 0.05 | 113 |
| 8 | BIN1 3′ ss | Compensated | 2, 3, 4 | 0.37075 | 0.67875 | 0.687 | 0.04275 | 0.094 | 0.13575 | 114 |
| 9 | BIN1 3′ ss | Compensated | 1 | 0.34075 | 0.665 | 0.66875 | 0.038 | 0.104 | 0.08625 | 115 |
| 10 | BIN1 3′ ss | Compensated | 1, 4 | 0.33925 | 0.5715 | 0.62775 | 0.0435 | 0.08375 | 0.196333333 | 116 |
| 11 | BIN1 3′ ss | Compensated | 1, 3 | 0.373 | 0.6905 | 0.527 | 0.0545 | 0.06275 | 0.11175 | 117 |
| 12 | BIN1 3′ ss | Compensated | 1, 3, 4 | 0.49075 | 0.76675 | 0.7275 | 0.06975 | 0.0835 | 0.15725 | 118 |
| 13 | BIN1 3′ ss | Compensated | 1, 2 | 0.30625 | 0.6965 | 0.709 | 0.037 | 0.16525 | 0.11 | 119 |
| 14 | BIN1 3′ ss | Compensated | 1, 2, 4 | 0.3455 | 0.54075 | 0.575 | 0.037 | 0.0785 | 0.2615 | 120 |
| 15 | BIN1 3′ ss | Compensated | 1, 2, 3 | 0.26775 | 0.6765 | 0.61975 | 0.0465 | 0.19175 | 0.158 | 121 |
| 16 | BIN1 3′ ss | Compensated | 1, 2, 3, 4 | 0.38275 | 0.67925 | 0.48825 | 0.06725 | 0.12175 | 0.1995 | 122 |
| 17 | BIN1 3′ ss | BIN1 5′ ss 1 | None | 0.44025 | 0.7855 | 0.74575 | 0.0805 | 0.17825 | 0.18975 | 123 |
| 18 | BIN1 3′ ss | BIN1 5′ ss 1 | 4 | 0.503 | 0.7605 | 0.772 | 0.11775 | 0.4615 | 0.299 | 124 |
| 19 | BIN1 3′ ss | BIN1 5′ ss 1 | 3 | 0.497 | 0.77675 | 0.81 | 0.117 | 0.18375 | 0.13425 | 125 |
| 20 | BIN1 3′ ss | BIN1 5′ ss 1 | 3, 4 | 0.60025 | 0.813 | 0.76625 | 0.131 | 0.165 | 0.188 | 126 |
| 21 | BIN1 3′ ss | BIN1 5′ ss 1 | 2 | 0.4245 | 0.68775 | 0.729 | 0.07725 | 0.14775 | 0.056 | 127 |
| 22 | BIN1 3′ ss | BIN1 5′ ss 1 | 2, 4 | 0.43725 | 0.72175 | 0.81475 | 0.123 | 0.18175 | 0.2365 | 128 |
| 23 | BIN1 3′ ss | BIN1 5′ ss 1 | 2, 3 | 0.5275 | 0.83625 | 0.79425 | 0.09025 | 0.18025 | 0.19675 | 129 |
| 24 | BIN1 3′ ss | BIN1 5′ ss 1 | 2, 3, 4 | 0.61775 | 0.8395 | 0.82 | 0.13625 | 0.20475 | 0.24975 | 130 |
| 25 | BIN1 3′ ss | BIN1 5′ ss 1 | 1 | 0.5635 | 0.772 | 0.75325 | 0.08975 | 0.0805 | 0.085 | 131 |
| 26 | BIN1 3′ ss | BIN1 5′ ss 1 | 1, 4 | 0.6225 | 0.822 | 0.845 | 0.11625 | 0.1365 | 0.23875 | 132 |
| 27 | BIN1 3′ ss | BIN1 5′ ss 1 | 1, 3 | 0.65975 | 0.8515 | 0.85875 | 0.1645 | 0.395666667 | 0.221 | 133 |
| 28 | BIN1 3′ ss | BIN1 5′ ss 1 | 1, 3, 4 | 0.6495 | 0.8405 | 0.77725 | 0.16375 | 0.137333333 | 0.096 | 134 |
| 29 | BIN1 3′ ss | BIN1 5′ ss 1 | 1, 2 | 0.584 | 0.83175 | 0.7405 | 0.0915 | 0.18025 | 0.11375 | 135 |
| 30 | BIN1 3′ ss | BIN1 5′ ss 1 | 1, 2, 4 | 0.69575 | 0.896 | 0.571 | 0.12275 | 0.3605 | 0.19475 | 136 |
| 31 | BIN1 3′ ss | BIN1 5′ ss 1 | 1, 2, 3 | 0.56625 | 0.80625 | 0.73625 | 0.1245 | 0.2595 | 0.23925 | 137 |
| 32 | BIN1 3′ ss | BIN1 5′ ss 1 | 1, 2, 3, 4 | 0.6595 | 0.7995 | 0.91875 | 0.11975 | 0.09375 | 0.2685 | 138 |
| 33 | BIN1 3′ ss | BIN1 5′ ss 2 | None | 0.23525 | 0.5955 | 0.5705 | 0.0295 | 0.056 | 0.06675 | 139 |
| 34 | BIN1 3′ ss | BIN1 5′ ss 2 | 4 | 0.279 | 0.61275 | 0.65775 | 0.03175 | 0.154 | 0.06425 | 140 |
| 35 | BIN1 3′ ss | BIN1 5′ ss 2 | 3 | 0.1865 | 0.5205 | 0.554 | 0.0355 | 0.11725 | 0.101 | 141 |
| 36 | BIN1 3′ ss | BIN1 5′ ss 2 | 3, 4 | 0.30075 | 0.623 | 0.602 | 0.03225 | 0.073 | 0.076 | 142 |
| 37 | BIN1 3′ ss | BIN1 5′ ss 2 | 2 | 0.19725 | 0.62725 | 0.65425 | 0.02075 | 0.0425 | 0.0275 | 143 |
| 38 | BIN1 3′ ss | BIN1 5′ ss 2 | 2, 4 | 0.24975 | 0.6075 | 0.647 | 0.0265 | 0.045 | 0.02425 | 144 |
| 39 | BIN1 3′ ss | BIN1 5′ ss 2 | 2, 3 | 0.1995 | 0.682 | 0.693 | 0.02225 | 0.06425 | 0.028 | 145 |
| 40 | BIN1 3′ ss | BIN1 5′ ss 2 | 2, 3, 4 | 0.27075 | 0.6875 | 0.675 | 0.03825 | 0.07375 | 0.10475 | 146 |
| 41 | BIN1 3′ ss | BIN1 5′ ss 2 | 1 | 0.2655 | 0.6125 | 0.68575 | 0.0315 | 0.08425 | 0.099 | 147 |
| 42 | BIN1 3′ ss | BIN1 5′ ss 2 | 1, 4 | 0.31925 | 0.6395 | 0.5975 | 0.0445 | 0.0995 | 0.10475 | 148 |
| 43 | BIN1 3′ ss | BIN1 5′ ss 2 | 1, 3 | 0.3245 | 0.67225 | 0.7045 | 0.03 | 0.0835 | 0.104 | 149 |
| 44 | BIN1 3′ ss | BIN1 5′ ss 2 | 1, 3, 4 | 0.33875 | 0.628 | 0.64725 | 0.0485 | 0.08275 | 0.10775 | 150 |
| 45 | BIN1 3′ ss | BIN1 5′ ss 2 | 1, 2 | 0.17525 | 0.58175 | 0.677 | 0.019 | 0.1345 | 0.0945 | 151 |
| 46 | BIN1 3′ ss | BIN1 5′ ss 2 | 1, 2, 4 | 0.29675 | 0.73175 | 0.7265 | 0.0385 | 0.154 | 0.22675 | 152 |
| 47 | BIN1 3′ ss | BIN1 5′ ss 2 | 1, 2, 3 | 0.22975 | 0.69275 | 0.615 | 0.026 | 0.133 | 0.12825 | 153 |
| 48 | BIN1 3′ ss | BIN1 5′ ss 2 | 1, 2, 3, 4 | 0.4445 | 0.81275 | 0.69475 | 0.051 | 0.167 | 0.10325 | 154 |
| 49 | BIN1 3′ ss | BIN1 5′ ss 3 | None | 0.27525 | 0.5135 | 0.522 | 0.029 | 0.0735 | 0.119 | 155 |
| 50 | BIN1 3′ ss | BIN1 5′ ss 3 | 4 | 0.2615 | 0.51225 | 0.5445 | 0.02975 | 0.0875 | 0.0645 | 156 |
| 51 | BIN1 3′ ss | BIN1 5′ ss 3 | 3 | 0.2595 | 0.56425 | 0.63 | 0.0335 | 0.1845 | 0.12775 | 157 |
| 52 | BIN1 3′ ss | BIN1 5′ ss 3 | 3, 4 | 0.3565 | 0.5915 | 0.6615 | 0.05225 | 0.0955 | 0.08575 | 158 |
| 53 | BIN1 3′ ss | BIN1 5′ ss 3 | 2 | 0.222 | 0.5615 | 0.61725 | 0.02425 | 0.05575 | 0.053 | 159 |
| 54 | BIN1 3′ ss | BIN1 5′ ss 3 | 2, 4 | 0.24225 | 0.50025 | 0.492 | 0.0405 | 0.06625 | 0.0925 | 160 |
| 55 | BIN1 3′ ss | BIN1 5′ ss 3 | 2, 3 | 0.199 | 0.5735 | 0.51875 | 0.0185 | 0.194 | 0.07525 | 16 |
| 56 | BIN1 3′ ss | BIN1 5′ ss 3 | 2, 3, 4 | 0.28375 | 0.5335 | 0.6195 | 0.04525 | 0.14725 | 0.0605 | 162 |
| 57 | BIN1 3′ ss | BIN1 5′ ss 3 | 1 | 0.28525 | 0.621 | 0.622 | 0.03825 | 0.16625 | 0.11175 | 163 |
| 58 | BIN1 3′ ss | BIN1 5′ ss 3 | 1, 4 | 0.341 | 0.5825 | 0.573 | 0.0405 | 0.0525 | 0.09175 | 164 |
| 59 | BIN1 3′ ss | BIN1 5′ ss 3 | 1, 3 | 0.33825 | 0.66 | 0.6695 | 0.05575 | 0.159 | 0.1125 | 165 |
| 60 | BIN1 3′ ss | BIN1 5′ ss 3 | 1, 3, 4 | 0.41175 | 0.659 | 0.585 | 0.041 | 0.06025 | 0.0295 | 166 |
| 61 | BIN1 3′ ss | BIN1 5′ ss 3 | 1, 2 | 0.3115 | 0.6545 | 0.62475 | 0.02575 | 0.03725 | 0.0425 | 167 |
| 62 | BIN1 3′ ss | BIN1 5′ ss 3 | 1, 2, 4 | 0.28625 | 0.61425 | 0.56325 | 0.0375 | 0.09825 | 0.10625 | 168 |
| 63 | BIN1 3′ ss | BIN1 5′ ss 3 | 1, 2, 3 | 0.27025 | 0.70475 | 0.648 | 0.02925 | 0.06975 | 0.045 | 169 |
| 64 | BIN1 3′ ss | BIN1 5′ ss 3 | 1, 2, 3, 4 | 0.3995 | 0.7205 | 0.566 | 0.02825 | 0.042 | 0.06025 | 170 |
| 65 | BIN1 3′ ss | BIN1 5′ ss 4 | None | 0.234 | 0.51125 | 0.501 | 0.02 | 0.05225 | 0.03525 | 171 |
| 66 | BIN1 3′ ss | BIN1 5′ ss 4 | 4 | 0.248 | 0.507 | 0.55575 | 0.02525 | 0.055 | 0.04 | 172 |
| 67 | BIN1 3′ ss | BIN1 5′ ss 4 | 3 | 0.195 | 0.4575 | 0.53675 | 0.02025 | 0.06125 | 0.14625 | 173 |
| 68 | BIN1 3′ ss | BIN1 5′ ss 4 | 3, 4 | 0.281 | 0.5025 | 0.608 | 0.028 | 0.0575 | 0.087 | 174 |
| 69 | BIN1 3′ ss | BIN1 5′ ss 4 | 2 | 0.19375 | 0.486 | 0.55925 | 0.01875 | 0.0415 | 0.031 | 175 |
| 70 | BIN1 3′ ss | BIN1 5′ ss 4 | 2, 4 | 0.1675 | 0.4305 | 0.40925 | 0.0165 | 0.036 | 0.04925 | 176 |
| 71 | BIN1 3′ ss | BIN1 5′ ss 4 | 2, 3 | 0.151 | 0.47375 | 0.507 | 0.0155 | 0.074 | 0.03925 | 177 |
| 72 | BIN1 3′ ss | BIN1 5′ ss 4 | 2, 3, 4 | 0.1875 | 0.51275 | 0.45325 | 0.015 | 0.02375 | 0.02875 | 178 |
| 73 | BIN1 3′ ss | BIN1 5′ ss 4 | 1 | 0.236 | 0.54375 | 0.56025 | 0.03125 | 0.06825 | 0.0575 | 179 |
| 74 | BIN1 3′ ss | BIN1 5′ ss 4 | 1, 4 | 0.237 | 0.47725 | 0.50675 | 0.0335 | 0.05225 | 0.09525 | 180 |
| 75 | BIN1 3′ ss | BIN1 5′ ss 4 | 1, 3 | 0.22225 | 0.54 | 0.57275 | 0.0395 | 0.225 | 0.1115 | 181 |
| 76 | BIN1 3′ ss | BIN1 5′ ss 4 | 1, 3, 4 | 0.32675 | 0.57525 | 0.593 | 0.03475 | 0.098 | 0.04425 | 182 |
| 77 | BIN1 3′ ss | BIN1 5′ ss 4 | 1, 2 | 0.197 | 0.55275 | 0.55925 | 0.024 | 0.02775 | 0.01325 | 183 |
| 78 | BIN1 3′ ss | BIN1 5′ ss 4 | 1, 2, 4 | 0.2015 | 0.49175 | 0.54575 | 0.0225 | 0.0495 | 0.06075 | 184 |
| 79 | BIN1 3′ ss | BIN1 5′ ss 4 | 1, 2, 3 | 0.17375 | 0.512 | 0.5245 | 0.018 | 0.02425 | 0.04725 | 185 |
| 80 | BIN1 3′ ss | BIN1 5′ ss 4 | 1, 2, 3, 4 | 0.17 | 0.5355 | 0.337 | 0.0275 | 0.085 | 0.01975 | 186 |
| 81 | BIN1 3′ ss | BIN1 5′ ss 5 | None | 0.17 | 0.4725 | 0.51325 | 0.017 | 0.02225 | 0.01675 | 187 |
| 82 | BIN1 3′ ss | BIN1 5′ ss 5 | 4 | 0.2395 | 0.47925 | 0.50775 | 0.02175 | 0.014 | 0.019 | 188 |
| 83 | BIN1 3′ ss | BIN1 5′ ss 5 | 3 | 0.167 | 0.548 | 0.333 | 0.0145 | 0.036 | 0.031 | 189 |
| 84 | BIN1 3′ ss | BIN1 5′ ss 5 | 3, 4 | 0.23325 | 0.44375 | 0.491 | 0.0145 | 0.04175 | 0.011 | 190 |
| 85 | BIN1 3′ ss | BIN1 5′ ss 5 | 2 | 0.1425 | 0.3805 | 0.48225 | 0.009 | 0.00725 | 0.021 | 191 |
| 86 | BIN1 3′ ss | BIN1 5′ ss 5 | 2, 4 | 0.15425 | 0.4395 | 0.41275 | 0.01825 | 0.03825 | 0.12775 | 192 |
| 87 | BIN1 3′ ss | BIN1 5′ ss 5 | 2, 3 | 0.15125 | 0.39975 | 0.37475 | 0.0135 | 0.012 | 0.01075 | 193 |
| 88 | BIN1 3′ ss | BIN1 5′ ss 5 | 2, 3, 4 | 0.14375 | 0.396 | 0.41175 | 0.0115 | 0.014 | 0.034 | 194 |
| 89 | BIN1 3′ ss | BIN1 5′ ss 5 | 1 | 0.18325 | 0.44375 | 0.43525 | 0.02025 | 0.03925 | 0.10725 | 195 |
| 90 | BIN1 3′ ss | BIN1 5′ ss 5 | 1, 4 | 0.256 | 0.45675 | 0.59375 | 0.0195 | 0.03225 | 0.03175 | 196 |
| 91 | BIN1 3′ ss | BIN1 5′ ss 5 | 1, 3 | 0.23325 | 0.53275 | 0.50925 | 0.01875 | 0.03225 | 0.06375 | 197 |
| 92 | BIN1 3′ ss | BIN1 5′ ss 5 | 1, 3, 4 | 0.2325 | 0.39625 | 0.43625 | 0.01475 | 0.01175 | 0.025 | 198 |
| 93 | BIN1 3′ ss | BIN1 5′ ss 5 | 1, 2 | ND | ND | ND | 0.01025 | 0.00425 | 0.007 | 199 |
| 94 | BIN1 3′ ss | BIN1 5′ ss 5 | 1, 2, 4 | 0.13575 | 0.4355 | 0.388 | 0.01925 | 0.03875 | 0.142 | 200 |
| 95 | BIN1 3′ ss | BIN1 5′ ss 5 | 1, 2, 3 | 0.1185 | 0.34775 | 0.39975 | 0.0135 | 0.054 | 0.036 | 201 |
| 96 | BIN1 3′ ss | BIN1 5′ ss 5 | 1, 2, 3, 4 | 0.15425 | 0.44525 | 0.40675 | 0.01075 | 0.01125 | 0.0065 | 202 |
| 97 | BIN1 3′ ss 1 | Compensated | None | 0.43725 | 0.786 | 0.69575 | 0.0495 | 0.053 | 0.0565 | 203 |
| 98 | BIN1 3′ ss 1 | Compensated | 4 | 0.4675 | 0.75975 | 0.65775 | 0.0695 | 0.193 | 0.366 | 204 |
| 99 | BIN1 3′ ss 1 | Compensated | 3 | 0.46025 | 0.74575 | 0.82025 | 0.12225 | 0.0545 | 0.314 | 205 |
| 100 | BIN1 3′ ss 1 | Compensated | 3, 4 | 0.57525 | 0.78675 | 0.8045 | 0.07875 | 0.2075 | 0.06375 | 206 |
| 101 | BIN1 3′ ss 1 | Compensated | 2 | 0.38725 | 0.72125 | 0.64 | 0.042 | 0.16275 | 0.08925 | 207 |
| 102 | BIN1 3′ ss 1 | Compensated | 2, 4 | 0.433 | 0.648 | 0.73875 | 0.07175 | 0.22675 | 0.05125 | 208 |
| 103 | BIN1 3′ ss 1 | Compensated | 2, 3 | 0.36725 | 0.724 | 0.491 | 0.08125 | 0.14275 | 0.1815 | 209 |
| 104 | BIN1 3′ ss 1 | Compensated | 2, 3, 4 | 0.52725 | 0.76 | 0.84175 | 0.094 | 0.1485 | 0.301 | 210 |
| 105 | BIN1 3′ ss 1 | Compensated | 1 | 0.5 | 0.739 | 0.6785 | 0.07125 | 0.25825 | 0.2725 | 211 |
| 106 | BIN1 3′ ss 1 | Compensated | 1, 4 | 0.57475 | 0.74125 | 0.855 | 0.11375 | 0.07175 | 0.09625 | 212 |
| 107 | BIN1 3′ ss 1 | Compensated | 1, 3 | 0.51875 | 0.7765 | 0.74725 | 0.08275 | 0.32525 | 0.114 | 213 |
| 108 | BIN1 3′ ss 1 | Compensated | 1, 3, 4 | 0.50175 | 0.62025 | 0.76425 | 0.14625 | 0.26625 | 0.12825 | 214 |
| 109 | BIN1 3′ ss 1 | Compensated | 1, 2 | 0.56525 | 0.85525 | 0.67775 | 0.0995 | 0.15975 | 0.39 | 215 |
| 110 | BIN1 3′ ss 1 | Compensated | 1, 2, 4 | 0.47575 | 0.76575 | 0.86275 | 0.104 | 0.287 | 0.1315 | 216 |
| 111 | BIN1 3′ ss 1 | Compensated | 1, 2, 3 | 0.52275 | 0.7575 | 0.7185 | 0.08425 | 0.2305 | 0.20425 | 217 |
| 112 | BIN1 3′ ss 1 | Compensated | 1, 2, 3, 4 | 0.6245 | 0.795 | 0.84475 | 0.111 | 0.2005 | 0.1635 | 218 |
| 113 | BIN1 3′ ss 1 | BIN1 5′ ss 1 | None | 0.484 | 0.71175 | 0.64525 | 0.1715 | 0.04175 | 0.0825 | 219 |
| 114 | BIN1 3′ ss 1 | BIN1 5′ ss 1 | 4 | 0.62375 | 0.805 | 0.8095 | 0.149 | 0.19775 | 0.17875 | 220 |
| 115 | BIN1 3′ ss 1 | BIN1 5′ ss 1 | 3 | 0.66175 | 0.83025 | 0.87875 | 0.168 | 0.24525 | 0.23075 | 221 |
| 116 | BIN1 3′ ss 1 | BIN1 5′ ss 1 | 3, 4 | 0.677 | 0.85425 | 0.83375 | 0.206 | 0.18175 | 0.11175 | 222 |
| 117 | BIN1 3′ ss 1 | BIN1 5′ ss 1 | 2 | 0.56975 | 0.781 | 0.8175 | 0.1155 | 0.18325 | 0.39675 | 223 |
| 118 | BIN1 3′ ss 1 | BIN1 5′ ss 1 | 2, 4 | 0.578 | 0.789 | 0.68175 | 0.13375 | 0.2405 | 0.20525 | 224 |
| 119 | BIN1 3′ ss 1 | BIN1 5′ ss 1 | 2, 3 | 0.6865 | 0.86175 | 0.87 | 0.20125 | 0.29525 | 0.33 | 225 |
| 120 | BIN1 3′ ss 1 | BIN1 5′ ss | 2, 3, 4 | 0.70275 | 0.85425 | 0.8515 | 0.22425 | 0.18475 | 0.2335 | 226 |
| 121 | BIN1 3′ ss 1 | BIN1 5′ ss 1 | 1 | 0.67675 | 0.85325 | 0.85625 | 0.14525 | 0.25075 | 0.108 | 227 |
| 122 | BIN1 3′ ss 1 | BIN1 5′ ss 1 | 1, 4 | 0.6975 | 0.82875 | 0.83625 | 0.2 | 0.21925 | 0.3565 | 228 |
| 123 | BIN1 3′ ss 1 | BIN1 5′ ss 1 | 1, 3 | 0.71825 | 0.8615 | 0.8565 | 0.21175 | 0.2795 | 0.22575 | 229 |
| 124 | BIN1 3′ ss 1 | BIN1 5′ ss 1 | 1, 3, 4 | 0.7845 | 0.89025 | 0.8985 | 0.2345 | 0.266 | 0.1455 | 230 |
| 125 | BIN1 3′ ss 1 | BIN1 5′ ss 1 | 1, 2 | 0.64275 | 0.8745 | 0.74175 | 0.191 | 0.1865 | 0.15 | 231 |
| 126 | BIN1 3′ ss 1 | BIN1 5′ ss 1 | 1, 2, 4 | 0.626 | 0.86425 | 0.76925 | 0.25475 | 0.545333333 | 0.573666667 | 232 |
| 127 | BIN1 3′ ss 1 | BIN1 5′ ss 1 | 1, 2, 3 | 0.69325 | 0.8435 | 0.846 | 0.1775 | 0.33175 | 0.4265 | 233 |
| 128 | BIN1 3′ ss 1 | BIN1 5′ ss 1 | 1, 2, 3, 4 | 0.71475 | 0.79475 | 0.815 | 0.1595 | 0.147 | 0.1605 | 234 |
| 129 | BIN1 3′ ss 1 | BIN1 5′ ss 2 | None | 0.3525 | 0.71125 | 0.74075 | 0.03925 | 0.10675 | 0.05225 | 235 |
| 130 | BIN1 3′ ss 1 | BIN1 5′ ss 2 | 4 | 0.39425 | 0.703 | 0.69675 | 0.058 | 0.09475 | 0.219 | 236 |
| 131 | BIN1 3′ ss 1 | BIN1 5′ ss 2 | 3 | 0.403 | 0.748 | 0.7475 | 0.057 | 0.08625 | 0.114 | 237 |
| 132 | BIN1 3′ ss 1 | BIN1 5′ ss 2 | 3, 4 | 0.5045 | 0.7365 | 0.74425 | 0.075 | 0.15 | 0.08875 | 238 |
| 133 | BIN1 3′ ss 1 | BIN1 5′ ss 2 | 2 | 0.34225 | 0.71075 | 0.653 | 0.04575 | 0.1305 | 0.12525 | 239 |
| 134 | BIN1 3′ ss 1 | BIN1 5′ ss 2 | 2, 4 | 0.31525 | 0.60475 | 0.5855 | 0.052 | 0.101 | 0.1585 | 240 |
| 135 | BIN1 3′ ss 1 | BIN1 5′ ss 2 | 2, 3 | 0.34075 | 0.77425 | 0.73725 | 0.04725 | 0.19825 | 0.088 | 241 |
| 136 | BIN1 3′ ss 1 | BIN1 5′ ss 2 | 2, 3, 4 | 0.47325 | 0.7045 | 0.73325 | 0.0645 | 0.16825 | 0.07775 | 242 |
| 137 | BIN1 3′ ss 1 | BIN1 5′ ss 2 | 1 | 0.41525 | 0.77325 | 0.691 | 0.06975 | 0.216 | 0.119 | 243 |
| 138 | BIN1 3′ ss 1 | BIN1 5′ ss 2 | 1, 4 | 0.49725 | 0.7735 | 0.7935 | 0.0865 | 0.13525 | 0.2075 | 244 |
| 139 | BIN1 3′ ss 1 | BIN1 5′ ss 2 | 1, 3 | 0.4615 | 0.74375 | 0.749 | 0.08075 | 0.19575 | 0.0615 | 245 |
| 140 | BIN1 3′ ss 1 | BIN1 5′ ss 2 | 1, 3, 4 | 0.4655 | 0.69925 | 0.76425 | 0.085 | 0.246 | 0.153 | 246 |
| 141 | BIN1 3′ ss 1 | BIN1 5′ ss 2 | 1, 2 | 0.40175 | 0.7775 | 0.68425 | 0.052 | 0.1045 | 0.10075 | 247 |
| 142 | BIN1 3′ ss 1 | BIN1 5′ ss 2 | 1, 2, 4 | 0.4115 | 0.76075 | 0.7365 | 0.079 | 0.27775 | 0.10975 | 248 |
| 143 | BIN1 3′ ss 1 | BIN1 5′ ss 2 | 1, 2, 3 | 0.4705 | 0.808 | 0.82875 | 0.04375 | 0.14325 | 0.1445 | 249 |
| 144 | BIN1 3′ ss 1 | BIN1 5′ ss 2 | 1, 2, 3, 4 | 0.548 | 0.87675 | 0.66925 | 0.08775 | 0.10275 | 0.181 | 250 |
| 145 | BIN1 3′ ss 1 | BIN1 5′ ss 3 | None | 0.355 | 0.57775 | 0.686 | 0.06575 | 0.114 | 0.14175 | 251 |
| 146 | BIN1 3′ ss 1 | BIN1 5′ ss 3 | 4 | 0.40425 | 0.695 | 0.63925 | 0.06125 | 0.039 | 0.0855 | 252 |
| 147 | BIN1 3′ ss 1 | BIN1 5′ ss 3 | 3 | 0.44725 | 0.7505 | 0.727 | 0.0555 | 0.17125 | 0.21025 | 253 |
| 148 | BIN1 3′ ss 1 | BIN1 5′ ss 3 | 3, 4 | 0.4375 | 0.69425 | 0.60025 | 0.0835 | 0.1375 | 0.0875 | 254 |
| 149 | BIN1 3′ ss 1 | BIN1 5′ ss 3 | 2 | 0.3835 | 0.73775 | 0.71375 | 0.0385 | 0.04825 | 0.17675 | 255 |
| 150 | BIN1 3′ ss 1 | BIN1 5′ ss 3 | 2, 4 | 0.3975 | 0.6575 | 0.7285 | 0.058 | 0.09975 | 0.135 | 256 |
| 151 | BIN1 3′ ss 1 | BIN1 5′ ss 3 | 2, 3 | 0.33075 | 0.6775 | 0.6735 | 0.03975 | 0.273 | 0.052 | 257 |
| 152 | BIN1 3′ ss 1 | BIN1 5′ ss 3 | 2, 3, 4 | 0.509 | 0.67225 | 0.69875 | 0.066 | 0.126 | 0.08425 | 258 |
| 153 | BIN1 3′ ss 1 | BIN1 5′ ss 3 | 1 | 0.44375 | 0.70925 | 0.69925 | 0.0655 | 0.13375 | 0.1345 | 259 |
| 154 | BIN1 3′ ss 1 | BIN1 5′ ss 3 | 1, 4 | 0.49475 | 0.7025 | 0.73925 | 0.06625 | 0.102 | 0.0665 | 260 |
| 155 | BIN1 3′ ss 1 | BIN1 5′ ss 3 | 1, 3 | 0.49175 | 0.6745 | 0.76475 | 0.08725 | 0.101 | 0.222 | 261 |
| 156 | BIN1 3′ ss 1 | BIN1 5′ ss 3 | 1, 3, 4 | 0.49925 | 0.71675 | 0.769 | 0.1155 | 0.21275 | 0.1745 | 262 |
| 157 | BIN1 3′ ss 1 | BIN1 5′ ss 3 | 1, 2 | 0.49075 | 0.8445 | 0.61425 | 0.0745 | 0.25525 | 0.068 | 263 |
| 158 | BIN1 3′ ss 1 | BIN1 5′ ss 3 | 1, 2, 4 | 0.39225 | 0.62725 | 0.6535 | 0.06725 | 0.17275 | 0.13125 | 264 |
| 159 | BIN1 3′ ss 1 | BIN1 5′ ss 3 | 1, 2, 3 | 0.40325 | 0.71375 | 0.7035 | 0.05425 | 0.236 | 0.1505 | 265 |
| 160 | BIN1 3′ ss 1 | BIN1 5′ ss 3 | 1, 2, 3, 4 | 0.59 | 0.822 | 0.81675 | 0.054 | 0.0795 | 0.0355 | 266 |
| 161 | BIN1 3′ ss 1 | BIN1 5′ ss 4 | None | 0.2885 | 0.6205 | 0.60025 | 0.03325 | 0.0455 | 0.0595 | 267 |
| 162 | BIN1 3′ ss 1 | BIN1 5′ ss 4 | 4 | 0.30275 | 0.574 | 0.503 | 0.04775 | 0.08725 | 0.0275 | 268 |
| 163 | BIN1 3′ ss 1 | BIN1 5′ ss 4 | 3 | 0.26975 | 0.61175 | 0.59675 | 0.0275 | 0.13775 | 0.05025 | 269 |
| 164 | BIN1 3′ ss 1 | BIN1 5′ ss 4 | 3, 4 | 0.39025 | 0.64575 | 0.65325 | 0.06275 | 0.12025 | 0.08775 | 270 |
| 165 | BIN1 3′ ss 1 | BIN1 5′ ss 4 | 2 | 0.27225 | 0.58775 | 0.571 | 0.02175 | 0.08625 | 0.0745 | 271 |
| 166 | BIN1 3′ ss 1 | BIN1 5′ ss 4 | 2, 4 | 0.29875 | 0.6475 | 0.611 | 0.0335 | 0.055 | 0.0145 | 272 |
| 167 | BIN1 3′ ss 1 | BIN1 5′ ss 4 | 2, 3 | 0.20225 | 0.54625 | 0.503 | 0.03375 | 0.17 | 0.10175 | 273 |
| 168 | BIN1 3′ ss 1 | BIN1 5′ ss 4 | 2, 3, 4 | 0.3425 | 0.63175 | 0.59675 | 0.04275 | 0.13675 | 0.0945 | 274 |
| 169 | BIN1 3′ ss 1 | BIN1 5′ ss 4 | 1 | 0.31325 | 0.63375 | 0.67575 | 0.045 | 0.13725 | 0.1195 | 275 |
| 170 | BIN1 3′ ss 1 | BIN1 5′ ss 4 | 1, 4 | 0.35525 | 0.59725 | 0.64625 | 0.05625 | 0.17825 | 0.0885 | 276 |
| 171 | BIN1 3′ ss 1 | BIN1 5′ ss 4 | 1, 3 | 0.429 | 0.696 | 0.7905 | 0.0545 | 0.22325 | 0.0705 | 277 |
| 172 | BIN1 3′ ss 1 | BIN1 5′ ss 4 | 1, 3, 4 | 0.4785 | 0.70725 | 0.715 | 0.069 | 0.17575 | 0.17025 | 278 |
| 173 | BIN1 3′ ss 1 | BIN1 5′ ss 4 | 1, 2 | 0.2855 | 0.6385 | 0.6785 | 0.03875 | 0.0385 | 0.0945 | 279 |
| 174 | BIN1 3′ ss 1 | BIN1 5′ ss 4 | 1, 2, 4 | 0.31325 | 0.66275 | 0.63675 | 0.05675 | 0.1125 | 0.067 | 280 |
| 175 | BIN1 3′ ss 1 | BIN1 5′ ss 4 | 1, 2, 3 | 0.305 | 0.72275 | 0.6155 | 0.05975 | 0.23475 | 0.1755 | 281 |
| 176 | BIN1 3′ ss 1 | BIN1 5′ ss 4 | 1, 2, 3, 4 | 0.4185 | 0.69525 | 0.68775 | 0.038 | 0.0525 | 0.0525 | 282 |
| 177 | BIN1 3′ ss 1 | BIN1 5′ ss 5 | None | 0.21 | 0.45575 | 0.46175 | 0.01475 | 0.037 | 0.13675 | 283 |
| 178 | BIN1 3′ ss 1 | BIN1 5′ ss 5 | 4 | 0.2845 | 0.59875 | 0.48525 | 0.0265 | 0.04525 | 0.013 | 284 |
| 179 | BIN1 3′ ss 1 | BIN1 5′ ss 5 | 3 | 0.224 | 0.53525 | 0.49025 | 0.02575 | 0.059 | 0.03925 | 285 |
| 180 | BIN1 3′ ss 1 | BIN1 5′ ss 5 | 3, 4 | 0.338 | 0.645 | 0.504 | 0.0425 | 0.068 | 0.158 | 286 |
| 181 | BIN1 3′ ss 1 | BIN1 5′ ss 5 | 2 | 0.17525 | 0.575 | 0.553 | 0.01775 | 0.1015 | 0.09225 | 287 |
| 182 | BIN1 3′ ss 1 | BIN1 5′ ss 5 | 2, 4 | 0.1705 | 0.569 | 0.486 | 0.019 | 0.116 | 0.04725 | 288 |
| 183 | BIN1 3′ ss 1 | BIN1 5′ ss 5 | 2, 3 | 0.13425 | 0.56025 | 0.50575 | 0.0175 | 0.064 | 0.02675 | 289 |
| 184 | BIN1 3′ ss 1 | BIN1 5′ ss 5 | 2, 3, 4 | 0.21925 | 0.57875 | 0.59475 | 0.0185 | 0.042 | 0.0215 | 290 |
| 185 | BIN1 3′ ss 1 | BIN1 5′ ss 5 | 1 | 0.2435 | 0.6355 | 0.72925 | 0.01875 | 0.0305 | 0.0375 | 291 |
| 186 | BIN1 3′ ss 1 | BIN1 5′ ss 5 | 1, 4 | 0.32075 | 0.64325 | 0.579 | 0.0335 | 0.0645 | 0.02125 | 292 |
| 187 | BIN1 3′ ss 1 | BIN1 5′ ss 5 | 1, 3 | 0.34075 | 0.606 | 0.72625 | 0.0285 | 0.0365 | 0.05975 | 293 |
| 188 | BIN1 3′ ss 1 | BIN1 5′ ss 5 | 1, 3, 4 | 0.41475 | 0.632 | 0.6655 | 0.04225 | 0.14875 | 0.083 | 294 |
| 189 | BIN1 3′ ss 1 | BIN1 5′ ss 5 | 1, 2 | 0.1795 | 0.54125 | 0.54875 | 0.02225 | 0.02225 | 0.0545 | 295 |
| 190 | BIN1 3′ ss 1 | BIN1 5′ ss 5 | 1, 2, 4 | 0.2505 | 0.6065 | 0.58125 | ND | ND | ND | 296 |
| 191 | BIN1 3′ ss 1 | BIN1 5′ ss 5 | 1, 2, 3 | 0.16975 | 0.50825 | 0.506 | 0.013 | 0.04375 | 0.22075 | 297 |
| 192 | BIN1 3′ ss 1 | BIN1 5′ ss 5 | 1, 2, 3, 4 | 0.3175 | 0.54425 | 0.68025 | ND | ND | ND | 298 |
| 193 | BIN1 3′ ss 2 | Compensated | None | 0.2365 | 0.497 | 0.4785 | 0.0225 | 0.0335 | 0.031 | 299 |
| 194 | BIN1 3′ ss 2 | Compensated | 4 | 0.2795 | 0.59775 | 0.5505 | 0.0255 | 0.024 | 0.0045 | 300 |
| 195 | BIN1 3′ ss 2 | Compensated | 3 | 0.27975 | 0.64275 | 0.637 | 0.0255 | 0.06575 | 0.0925 | 301 |
| 196 | BIN1 3′ ss 2 | Compensated | 3, 4 | 0.229 | 0.483 | 0.39025 | 0.03325 | 0.1235 | 0.03775 | 302 |
| 197 | BIN1 3′ ss 2 | Compensated | 2 | 0.1945 | 0.56775 | 0.50725 | 0.02575 | 0.13 | 0.021 | 303 |
| 198 | BIN1 3′ ss 2 | Compensated | 2, 4 | 0.21975 | 0.45775 | 0.65025 | 0.0225 | 0.05925 | 0.1095 | 304 |
| 199 | BIN1 3′ ss 2 | Compensated | 2, 3 | 0.17475 | 0.56575 | 0.61625 | 0.018 | 0.03325 | 0.055 | 305 |
| 200 | BIN1 3′ ss 2 | Compensated | 2, 3, 4 | 0.255 | 0.667 | 0.48075 | 0.02825 | 0.0295 | 0.0615 | 306 |
| 201 | BIN1 3′ ss 2 | Compensated | 1 | 0.26525 | 0.56725 | 0.646 | 0.024 | 0.04725 | 0.0355 | 307 |
| 202 | BIN1 3′ ss 2 | Compensated | 1, 4 | 0.306 | 0.64925 | 0.55225 | 0.03025 | 0.033 | 0.0825 | 308 |
| 203 | BIN1 3′ ss 2 | Compensated | 1, 3 | 0.2785 | 0.604 | 0.6515 | 0.0235 | 0.17025 | 0.027 | 309 |
| 204 | BIN1 3′ ss 2 | Compensated | 1, 3, 4 | 0.23375 | 0.53225 | 0.48875 | 0.036 | 0.06475 | 0.14025 | 310 |
| 205 | BIN1 3′ ss 2 | Compensated | 1, 2 | 0.169 | 0.48625 | 0.46575 | 0.0175 | 0.02375 | 0.0445 | 311 |
| 206 | BIN1 3′ ss 2 | Compensated | 1, 2, 4 | 0.2625 | 0.60375 | 0.46825 | 0.03325 | 0.04725 | 0.06975 | 312 |
| 207 | BIN1 3′ ss 2 | Compensated | 1, 2, 3 | 0.22825 | 0.60325 | 0.6235 | 0.0235 | 0.10325 | 0.0825 | 313 |
| 208 | BIN1 3′ ss 2 | Compensated | 1, 2, 3, 4 | 0.2865 | 0.6965 | 0.6075 | 0.033 | 0.0275 | 0.08625 | 314 |
| 209 | BIN1 3′ ss 2 | BIN1 5′ ss 1 | None | 0.2145 | 0.566 | 0.538 | 0.04675 | 0.13825 | 0.11875 | 315 |
| 210 | BIN1 3′ ss 2 | BIN1 5′ ss 1 | 4 | 0.41775 | 0.708 | 0.706 | 0.05775 | 0.1225 | 0.10225 | 316 |
| 211 | BIN1 3′ ss 2 | BIN1 5′ ss 1 | 3 | 0.369 | 0.64275 | 0.636 | 0.08875 | 0.19025 | 0.15425 | 317 |
| 212 | BIN1 3′ ss 2 | BIN1 5′ ss 1 | 3, 4 | 0.5575 | 0.73625 | 0.78425 | 0.085 | 0.2525 | 0.1065 | 318 |
| 213 | BIN1 3′ ss 2 | BIN1 5′ ss 1 | 2 | 0.32225 | 0.77225 | 0.638 | 0.0435 | 0.12275 | 0.082 | 319 |
| 214 | BIN1 3′ ss 2 | BIN1 5′ ss 1 | 2, 4 | 0.401 | 0.726 | 0.7815 | 0.05825 | 0.16225 | 0.10975 | 320 |
| 215 | BIN1 3′ ss 2 | BIN1 5′ ss 1 | 2, 3 | 0.3575 | 0.77175 | 0.72475 | 0.057 | 0.08875 | 0.21175 | 321 |
| 216 | BIN1 3′ ss 2 | BIN1 5′ ss 1 | 2, 3, 4 | 0.423 | 0.6915 | 0.77475 | 0.0845 | 0.212 | 0.0715 | 322 |
| 217 | BIN1 3′ ss 2 | BIN1 5′ ss 1 | 1 | 0.417 | 0.7905 | 0.74175 | 0.07825 | 0.58675 | 0.2045 | 323 |
| 218 | BIN1 3′ ss 2 | BIN1 5′ ss 1 | 1, 4 | 0.50425 | 0.7745 | 0.8395 | 0.106 | 0.25525 | 0.3215 | 324 |
| 219 | BIN1 3′ ss 2 | BIN1 5′ ss 1 | 1, 3 | 0.557 | 0.8265 | 0.7505 | 0.1035 | 0.23 | 0.194 | 325 |
| 220 | BIN1 3′ ss 2 | BIN1 5′ ss 1 | 1, 3, 4 | 0.52425 | 0.789 | 0.73625 | 0.1325 | 0.5445 | 0.126 | 326 |
| 221 | BIN1 3′ ss 2 | BIN1 5′ ss 1 | 1, 2 | 0.40825 | 0.80125 | 0.738 | 0.0615 | 0.2335 | 0.106 | 327 |
| 222 | BIN1 3′ ss 2 | BIN1 5′ ss 1 | 1, 2, 4 | 0.5445 | 0.832 | 0.82375 | 0.08875 | 0.07975 | 0.513 | 328 |
| 223 | BIN1 3′ ss 2 | BIN1 5′ ss 1 | 1, 2, 3 | 0.4365 | 0.89225 | 0.871 | 0.097 | 0.3415 | 0.25675 | 329 |
| 224 | BIN1 3′ ss 2 | BIN1 5′ ss 1 | 1, 2, 3, 4 | 0.551 | 0.7825 | 0.811 | 0.056 | 0.09475 | 0.13625 | 330 |
| 225 | BIN1 3′ ss 2 | BIN1 5′ ss 2 | None | 0.20025 | 0.55475 | 0.622 | 0.019 | 0.052 | 0.120333333 | 331 |
| 226 | BIN1 3′ ss 2 | BIN1 5′ ss 2 | 4 | 0.20225 | 0.54075 | 0.58 | 0.02825 | 0.05875 | 0.07625 | 332 |
| 227 | BIN1 3′ ss 2 | BIN1 5′ ss 2 | 3 | 0.1715 | 0.53675 | 0.56275 | 0.01625 | 0.0755 | 0.17625 | 333 |
| 228 | BIN1 3′ ss 2 | BIN1 5′ ss 2 | 3, 4 | 0.241 | 0.5605 | 0.68875 | 0.03025 | 0.0475 | 0.08025 | 334 |
| 229 | BIN1 3′ ss 2 | BIN1 5′ ss 2 | 2 | 0.14475 | 0.5525 | 0.59 | 0.019 | 0.10525 | 0.06875 | 335 |
| 230 | BIN1 3′ ss 2 | BIN1 5′ ss 2 | 2, 4 | 0.15875 | 0.5465 | 0.43225 | 0.01875 | 0.02275 | 0.0485 | 336 |
| 231 | BIN1 3′ ss 2 | BIN1 5′ ss 2 | 2, 3 | 0.11275 | 0.49575 | 0.51025 | 0.016 | 0.0605 | 0.102 | 337 |
| 232 | BIN1 3′ ss 2 | BIN1 5′ ss 2 | 2, 3, 4 | 0.20075 | 0.60725 | 0.5005 | 0.021 | 0.08925 | 0.04175 | 338 |
| 233 | BIN1 3′ ss 2 | BIN1 5′ ss 2 | 1 | 0.19975 | 0.58275 | 0.49225 | 0.0195 | 0.0615 | 0.03625 | 339 |
| 234 | BIN1 3′ ss 2 | BIN1 5′ ss 2 | 1, 4 | 0.22725 | 0.61225 | 0.5195 | 0.0285 | 0.0535 | 0.09975 | 340 |
| 235 | BIN1 3′ ss 2 | BIN1 5′ ss 2 | 1, 3 | 0.2385 | 0.591 | 0.7225 | 0.02525 | 0.10025 | 0.077 | 341 |
| 236 | BIN1 3′ ss 2 | BIN1 5′ ss 2 | 1, 3, 4 | 0.22225 | 0.5415 | 0.60325 | 0.04 | 0.078 | 0.1725 | 342 |
| 237 | BIN1 3′ ss 2 | BIN1 5′ ss 2 | 1, 2 | 0.16775 | 0.564 | 0.64925 | 0.0205 | 0.0845 | 0.06 | 343 |
| 238 | BIN1 3′ ss 2 | BIN1 5′ ss 2 | 1, 2, 4 | 0.1775 | 0.512 | 0.513 | 0.033 | 0.06 | 0.01725 | 344 |
| 239 | BIN1 3′ ss 2 | BIN1 5′ ss 2 | 1, 2, 3 | 0.1985 | 0.632 | 0.7475 | 0.018 | 0.039 | 0.0435 | 345 |
| 240 | BIN1 3′ ss 2 | BIN1 5′ ss 2 | 1, 2, 3, 4 | ND | ND | ND | 0.03675 | 0.114 | 0.11925 | 346 |
| 241 | BIN1 3′ ss 2 | BIN1 5′ ss 3 | None | 0.225 | 0.5215 | 0.5345 | 0.01825 | 0.0205 | 0.029 | 347 |
| 242 | BIN1 3′ ss 2 | BIN1 5′ ss 3 | 4 | 0.2435 | 0.53125 | 0.5045 | 0.02825 | 0.037 | 0.027 | 348 |
| 243 | BIN1 3′ ss 2 | BIN1 5′ ss 3 | 3 | 0.2445 | 0.594 | 0.542 | 0.02375 | 0.0625 | 0.0555 | 349 |
| 244 | BIN1 3′ ss 2 | BIN1 5′ ss 3 | 3, 4 | 0.23025 | 0.53875 | 0.482 | 0.029 | 0.0375 | 0.09475 | 350 |
| 245 | BIN1 3′ ss 2 | BIN1 5′ ss 3 | 2 | 0.188 | 0.5165 | 0.57325 | 0.0175 | 0.063 | 0.05775 | 351 |
| 246 | BIN1 3′ ss 2 | BIN1 5′ ss 3 | 2, 4 | 0.219 | 0.53875 | 0.50825 | 0.026 | 0.04575 | 0.065 | 352 |
| 247 | BIN1 3′ ss 2 | BIN1 5′ ss 3 | 2, 3 | 0.17575 | 0.528 | 0.55075 | 0.01725 | 0.03225 | 0.01875 | 353 |
| 248 | BIN1 3′ ss 2 | BIN1 5′ ss 3 | 2, 3, 4 | 0.211 | 0.45725 | 0.49375 | 0.02625 | 0.03975 | 0.036 | 354 |
| 249 | BIN1 3′ ss 2 | BIN1 5′ ss 3 | 1 | 0.2775 | 0.582 | 0.62475 | 0.026 | 0.035333333 | 0.023 | 355 |
| 250 | BIN1 3′ ss 2 | BIN1 5′ ss 3 | 1, 4 | 0.2455 | 0.53875 | 0.63875 | 0.03425 | 0.082 | 0.08875 | 356 |
| 251 | BIN1 3′ ss 2 | BIN1 5′ ss 3 | 1, 3 | 0.228 | 0.51625 | 0.5525 | 0.031 | 0.0225 | 0.02475 | 357 |
| 252 | BIN1 3′ ss 2 | BIN1 5′ ss 3 | 1, 3, 4 | 0.2385 | 0.51175 | 0.5585 | 0.03475 | 0.06425 | 0.05175 | 358 |
| 253 | BIN1 3′ ss 2 | BIN1 5′ ss 3 | 1, 2 | 0.1935 | 0.60975 | 0.58375 | 0.015 | 0.07375 | 0.01925 | 359 |
| 254 | BIN1 3′ ss 2 | BIN1 5′ ss 3 | 1, 2, 4 | 0.18725 | 0.405 | 0.5965 | 0.03175 | 0.1155 | 0.09775 | 360 |
| 255 | BIN1 3′ ss 2 | BIN1 5′ ss 3 | 1, 2, 3 | 0.23525 | 0.617 | 0.602 | 0.024 | 0.048 | 0.02075 | 361 |
| 256 | BIN1 3′ ss 2 | BIN1 5′ ss 3 | 1, 2, 3, 4 | 0.21225 | 0.52375 | 0.559 | 0.02 | 0.0195 | 0.0105 | 362 |
| 257 | BIN1 3′ ss 2 | BIN1 5′ ss 4 | None | 0.172 | 0.45325 | 0.50475 | 0.022 | 0.008 | 0.01 | 363 |
| 258 | BIN1 3′ ss 2 | BIN1 5′ ss 4 | 4 | 0.20375 | 0.6005 | 0.54375 | 0.02625 | 0.05525 | 0.074 | 364 |
| 259 | BIN1 3′ ss 2 | BIN1 5′ ss 4 | 3 | 0.17575 | 0.55775 | 0.51475 | 0.00775 | 0.061 | 0.01025 | 365 |
| 260 | BIN1 3′ ss 2 | BIN1 5′ ss 4 | 3, 4 | 0.19225 | 0.486 | 0.438 | 0.025 | 0.0345 | 0.03675 | 366 |
| 261 | BIN1 3′ ss 2 | BIN1 5′ ss 4 | 2 | 0.1805 | 0.4355 | 0.37025 | 0.0145 | 0.04525 | 0.0795 | 367 |
| 262 | BIN1 3′ ss 2 | BIN1 5′ ss 4 | 2, 4 | 0.15325 | 0.426 | 0.38925 | 0.01775 | 0.045 | 0.054 | 368 |
| 263 | BIN1 3′ ss 2 | BIN1 5′ ss 4 | 2, 3 | 0.1 | 0.335 | 0.3005 | 0.013 | 0.0165 | 0.003 | 369 |
| 264 | BIN1 3′ ss 2 | BIN1 5′ ss 4 | 2, 3, 4 | 0.1695 | 0.44825 | 0.37975 | 0.0215 | 0.0305 | 0.03925 | 370 |
| 265 | BIN1 3′ ss 2 | BIN1 5′ ss 4 | 1 | 0.201 | 0.539 | 0.52425 | 0.0185 | 0.0595 | 0.0445 | 371 |
| 266 | BIN1 3′ ss 2 | BIN1 5′ ss 4 | 1, 4 | 0.22525 | 0.50575 | 0.53425 | 0.0235 | 0.021 | 0.054 | 372 |
| 267 | BIN1 3′ ss 2 | BIN1 5′ ss 4 | 1, 3 | 0.20625 | 0.549 | 0.551 | 0.036 | 0.013 | 0.0415 | 373 |
| 268 | BIN1 3′ ss 2 | BIN1 5′ ss 4 | 1, 3, 4 | 0.22775 | 0.50025 | 0.504 | 0.02625 | 0.0555 | 0.1045 | 374 |
| 269 | BIN1 3′ ss 2 | BIN1 5′ ss 4 | 1, 2 | 0.136 | 0.37175 | 0.459 | 0.02 | 0.05225 | 0.07925 | 375 |
| 270 | BIN1 3′ ss 2 | BIN1 5′ ss 4 | 1, 2, 4 | 0.15275 | 0.404 | 0.38225 | 0.02 | 0.03625 | 0.01725 | 376 |
| 271 | BIN1 3′ ss 2 | BIN1 5′ ss 4 | 1, 2, 3 | 0.14975 | 0.51275 | 0.4445 | 0.01025 | 0.017 | 0.04925 | 377 |
| 272 | BIN1 3′ ss 2 | BIN1 5′ ss 4 | 1, 2, 3, 4 | 0.15675 | 0.423 | 0.44825 | 0.02675 | 0.0365 | 0.034 | 378 |
| 273 | BIN1 3′ ss 2 | BIN1 5′ ss 5 | None | 0.18325 | 0.36175 | 0.464 | 0.026 | 0.0725 | 0.038 | 379 |
| 274 | BIN1 3′ ss 2 | BIN1 5′ ss 5 | 4 | 0.19075 | 0.428 | 0.45725 | 0.01375 | 0.0245 | 0.0135 | 380 |
| 275 | BIN1 3′ ss 2 | BIN1 5′ ss 5 | 3 | 0.14975 | 0.39725 | 0.31525 | ND | ND | ND | 381 |
| 276 | BIN1 3′ ss 2 | BIN1 5′ ss 5 | 3, 4 | 0.15525 | 0.36325 | 0.4015 | 0.018 | 0.05475 | 0.07075 | 382 |
| 277 | BIN1 3′ ss 2 | BIN1 5′ ss 5 | 2 | 0.1375 | 0.35075 | 0.3915 | 0.01775 | 0.03275 | 0.05875 | 383 |
| 278 | BIN1 3′ ss 2 | BIN1 5′ ss 5 | 2, 4 | 0.16275 | 0.376 | 0.47075 | 0.01375 | 0.049 | 0.00075 | 384 |
| 279 | BIN1 3′ ss 2 | BIN1 5′ ss 5 | 2, 3 | 0.1135 | 0.2905 | 0.33525 | 0.01325 | 0.0175 | 0.0065 | 385 |
| 280 | BIN1 3′ ss 2 | BIN1 5′ ss 5 | 2, 3, 4 | 0.1415 | 0.35475 | 0.39325 | 0.0135 | 0.03425 | 0.10425 | 386 |
| 281 | BIN1 3′ ss 2 | BIN1 5′ ss 5 | 1 | 0.15675 | 0.41025 | 0.4085 | 0.0215 | 0.02 | 0.00975 | 387 |
| 282 | BIN1 3′ ss 2 | BIN1 5′ ss 5 | 1, 4 | 0.17875 | 0.43725 | 0.545 | 0.0195 | 0.03975 | 0.12475 | 388 |
| 283 | BIN1 3′ ss 2 | BIN1 5′ ss 5 | 1, 3 | 0.17925 | 0.44325 | 0.41725 | 0.01225 | 0.0125 | 0.0155 | 389 |
| 284 | BIN1 3′ ss 2 | BIN1 5′ ss 5 | 1, 3, 4 | 0.17675 | 0.4345 | 0.318 | 0.01425 | 0.0195 | 0.0075 | 390 |
| 285 | BIN1 3′ ss 2 | BIN1 5′ ss 5 | 1, 2 | 0.10875 | 0.325 | 0.31975 | 0.01 | 0.0105 | 0.0195 | 391 |
| 286 | BIN1 3′ ss 2 | BIN1 5′ ss 5 | 1, 2, 4 | 0.11675 | 0.28175 | 0.34325 | 0.0135 | 0.0615 | 0.00825 | 392 |
| 287 | BIN1 3′ ss 2 | BIN1 5′ ss 5 | 1, 2, 3 | 0.08975 | 0.331 | 0.368 | 0.01 | 0.01825 | 0.013 | 393 |
| 288 | BIN1 3′ ss 2 | BIN1 5′ ss 5 | 1, 2, 3, 4 | 0.11175 | 0.36725 | 0.32225 | 0.01475 | 0.0205 | 0.0105 | 394 |
| 289 | BIN1 3′ ss 3 | Compensated | None | ND | ND | ND | 0.02475 | 0.095666667 | 0 | 395 |
| 290 | BIN1 3′ ss 3 | Compensated | 4 | 0.336 | 0.6205 | 0.662 | 0.0485 | 0.09975 | 0.1135 | 396 |
| 291 | BIN1 3′ ss 3 | Compensated | 3 | 0.259 | 0.60775 | 0.54375 | 0.0505 | 0.062 | 0.04525 | 397 |
| 292 | BIN1 3′ ss 3 | Compensated | 3, 4 | 0.4175 | 0.756 | 0.731 | 0.05825 | 0.084 | 0.1285 | 398 |
| 293 | BIN1 3′ ss 3 | Compensated | 2 | 0.258 | 0.67725 | 0.64875 | 0.037 | 0.07075 | 0.056 | 399 |
| 294 | BIN1 3′ ss 3 | Compensated | 2, 4 | 0.3415 | 0.56125 | 0.714 | 0.05225 | 0.09225 | 0.048 | 400 |
| 295 | BIN1 3′ ss 3 | Compensated | 2, 3 | 0.26675 | 0.747 | 0.714 | 0.0205 | 0.09525 | 0.145 | 401 |
| 296 | BIN1 3′ ss 3 | Compensated | 2, 3, 4 | 0.4195 | 0.70525 | 0.73525 | 0.077 | 0.11175 | 0.09825 | 402 |
| 297 | BIN1 3′ ss 3 | Compensated | 1 | 0.323 | 0.72825 | 0.69475 | 0.06175 | 0.05375 | 0.20475 | 403 |
| 298 | BIN1 3′ ss 3 | Compensated | 1, 4 | 0.387 | 0.6905 | 0.69125 | 0.058 | 0.174 | 0.07575 | 404 |
| 299 | BIN1 3′ ss 3 | Compensated | 1, 3 | 0.4155 | 0.7055 | 0.76375 | 0.05475 | 0.12375 | 0.10225 | 405 |
| 300 | BIN1 3′ ss 3 | Compensated | 1, 3, 4 | 0.41875 | 0.7005 | 0.655 | 0.08425 | 0.099 | 0.059 | 406 |
| 301 | BIN1 3′ ss 3 | Compensated | 1, 2 | 0.29025 | 0.6815 | 0.56525 | 0.042 | 0.1705 | 0.15075 | 407 |
| 302 | BIN1 3′ ss 3 | Compensated | 1, 2, 4 | 0.276 | 0.56625 | 0.646 | 0.07775 | 0.226 | 0.158 | 408 |
| 303 | BIN1 3′ ss 3 | Compensated | 1, 2, 3 | 0.353 | 0.7475 | 0.76025 | ND | ND | ND | 409 |
| 304 | BIN1 3′ ss 3 | Compensated | 1, 2, 3, 4 | 0.3755 | 0.75325 | 0.61425 | 0.05975 | 0.3265 | 0.310333333 | 410 |
| 305 | BIN1 3′ ss 3 | BIN1 5′ ss 1 | None | 0.3765 | 0.69325 | 0.70375 | 0.11525 | 0.288333333 | 0.2175 | 411 |
| 306 | BIN1 3′ ss 3 | BIN1 5′ ss 1 | 4 | 0.481 | 0.7845 | 0.84075 | 0.10975 | 0.25875 | 0.21625 | 412 |
| 307 | BIN1 3′ ss 3 | BIN1 5′ ss 1 | 3 | 0.514 | 0.824 | 0.8435 | 0.101 | 0.332 | 0.166 | 413 |
| 308 | BIN1 3′ ss 3 | BIN1 5′ ss 1 | 3, 4 | 0.59425 | 0.84325 | 0.7985 | 0.12625 | 0.3295 | 0.26375 | 414 |
| 309 | BIN1 3′ ss 3 | BIN1 5′ ss 1 | 2 | 0.434 | 0.72825 | 0.739 | 0.06525 | 0.13975 | 0.0365 | 415 |
| 310 | BIN1 3′ ss 3 | BIN1 5′ ss 1 | 2, 4 | 0.5655 | 0.79175 | 0.79325 | 0.111 | 0.18475 | 0.3355 | 416 |
| 311 | BIN1 3′ ss 3 | BIN1 5′ ss 1 | 2, 3 | 0.50075 | 0.9005 | 0.7855 | 0.11525 | 0.237 | 0.077 | 417 |
| 312 | BIN1 3′ ss 3 | BIN1 5′ ss 1 | 2, 3, 4 | 0.604 | 0.867 | 0.83 | 0.1235 | 0.1865 | 0.18725 | 418 |
| 313 | BIN1 3′ ss 3 | BIN1 5′ ss 1 | 1 | 0.52875 | 0.797 | 0.7745 | 0.15225 | 0.267 | 0.17 | 419 |
| 314 | BIN1 3′ ss 3 | BIN1 5′ ss 1 | 1, 4 | 0.59225 | 0.779 | 0.78675 | 0.13675 | 0.21425 | 0.35675 | 420 |
| 315 | BIN1 3′ ss 3 | BIN1 5′ ss 1 | 1, 3 | 0.59775 | 0.804 | 0.8085 | 0.20825 | 0.33375 | 0.404 | 421 |
| 316 | BIN1 3′ ss 3 | BIN1 5′ ss 1 | 1, 3, 4 | 0.59375 | 0.816 | 0.75675 | 0.19375 | 0.28275 | 0.29475 | 422 |
| 317 | BIN1 3′ ss 3 | BIN1 5′ ss 1 | 1, 2 | 0.5455 | 0.8125 | 0.8055 | 0.10775 | 0.19425 | 0.1765 | 423 |
| 318 | BIN1 3′ ss 3 | BIN1 5′ ss 1 | 1, 2, 4 | 0.62975 | 0.91 | 0.843 | 0.16075 | 0.25425 | 0.726333333 | 424 |
| 319 | BIN1 3′ ss 3 | BIN1 5′ ss 1 | 1, 2, 3 | 0.5955 | 0.826 | 0.80975 | 0.1185 | 0.256 | 0.1655 | 425 |
| 320 | BIN1 3′ ss 3 | BIN1 5′ ss 1 | 1, 2, 3, 4 | 0.651 | 0.87375 | 0.75725 | 0.15075 | 0.3655 | 0.12075 | 426 |
| 321 | BIN1 3′ ss 3 | BIN1 5′ ss 2 | None | 0.23275 | 0.5555 | 0.633 | 0.027 | 0.092 | 0.0945 | 427 |
| 322 | BIN1 3′ ss 3 | BIN1 5′ ss 2 | 4 | 0.29975 | 0.64825 | 0.56375 | 0.03975 | 0.1175 | 0.08525 | 428 |
| 323 | BIN1 3′ ss 3 | BIN1 5′ ss 2 | 3 | 0.254 | 0.648 | 0.6115 | 0.03575 | 0.106 | 0.11725 | 429 |
| 324 | BIN1 3′ ss 3 | BIN1 5′ ss 2 | 3, 4 | 0.30625 | 0.6575 | 0.629 | 0.04775 | 0.1235 | 0.08825 | 430 |
| 325 | BIN1 3′ ss 3 | BIN1 5′ ss 2 | 2 | 0.1895 | 0.57025 | 0.63225 | 0.02275 | 0.0825 | 0.04275 | 431 |
| 326 | BIN1 3′ ss 3 | BIN1 5′ ss 2 | 2, 4 | 0.25 | 0.618 | 0.67775 | 0.02325 | 0.055 | 0.026 | 432 |
| 327 | BIN1 3′ ss 3 | BIN1 5′ ss 2 | 2, 3 | 0.19375 | 0.6935 | 0.64425 | 0.02025 | 0.09225 | 0.15475 | 433 |
| 328 | BIN1 3′ ss 3 | BIN1 5′ ss 2 | 2, 3, 4 | 0.31175 | 0.71875 | 0.68875 | 0.04375 | 0.048 | 0.05575 | 434 |
| 329 | BIN1 3′ ss 3 | BIN1 5′ ss 2 | 1 | 0.30475 | 0.726 | 0.723 | 0.03075 | 0.0715 | 0.12725 | 435 |
| 330 | BIN1 3′ ss 3 | BIN1 5′ ss 2 | 1, 4 | 0.30525 | 0.65075 | 0.636 | 0.056 | 0.13525 | 0.063 | 436 |
| 331 | BIN1 3′ ss 3 | BIN1 5′ ss 2 | 1, 3 | 0.37925 | 0.6845 | 0.76075 | 0.0555 | 0.09425 | 0.143 | 437 |
| 332 | BIN1 3′ ss 3 | BIN1 5′ ss 2 | 1, 3, 4 | 0.3515 | 0.71 | 0.6205 | 0.0495 | 0.1435 | 0.1095 | 438 |
| 333 | BIN1 3′ ss 3 | BIN1 5′ ss 2 | 1, 2 | 0.20775 | 0.61875 | 0.65175 | 0.03 | 0.13275 | 0.163 | 439 |
| 334 | BIN1 3′ ss 3 | BIN1 5′ ss 2 | 1, 2, 4 | 0.30025 | 0.721 | 0.607 | 0.0475 | 0.20775 | 0.08 | 440 |
| 335 | BIN1 3′ ss 3 | BIN1 5′ ss 2 | 1, 2, 3 | 0.25425 | 0.7285 | 0.685 | 0.0285 | 0.05025 | 0.1175 | 441 |
| 336 | BIN1 3′ ss 3 | BIN1 5′ ss 2 | 1, 2, 3, 4 | 0.333 | 0.7165 | 0.77075 | 0.03975 | 0.1235 | 0.09475 | 442 |
| 337 | BIN1 3′ ss 3 | BIN1 5′ ss 3 | None | 0.2415 | 0.5195 | 0.45375 | 0.03225 | 0.1425 | 0.0395 | 443 |
| 338 | BIN1 3′ ss 3 | BIN1 5′ ss 3 | 4 | 0.26875 | 0.5795 | 0.58125 | 0.03725 | 0.0435 | 0.0485 | 444 |
| 339 | BIN1 3′ ss 3 | BIN1 5′ ss 3 | 3 | 0.2935 | 0.64175 | 0.60125 | 0.026 | 0.1175 | 0.159 | 445 |
| 340 | BIN1 3′ ss 3 | BIN1 5′ ss 3 | 3, 4 | 0.33175 | 0.501 | 0.64475 | 0.0535 | 0.08325 | 0.1 | 446 |
| 341 | BIN1 3′ ss 3 | BIN1 5′ ss 3 | 2 | 0.27775 | 0.6295 | 0.6805 | 0.0255 | 0.0845 | 0.093 | 447 |
| 342 | BIN1 3′ ss 3 | BIN1 5′ ss 3 | 2, 4 | 0.286 | 0.61275 | 0.55025 | 0.035 | 0.174 | 0.14275 | 448 |
| 343 | BIN1 3′ ss 3 | BIN1 5′ ss 3 | 2, 3 | 0.24775 | 0.61325 | 0.5535 | 0.033 | 0.27925 | 0.01525 | 449 |
| 344 | BIN1 3′ ss 3 | BIN1 5′ ss 3 | 2, 3, 4 | 0.33025 | 0.638 | 0.665 | 0.057 | 0.09925 | 0.0255 | 450 |
| 345 | BIN1 3′ ss 3 | BIN1 5′ ss 3 | 1 | 0.30325 | 0.673 | 0.63925 | 0.041 | 0.10525 | 0.108 | 451 |
| 346 | BIN1 3′ ss 3 | BIN1 5′ ss 3 | 1, 4 | 0.344 | 0.66225 | 0.6455 | 0.049 | 0.14575 | 0.048 | 452 |
| 347 | BIN1 3′ ss 3 | BIN1 5′ ss 3 | 1, 3 | 0.3855 | 0.681 | 0.68825 | 0.0565 | 0.081 | 0.09825 | 453 |
| 348 | BIN1 3′ ss 3 | BIN1 5′ ss 3 | 1, 3, 4 | 0.3625 | 0.58425 | 0.62375 | 0.08425 | 0.0785 | 0.1025 | 454 |
| 349 | BIN1 3′ ss 3 | BIN1 5′ ss 3 | 1, 2 | 0.25875 | 0.6055 | 0.6635 | 0.02625 | 0.1155 | 0.0745 | 455 |
| 350 | BIN1 3′ ss 3 | BIN1 5′ ss 3 | 1, 2, 4 | 0.3655 | 0.731 | 0.7205 | 0.058 | 0.156 | 0.14475 | 456 |
| 351 | BIN1 3′ ss 3 | BIN1 5′ ss 3 | 1, 2, 3 | 0.30325 | 0.65825 | 0.65025 | 0.036 | 0.18425 | 0.15825 | 457 |
| 352 | BIN1 3′ ss 3 | BIN1 5′ ss 3 | 1, 2, 3, 4 | 0.366 | 0.6145 | 0.68 | ND | ND | ND | 458 |
| 353 | BIN1 3′ ss 3 | BIN1 5′ ss 4 | None | 0.2335 | 0.56875 | 0.5565 | 0.02075 | 0.0315 | 0.06375 | 459 |
| 354 | BIN1 3′ ss 3 | BIN1 5′ ss 4 | 4 | 0.25 | 0.531 | 0.515 | 0.03875 | 0.0625 | 0.022 | 460 |
| 355 | BIN1 3′ ss 3 | BIN1 5′ ss 4 | 3 | 0.17875 | 0.55625 | 0.4085 | 0.01975 | 0.0545 | 0.0865 | 461 |
| 356 | BIN1 3′ ss 3 | BIN1 5′ ss 4 | 3, 4 | 0.2845 | 0.56675 | 0.588 | 0.03775 | 0.03825 | 0.14 | 462 |
| 357 | BIN1 3′ ss 3 | BIN1 5′ ss 4 | 2 | 0.15875 | 0.499 | 0.54375 | 0.02225 | 0.07175 | 0.033 | 463 |
| 358 | BIN1 3′ ss 3 | BIN1 5′ ss 4 | 2, 4 | 0.1535 | 0.441 | 0.424 | 0.02825 | 0.05225 | 0.01625 | 464 |
| 359 | BIN1 3′ ss 3 | BIN1 5′ ss 4 | 2, 3 | 0.2575 | 0.64225 | 0.47325 | 0.013 | 0.082 | 0.12475 | 465 |
| 360 | BIN1 3′ ss 3 | BIN1 5′ ss 4 | 2, 3, 4 | 0.214 | 0.495 | 0.52425 | 0.01725 | 0.046 | 0.04025 | 466 |
| 361 | BIN1 3′ ss 3 | BIN1 5′ ss 4 | 1 | 0.23625 | 0.524 | 0.58675 | 0.021 | 0.05625 | 0.04225 | 467 |
| 362 | BIN1 3′ ss 3 | BIN1 5′ ss 4 | 1, 4 | 0.304 | 0.61675 | 0.551 | 0.04525 | 0.12625 | 0.10275 | 468 |
| 363 | BIN1 3′ ss 3 | BIN1 5′ ss 4 | 1, 3 | 0.269 | 0.597 | 0.495 | 0.0305 | 0.06375 | 0.106 | 469 |
| 364 | BIN1 3′ ss 3 | BIN1 5′ ss 4 | 1, 3, 4 | 0.29 | 0.55725 | 0.56925 | 0.043 | 0.039 | 0.11825 | 470 |
| 365 | BIN1 3′ ss 3 | BIN1 5′ ss 4 | 1, 2 | 0.1965 | 0.57 | 0.603 | 0.0155 | 0.01275 | 0.019 | 471 |
| 366 | BIN1 3′ ss 3 | BIN1 5′ ss 4 | 1, 2, 4 | 0.2615 | 0.616 | 0.5595 | ND | ND | ND | 472 |
| 367 | BIN1 3′ ss 3 | BIN1 5′ ss 4 | 1, 2, 3 | 0.1825 | 0.58475 | 0.57625 | 0.02275 | 0.08925 | 0.16075 | 473 |
| 368 | BIN1 3′ ss 3 | BIN1 5′ ss 4 | 1, 2, 3, 4 | 0.26525 | 0.5455 | 0.62525 | 0.01975 | 0.261 | 0.018 | 474 |
| 369 | BIN1 3′ ss 3 | BIN1 5′ ss 5 | None | 0.1985 | 0.4355 | 0.48025 | 0.025 | 0.0425 | 0.052 | 475 |
| 370 | BIN1 3′ ss 3 | BIN1 5′ ss 5 | 4 | 0.168 | 0.42 | 0.325 | 0.025 | 0.04175 | 0.01325 | 476 |
| 371 | BIN1 3′ ss 3 | BIN1 5′ ss 5 | 3 | 0.12925 | 0.436 | 0.41975 | 0.02125 | 0.04 | 0.0925 | 477 |
| 372 | BIN1 3′ ss 3 | BIN1 5′ ss 5 | 3, 4 | 0.237 | 0.49375 | 0.49 | 0.0205 | 0.049 | 0.0145 | 478 |
| 373 | BIN1 3′ ss 3 | BIN1 5′ ss 5 | 2 | 0.15825 | 0.41025 | 0.47575 | 0.0145 | 0.02025 | 0.01325 | 479 |
| 374 | BIN1 3′ ss 3 | BIN1 5′ ss 5 | 2, 4 | 0.14775 | 0.36575 | 0.47625 | 0.0225 | 0.12125 | 0.0215 | 480 |
| 375 | BIN1 3′ ss 3 | BIN1 5′ ss 5 | 2, 3 | 0.17075 | 0.47775 | 0.7005 | 0.01175 | 0.02475 | 0.05375 | 481 |
| 376 | BIN1 3′ ss 3 | BIN1 5′ ss 5 | 2, 3, 4 | 0.13975 | 0.39 | 0.43225 | 0.021 | 0.0285 | 0.1405 | 482 |
| 377 | BIN1 3′ ss 3 | BIN1 5′ ss 5 | 1 | 0.21625 | 0.53 | 0.498 | 0.017 | 0.10325 | 0.03175 | 483 |
| 378 | BIN1 3′ ss 3 | BIN1 5′ ss 5 | 1, 4 | 0.2 | 0.46575 | 0.44075 | 0.018 | 0.04725 | 0.0725 | 484 |
| 379 | BIN1 3′ ss 3 | BIN1 5′ ss 5 | 1, 3 | 0.19475 | 0.481 | 0.6345 | 0.01475 | 0.01325 | 0.014 | 485 |
| 380 | BIN1 3′ ss 3 | BIN1 5′ ss 5 | 1, 3, 4 | 0.20575 | 0.30275 | 0.486 | 0.02275 | 0.077 | 0.0205 | 486 |
| 381 | BIN1 3′ ss 3 | BIN1 5′ ss 5 | 1, 2 | 0.11975 | 0.44325 | 0.44775 | 0.0105 | 0.022 | 0.01975 | 487 |
| 382 | BIN1 3′ ss 3 | BIN1 5′ ss 5 | 1, 2, 4 | 0.14975 | 0.44525 | 0.35375 | 0.02425 | 0.0235 | 0.0955 | 488 |
| 383 | BIN1 3′ ss 3 | BIN1 5′ ss 5 | 1, 2, 3 | 0.1035 | 0.41225 | 0.33075 | 0.018 | 0.11525 | 0.0255 | 489 |
| 384 | BIN1 3′ ss 3 | BIN1 5′ ss 5 | 1, 2, 3, 4 | 0.17825 | 0.46625 | 0.52225 | 0.012 | 0.02825 | 0.01275 | 490 |
| 385 | BIN1 3′ ss 4 | Compensated | None | 0.2995 | 0.602 | 0.75 | 0.02675 | 0.0465 | 0.03075 | 491 |
| 386 | BIN1 3′ ss 4 | Compensated | 4 | 0.334 | 0.55725 | 0.64825 | 0.03325 | 0.05225 | 0.0505 | 492 |
| 387 | BIN1 3′ ss 4 | Compensated | 3 | 0.31525 | 0.674 | 0.6655 | 0.0425 | 0.10775 | 0.103 | 493 |
| 388 | BIN1 3′ ss 4 | Compensated | 3, 4 | 0.33225 | 0.56475 | 0.51175 | 0.0485 | 0.09025 | 0.05225 | 494 |
| 389 | BIN1 3′ ss 4 | Compensated | 2 | 0.23675 | 0.55425 | 0.4795 | 0.02875 | 0.13425 | 0.089 | 495 |
| 390 | BIN1 3′ ss 4 | Compensated | 2, 4 | 0.34675 | 0.64875 | 0.73775 | 0.02775 | 0.00975 | 0.0125 | 496 |
| 391 | BIN1 3′ ss 4 | Compensated | 2, 3 | 0.27125 | 0.60525 | 0.63 | 0.041 | 0.0905 | 0.06575 | 497 |
| 392 | BIN1 3′ ss 4 | Compensated | 2, 3, 4 | 0.339 | 0.6005 | 0.62275 | 0.0385 | 0.10425 | 0.136 | 498 |
| 393 | BIN1 3′ ss 4 | Compensated | 1 | 0.341 | 0.67875 | 0.67175 | 0.046 | 0.1055 | 0.1085 | 499 |
| 394 | BIN1 3′ ss 4 | Compensated | 1, 4 | 0.3515 | 0.617 | 0.5075 | 0.055 | 0.09375 | 0.057 | 500 |
| 395 | BIN1 3′ ss 4 | Compensated | 1, 3 | 0.387 | 0.74425 | 0.77975 | 0.052 | 0.0675 | 0.037 | 501 |
| 396 | BIN1 3′ ss 4 | Compensated | 1, 3, 4 | 0.37725 | 0.66175 | 0.59025 | 0.06325 | 0.0755 | 0.0365 | 502 |
| 397 | BIN1 3′ ss 4 | Compensated | 1, 2 | 0.31675 | 0.65125 | 0.7605 | ND | ND | ND | 503 |
| 398 | BIN1 3′ ss 4 | Compensated | 1, 2, 4 | 0.36525 | 0.63425 | 0.84325 | ND | ND | ND | 504 |
| 399 | BIN1 3′ ss 4 | Compensated | 1, 2, 3 | ND | ND | ND | 0.03925 | 0.1145 | 0.15475 | 505 |
| 400 | BIN1 3′ ss 4 | Compensated | 1, 2, 3, 4 | 0.41 | 0.649 | 0.55425 | 0.04675 | 0.11425 | 0.06275 | 506 |
| 401 | BIN1 3′ ss 4 | BIN1 5′ ss 1 | None | 0.385 | 0.67325 | 0.623 | 0.08875 | 0.1505 | 0.207 | 507 |
| 402 | BIN1 3′ ss 4 | BIN1 5′ ss 1 | 4 | 0.527 | 0.84075 | 0.8905 | 0.06975 | 0.19 | 0.04075 | 508 |
| 403 | BIN1 3′ ss 4 | BIN1 5′ ss 1 | 3 | 0.561 | 0.83 | 0.707 | 0.1165 | 0.27325 | 0.354 | 509 |
| 404 | BIN1 3′ ss 4 | BIN1 5′ ss 1 | 3, 4 | 0.6205 | 0.80525 | 0.812 | 0.181 | 0.2015 | 0.31225 | 510 |
| 405 | BIN1 3′ ss 4 | BIN1 5′ ss 1 | 2 | 0.44625 | 0.76675 | 0.83875 | 0.10175 | 0.232 | 0.13875 | 511 |
| 406 | BIN1 3′ ss 4 | BIN1 5′ ss 1 | 2, 4 | 0.528 | 0.77575 | 0.7865 | 0.1055 | 0.36525 | 0.61825 | 512 |
| 407 | BIN1 3′ ss 4 | BIN1 5′ ss 1 | 2, 3 | 0.49575 | 0.82675 | 0.721 | 0.08575 | 0.17175 | 0.29325 | 513 |
| 408 | BIN1 3′ ss 4 | BIN1 5′ ss 1 | 2, 3, 4 | 0.6105 | 0.81725 | 0.79825 | 0.1645 | 0.30125 | 0.25175 | 514 |
| 409 | BIN1 3′ ss 4 | BIN1 5′ ss 1 | 1 | 0.60325 | 0.81325 | 0.82 | 0.111 | 0.17175 | 0.3355 | 515 |
| 410 | BIN1 3′ ss 4 | BIN1 5′ ss 1 | 1, 4 | 0.54525 | 0.73175 | 0.78425 | 0.13675 | 0.12975 | 0.143 | 516 |
| 411 | BIN1 3′ ss 4 | BIN1 5′ ss 1 | 1, 3 | 0.6535 | 0.8345 | 0.87375 | 0.14975 | 0.26625 | 0.594 | 517 |
| 412 | BIN1 3′ ss 4 | BIN1 5′ ss 1 | 1, 3, 4 | 0.65825 | 0.804 | 0.7675 | 0.145 | 0.22425 | 0.31175 | 518 |
| 413 | BIN1 3′ ss 4 | BIN1 5′ ss 1 | 1, 2 | 0.5815 | 0.888 | 0.85525 | 0.12 | 0.15 | 0.1715 | 519 |
| 414 | BIN1 3′ ss 4 | BIN1 5′ ss 1 | 1, 2, 4 | 0.5465 | 0.73225 | 0.8115 | 0.184 | 0.247 | 0.43375 | 520 |
| 415 | BIN1 3′ ss 4 | BIN1 5′ ss 1 | 1, 2, 3 | 0.63875 | 0.82275 | 0.852 | 0.10925 | 0.238 | 0.37575 | 521 |
| 416 | BIN1 3′ ss 4 | BIN1 5′ ss 1 | 1, 2, 3, 4 | 0.67825 | 0.897 | 0.87675 | 0.135 | 0.244 | 0.019 | 522 |
| 417 | BIN1 3′ ss 4 | BIN1 5′ ss 2 | None | 0.208 | 0.6065 | 0.62525 | 0.037 | 0.07925 | 0.17525 | 523 |
| 418 | BIN1 3′ ss 4 | BIN1 5′ ss 2 | 4 | 0.25675 | 0.54025 | 0.6125 | 0.02825 | 0.06875 | 0.0855 | 524 |
| 419 | BIN1 3′ ss 4 | BIN1 5′ ss 2 | 3 | 0.228 | 0.56475 | 0.5875 | 0.0315 | 0.0875 | 0.06325 | 525 |
| 420 | BIN1 3′ ss 4 | BIN1 5′ ss 2 | 3, 4 | 0.31275 | 0.632 | 0.7275 | 0.04975 | 0.1005 | 0.19025 | 526 |
| 421 | BIN1 3′ ss 4 | BIN1 5′ ss 2 | 2 | 0.1845 | 0.534 | 0.57575 | 0.0225 | 0.07475 | 0.06125 | 527 |
| 422 | BIN1 3′ ss 4 | BIN1 5′ ss 2 | 2, 4 | 0.23 | 0.58475 | 0.655 | 0.02775 | 0.20075 | 0.093 | 528 |
| 423 | BIN1 3′ ss 4 | BIN1 5′ ss 2 | 2, 3 | 0.1515 | 0.558 | 0.6385 | 0.01375 | 0.0885 | 0.0395 | 529 |
| 424 | BIN1 3′ ss 4 | BIN1 5′ ss 2 | 2, 3, 4 | 0.2645 | 0.658 | 0.6285 | 0.03825 | 0.1585 | 0.0485 | 530 |
| 425 | BIN1 3′ ss 4 | BIN1 5′ ss 2 | 1 | 0.253 | 0.6305 | 0.5935 | 0.0565 | 0.093 | 0.06475 | 531 |
| 426 | BIN1 3′ ss 4 | BIN1 5′ ss 2 | 1, 4 | 0.329 | 0.6635 | 0.6905 | 0.0405 | 0.10375 | 0.13025 | 532 |
| 427 | BIN1 3′ ss 4 | BIN1 5′ ss 2 | 1, 3 | 0.3155 | 0.60225 | 0.64575 | 0.04725 | 0.0765 | 0.12625 | 533 |
| 428 | BIN1 3′ ss 4 | BIN1 5′ ss 2 | 1, 3, 4 | 0.297 | 0.6175 | 0.55225 | 0.04275 | 0.079 | 0.1195 | 534 |
| 429 | BIN1 3′ ss 4 | BIN1 5′ ss 2 | 1, 2 | 0.2185 | 0.579 | 0.6555 | 0.0235 | 0.081 | 0.111 | 535 |
| 430 | BIN1 3′ ss 4 | BIN1 5′ ss 2 | 1, 2, 4 | 0.21325 | 0.5495 | 0.43375 | 0.0375 | 0.05475 | 0.04325 | 536 |
| 431 | BIN1 3′ ss 4 | BIN1 5′ ss 2 | 1, 2, 3 | 0.27175 | 0.74775 | 0.702 | 0.0295 | 0.05575 | 0.0565 | 537 |
| 432 | BIN1 3′ ss 4 | BIN1 5′ ss 2 | 1, 2, 3, 4 | 0.36075 | 0.7215 | 0.77375 | ND | ND | ND | 538 |
| 433 | BIN1 3′ ss 4 | BIN1 5′ ss 3 | None | 0.25925 | 0.54575 | 0.73275 | ND | ND | ND | 539 |
| 434 | BIN1 3′ ss 4 | BIN1 5′ ss 3 | 4 | 0.25475 | 0.42375 | 0.449 | 0.0505 | 0.11325 | 0.0955 | 540 |
| 435 | BIN1 3′ ss 4 | BIN1 5′ ss 3 | 3 | 0.24675 | 0.6015 | 0.669 | 0.01475 | 0.013 | 0.01525 | 541 |
| 436 | BIN1 3′ ss 4 | BIN1 5′ ss 3 | 3, 4 | 0.3265 | 0.59175 | 0.559 | 0.04775 | 0.1115 | 0.039 | 542 |
| 437 | BIN1 3′ ss 4 | BIN1 5′ ss 3 | 2 | 0.1995 | 0.56675 | 0.6315 | 0.03825 | 0.09775 | 0.134 | 543 |
| 438 | BIN1 3′ ss 4 | BIN1 5′ ss 3 | 2, 4 | 0.2975 | 0.528 | 0.59575 | 0.0245 | 0.141 | 0.08325 | 544 |
| 439 | BIN1 3′ ss 4 | BIN1 5′ ss 3 | 2, 3 | 0.24 | 0.67875 | 0.636 | 0.02 | 0.03 | 0.10425 | 545 |
| 440 | BIN1 3′ ss 4 | BIN1 5′ ss 3 | 2, 3, 4 | 0.346 | 0.58575 | 0.63875 | 0.0315 | 0.12225 | 0.12075 | 546 |
| 441 | BIN1 3′ ss 4 | BIN1 5′ ss 3 | 1 | 0.28325 | 0.5835 | 0.6165 | 0.08325 | 0.1355 | 0.08375 | 547 |
| 442 | BIN1 3′ ss 4 | BIN1 5′ ss 3 | 1, 4 | 0.27025 | 0.53825 | 0.59675 | 0.047 | 0.03775 | 0.06375 | 548 |
| 443 | BIN1 3′ ss 4 | BIN1 5′ ss 3 | 1, 3 | 0.35275 | 0.61425 | 0.65775 | 0.03925 | 0.04475 | 0.091 | 549 |
| 444 | BIN1 3′ ss 4 | BIN1 5′ ss 3 | 1, 3, 4 | 0.36725 | 0.57875 | 0.61925 | 0.05375 | 0.0945 | 0.10475 | 550 |
| 445 | BIN1 3′ ss 4 | BIN1 5′ ss 3 | 1, 2 | 0.28975 | 0.6955 | 0.5445 | 0.02625 | 0.073 | 0.0625 | 551 |
| 446 | BIN1 3′ ss 4 | BIN1 5′ ss 3 | 1, 2, 4 | 0.307 | 0.64175 | 0.51125 | 0.04525 | 0.07575 | 0.039 | 552 |
| 447 | BIN1 3′ ss 4 | BIN1 5′ ss 3 | 1, 2, 3 | 0.3065 | 0.67475 | 0.713 | 0.02375 | 0.0655 | 0.0185 | 553 |
| 448 | BIN1 3′ ss 4 | BIN1 5′ ss 3 | 1, 2, 3, 4 | 0.39725 | 0.68875 | 0.6825 | 0.05525 | 0.13725 | 0.05 | 554 |
| 449 | BIN1 3′ ss 4 | BIN1 5′ ss 4 | None | 0.21075 | 0.57975 | 0.49225 | 0.0195 | 0.033 | 0.034 | 555 |
| 450 | BIN1 3′ ss 4 | BIN1 5′ ss 4 | 4 | 0.269 | 0.54625 | 0.50375 | 0.02025 | 0.03325 | 0.05525 | 556 |
| 451 | BIN1 3′ ss 4 | BIN1 5′ ss 4 | 3 | 0.15325 | 0.4695 | 0.56325 | 0.019 | 0.0395 | 0.02275 | 557 |
| 452 | BIN1 3′ ss 4 | BIN1 5′ ss 4 | 3, 4 | 0.26975 | 0.53925 | 0.569 | 0.0305 | 0.0285 | 0.10225 | 558 |
| 453 | BIN1 3′ ss 4 | BIN1 5′ ss 4 | 2 | 0.17175 | 0.434 | 0.52125 | 0.02 | 0.04675 | 0.06475 | 559 |
| 454 | BIN1 3′ ss 4 | BIN1 5′ ss 4 | 2, 4 | 0.17825 | 0.40275 | 0.481 | 0.01475 | 0.086 | 0.02125 | 560 |
| 455 | BIN1 3′ ss 4 | BIN1 5′ ss 4 | 2, 3 | 0.12625 | 0.44375 | 0.449 | 0.01125 | 0.0295 | 0.01225 | 561 |
| 456 | BIN1 3′ ss 4 | BIN1 5′ ss 4 | 2, 3, 4 | 0.21125 | 0.49275 | 0.51775 | 0.0205 | 0.06475 | 0.02725 | 562 |
| 457 | BIN1 3′ ss 4 | BIN1 5′ ss 4 | 1 | 0.20325 | 0.5025 | 0.474 | 0.03175 | 0.00925 | 0.0235 | 563 |
| 458 | BIN1 3′ ss 4 | BIN1 5′ ss 4 | 1, 4 | 0.29775 | 0.56375 | 0.531 | 0.0175 | 0.02275 | 0.0345 | 564 |
| 459 | BIN1 3′ ss 4 | BIN1 5′ ss 4 | 1, 3 | 0.17825 | 0.45625 | 0.40725 | 0.027 | 0.085 | 0.04525 | 565 |
| 460 | BIN1 3′ ss 4 | BIN1 5′ ss 4 | 1, 3, 4 | 0.24575 | 0.4815 | 0.48725 | 0.03525 | 0.03975 | 0.03775 | 566 |
| 461 | BIN1 3′ ss 4 | BIN1 5′ ss 4 | 1, 2 | 0.2015 | 0.55925 | 0.51675 | 0.02075 | 0.049 | 0.02775 | 567 |
| 462 | BIN1 3′ ss 4 | BIN1 5′ ss 4 | 1, 2, 4 | 0.22825 | 0.4895 | 0.555 | 0.0225 | 0.14075 | 0.08075 | 568 |
| 463 | BIN1 3′ ss 4 | BIN1 5′ ss 4 | 1, 2, 3 | 0.20375 | 0.54275 | 0.62225 | 0.02275 | 0.06075 | 0.09375 | 569 |
| 464 | BIN1 3′ ss 4 | BIN1 5′ ss 4 | 1, 2, 3, 4 | 0.242 | 0.543 | 0.63975 | 0.04925 | 0.086 | 0.03225 | 570 |
| 465 | BIN1 3′ ss 4 | BIN1 5′ ss 5 | None | 0.17325 | 0.4235 | 0.354 | 0.02325 | 0.02 | 0.10425 | 571 |
| 466 | BIN1 3′ ss 4 | BIN1 5′ ss 5 | 4 | 0.1995 | 0.38225 | 0.41275 | 0.02 | 0.06175 | 0.0945 | 572 |
| 467 | BIN1 3′ ss 4 | BIN1 5′ ss 5 | 3 | 0.1685 | 0.3965 | 0.415 | 0.01775 | 0.03625 | 0.042 | 573 |
| 468 | BIN1 3′ ss 4 | BIN1 5′ ss 5 | 3, 4 | 0.21875 | 0.427 | 0.4885 | 0.022 | 0.02025 | 0.05625 | 574 |
| 469 | BIN1 3′ ss 4 | BIN1 5′ ss 5 | 2 | 0.14475 | 0.4135 | 0.3605 | 0.017 | 0.0405 | 0.075 | 575 |
| 470 | BIN1 3′ ss 4 | BIN1 5′ ss 5 | 2, 4 | 0.1875 | 0.58575 | 0.49375 | 0.01375 | 0.066 | 0.00625 | 576 |
| 471 | BIN1 3′ ss 4 | BIN1 5′ ss 5 | 2, 3 | 0.04775 | 0.21725 | 0.16325 | 0.00925 | 0.021 | 0.01425 | 577 |
| 472 | BIN1 3′ ss 4 | BIN1 5′ ss 5 | 2, 3, 4 | 0.11875 | 0.3245 | 0.32225 | 0.01575 | 0.02225 | 0.0735 | 578 |
| 473 | BIN1 3′ ss 4 | BIN1 5′ ss 5 | 1 | 0.15775 | 0.37675 | 0.43575 | 0.01425 | 0.02175 | 0.03825 | 579 |
| 474 | BIN1 3′ ss 4 | BIN1 5′ ss 5 | 1, 4 | 0.202 | 0.501 | 0.41075 | 0.01825 | 0.0095 | 0.02475 | 580 |
| 475 | BIN1 3′ ss 4 | BIN1 5′ ss 5 | 1, 3 | 0.18825 | 0.4165 | 0.46575 | 0.01475 | 0.01225 | 0.03175 | 581 |
| 476 | BIN1 3′ ss 4 | BIN1 5′ ss 5 | 1, 3, 4 | 0.267 | 0.45225 | 0.523 | 0.02525 | 0.01125 | 0.09275 | 582 |
| 477 | BIN1 3′ ss 4 | BIN1 5′ ss 5 | 1, 2 | 0.12325 | 0.3665 | 0.2145 | 0.01175 | 0.05075 | 0.023 | 583 |
| 478 | BIN1 3′ ss 4 | BIN1 5′ ss 5 | 1, 2, 4 | 0.133 | 0.42975 | 0.3285 | 0.01925 | 0.01825 | 0.0515 | 584 |
| 479 | BIN1 3′ ss 4 | BIN1 5′ ss 5 | 1, 2, 3 | 0.11075 | 0.40875 | 0.40475 | 0.01675 | 0.091 | 0.02375 | 585 |
| 480 | BIN1 3′ ss 4 | BIN1 5′ ss 5 | 1, 2, 3, 4 | 0.1515 | 0.3675 | 0.352 | 0.01725 | 0.003 | 0.00275 | 586 |
| 481 | BIN1 3′ ss 5 | Compensated | None | 0.17925 | 0.399 | 0.446 | 0.0185 | 0.02725 | 0.047 | 587 |
| 482 | BIN1 3′ ss 5 | Compensated | 4 | 0.20425 | 0.50875 | 0.44275 | 0.024 | 0.039 | 0.08875 | 588 |
| 483 | BIN1 3′ ss 5 | Compensated | 3 | 0.16475 | 0.463 | 0.417 | 0.02925 | 0.0475 | 0.0595 | 589 |
| 484 | BIN1 3′ ss 5 | Compensated | 3, 4 | 0.17175 | 0.355 | 0.48525 | 0.0245 | 0.008 | 0.046 | 590 |
| 485 | BIN1 3′ ss 5 | Compensated | 2 | 0.14725 | 0.42475 | 0.341 | 0.01475 | 0.0325 | 0.0255 | 59 |
| 486 | BIN1 3′ ss 5 | Compensated | 2, 4 | 0.13725 | 0.33325 | 0.35375 | 0.016 | 0.0285 | 0.01525 | 592 |
| 487 | BIN1 3′ ss 5 | Compensated | 2, 3 | 0.107 | 0.3575 | 0.4065 | 0.01275 | 0.0345 | 0.02 | 593 |
| 488 | BIN1 3′ ss 5 | Compensated | 2, 3, 4 | 0.13475 | 0.34925 | 0.399 | 0.01175 | 0.05575 | 0.05825 | 594 |
| 489 | BIN1 3′ ss 5 | Compensated | 1 | 0.18375 | 0.43475 | 0.51475 | 0.019 | 0.04025 | 0.02125 | 595 |
| 490 | BIN1 3′ ss 5 | Compensated | 1, 4 | 0.156 | 0.40225 | 0.4335 | 0.0205 | 0.0295 | 0.043 | 596 |
| 491 | BIN1 3′ ss 5 | Compensated | 1, 3 | 0.17325 | 0.39075 | 0.4365 | 0.014 | 0.0145 | 0.02475 | 597 |
| 492 | BIN1 3′ ss 5 | Compensated | 1, 3, 4 | 0.16725 | 0.36875 | 0.4505 | 0.0135 | 0.02175 | 0.0965 | 598 |
| 493 | BIN1 3′ ss 5 | Compensated | 1, 2 | 0.14325 | 0.39375 | 0.46075 | 0.0165 | 0.067 | 0.02025 | 599 |
| 494 | BIN1 3′ ss 5 | Compensated | 1, 2, 4 | 0.14625 | 0.438 | 0.41625 | 0.01975 | 0.05925 | 0.0555 | 600 |
| 495 | BIN1 3′ ss 5 | Compensated | 1, 2, 3 | 0.13625 | 0.484 | 0.38575 | 0.01475 | 0.16075 | 0.0315 | 601 |
| 496 | BIN1 3′ ss 5 | Compensated | 1, 2, 3, 4 | 0.108 | 0.25925 | 0.28375 | 0.01675 | 0.076 | 0.031 | 602 |
| 497 | BIN1 3′ ss 5 | BIN1 5′ ss 1 | None | 0.25625 | 0.61325 | 0.6505 | 0.027 | 0.0985 | 0.03375 | 603 |
| 498 | BIN1 3′ ss 5 | BIN1 5′ ss 1 | 4 | 0.288 | 0.6305 | 0.6815 | 0.0455 | 0.06675 | 0.178 | 604 |
| 499 | BIN1 3′ ss 5 | BIN1 5′ ss 1 | 3 | 0.2935 | 0.72425 | 0.7125 | 0.054 | 0.16275 | 0.161 | 605 |
| 500 | BIN1 3′ ss 5 | BIN1 5′ ss 1 | 3, 4 | 0.35575 | 0.67775 | 0.68225 | 0.064 | 0.20125 | 0.107 | 606 |
| 501 | BIN1 3′ ss 5 | BIN1 5′ ss 1 | 2 | 0.226 | 0.60175 | 0.643 | 0.03425 | 0.1315 | 0.20925 | 607 |
| 502 | BIN1 3′ ss 5 | BIN1 5′ ss 1 | 2, 4 | 0.23875 | 0.65875 | 0.71575 | 0.04575 | 0.20625 | 0.2745 | 608 |
| 503 | BIN1 3′ ss 5 | BIN1 5′ ss 1 | 2, 3 | 0.2085 | 0.579 | 0.8805 | 0.0335 | 0.12975 | 0.07925 | 609 |
| 504 | BIN1 3′ ss 5 | BIN1 5′ ss 1 | 2, 3, 4 | 0.2775 | 0.62625 | 0.67475 | 0.0515 | 0.19725 | 0.14675 | 610 |
| 505 | BIN1 3′ ss 5 | BIN1 5′ ss 1 | 1 | 0.27925 | 0.69075 | 0.65 | 0.05575 | 0.069 | 0.0415 | 611 |
| 506 | BIN1 3′ ss 5 | BIN1 5′ ss 1 | 1, 4 | 0.34525 | 0.62 | 0.63525 | 0.0625 | 0.17425 | 0.07825 | 612 |
| 507 | BIN1 3′ ss 5 | BIN1 5′ ss 1 | 1, 3 | 0.382 | 0.697 | 0.74125 | 0.067 | 0.11975 | 0.167 | 613 |
| 508 | BIN1 3′ ss 5 | BIN1 5′ ss 1 | 1, 3, 4 | 0.38075 | 0.7095 | 0.523 | 0.07325 | 0.165 | 0.24475 | 614 |
| 509 | BIN1 3′ ss 5 | BIN1 5′ ss 1 | 1, 2 | 0.238 | 0.6235 | 0.538 | 0.03525 | 0.08925 | 0.1295 | 615 |
| 510 | BIN1 3′ ss 5 | BIN1 5′ ss 1 | 1, 2, 4 | 0.2495 | 0.6275 | 0.69475 | 0.05775 | 0.08925 | 0.08825 | 616 |
| 511 | BIN1 3′ ss 5 | BIN1 5′ ss 1 | 1, 2, 3 | 0.27 | 0.74825 | 0.71 | 0.0325 | 0.04625 | 0.00625 | 617 |
| 512 | BIN1 3′ ss 5 | BIN1 5′ ss 1 | 1, 2, 3, 4 | 0.40275 | 0.75425 | 0.77775 | 0.08375 | 0.2175 | 0.3375 | 618 |
| 513 | BIN1 3′ ss 5 | BIN1 5′ ss 2 | None | 0.143 | 0.4005 | 0.44525 | 0.01725 | 0.04625 | 0.03275 | 619 |
| 514 | BIN1 3′ ss 5 | BIN1 5′ ss 2 | 4 | 0.19075 | 0.48575 | 0.44875 | 0.0195 | 0.02475 | 0.03875 | 620 |
| 515 | BIN1 3′ ss 5 | BIN1 5′ ss 2 | 3 | 0.1345 | 0.4055 | 0.3615 | 0.016 | 0.0275 | 0.05525 | 621 |
| 516 | BIN1 3′ ss 5 | BIN1 5′ ss 2 | 3, 4 | 0.1465 | 0.39225 | 0.43075 | 0.017 | 0.04925 | 0.043 | 622 |
| 517 | BIN1 3′ ss 5 | BIN1 5′ ss 2 | 2 | 0.117 | 0.408 | 0.348 | 0.01175 | 0.04475 | 0.0335 | 623 |
| 518 | BIN1 3′ ss 5 | BIN1 5′ ss 2 | 2, 4 | 0.13525 | 0.38625 | 0.29575 | 0.01475 | 0.04625 | 0.101 | 624 |
| 519 | BIN1 3′ ss 5 | BIN1 5′ ss 2 | 2, 3 | 0.091 | 0.31075 | 0.375 | 0.0105 | 0.01375 | 0.03525 | 625 |
| 520 | BIN1 3′ ss 5 | BIN1 5′ ss 2 | 2, 3, 4 | 0.1335 | 0.447 | 0.40475 | 0.01275 | 0.03625 | 0.03875 | 626 |
| 521 | BIN1 3′ ss 5 | BIN1 5′ ss 2 | 1 | 0.13075 | 0.3625 | 0.398 | 0.0225 | 0.05725 | 0.0415 | 627 |
| 522 | BIN1 3′ ss 5 | BIN1 5′ ss 2 | 1, 4 | 0.1555 | 0.406 | 0.476 | 0.01375 | 0.029 | 0.01375 | 628 |
| 523 | BIN1 3′ ss 5 | BIN1 5′ ss 2 | 1, 3 | 0.13975 | 0.40875 | 0.478 | 0.02175 | 0.12525 | 0.057 | 629 |
| 524 | BIN1 3′ ss 5 | BIN1 5′ ss 2 | 1, 3, 4 | 0.14025 | 0.447 | 0.43475 | 0.0165 | 0.03525 | 0.02825 | 630 |
| 525 | BIN1 3′ ss 5 | BIN1 5′ ss 2 | 1, 2 | 0.11375 | 0.38225 | 0.42125 | 0.0105 | 0.013 | 0.02925 | 631 |
| 526 | BIN1 3′ ss 5 | BIN1 5′ ss 2 | 1, 2, 4 | 0.0715 | 0.21625 | 0.40075 | 0.01525 | 0.03625 | 0.12125 | 632 |
| 527 | BIN1 3′ ss 5 | BIN1 5′ ss 2 | 1, 2, 3 | 0.09525 | 0.37275 | 0.5185 | 0.01475 | 0.037 | 0.01575 | 633 |
| 528 | BIN1 3′ ss 5 | BIN1 5′ ss 2 | 1, 2, 3, 4 | 0.1345 | 0.37175 | 0.5055 | 0.02225 | 0.07625 | 0.0255 | 634 |
| 529 | BIN1 3′ ss 5 | BIN1 5′ ss 3 | None | 0.1485 | 0.34425 | 0.41775 | 0.019 | 0.03075 | 0.072 | 635 |
| 530 | BIN1 3′ ss 5 | BIN1 5′ ss 3 | 4 | 0.15625 | 0.3665 | 0.4135 | 0.01975 | 0.0405 | 0.019 | 636 |
| 531 | BIN1 3′ ss 5 | BIN1 5′ ss 3 | 3 | 0.14275 | 0.3825 | 0.3975 | 0.017 | 0.0855 | 0.02625 | 637 |
| 532 | BIN1 3′ ss 5 | BIN1 5′ ss 3 | 3, 4 | 0.15475 | 0.34475 | 0.3715 | 0.0185 | 0.025 | 0.08375 | 638 |
| 533 | BIN1 3′ ss 5 | BIN1 5′ ss 3 | 2 | 0.124 | 0.3155 | 0.30775 | 0.01325 | 0.04325 | 0.01475 | 639 |
| 534 | BIN1 3′ ss 5 | BIN1 5′ ss 3 | 2, 4 | 0.13575 | 0.30275 | 0.38675 | 0.0135 | 0.051 | 0.00575 | 640 |
| 535 | BIN1 3′ ss 5 | BIN1 5′ ss 3 | 2, 3 | 0.097 | 0.32325 | 0.354 | 0.01225 | 0.0185 | 0.016 | 641 |
| 536 | BIN1 3′ ss 5 | BIN1 5′ ss 3 | 2, 3, 4 | 0.1075 | 0.308 | 0.33 | 0.01725 | 0.0415 | 0.0645 | 642 |
| 537 | BIN1 3′ ss 5 | BIN1 5′ ss 3 | 1 | 0.172 | 0.4205 | 0.4665 | 0.01475 | 0.03775 | 0.024 | 643 |
| 538 | BIN1 3′ ss 5 | BIN1 5′ ss 3 | 1, 4 | 0.16725 | 0.40375 | 0.45225 | 0.0225 | 0.0325 | 0.049 | 644 |
| 539 | BIN1 3′ ss 5 | BIN1 5′ ss 3 | 1, 3 | 0.14175 | 0.3845 | 0.45375 | 0.015 | 0.01475 | 0.01025 | 645 |
| 540 | BIN1 3′ ss 5 | BIN1 5′ ss 3 | 1, 3, 4 | 0.1435 | 0.28475 | 0.35975 | 0.01425 | 0.022 | 0.0135 | 646 |
| 541 | BIN1 3′ ss 5 | BIN1 5′ ss 3 | 1, 2 | 0.1415 | 0.39625 | 0.466 | 0.0115 | 0.0765 | 0.01525 | 647 |
| 542 | BIN1 3′ ss 5 | BIN1 5′ ss 3 | 1, 2, 4 | 0.16325 | 0.398 | 0.498 | 0.01925 | 0.06 | 0.0475 | 648 |
| 543 | BIN1 3′ ss 5 | BIN1 5′ ss 3 | 1, 2, 3 | 0.1185 | 0.403 | 0.412 | 0.01425 | 0.03575 | 0.036 | 649 |
| 544 | BIN1 3′ ss 5 | BIN1 5′ ss 3 | 1, 2, 3, 4 | 0.12925 | 0.2865 | 0.31175 | 0.01875 | 0.02025 | 0.194 | 650 |
| 545 | BIN1 3′ ss 5 | BIN1 5′ ss 4 | None | 0.14925 | 0.35325 | 0.39075 | 0.0155 | 0.0185 | 0.04825 | 651 |
| 546 | BIN1 3′ ss 5 | BIN1 5′ ss 4 | 4 | 0.16375 | 0.3555 | 0.39775 | 0.0135 | 0.037 | 0.01425 | 652 |
| 547 | BIN1 3′ ss 5 | BIN1 5′ ss 4 | 3 | 0.10525 | 0.29225 | 0.29425 | 0.01725 | 0.0115 | 0.07925 | 653 |
| 548 | BIN1 3′ ss 5 | BIN1 5′ ss 4 | 3, 4 | 0.138 | 0.36725 | 0.346 | 0.0135 | 0.064 | 0.03525 | 654 |
| 549 | BIN1 3′ ss 5 | BIN1 5′ ss 4 | 2 | 0.1205 | 0.24725 | 0.33075 | 0.0165 | 0.03975 | 0.02325 | 655 |
| 550 | BIN1 3′ ss 5 | BIN1 5′ ss 4 | 2, 4 | 0.155 | 0.318 | 0.34475 | 0.01675 | 0.074 | 0.01475 | 656 |
| 551 | BIN1 3′ ss 5 | BIN1 5′ ss 4 | 2, 3 | 0.10425 | 0.30775 | 0.2425 | 0.01225 | 0.03525 | 0.07475 | 657 |
| 552 | BIN1 3′ ss 5 | BIN1 5′ ss 4 | 2, 3, 4 | 0.09925 | 0.277 | 0.33425 | 0.01325 | 0.03575 | 0.0185 | 658 |
| 553 | BIN1 3′ ss 5 | BIN1 5′ ss 4 | 1 | 0.1395 | 0.3405 | 0.35925 | 0.017 | 0.0385 | 0.074 | 659 |
| 554 | BIN1 3′ ss 5 | BIN1 5′ ss 4 | 1, 4 | 0.12575 | 0.30075 | 0.3425 | 0.0205 | 0.028 | 0.02875 | 660 |
| 555 | BIN1 3′ ss 5 | BIN1 5′ ss 4 | 1, 3 | 0.12575 | 0.34575 | 0.42225 | 0.0135 | 0.0105 | 0.01375 | 661 |
| 556 | BIN1 3′ ss 5 | BIN1 5′ ss 4 | 1, 3, 4 | 0.1215 | 0.34325 | 0.306 | 0.019 | 0.0565 | 0.02875 | 662 |
| 557 | BIN1 3′ ss 5 | BIN1 5′ ss 4 | 1, 2 | 0.106 | 0.3285 | 0.3175 | 0.0135 | 0.01575 | 0.04125 | 663 |
| 558 | BIN1 3′ ss 5 | BIN1 5′ ss 4 | 1, 2, 4 | 0.14175 | 0.28475 | 0.39025 | 0.01375 | 0.008 | 0.0385 | 664 |
| 559 | BIN1 3′ ss 5 | BIN1 5′ ss 4 | 1, 2, 3 | 0.07375 | 0.25875 | 0.208 | 0.00925 | 0.00625 | 0.0215 | 665 |
| 560 | BIN1 3′ ss 5 | BIN1 5′ ss 4 | 1, 2, 3, 4 | 0.131 | 0.317 | 0.29975 | 0.01725 | 0.09925 | 0.019 | 666 |
| 561 | BIN1 3′ ss 5 | BIN1 5′ ss 5 | None | 0.1545 | 0.34175 | 0.432 | 0.01675 | 0.023 | 0.03675 | 667 |
| 562 | BIN1 3′ ss 5 | BIN1 5′ ss 5 | 4 | 0.1545 | 0.2495 | 0.35525 | 0.018 | 0.021 | 0.0405 | 668 |
| 563 | BIN1 3′ ss 5 | BIN1 5′ ss 5 | 3 | 0.11 | 0.21775 | 0.278 | 0.01 | 0.039 | 0.0145 | 669 |
| 564 | BIN1 3′ ss 5 | BIN1 5′ ss 5 | 3, 4 | 0.11525 | 0.25325 | 0.28125 | 0.0165 | 0.0125 | 0.0145 | 670 |
| 565 | BIN1 3′ ss 5 | BIN1 5′ ss 5 | 2 | 0.10825 | 0.2155 | 0.27025 | 0.01475 | 0.03375 | 0.07025 | 671 |
| 566 | BIN1 3′ ss 5 | BIN1 5′ ss 5 | 2, 4 | 0.13 | 0.231 | 0.22025 | 0.0205 | 0.01525 | 0.0285 | 672 |
| 567 | BIN1 3′ ss 5 | BIN1 5′ ss 5 | 2, 3 | 0.106 | 0.22075 | 0.239 | 0.01325 | 0.0205 | 0.047 | 673 |
| 568 | BIN1 3′ ss 5 | BIN1 5′ ss 5 | 2, 3, 4 | 0.06825 | 0.10925 | 0.1765 | 0.00875 | 0.03025 | 0.0115 | 674 |
| 569 | BIN1 3′ ss 5 | BIN1 5′ ss 5 | 1 | 0.11325 | 0.26 | 0.2895 | 0.01575 | 0.04325 | 0.03975 | 675 |
| 570 | BIN1 3′ ss 5 | BIN1 5′ ss 5 | 1, 4 | 0.11975 | 0.24825 | 0.3225 | 0.0145 | 0.01325 | 0.0115 | 676 |
| 571 | BIN1 3′ ss 5 | BIN1 5′ ss 5 | 1, 3 | 0.117 | 0.2405 | 0.26775 | 0.01125 | 0.00625 | 0.0685 | 677 |
| 572 | BIN1 3′ ss 5 | BIN1 5′ ss 5 | 1, 3, 4 | 0.093 | 0.17225 | 0.259 | 0.0105 | 0.00825 | 0.011 | 678 |
| 573 | BIN1 3′ ss 5 | BIN1 5′ ss 5 | 1, 2 | 0.1075 | 0.21175 | 0.23775 | 0.01325 | 0.00525 | 0.08925 | 679 |
| 574 | BIN1 3′ ss 5 | BIN1 5′ ss 5 | 1, 2, 4 | 0.10075 | 0.19875 | 0.284 | 0.00825 | 0.006 | 0.0205 | 680 |
| 575 | BIN1 3′ ss 5 | BIN1 5′ ss 5 | 1, 2, 3 | 0.08025 | 0.2045 | 0.30025 | 0.0085 | 0.01375 | 0.0185 | 681 |
| 576 | BIN1 3′ ss 5 | BIN1 5′ ss 5 | 1, 2, 3, 4 | 0.07125 | 0.13475 | 0.1995 | 0.01325 | 0.035 | 0.06275 | 682 |
| 577 | BIN1 3′ ss 6 | Compensated | None | 0.2045 | 0.53725 | 0.601 | 0.023 | 0.0275 | 0.0495 | 683 |
| 578 | BIN1 3′ ss 6 | Compensated | 4 | 0.25275 | 0.7255 | 0.56525 | 0.02125 | 0.03125 | 0.0065 | 684 |
| 579 | BIN1 3′ ss 6 | Compensated | 3 | 0.14 | 0.37825 | 0.3735 | 0.02075 | 0.06375 | 0.05025 | 685 |
| 580 | BIN1 3′ ss 6 | Compensated | 3, 4 | 0.234 | 0.535 | 0.5115 | 0.03125 | 0.05775 | 0.05625 | 686 |
| 581 | BIN1 3′ ss 6 | Compensated | 2 | 0.14675 | 0.46175 | 0.39125 | ND | ND | ND | 687 |
| 582 | BIN1 3′ ss 6 | Compensated | 2, 4 | 0.16225 | 0.41475 | 0.4525 | 0.0215 | 0.0555 | 0.04425 | 688 |
| 583 | BIN1 3′ ss 6 | Compensated | 2, 3 | 0.16375 | 0.5155 | 0.7095 | 0.01575 | 0.01725 | 0.009 | 689 |
| 584 | BIN1 3′ ss 6 | Compensated | 2, 3, 4 | 0.15875 | 0.44025 | 0.4205 | 0.01425 | 0.0095 | 0.02375 | 690 |
| 585 | BIN1 3′ ss 6 | Compensated | 1 | 0.13625 | 0.39725 | 0.4145 | 0.0265 | 0.09325 | 0.0605 | 691 |
| 586 | BIN1 3′ ss 6 | Compensated | 1, 4 | 0.22425 | 0.51825 | 0.69275 | 0.03125 | 0.05325 | 0.123 | 692 |
| 587 | BIN1 3′ ss 6 | Compensated | 1, 3 | ND | ND | ND | 0.026 | 0.03525 | 0.0335 | 693 |
| 588 | BIN1 3′ ss 6 | Compensated | 1, 3, 4 | 0.24575 | 0.53475 | 0.593 | 0.0315 | 0.03025 | 0.0135 | 694 |
| 589 | BIN1 3′ ss 6 | Compensated | 1, 2 | 0.20025 | 0.532 | 0.4635 | 0.017 | 0.08825 | 0.083 | 695 |
| 590 | BIN1 3′ ss 6 | Compensated | 1, 2, 4 | 0.21175 | 0.5855 | 0.50425 | ND | ND | ND | 696 |
| 591 | BIN1 3′ ss 6 | Compensated | 1, 2, 3 | 0.202 | 0.59375 | 0.5045 | ND | ND | ND | 697 |
| 592 | BIN1 3′ ss 6 | Compensated | 1, 2, 3, 4 | 0.2215 | 0.57275 | 0.64975 | ND | ND | ND | 698 |
| 593 | BIN1 3′ ss 6 | BIN1 5′ ss 1 | None | 0.266 | 0.727 | 0.52925 | 0.04025 | 0.07675 | 0.03525 | 699 |
| 594 | BIN1 3′ ss 6 | BIN1 5′ ss 1 | 4 | 0.33975 | 0.6995 | 0.6715 | ND | ND | ND | 700 |
| 595 | BIN1 3′ ss 6 | BIN1 5′ ss 1 | 3 | 0.3185 | 0.6365 | 0.61675 | 0.064 | 0.158333333 | 0.0995 | 701 |
| 596 | BIN1 3′ ss 6 | BIN1 5′ ss 1 | 3, 4 | 0.40775 | 0.628 | 0.7695 | 0.0795 | 0.2155 | 0.26125 | 702 |
| 597 | BIN1 3′ ss 6 | BIN1 5′ ss 1 | 2 | 0.21 | 0.64975 | 0.59875 | 0.03725 | 0.18725 | 0.0815 | 703 |
| 598 | BIN1 3′ ss 6 | BIN1 5′ ss 1 | 2, 4 | 0.286 | 0.759 | 0.63725 | 0.05325 | 0.136 | 0.21125 | 704 |
| 599 | BIN1 3′ ss 6 | BIN1 5′ ss 1 | 2, 3 | 0.1865 | 0.694 | 0.721 | 0.02575 | 0.07125 | 0.36125 | 705 |
| 600 | BIN1 3′ ss 6 | BIN1 5′ ss 1 | 2, 3, 4 | 0.3825 | 0.667 | 0.69375 | 0.0565 | 0.1835 | 0.1665 | 706 |
| 601 | BIN1 3′ ss 6 | BIN1 5′ ss 1 | 1 | 0.383 | 0.75125 | 0.68525 | 0.065 | 0.20525 | 0.198 | 707 |
| 602 | BIN1 3′ ss 6 | BIN1 5′ ss 1 | 1, 4 | 0.38225 | 0.6425 | 0.69625 | 0.0725 | 0.078 | 0.1245 | 708 |
| 603 | BIN1 3′ ss 6 | BIN1 5′ ss 1 | 1, 3 | 0.28825 | 0.6565 | 0.2265 | 0.0785 | 0.093 | 0.16425 | 709 |
| 604 | BIN1 3′ ss 6 | BIN1 5′ ss 1 | 1, 3, 4 | 0.49825 | 0.77425 | 0.807 | 0.098 | 0.3345 | 0.22875 | 710 |
| 605 | BIN1 3′ ss 6 | BIN1 5′ ss 1 | 1, 2 | 0.295 | 0.619 | 0.67825 | 0.0405 | 0.116 | 0.1095 | 711 |
| 606 | BIN1 3′ ss 6 | BIN1 5′ ss 1 | 1, 2, 4 | ND | ND | ND | 0.0715 | 0.1135 | 0.2445 | 712 |
| 607 | BIN1 3′ ss 6 | BIN1 5′ ss 1 | 1, 2, 3 | 0.365 | 0.75975 | 0.861 | 0.06975 | 0.13475 | 0.362 | 713 |
| 608 | BIN1 3′ ss 6 | BIN1 5′ ss 1 | 1, 2, 3, 4 | 0.4105 | 0.73425 | 0.79475 | 0.07375 | 0.0255 | 0.040666667 | 714 |
| 609 | BIN1 3′ ss 6 | BIN1 5′ ss 2 | None | 0.17325 | 0.534 | 0.45675 | 0.01825 | 0.03275 | 0.05125 | 715 |
| 610 | BIN1 3′ ss 6 | BIN1 5′ ss 2 | 4 | 0.15525 | 0.41775 | 0.4715 | 0.02925 | 0.0175 | 0.068 | 716 |
| 611 | BIN1 3′ ss 6 | BIN1 5′ ss 2 | 3 | 0.10975 | 0.54975 | 0.521 | 0.01925 | 0.18625 | 0.04875 | 717 |
| 612 | BIN1 3′ ss 6 | BIN1 5′ ss 2 | 3, 4 | 0.17925 | 0.49275 | 0.43325 | 0.02075 | 0.11 | 0.07675 | 718 |
| 613 | BIN1 3′ ss 6 | BIN1 5′ ss 2 | 2 | 0.157 | 0.44875 | 0.45925 | 0.00925 | 0.332666667 | 0.026 | 719 |
| 614 | BIN1 3′ ss 6 | BIN1 5′ ss 2 | 2, 4 | 0.1645 | 0.4705 | 0.59575 | 0.0175 | 0.01775 | 0.128 | 720 |
| 615 | BIN1 3′ ss 6 | BIN1 5′ ss 2 | 2, 3 | 0.1075 | 0.45075 | 0.457 | 0.01375 | 0.187 | 0.09025 | 721 |
| 616 | BIN1 3′ ss 6 | BIN1 5′ ss 2 | 2, 3, 4 | 0.126 | 0.5125 | 0.4895 | 0.02875 | 0.10775 | 0.0315 | 722 |
| 617 | BIN1 3′ ss 6 | BIN1 5′ ss 2 | 1 | 0.1645 | 0.45775 | 0.4685 | 0.015 | 0.109 | 0.052 | 723 |
| 618 | BIN1 3′ ss 6 | BIN1 5′ ss 2 | 1, 4 | 0.16825 | 0.58375 | 0.54 | 0.0205 | 0.0255 | 0.138 | 724 |
| 619 | BIN1 3′ ss 6 | BIN1 5′ ss 2 | 1, 3 | 0.152 | 0.43275 | 0.5465 | 0.016 | 0.024 | 0.035 | 725 |
| 620 | BIN1 3′ ss 6 | BIN1 5′ ss 2 | 1, 3, 4 | 0.1715 | 0.529 | 0.44375 | 0.02125 | 0.03775 | 0.037 | 726 |
| 621 | BIN1 3′ ss 6 | BIN1 5′ ss 2 | 1, 2 | 0.13875 | 0.5345 | 0.5565 | 0.01225 | 0.11025 | 0.03175 | 727 |
| 622 | BIN1 3′ ss 6 | BIN1 5′ ss 2 | 1, 2, 4 | 0.14575 | 0.474 | 0.45075 | 0.02675 | 0.02725 | 0.0635 | 728 |
| 623 | BIN1 3′ ss 6 | BIN1 5′ ss 2 | 1, 2, 3 | 0.0845 | 0.5185 | 0.411 | 0.0155 | 0.07025 | 0.13625 | 729 |
| 624 | BIN1 3′ ss 6 | BIN1 5′ ss 2 | 1, 2, 3, 4 | 0.1835 | 0.59 | 0.62525 | 0.0165 | 0.18825 | 0.012 | 730 |
| 625 | BIN1 3′ ss 6 | BIN1 5′ ss 3 | None | 0.1945 | 0.44675 | 0.39975 | 0.021 | 0.031 | 0.0805 | 731 |
| 626 | BIN1 3′ ss 6 | BIN1 5′ ss 3 | 4 | 0.197 | 0.47725 | 0.43425 | 0.02325 | 0.0505 | 0.0265 | 732 |
| 627 | BIN1 3′ ss 6 | BIN1 5′ ss 3 | 3 | 0.1655 | 0.37425 | 0.5695 | 0.01325 | 0.02825 | 0.018 | 733 |
| 628 | BIN1 3′ ss 6 | BIN1 5′ ss 3 | 3, 4 | 0.19625 | 0.4055 | 0.47 | 0.0185 | 0.08025 | 0.024 | 734 |
| 629 | BIN1 3′ ss 6 | BIN1 5′ ss 3 | 2 | 0.109 | 0.322 | 0.342 | 0.014 | 0.03825 | 0.02375 | 735 |
| 630 | BIN1 3′ ss 6 | BIN1 5′ ss 3 | 2, 4 | 0.1775 | 0.51225 | 0.45325 | 0.0205 | 0.01275 | 0.01875 | 736 |
| 631 | BIN1 3′ ss 6 | BIN1 5′ ss 3 | 2, 3 | 0.11225 | 0.40175 | 0.4355 | 0.028 | 0.03725 | 0.024 | 737 |
| 632 | BIN1 3′ ss 6 | BIN1 5′ ss 3 | 2, 3, 4 | 0.2115 | 0.4055 | 0.45075 | 0.0215 | 0.08225 | 0.02125 | 738 |
| 633 | BIN1 3′ ss 6 | BIN1 5′ ss 3 | 1 | 0.1935 | 0.40925 | 0.546 | 0.0235 | 0.0335 | 0.01625 | 739 |
| 634 | BIN1 3′ ss 6 | BIN1 5′ ss 3 | 1, 4 | 0.1775 | 0.40325 | 0.41975 | 0.0285 | 0.06075 | 0.054 | 740 |
| 635 | BIN1 3′ ss 6 | BIN1 5′ ss 3 | 1, 3 | 0.1935 | 0.465 | 0.461 | 0.02925 | 0.058 | 0.087 | 741 |
| 636 | BIN1 3′ ss 6 | BIN1 5′ ss 3 | 1, 3, 4 | 0.23575 | 0.45425 | 0.483 | 0.02825 | 0.01725 | 0.059 | 742 |
| 637 | BIN1 3′ ss 6 | BIN1 5′ ss 3 | 1, 2 | 0.0655 | 0.2265 | 0.262 | 0.0155 | 0.02075 | 0.042 | 743 |
| 638 | BIN1 3′ ss 6 | BIN1 5′ ss 3 | 1, 2, 4 | 0.19425 | 0.53525 | 0.571 | 0.03575 | 0.11175 | 0.029 | 744 |
| 639 | BIN1 3′ ss 6 | BIN1 5′ ss 3 | 1, 2, 3 | 0.1205 | 0.413 | 0.4555 | 0.00625 | 0.0635 | 0.01075 | 745 |
| 640 | BIN1 3′ ss 6 | BIN1 5′ ss 3 | 1, 2, 3, 4 | 0.161 | 0.45025 | 0.38875 | 0.02525 | 0.08275 | 0.02275 | 746 |
| 641 | BIN1 3′ ss 6 | BIN1 5′ ss 4 | None | 0.159 | 0.44725 | 0.4755 | 0.0135 | 0.00725 | 0.0135 | 747 |
| 642 | BIN1 3′ ss 6 | BIN1 5′ ss 4 | 4 | 0.167 | 0.4315 | 0.346 | 0.0175 | 0.01 | 0.0525 | 748 |
| 643 | BIN1 3′ ss 6 | BIN1 5′ ss 4 | 3 | 0.1275 | 0.33675 | 0.4195 | 0.01075 | 0.01775 | 0.01375 | 749 |
| 644 | BIN1 3′ ss 6 | BIN1 5′ ss 4 | 3, 4 | 0.16375 | 0.429 | 0.3595 | 0.01725 | 0.018 | 0.045 | 750 |
| 645 | BIN1 3′ ss 6 | BIN1 5′ ss 4 | 2 | 0.134 | 0.359 | 0.4825 | 0.01575 | 0.01575 | 0.00825 | 751 |
| 646 | BIN1 3′ ss 6 | BIN1 5′ ss 4 | 2, 4 | 0.14175 | 0.34925 | 0.29025 | ND | ND | ND | 752 |
| 647 | BIN1 3′ ss 6 | BIN1 5′ ss 4 | 2, 3 | 0.08175 | 0.293 | 0.44025 | 0.0095 | 0.0195 | 0.0065 | 753 |
| 648 | BIN1 3′ ss 6 | BIN1 5′ ss 4 | 2, 3, 4 | 0.117 | 0.3805 | 0.42775 | 0.0145 | 0.013 | 0.111 | 754 |
| 649 | BIN1 3′ ss 6 | BIN1 5′ ss 4 | 1 | 0.1705 | 0.39625 | 0.393 | 0.015 | 0.04525 | 0.0065 | 755 |
| 650 | BIN1 3′ ss 6 | BIN1 5′ ss 4 | 1, 4 | 0.177 | 0.43 | 0.39275 | 0.02225 | 0.13275 | 0.014 | 756 |
| 651 | BIN1 3′ ss 6 | BIN1 5′ ss 4 | 1, 3 | 0.1255 | 0.32375 | 0.27275 | 0.00975 | 0.01375 | 0.025 | 757 |
| 652 | BIN1 3′ ss 6 | BIN1 5′ ss 4 | 1, 3, 4 | 0.147 | 0.32075 | 0.3555 | 0.01975 | 0.04675 | 0.0345 | 758 |
| 653 | BIN1 3′ ss 6 | BIN1 5′ ss 4 | 1, 2 | 0.13075 | 0.4335 | 0.4885 | 0.01725 | 0.048 | 0.02425 | 759 |
| 654 | BIN1 3′ ss 6 | BIN1 5′ ss 4 | 1, 2, 4 | ND | ND | ND | 0.01275 | 0.022 | 0.01875 | 760 |
| 655 | BIN1 3′ ss 6 | BIN1 5′ ss 4 | 1, 2, 3 | 0.117 | 0.416 | 0.46 | 0.01275 | 0.03725 | 0.00775 | 761 |
| 656 | BIN1 3′ ss 6 | BIN1 5′ ss 4 | 1, 2, 3, 4 | 0.155 | 0.49075 | 0.465 | 0.0185 | 0.02475 | 0.02425 | 762 |
| 657 | BIN1 3′ ss 6 | BIN1 5′ ss 5 | None | 0.1105 | 0.25275 | 0.376 | 0.0245 | 0.02525 | 0.01475 | 763 |
| 658 | BIN1 3′ ss 6 | BIN1 5′ ss 5 | 4 | 0.106 | 0.26475 | 0.2935 | 0.0215 | 0.01975 | 0.02175 | 764 |
| 659 | BIN1 3′ ss 6 | BIN1 5′ ss 5 | 3 | 0.101 | 0.244 | 0.2915 | 0.013 | 0.0505 | 0.00825 | 765 |
| 660 | BIN1 3′ ss 6 | BIN1 5′ ss 5 | 3, 4 | 0.13475 | 0.23175 | 0.4885 | 0.01025 | 0.0065 | 0.09375 | 766 |
| 661 | BIN1 3′ ss 6 | BIN1 5′ ss 5 | 2 | 0.06725 | 0.289 | 0.386 | 0.01275 | 0.02875 | 0.00675 | 767 |
| 662 | BIN1 3′ ss 6 | BIN1 5′ ss 5 | 2, 4 | 0.1085 | 0.3745 | 0.249 | 0.014 | 0.00525 | 0.00375 | 768 |
| 663 | BIN1 3′ ss 6 | BIN1 5′ ss 5 | 2, 3 | 0.09625 | 0.2725 | 0.251 | 0.011 | 0.015 | 0.00975 | 769 |
| 664 | BIN1 3′ ss 6 | BIN1 5′ ss 5 | 2, 3, 4 | ND | ND | ND | 0.0075 | 0.0135 | 0.04325 | 770 |
| 665 | BIN1 3′ ss 6 | BIN1 5′ ss 5 | 1 | 0.13 | 0.358 | 0.361 | 0.01775 | 0.00625 | 0.022 | 771 |
| 666 | BIN1 3′ ss 6 | BIN1 5′ ss 5 | 1, 4 | 0.133 | 0.39375 | 0.26025 | 0.01925 | 0.00825 | 0.01975 | 772 |
| 667 | BIN1 3′ ss 6 | BIN1 5′ ss 5 | 1, 3 | 0.11125 | 0.25725 | 0.32975 | 0.01675 | 0.009 | 0.015 | 773 |
| 668 | BIN1 3′ ss 6 | BIN1 5′ ss 5 | 1, 3, 4 | 0.0775 | 0.23325 | 0.3105 | 0.0125 | 0.00575 | 0.0095 | 774 |
| 669 | BIN1 3′ ss 6 | BIN1 5′ ss 5 | 1, 2 | 0.08325 | 0.32275 | 0.287 | 0.0065 | 0.014 | 0.06825 | 775 |
| 670 | BIN1 3′ ss 6 | BIN1 5′ ss 5 | 1, 2, 4 | 0.09725 | 0.183 | 0.166666667 | 0.0105 | 0.0055 | 0.014 | 776 |
| 671 | BIN1 3′ ss 6 | BIN1 5′ ss 5 | 1, 2, 3 | ND | ND | ND | 0.0095 | 0.01125 | 0.02125 | 777 |
| 672 | BIN1 3′ ss 6 | BIN1 5′ ss 5 | 1, 2, 3, 4 | 0.1015 | 0.295 | 0.36275 | 0.009 | 0.004 | 0.051 | 778 |

**The columns for Table 4 are further described as follows:
ID: (Variant ID): an identification number for each specific BIN1 alternative exon cassette variant.
3′ splice site ID: an identification number for each 3′ splice site, as indicated in FIG. 19.
3′ splice site ID: an identification number for each 5′ splice site, as indicated in FIG. 19.
Intron insertions: The locations of specific intronic modifications within each variant, as listed in FIG. 20.
MTM1_Heart: psi of the variant in heart when linked to the MTM1 cargo.
MTM1_Gastroc: psi of the variant in gastrocnemius when linked to the MTM1 cargo.
MTM1_Tibialis: psi of the variant in tibialis when linked to the MTM1 cargo.
CAPN3_Heart: psi of the variant in heart when linked to the CAPN3 cargo
CAPN3_Gastroc: psi of the variant in gastrocnemius when linked to the CAPN3 cargo
CAPN3_Tibialis: psi of the variant in tibialis when linked to the CAPN3 cargo
SEQ ID NO: the sequence identifier associated with the intron-exon-intron sequence of the particular cassette variant.

Application of this High Throughput Screening Approach to Identify Alternative Exon Cassettes with Regulated Splicing Patterns in Additional Tissues

The ability to limit or augment gene expression in a variety of tissues would be useful for gene therapies, and some notable tissues include the liver, different brain regions, dorsal root ganglia (DRG), skeletal muscle, cardiac muscle, and smooth muscle. GTEX data was mined as well as a human DRG-specific dataset (SRA runs SRR8533960-SRR8533986) to identify 110 alternative exons that show differential inclusion in these tissues (Table 5), and 96 exon cassettes were selected to test for splicing behavior within these tissues. A similar procedure as outlined above was followed; alternative exons that are <200 nucleotides in length were selected, all ATGs within the alternative exon body were removed, and the end of each alternative exon was modified to terminate in ATG. The 5′ splice sites of the new exons were scored and new variants for each alternative exon cassette were designed that were 1 bit weaker, similar, and 1 bit stronger than the endogenous 5′ splice site in the absence of adjustments to generate a new ATG. −500 nucleotides of total sequence were included from each alternative exon cassette, including the alternative exon itself and immediately flanking intronic regions, and were cloned into the SMN1 exon 6/intron 7 context (as above). EGFP was used as the downstream cargo (rather than MTM1). A similar 10 nucleotide barcode was incorporated into the EGFP coding sequence to allow for identification of each alternative exon cassette. Two versions of the library were generated; one driven by an MHCK7 promoter to bias expression towards cardiac, smooth, and skeletal muscles, and the other driven by a CBh promoter to drive ubiquitous expression. The MHCK7 promoter-driven construct will be packaged by the eMyoAAV capsid to bias delivery to muscle, whereas the CBh promoter-driven construct will be packaged by the PHP.eB capsid (25) to bias delivery to the nervous system, including DRG.

TABLE 5
Part 1 (Columns 1-18): Table of exons screened to characterize behavior across multiple tissues.

| | | | | | | | Exon | | |
|---|---|---|---|---|---|---|---|---|---|
| | | | | | | | sequence | | |
| | | | | | | | (with | | |
| | | | | | | | internal | | |
| | | | | | Native | | ATGs | | |
| | | | | | 5′ | | removed | Compensated | |
| | | | Upstream | | splice | Native | and ATG | 5′ splice | |
| | | | intron | Exon | site | 5′ | at the | site | |
| | | | sequence | sequence | sequence | splice | end) | sequence | Compensated |
| | | Exon | by SEQ | by SEQ | by SEQ | site | by SEQ | by SEQ | 5′ splice |
| Coordinates | Gene | Length | ID NO: | ID NO: | ID NO: | score | ID NO: | ID NO: | site score |
| chr1_114739891_114741526_114741674_114749820 | CSDE1 | 147 | 779 | 889 | 999 | 7.96 | 1109 | 1219 | 7.82 |
| chr1_149010596_149012590_149012777_149016298 | PDE4DIP | 186 | 780 | 890 | 1000 | 5.89 | 1110 | 1220 | 5.88 |
| chr1_150625809_150626478_150626527_150627311 | ENSA | 48 | 781 | 891 | 1001 | 6.18 | 1111 | 1221 | 6.18 |
| chr1_154157722_154169304_154169384_154170399 | TPM3 | 79 | 782 | 892 | 1002 | 2.73 | 1112 | 1222 | 2.73 |
| chr1_168004794_168015780_168015952_168019544 | DCAF6 | 171 | 783 | 893 | 1003 | 3.49 | 1113 | 1223 | 3.5 |
| chr1_168015952_168019544_168019668_168022987 | DCAF6 | 123 | 784 | 894 | 1004 | 9.79 | 1114 | 1224 | 9.89 |
| chr1_19148627_19149762_19149796_19150576 | UBR4 | 33 | 785 | 895 | 1005 | 6.1 | 1115 | 1225 | 6.1 |
| chr1_19339618_19342751_19342865_19344357 | CAPZB | 113 | 786 | 896 | 1006 | 8.63 | 1116 | 1226 | 8.6 |
| chr1_29053313_29058588_29058646_29060421 | EPB41 | 57 | 787 | 897 | 1007 | 7.44 | 1117 | 1227 | 7.42 |
| chr1_46583330_46585873_46585975_46594112 | MKNK1 | 101 | 788 | 898 | 1008 | 7.04 | 1118 | 1228 | 7.07 |
| chr1_51750196_51756319_51756359_51760689 | OSBPL9 | 39 | 789 | 899 | 1009 | 8.65 | 1119 | 1229 | 8.6 |
| chr10_122228009_122229345_122229487_122237394 | TACC2 | 141 | 790 | 900 | 1010 | 7.44 | 1120 | 1230 | 7.42 |
| chr10_13597536_13597644_13597672_13600243 | PRPF18 | 27 | 791 | 901 | 1011 | 8.55 | 1121 | 1231 | 8.57 |
| chr10_24474061_24494499_24494605_24495146 | KIAA1217 | 105 | 792 | 902 | 1012 | 6.02 | 1122 | 1232 | 6.05 |
| chr10_24536894_24542506_24542543_24542692 | KIAA1217 | 36 | 793 | 903 | 1013 | 8.14 | 1123 | 1233 | 8.21 |
| chr10_29524716_29526960_29527057_29529704 | SVIL | 96 | 794 | 904 | 1014 | 9.04 | 1124 | 1234 | 9.26 |
| chr10_310093_311536_311567_327005 | DIP2C | 30 | 795 | 905 | 1015 | 9.46 | 1125 | 1235 | 9.48 |
| chr10_60042760_60053676_60053728_60055657 | ANK3 | 51 | 796 | 906 | 1016 | 8.38 | 1126 | 1236 | 8.35 |
| chr10_73388415_73396043_73396110_73396518 | ANXA7 | 66 | 797 | 907 | 1017 | 7.97 | 1127 | 1237 | 7.82 |
| chr10_76877891_76884988_76885018_76887290 | KCNMA1 | 29 | 798 | 908 | 1018 | 9.11 | 1128 | 1238 | 9.26 |
| chr10_7802855_7806973_7807011_7807658 | ATP5C1 | 37 | 799 | 909 | 1019 | 8.27 | 1129 | 1239 | 8.27 |
| chr10_95367700_95371325_95371428_95375972 | SORBS1 | 102 | 800 | 910 | 1020 | 7.51 | 1130 | 1240 | 7.42 |
| chr10_95367700_95371983_95372050_95375972 | SORBS1 | 66 | 801 | 911 | 1021 | 8.83 | 1131 | 1241 | 8.6 |
| chr10_95384286_95395000_95395076_95397219 | SORBS1 | 75 | 802 | 912 | 1022 | 7.87 | 1132 | 1242 | 7.82 |
| chr10_95432577_95434621_95434718_95437489 | SORBS1 | 96 | 803 | 913 | 1023 | 7.33 | 1133 | 1243 | 7.42 |
| chr11_113805047_113806488_113806585_113808297 | USP28 | 96 | 804 | 914 | 1024 | 7.56 | 1134 | 1244 | 7.61 |
| chr11_115178776_115198405_115198439_115214607 | CADM1 | 33 | 805 | 915 | 1025 | 6.35 | 1135 | 1245 | 6.41 |
| chr11_12239586_12243986_12244113_12249183 | MICAL2 | 126 | 806 | 916 | 1026 | 6.44 | 1136 | 1246 | 6.41 |
| chr11_65344070_65344579_65344622_65345665 | DPF2 | 42 | 807 | 917 | 1027 | 5.82 | 1137 | 1247 | 5.88 |
| chr11_70378196_70382087_70382159_70383002 | PPFIA1 | 71 | 808 | 918 | 1028 | 9.27 | 1138 | 1248 | 9.26 |
| chr11_73420000_73425021_73425049_73430689 | FAM168A | 27 | 809 | 919 | 1029 | 6.66 | 1139 | 1249 | 6.66 |
| chr11_7639871_7641032_7641066_7641478 | PPFIBP2 | 33 | 810 | 920 | 1030 | 5.9 | 1140 | 1250 | 5.88 |
| chr12_101680530_101684381_101684425_101685581 | MYBPC1 | 43 | 811 | 921 | 1031 | 10.22 | 1141 | 1251 | 10.13 |
| chr12_122341698_122347374_122347480_122352725 | CLIP1 | 105 | 812 | 922 | 1032 | 5.98 | 1142 | 1252 | 6.05 |
| chr12_128899288_128906906_128906955_128912423 | GLT1D1 | 48 | 813 | 923 | 1033 | 9.6 | 1143 | 1253 | 9.48 |
| chr12_39311554_39315931_39315971_39319905 | KIF21A | 39 | 814 | 924 | 1034 | 8.17 | 1144 | 1254 | 8.21 |
| chr12_39315971_39318072_39318202_39319905 | KIF21A | 129 | 815 | 925 | 1035 | 8.07 | 1145 | 1255 | 8.21 |
| chr12_55699990_55700261_55700394_55700898 | ITGA7 | 132 | 816 | 926 | 1036 | 4.44 | 1146 | 1256 | 4.52 |
| chr12_71157788_71277350_71277413_71282715 | TSPAN8 | 62 | 817 | 927 | 1037 | 8.34 | 1147 | 1257 | 8.35 |
| chr12_89591296_89598656_89598744_89599116 | ATP2B1 | 87 | 818 | 928 | 1038 | 2.78 | 1148 | 1258 | 2.77 |
| chr13_75312899_75324236_75324402_75326196 | TBC1D4 | 165 | 819 | 929 | 1039 | 9.22 | 1149 | 1259 | 9.26 |
| chr13_75836458_75838139_75838197_75840084 | LMO7 | 57 | 820 | 930 | 1040 | 8.14 | 1150 | 1260 | 8.21 |
| chr13_75838197_75838346_75838389_75840084 | LMO7 | 42 | 821 | 931 | 1041 | 4.46 | 1151 | 1261 | 4.52 |
| chr14_100831535_100835738_100835880_100836166 | MEG3 | 141 | 822 | 932 | 1042 | 6.29 | 1152 | 1262 | 6.18 |
| chr14_35690004_35700161_35700303_35721687 | RALGAPA1 | 141 | 823 | 933 | 1043 | 6.13 | 1153 | 1263 | 6.1 |
| chr14_90593627_90603252_90603304_90610741 | TTC7B | 51 | 824 | 934 | 1044 | 7.79 | 1154 | 1264 | 7.82 |
| chr15_20458448_20461191_20461343_20462342 | HERC2P3 | 151 | 825 | 935 | 1045 | 5.9 | 1155 | 1265 | 5.88 |
| chr15_33842036_33843487_33843575_33844861 | RYR3 | 87 | 826 | 936 | 1046 | 4.18 | 1156 | 1266 | 4.16 |
| chr15_47766678_47768580_47768749_47770496 | SEMA6D | 168 | 827 | 937 | 1047 | 9.16 | 1157 | 1267 | 9.26 |
| chr16_119256_123506_123546_124936 | NPRL3 | 39 | 828 | 938 | 1048 | 9.21 | 1158 | 1268 | 9.26 |
| chr16_2264761_2266109_22662172267662 | RNPS1 | 107 | 829 | 939 | 1049 | 8.68 | 1159 | 1269 | 8.6 |
| chr16_30201107_30201195_30201294_30202689 | SLX1A- | 98 | 830 | 940 | 1050 | 9.16 | 1160 | 1270 | 9.26 |
| | SULT1A3 | | | | | | | | |
| chr16_58509353_58510644_58510684_58511421 | NDRG4 | 39 | 831 | 941 | 1051 | 7.13 | 1161 | 1271 | 7.14 |
| chr17_17850953_17857768_17857856_17861475 | TOM1L2 | 87 | 832 | 942 | 1052 | 7.61 | 1162 | 1272 | 7.61 |
| chr17_73207257_73207701_73207740_73208313 | COG1 | 38 | 833 | 943 | 1053 | 3.4 | 1163 | 1273 | 3.39 |
| chr17_76089321_76090328_76090398_76091142 | EXOC7 | 69 | 834 | 944 | 1054 | 10.9 | 1164 | 1274 | 11.01 |
| chr17_76089321_76090328_76090482_76091142 | EXOC7 | 153 | 835 | 945 | 1055 | 10.9 | 1165 | 1275 | 11.01 |
| chr18_34882202_34884727_34884777_34890299 | DTNA | 49 | 836 | 946 | 1056 | 10.08 | 1166 | 1276 | 10.13 |
| chr18_5397427_5398020_5398144_5406776 | EPB41L3 | 123 | 837 | 947 | 1057 | 3.48 | 1167 | 1277 | 3.5 |
| chr18_58333893_58341677_58341798_58342905 | NEDD4L | 120 | 838 | 948 | 1058 | 7.66 | 1168 | 1278 | 7.61 |
| chr19_45297955_45298142_45298223_45299810 | MARK4 | 80 | 839 | 949 | 1059 | 9.14 | 1169 | 1279 | 9.26 |
| chr19_49146006_49146165_49146193_49148082 | PPFIA3 | 27 | 840 | 950 | 1060 | 9.46 | 1170 | 1280 | 9.48 |
| chr2_121410970_121418621_121418730_121425138 | CLASP1 | 108 | 841 | 951 | 1061 | 9.27 | 1171 | 1281 | 9.26 |
| chr2_127059156_127060595_127060641_127062114 | BIN1 | 45 | 842 | 952 | 1062 | 10.05 | 1172 | 1282 | 10.13 |
| chr2_171707378_171712766_171712827_171715327 | DYNC112 | 60 | 843 | 953 | 1063 | 2.65 | 1173 | 1283 | 2.63 |
| chr2_178535829_178536333_178536463_178537603 | TTN-AS1 | 129 | 844 | 954 | 1064 | 5.69 | 1174 | 1284 | 5.75 |
| chr2_237749325_237751199_237751272_237753308 | LRRFIP1 | 72 | 845 | 955 | 1065 | 7.87 | 1175 | 1285 | 7.82 |
| chr2_240771105_240772569_240772597_240773113 | KIF1A | 27 | 846 | 956 | 1066 | 2.42 | 1176 | 1286 | 2.42 |
| chr2_27094612_27094799_27094935_27096728 | KHK | 135 | 847 | 957 | 1067 | 7.47 | 1177 | 1287 | 7.42 |
| chr2_40177856_40178386_40178494_40428472 | SLC8A1 | 107 | 848 | 958 | 1068 | 5.87 | 1178 | 1288 | 5.88 |
| chr2_69350184_69354258_69354313_69354488 | GFPT1 | 54 | 849 | 959 | 1069 | 4.3 | 1179 | 1289 | 4.36 |
| chr2_86166645_86170748_86170842_86171207 | IMMT | 93 | 850 | 960 | 1070 | 4.8 | 1180 | 1290 | 4.79 |
| chr2_8737000_8747144_8747202_8747886 | KIDINS220 | 57 | 851 | 961 | 1071 | 5.98 | 1181 | 1291 | 6.05 |
| chr2_96605512_96606969_96607048_96608507 | KANSL3 | 78 | 852 | 962 | 1072 | 9.27 | 1182 | 1292 | 9.26 |
| chr3_111949076_111949701_111949891_111952571 | PHLDB2 | 189 | 853 | 963 | 1073 | 8.55 | 1183 | 1293 | 8.57 |
| chr3_111958296_111960144_111960181_111962107 | PHLDB2 | 36 | 854 | 964 | 1074 | 8.88 | 1184 | 1294 | 8.6 |
| chr3_121835392_121836644_121836795_121844452 | EAF2 | 150 | 855 | 965 | 1075 | 9.37 | 1185 | 1295 | 9.33 |
| chr3_180970359_180971074_180971156_180975312 | FXR1 | 81 | 856 | 966 | 1076 | 8.63 | 1186 | 1296 | 8.6 |
| chr3_37065943_37066223_37066326_37075023 | LRRFIP2 | 102 | 857 | 967 | 1077 | 9.81 | 1187 | 1297 | 9.89 |
| chr3_37066326_37072789_37072883_37075023 | LRRFIP2 | 93 | 858 | 968 | 1078 | 5.41 | 1188 | 1298 | 5.39 |
| chr3_37121692_37127629_37127681_37129062 | LRRFIP2 | 51 | 859 | 969 | 1079 | 6.99 | 1189 | 1299 | 7.07 |
| chr3_52679727_52681703_52681821_52682091 | PBRM1 | 117 | 860 | 970 | 1080 | 5.75 | 1190 | 1300 | 5.75 |
| chr3_62499269_62513637_62513707_62516058 | CADPS | 69 | 861 | 971 | 1081 | 7.9 | 1191 | 1301 | 7.82 |
| chr4_101026062_101029165_101029196_101032266 | PPP3CA | 30 | 862 | 972 | 1082 | 8.88 | 1192 | 1302 | 8.6 |
| chr4_113330471_113331971_113332071_113333053 | ANK2 | 99 | 863 | 973 | 1083 | 5.12 | 1193 | 1303 | 5.19 |
| chr4_113373450_113378089_113378182_113381456 | ANK2 | 92 | 864 | 974 | 1084 | 5.28 | 1194 | 1304 | 5.29 |
| chr4_113500512_113502935_113502978_113509637 | CAMK2D | 42 | 865 | 975 | 1085 | 6.8 | 1195 | 1305 | 6.85 |
| chr4_1802026_1802913_1803065_1803691 | FGFR3 | 151 | 866 | 976 | 1086 | 10.52 | 1196 | 1306 | 10.13 |
| chr4_185514338_185514702_185514891_185523361 | PDLIM3 | 188 | 867 | 977 | 1087 | 9.72 | 1197 | 1307 | 9.89 |
| chr4_42556041_42569160_42569206_42574618 | ATP8A1 | 45 | 868 | 978 | 1088 | 9.26 | 1198 | 1308 | 9.26 |
| chr4_8009103_8019617_8019672_8029655 | ABLIM2 | 54 | 869 | 979 | 1089 | 8.21 | 1199 | 1309 | 8.21 |
| chr4_82829059_82830936_82830976_82842139 | SEC31A | 39 | 870 | 980 | 1090 | 6.38 | 1200 | 1310 | 6.41 |
| chr5_103187377_103189166_103189227_103190841 | PPIP5K2 | 60 | 871 | 981 | 1091 | 7.64 | 1201 | 1311 | 7.61 |
| chr5_88823928_88882954_88883000_88887501 | MEF2C | 45 | 872 | 982 | 1092 | 8.31 | 1202 | 1312 | 8.35 |
| chr6_138433681_138441982_138442115_138447000 | NHSL1 | 132 | 873 | 983 | 1093 | 7.37 | 1203 | 1313 | 7.42 |
| chr6_160143650_160155974_160156075_160158515 | SLC22A1 | 100 | 874 | 984 | 1094 | 4.41 | 1204 | 1314 | 4.36 |
| chr6_29608734_29609228_29609380_29610923 | GABBR1 | 151 | 875 | 985 | 1095 | 10.91 | 1205 | 1315 | 11.01 |
| chr6_54160433_54160739_54160800_54169527 | MLIP | 60 | 876 | 986 | 1096 | 6.93 | 1206 | 1316 | 6.85 |
| chr6_56463765_56464684_56464757_56466077 | DST | 72 | 877 | 987 | 1097 | 2.04 | 1207 | 1317 | 2.04 |
| chr6_75892691_75894813_75894841_75895230 | MYO6 | 27 | 878 | 988 | 1098 | 5.17 | 1208 | 1318 | 5.19 |
| chr7_128849579_128849975_128850075_128850383 | FLNC | 99 | 879 | 989 | 1099 | 2.97 | 1209 | 1319 | 2.95 |
| chr7_44234677_44239588_44239664_44240706 | CAMK2B | 75 | 880 | 990 | 1100 | 7.24 | 1210 | 1320 | 7.23 |
| chr7_51184200_51187913_51187959_51190849 | COBL | 45 | 881 | 991 | 1101 | 8.04 | 1211 | 1321 | 8.21 |
| chr7_74743521_74744138_74744251_74744757 | GTF21 | 112 | 882 | 992 | 1102 | 6.38 | 1212 | 1322 | 6.41 |
| chr7_81997251_82001658_82001716_82005422 | CACNA2D1 | 57 | 883 | 993 | 1103 | 8.63 | 1213 | 1323 | 8.6 |
| chr8_11861480_11864375_11864484_11868000 | CTSB | 108 | 884 | 994 | 1104 | 8.01 | 1214 | 1324 | 7.82 |
| chr8_22528578_22531301_22531329_22532224 | PPP3CC | 27 | 885 | 995 | 1105 | 8.33 | 1215 | 1325 | 8.35 |
| chr9_105512937_105513598_105513632_105534495 | FSD1L | 33 | 886 | 996 | 1106 | 5.22 | 1216 | 1326 | 5.19 |
| chrX_103962724_103964227_103964295_103964505 | TMSB15B | 67 | 887 | 997 | 1107 | 9.48 | 1217 | 1327 | 9.48 |
| chrX_15827373_15840496_15840592_15845378 | AP1S2 | 95 | 888 | 998 | 1108 | 4.27 | 1218 | 1328 | 4.36 |
| | | | Downstream | | | | | | |
| | | | intron | | | 5′ | | 5′ | |
| | | | sequence | | | splice | | splice | |
| | | | (with | | | site | 5′ | site | 5′ |
| | | Downstream | compensated | | | (~1 bit | splice | (~1 bit | splice |
| | | intron | 5′ splice | Kozak | | stronger) | site | weaker) | site |
| | | sequence | site) | sequence | Kozak | sequence | score | sequence | score |
| | | by SEQ | by SEQ | by SEQ | sequence | by SEQ | (~1 bit | by SEQ | (~1 bit |
| Coordinates | Gene | ID NO: | ID NO: | ID NO: | score | ID NO: | stronger) | ID NO: | weaker) |
| chr1_114739891_114741526_114741674_114749820 | CSDE1 | 1329 | 1439 | 1549 | 75 | 1659 | 9.26 | 1769 | 7.07 |
| chr1_149010596_149012590_149012777_149016298 | PDE4DIP | 1330 | 1440 | 1550 | 74 | 1660 | 6.85 | 1770 | 4.88 |
| chr1_150625809_150626478_150626527_150627311 | ENSA | 1331 | 1441 | 1551 | 53 | 1661 | 7.14 | 1771 | 5.19 |
| chr1_154157722_154169304_154169384_154170399 | TPM3 | 1332 | 1442 | 1552 | 57 | 1662 | 3.85 | 1772 | 1.72 |
| chr1_168004794_168015780_168015952_168019544 | DCAF6 | 1333 | 1443 | 1553 | 38 | 1663 | 4.52 | 1773 | 2.49 |
| chr1_168015952_168019544_168019668_168022987 | DCAF6 | 1334 | 1444 | 1554 | 76 | 1664 | 11.01 | 1774 | 8.6 |
| chr1_19148627_19149762_19149796_19150576 | UBR4 | 1335 | 1445 | 1555 | 79 | 1665 | 7.07 | 1775 | 5.05 |
| chr1_19339618_19342751_19342865_19344357 | CAPZB | 1336 | 1446 | 1556 | 86 | 1666 | 9.48 | 1776 | 7.61 |
| chr1_29053313_29058588_29058646_29060421 | EPB41 | 1337 | 1447 | 1557 | 76 | 1667 | 8.35 | 1777 | 6.41 |
| chr1_46583330_46585873_46585975_46594112 | MKNK1 | 1338 | 1448 | 1558 | 57 | 1668 | 8.21 | 1778 | 6.05 |
| chr1_51750196_51756319_51756359_51760689 | OSBPL9 | 1339 | 1449 | 1559 | 81 | 1669 | 9.48 | 1779 | 7.61 |
| chr10_122228009_122229345_122229487_122237394 | TACC2 | 1340 | 1450 | 1560 | 59 | 1670 | 8.35 | 1780 | 6.41 |
| chr10_13597536_13597644_13597672_13600243 | PRPF18 | 1341 | 1451 | 1561 | 79 | 1671 | 9.48 | 1781 | 7.61 |
| chr10_24474061_24494499_24494605_24495146 | KIAA1217 | 1342 | 1452 | 1562 | 84 | 1672 | 7.07 | 1782 | 5.05 |
| chr10_24536894_24542506_24542543_24542692 | KIAA1217 | 1343 | 1453 | 1563 | 48 | 1673 | 9.26 | 1783 | 7.14 |
| chr10_29524716_29526960_29527057_29529704 | SVIL | 1344 | 1454 | 1564 | 39 | 1674 | 10.13 | 1784 | 8.21 |
| chr10_310093_311536_311567_327005 | DIP2C | 1345 | 1455 | 1565 | 76 | 1675 | 10.13 | 1785 | 8.57 |
| chr10_60042760_60053676_60053728_60055657 | ANK3 | 1346 | 1456 | 1566 | 27 | 1676 | 9.33 | 1786 | 7.42 |
| chr10_73388415_73396043_73396110_73396518 | ANXA7 | 1347 | 1457 | 1567 | 85 | 1677 | 9.26 | 1787 | 7.07 |
| chr10_76877891_76884988_76885018_76887290 | KCNMA1 | 1348 | 1458 | 1568 | 77 | 1678 | 10.13 | 1788 | 8.21 |
| chr10_7802855_7806973_7807011_7807658 | ATP5C1 | 1349 | 1459 | 1569 | 79 | 1679 | 9.26 | 1789 | 7.23 |
| chr10_95367700_95371325_95371428_95375972 | SORBS1 | 1350 | 1460 | 1570 | 71 | 1680 | 8.57 | 1790 | 6.49 |
| chr10_95367700_95371983_95372050_95375972 | SORBS1 | 1351 | 1461 | 1571 | 71 | 1681 | 9.89 | 1791 | 7.82 |
| chr10_95384286_95395000_95395076_95397219 | SORBS1 | 1352 | 1462 | 1572 | 63 | 1682 | 8.6 | 1792 | 6.85 |
| chr10_95432577_95434621_95434718_95437489 | SORBS1 | 1353 | 1463 | 1573 | 74 | 1683 | 8.35 | 1793 | 6.41 |
| chr11_113805047_113806488_113806585_113808297 | USP28 | 1354 | 1464 | 1574 | 70 | 1684 | 8.57 | 1794 | 6.56 |
| chr11_115178776_115198405_115198439_115214607 | CADM1 | 1355 | 1465 | 1575 | 30 | 1685 | 7.42 | 1795 | 5.31 |
| chr11_12239586_12243986_12244113_12249183 | MICAL2 | 1356 | 1466 | 1576 | 85 | 1686 | 7.42 | 1796 | 5.46 |
| chr11_65344070_65344579_65344622_65345665 | DPF2 | 1357 | 1467 | 1577 | 86 | 1687 | 6.85 | 1797 | 4.84 |
| chr11_70378196_70382087_70382159_70383002 | PPFIA1 | 1358 | 1468 | 1578 | 65 | 1688 | 10.13 | 1798 | 8.27 |
| chr11_73420000_73425021_73425049_73430689 | FAM168A | 1359 | 1469 | 1579 | 33 | 1689 | 7.61 | 1799 | 5.75 |
| chr11_7639871_7641032_7641066_7641478 | PPFIBP2 | 1360 | 1470 | 1580 | 32 | 1690 | 6.85 | 1800 | 4.9 |
| chr12_101680530_101684381_101684425_101685581 | MYBPC1 | 1361 | 1471 | 1581 | 53 | 1691 | 11.01 | 1801 | 9.26 |
| chr12_122341698_122347374_122347480_122352725 | CLIP1 | 1362 | 1472 | 1582 | 79 | 1692 | 7.07 | 1802 | 5.05 |
| chr12_128899288_128906906_128906955_128912423 | GLT1D1 | 1363 | 1473 | 1583 | 70 | 1693 | 11.01 | 1803 | 8.6 |
| chr12_39311554_39315931_39315971_39319905 | KIF21A | 1364 | 1474 | 1584 | 85 | 1694 | 9.26 | 1804 | 7.14 |
| chr12_39315971_39318072_39318202_39319905 | KIF21A | 1365 | 1475 | 1585 | 91 | 1695 | 9.26 | 1805 | 7.07 |
| chr12_55699990_55700261_55700394_55700898 | ITGA7 | 1366 | 1476 | 1586 | 79 | 1696 | 5.46 | 1806 | 3.39 |
| chr12_71157788_71277350_71277413_71282715 | TSPAN8 | 1367 | 1477 | 1587 | 69 | 1697 | 9.33 | 1807 | 7.42 |
| chr12_89591296_89598656_89598744_89599116 | ATP2B1 | 1368 | 1478 | 1588 | 36 | 1698 | 3.85 | 1808 | 1.72 |
| chr13_75312899_75324236_75324402_75326196 | TBC1D4 | 1369 | 1479 | 1589 | 79 | 1699 | 10.13 | 1809 | 8.21 |
| chr13_75836458_75838139_75838197_75840084 | LMO7 | 1370 | 1480 | 1590 | 83 | 1700 | 9.26 | 1810 | 7.14 |
| chr13_75838197_75838346_75838389_75840084 | LMO7 | 1371 | 1481 | 1591 | 50 | 1701 | 5.46 | 1811 | 3.5 |
| chr14_100831535_100835738_100835880_100836166 | MEG3 | 1372 | 1482 | 1592 | 71 | 1702 | 7.23 | 1812 | 5.29 |
| chr14_35690004_35700161_35700303_35721687 | RALGAPA1 | 1373 | 1483 | 1593 | 39 | 1703 | 7.14 | 1813 | 5.19 |
| chr14_90593627_90603252_90603304_90610741 | TTC7B | 1374 | 1484 | 1594 | 62 | 1704 | 8.6 | 1814 | 6.85 |
| chr15_20458448_20461191_20461343_20462342 | HERC2P3 | 1375 | 1485 | 1595 | 32 | 1705 | 6.85 | 1815 | 4.9 |
| chr15_33842036_33843487_33843575_33844861 | RYR3 | 1376 | 1486 | 1596 | 30 | 1706 | 5.19 | 1816 | 3.18 |
| chr15_47766678_47768580_47768749_47770496 | SEMA6D | 1377 | 1487 | 1597 | 80 | 1707 | 10.13 | 1817 | 8.21 |
| chr16_119256_123506_123546_124936 | NPRL3 | 1378 | 1488 | 1598 | 27 | 1708 | 10.13 | 1818 | 8.21 |
| chr16_2264761_2266109_22662172267662 | RNPS1 | 1379 | 1489 | 1599 | 80 | 1709 | 9.48 | 1819 | 7.61 |
| chr16_30201107_30201195_30201294_30202689 | SLX1A- | 1380 | 1490 | 1600 | 80 | 1710 | 10.13 | 1820 | 8.21 |
| | SULT1A3 | | | | | | | | |
| chr16_58509353_58510644_58510684_58511421 | NDRG4 | 1381 | 1491 | 1601 | 87 | 1711 | 8.21 | 1821 | 6.1 |
| chr17_17850953_17857768_17857856_17861475 | TOM1L2 | 1382 | 1492 | 1602 | 76 | 1712 | 8.6 | 1822 | 6.63 |
| chr17_73207257_73207701_73207740_73208313 | COG1 | 1383 | 1493 | 1603 | 54 | 1713 | 4.36 | 1823 | 2.4 |
| chr17_76089321_76090328_76090398_76091142 | EXOC7 | 1384 | 1494 | 1604 | 36 | 1714 | 11.01 | 1824 | 9.89 |
| chr17_76089321_76090328_76090482_76091142 | EXOC7 | 1385 | 1495 | 1605 | 36 | 1715 | 11.01 | 1825 | 9.89 |
| chr18_34882202_34884727_34884777_34890299 | DTNA | 1386 | 1496 | 1606 | 49 | 1716 | 11.01 | 1826 | 9.26 |
| chr18_5397427_5398020_5398144_5406776 | EPB41L3 | 1387 | 1497 | 1607 | 59 | 1717 | 4.52 | 1827 | 2.49 |
| chr18_58333893_58341677_58341798_58342905 | NEDD4L | 1388 | 1498 | 1608 | 55 | 1718 | 8.6 | 1828 | 6.66 |
| chr19_45297955_45298142_45298223_45299810 | MARK4 | 1389 | 1499 | 1609 | 87 | 1719 | 10.13 | 1829 | 8.21 |
| chr19_49146006_49146165_49146193_49148082 | PPFIA3 | 1390 | 1500 | 1610 | 74 | 1720 | 10.13 | 1830 | 8.57 |
| chr2_121410970_121418621_121418730_121425138 | CLASP1 | 1391 | 1501 | 1611 | 64 | 1721 | 10.13 | 1831 | 8.27 |
| chr2_127059156_127060595_127060641_127062114 | BIN1 | 1392 | 1502 | 1612 | 86 | 1722 | 11.01 | 1832 | 9.26 |
| chr2_171707378_171712766_171712827_171715327 | DYNC112 | 1393 | 1503 | 1613 | 33 | 1723 | 3.59 | 1833 | 1.69 |
| chr2_178535829_178536333_178536463_178537603 | TTN-AS1 | 1394 | 1504 | 1614 | 52 | 1724 | 6.66 | 1834 | 4.68 |
| chr2_237749325_237751199_237751272_237753308 | LRRFIP1 | 1395 | 1505 | 1615 | 76 | 1725 | 8.6 | 1835 | 6.85 |
| chr2_240771105_240772569_240772597_240773113 | KIF1A | 1396 | 1506 | 1616 | 64 | 1726 | 3.39 | 1836 | 1.35 |
| chr2_27094612_27094799_27094935_27096728 | KHK | 1397 | 1507 | 1617 | 52 | 1727 | 8.57 | 1837 | 6.49 |
| chr2_40177856_40178386_40178494_40428472 | SLC8A1 | 1398 | 1508 | 1618 | 87 | 1728 | 6.85 | 1838 | 4.87 |
| chr2_69350184_69354258_69354313_69354488 | GFPT1 | 1399 | 1509 | 1619 | 86 | 1729 | 5.31 | 1839 | 3.32 |
| chr2_86166645_86170748_86170842_86171207 | IMMT | 1400 | 1510 | 1620 | 41 | 1730 | 5.75 | 1840 | 3.85 |
| chr2_8737000_8747144_8747202_8747886 | KIDINS220 | 1401 | 1511 | 1621 | 60 | 1731 | 7.07 | 1841 | 5.05 |
| chr2_96605512_96606969_96607048_96608507 | KANSL3 | 1402 | 1512 | 1622 | 59 | 1732 | 10.13 | 1842 | 8.27 |
| chr3_111949076_111949701_111949891_111952571 | PHLDB2 | 1403 | 1513 | 1623 | 73 | 1733 | 9.48 | 1843 | 7.61 |
| chr3_111958296_111960144_111960181_111962107 | PHLDB2 | 1404 | 1514 | 1624 | 78 | 1734 | 9.89 | 1844 | 7.82 |
| chr3_121835392_121836644_121836795_121844452 | EAF2 | 1405 | 1515 | 1625 | 35 | 1735 | 10.13 | 1845 | 8.35 |
| chr3_180970359_180971074_180971156_180975312 | FXR1 | 1406 | 1516 | 1626 | 67 | 1736 | 9.48 | 1846 | 7.61 |
| chr3_37065943_37066223_37066326_37075023 | LRRFIP2 | 1407 | 1517 | 1627 | 96 | 1737 | 11.01 | 1847 | 8.6 |
| chr3_37066326_37072789_37072883_37075023 | LRRFIP2 | 1408 | 1518 | 1628 | 69 | 1738 | 6.41 | 1848 | 4.36 |
| chr3_37121692_37127629_37127681_37129062 | LRRFIP2 | 1409 | 1519 | 1629 | 28 | 1739 | 7.82 | 1849 | 6.05 |
| chr3_52679727_52681703_52681821_52682091 | PBRM1 | 1410 | 1520 | 1630 | 69 | 1740 | 6.66 | 1850 | 4.79 |
| chr3_62499269_62513637_62513707_62516058 | CADPS | 1411 | 1521 | 1631 | 63 | 1741 | 8.6 | 1851 | 6.85 |
| chr4_101026062_101029165_101029196_101032266 | PPP3CA | 1412 | 1522 | 1632 | 70 | 1742 | 9.89 | 1852 | 7.82 |
| chr4_113330471_113331971_113332071_113333053 | ANK2 | 1413 | 1523 | 1633 | 44 | 1743 | 6.1 | 1853 | 4.14 |
| chr4_113373450_113378089_113378182_113381456 | ANK2 | 1414 | 1524 | 1634 | 45 | 1744 | 6.18 | 1854 | 4.36 |
| chr4_113500512_113502935_113502978_113509637 | CAMK2D | 1415 | 1525 | 1635 | 78 | 1745 | 7.82 | 1855 | 5.75 |
| chr4_1802026_1802913_1803065_1803691 | FGFR3 | 1416 | 1526 | 1636 | 75 | 1746 | 11.01 | 1856 | 9.48 |
| chr4_185514338_185514702_185514891_185523361 | PDLIM3 | 1417 | 1527 | 1637 | 72 | 1747 | 11.0 | 1857 | 8.6 |
| chr4_42556041_42569160_42569206_42574618 | ATP8A1 | 1418 | 1528 | 1638 | 50 | 1748 | 10.13 | 1858 | 8.27 |
| chr4_8009103_8019617_8019672_8029655 | ABLIM2 | 1419 | 1529 | 1639 | 67 | 1749 | 9.26 | 1859 | 7.23 |
| chr4_82829059_82830936_82830976_82842139 | SEC31A | 1420 | 1530 | 1640 | 29 | 1750 | 7.42 | 1860 | 5.39 |
| chr5_103187377_103189166_103189227_103190841 | PPIP5K2 | 1421 | 1531 | 1641 | 74 | 1751 | 8.6 | 1861 | 6.63 |
| chr5_88823928_88882954_88883000_88887501 | MEF2C | 1422 | 1532 | 1642 | 52 | 1752 | 9.33 | 1862 | 7.23 |
| chr6_138433681_138441982_138442115_138447000 | NHSL1 | 1423 | 1533 | 1643 | 82 | 1753 | 8.35 | 1863 | 6.41 |
| chr6_160143650_160155974_160156075_160158515 | SLC22A1 | 1424 | 1534 | 1644 | 29 | 1754 | 5.39 | 1864 | 3.39 |
| chr6_29608734_29609228_29609380_29610923 | GABBR1 | 1425 | 1535 | 1645 | 40 | 1755 | 11.01 | 1865 | 9.89 |
| chr6_54160433_54160739_54160800_54169527 | MLIP | 1426 | 1536 | 1646 | 90 | 1756 | 7.82 | 1866 | 5.88 |
| chr6_56463765_56464684_56464757_56466077 | DST | 1427 | 1537 | 1647 | 70 | 1757 | 3.06 | 1867 | 1.05 |
| chr6_75892691_75894813_75894841_75895230 | MYO6 | 1428 | 1538 | 1648 | 44 | 1758 | 6.18 | 1868 | 4.16 |
| chr7_128849579_128849975_128850075_128850383 | FLNC | 1429 | 1539 | 1649 | 74 | 1759 | 4.01 | 1869 | 1.95 |
| chr7_44234677_44239588_44239664_44240706 | CAMK2B | 1430 | 1540 | 1650 | 76 | 1760 | 8.27 | 1870 | 6.18 |
| chr7_51184200_51187913_51187959_51190849 | COBL | 1431 | 1541 | 1651 | 73 | 1761 | 9.26 | 1871 | 7.07 |
| chr7_74743521_74744138_74744251_74744757 | GTF21 | 1432 | 1542 | 1652 | 87 | 1762 | 7.42 | 1872 | 5.39 |
| chr7_81997251_82001658_82001716_82005422 | CACNA2D1 | 1433 | 1543 | 1653 | 69 | 1763 | 9.48 | 1873 | 7.61 |
| chr8_11861480_11864375_11864484_11868000 | CTSB | 1434 | 1544 | 1654 | 63 | 1764 | 9.26 | 1874 | 7.07 |
| chr8_22528578_22531301_22531329_22532224 | PPP3CC | 1435 | 1545 | 1655 | 89 | 1765 | 9.33 | 1875 | 7.42 |
| chr9_105512937_105513598_105513632_105534495 | FSD1L | 1436 | 1546 | 1656 | 58 | 1766 | 6.18 | 1876 | 4.16 |
| chrX_103962724_103964227_103964295_103964505 | TMSB15B | 1437 | 1547 | 1657 | 75 | 1767 | 10.13 | 1877 | 8.57 |
| chrX_15827373_15840496_15840592_15845378 | AP1S2 | 1438 | 1548 | 1658 | 35 | 1768 | 5.29 | 1878 | 3.28 |

TABLE 5
Part 2 (Columns 1, 2, and 19-38): Table of exons screened to characterize behavior across multiple tissues.

| | | | Brain - | | | | | | | |
|---|---|---|---|---|---|---|---|---|---|---|
| | | | Anterior | Brain - | | | | Brain - | | |
| | | | cingulate | Caudate | Brain - | | | Frontal | | |
| | | Brain - | cortex | (basal | Cerebellar | Brain - | Brain - | Cortex | Brain - | Brain - |
| | | Amygdala | (BA24) | ganglia) | Hemisphere | Cerebellum | Cortex | (BA9) | Hippocampus | Hypothalamus |
| Coordinates | Gene | (psi) | (psi) | (psi) | (psi) | (psi) | (psi) | (psi) | (psi) | (psi) |
| chr1_114739891_114741526_114741674_114749820 | CSDE1 | 0.06 | 0.05 | 0.05 | 0.02 | 0.02 | 0.04 | 0.04 | 0.07 | 0.05 |
| chr1_149010596_149012590_149012777_149016298 | PDE4DIP | 0.29 | 0.39 | 0.43 | 0.83 | 0.79 | 0.51 | 0.58 | 0.43 | 0.34 |
| chr1_150625809_150626478_150626527_150627311 | ENSA | ND | ND | ND | ND | ND | ND | ND | 0.86 | 0.33 |
| chr1_154157722_154169304_154169384_154170399 | TPM3 | 0.06 | 0.08 | 0.06 | 0.35 | 0.32 | 0.11 | 0.12 | 0.08 | 0.09 |
| chr1_168004794_168015780_168015952_168019544 | DCAF6 | 0.82 | 0.88 | 0.82 | ND | 0.18 | 0.73 | 0.77 | 0.77 | 0.77 |
| chr1_168015952_168019544_168019668_168022987 | DCAF6 | 0.09 | 0.12 | 0.09 | ND | 0.4 | 0.11 | 0.09 | 0 | 0.14 |
| chr1_19148627_19149762_19149796_19150576 | UBR4 | 0.19 | 0.35 | 0.21 | 0.81 | 0.76 | 0.41 | 0.47 | 0.25 | 0.36 |
| chr1_19339618_19342751_19342865_19344357 | CAPZB | 0.33 | 0.26 | 0.37 | 0.08 | 0.11 | 0.22 | 0.18 | 0.3 | 0.23 |
| chr1_29053313_29058588_29058646_29060421 | EPB41 | ND | ND | 0.9 | 0.99 | 0.99 | 0.85 | ND | ND | 0.95 |
| chr1_46583330_46585873_46585975_46594112 | MKNK1 | 0.39 | 0.42 | 0.31 | 0.32 | 0.32 | 0.5 | 0.5 | 0.38 | 0.23 |
| chr1_51750196_51756319_51756359_51760689 | OSBPL9 | 0.46 | 0.37 | 0.4 | 0.18 | 0.21 | 0.35 | 0.33 | 0.46 | 0.35 |
| chr10_122228009_122229345_122229487_122237394 | TACC2 | 0.61 | 0.72 | 0.69 | 0.81 | 0.81 | 0.78 | 0.8 | 0.63 | 0.65 |
| chr10_13597536_13597644_13597672_13600243 | PRPF18 | 0.27 | 0.35 | 0.29 | 0.45 | 0.44 | 0.44 | 0.49 | 0.29 | 0.07 |
| chr10_24474061_24494499_24494605_24495146 | KIAA1217 | 0.9 | 0.93 | 0.91 | 0.93 | 0.27 | 0.89 | 0.96 | 0.85 | 0.78 |
| chr10_24536894_24542506_24542543_24542692 | KIAA1217 | ND | ND | ND | ND | ND | ND | ND | ND | ND |
| chr10_29524716_29526960_29527057_29529704 | SVIL | 0.03 | ND | ND | 0.22 | 0.18 | 0.33 | 0.13 | ND | 0.46 |
| chr10_310093_311536_311567_327005 | DIP2C | 0.08 | 0.05 | 0.11 | 0.04 | 0.04 | 0.06 | 0.07 | 0.1 | 0.07 |
| chr10_60042760_60053676_60053728_60055657 | ANK3 | 0.01 | 0 | 0.01 | 0 | 0 | 0.01 | 0 | 0.01 | 0.01 |
| chr10_73388415_73396043_73396110_73396518 | ANXA7 | 0.31 | 0.41 | 0.29 | 0.83 | 0.75 | 0.44 | 0.55 | 0.39 | 0.45 |
| chr10_76877891_76884988_76885018_76887290 | KCNMA1 | 0.28 | 0.29 | 0.48 | 0.51 | 0.53 | 0.31 | 0.35 | 0.28 | 0.16 |
| chr10_7802855_7806973_7807011_7807658 | ATP5C1 | 0.92 | 0.94 | 0.94 | 0.98 | 0.97 | 0.95 | 0.96 | 0.91 | 0.93 |
| chr10_95367700_95371325_95371428_95375972 | SORBS1 | 0.07 | 0.18 | 0.21 | 0.59 | 0.43 | 0.23 | 0.31 | 0.08 | 0.1 |
| chr10_95367700_95371983_95372050_95375972 | SORBS1 | 0.08 | 0.12 | 0.12 | 0.38 | 0.23 | 0.11 | 0.13 | 0.08 | 0.1 |
| chr10_95384286_95395000_95395076_95397219 | SORBS1 | 0.73 | 0.7 | 0.7 | 0.43 | 0.55 | 0.68 | 0.6 | 0.65 | 0.67 |
| chr10_95432577_95434621_95434718_95437489 | SORBS1 | 0.66 | 0.55 | 0.53 | 0.35 | 0.46 | 0.52 | 0.45 | 0.64 | 0.63 |
| chr11_113805047_113806488_113806585_113808297 | USP28 | 0.48 | 0.7 | 0.5 | 0.89 | 0.89 | 0.76 | 0.79 | 0.49 | 0.56 |
| chr11_115178776_115198405_115198439_115214607 | CADM1 | 0.66 | 0.56 | 0.7 | 0.5 | 0.5 | 0.56 | 0.51 | 0.6 | 0.52 |
| chr11_12239586_12243986_12244113_12249183 | MICAL2 | 0.99 | 0.99 | 0.96 | 1 | 0.99 | 0.99 | 0.99 | 0.98 | 0.94 |
| chr11_65344070_65344579_65344622_65345665 | DPF2 | 0.35 | 0.41 | 0.36 | 0.88 | 0.85 | 0.49 | 0.53 | 0.44 | 0.48 |
| chr11_70378196_70382087_70382159_70383002 | PPFIA1 | 0.98 | 0.98 | 0.98 | 1 | 1 | 0.99 | 0.99 | 0.98 | 0.98 |
| chr11_73420000_73425021_73425049_73430689 | FAM168A | 0.9 | 0.91 | 0.87 | 0.83 | 0.84 | 0.91 | 0.9 | 0.87 | 0.85 |
| chr11_7639871_7641032_7641066_7641478 | PPFIBP2 | 0.31 | 0.31 | 0.27 | 0.66 | 0.61 | 0.36 | 0.37 | 0.3 | 0.26 |
| chr12_101680530_101684381_101684425_101685581 | MYBPC1 | 0.04 | 0.05 | 0.03 | 0.1 | 0.12 | 0.11 | 0.12 | 0.04 | 0.02 |
| chr12_122341698_122347374_122347480_122352725 | CLIP1 | 0.49 | 0.62 | 0.45 | 0.79 | 0.78 | 0.6 | 0.65 | 0.48 | 0.58 |
| chr12_128899288_128906906_128906955_128912423 | GLT1D1 | 0.05 | 0.04 | 0.12 | 0.05 | 0.05 | 0.05 | 0.05 | 0.03 | 0.02 |
| chr12_39311554_39315931_39315971_39319905 | KIF21A | 0.42 | 0.41 | 0.37 | 0.72 | 0.59 | 0.38 | 0.47 | 0.48 | 0.65 |
| chr12_39315971_39318072_39318202_39319905 | KIF21A | 0.62 | 0.8 | 0.7 | 0.95 | 0.94 | 0.81 | 0.87 | 0.62 | 0.82 |
| chr12_55699990_55700261_55700394_55700898 | ITGA7 | 0.34 | 0.25 | 0.34 | 0.14 | 0.19 | 0.3 | 0.25 | 0.38 | 0.52 |
| chr12_71157788_71277350_71277413_71282715 | TSPAN8 | ND | ND | ND | ND | ND | ND | ND | ND | ND |
| chr12_89591296_89598656_89598744_89599116 | ATP2B1 | 0.46 | 0.69 | 0.67 | 0.78 | 0.76 | 0.78 | 0.8 | 0.57 | 0.47 |
| chr13_75312899_75324236_75324402_75326196 | TBC1D4 | 0.03 | 0.02 | 0.06 | 0.11 | 0.07 | 0.03 | 0.07 | 0.01 | 0.01 |
| chr13_75836458_75838139_75838197_75840084 | LMO7 | 0.97 | 0.98 | 0.99 | 0.99 | 0.99 | 0.99 | 0.99 | 0.97 | 0.97 |
| chr13_75838197_75838346_75838389_75840084 | LMO7 | 0.02 | 0.02 | 0.01 | 0.01 | 0.01 | 0.03 | 0.03 | 0.02 | 0.03 |
| chr14_100831535_100835738_100835880_100836166 | MEG3 | 0.03 | 0.03 | 0.02 | 0.02 | 0.02 | 0.03 | 0.03 | 0.02 | 0.01 |
| chr14_35690004_35700161_35700303_35721687 | RALGAPA1 | 0.71 | 0.86 | 0.71 | 0.94 | 0.92 | 0.82 | 0.85 | 0.8 | 0.81 |
| chr14_90593627_90603252_90603304_90610741 | TTC7B | 0.28 | 0.25 | 0.38 | 0.33 | 0.33 | 0.3 | 0.28 | 0.28 | 0.08 |
| chr15_20458448_20461191_20461343_20462342 | HERC2P3 | 0.04 | 0.04 | 0.03 | 0.11 | 0.07 | 0.03 | 0.03 | 0.06 | 0.04 |
| chr15_33842036_33843487_33843575_33844861 | RYR3 | 0.13 | 0.22 | 0.61 | 0.12 | 0.1 | 0.22 | 0.21 | 0.39 | 0.49 |
| chr15_47766678_47768580_47768749_47770496 | SEMA6D | 0.89 | 0.46 | 0.74 | 0.85 | 0.89 | 0.32 | 0.28 | 0.83 | 0.3 |
| chr16_119256_123506_123546_124936 | NPRL3 | 0.03 | 0.05 | 0.06 | 0.03 | 0.04 | 0.02 | 0.02 | 0.06 | 0.04 |
| chr16_2264761_2266109_22662172267662 | RNPS1 | 0.02 | 0.01 | 0.28 | 0.47 | 0.47 | 0.24 | 0.36 | ND | 0.11 |
| chr16_30201107_30201195_30201294_30202689 | SLX1A- | 0.02 | 0 | 0 | 0.06 | 0.05 | 0.03 | 0.04 | 0.01 | 0.02 |
| | SULT1A3 | | | | | | | | | |
| chr16_58509353_58510644_58510684_58511421 | NDRG4 | 0.05 | 0.06 | 0.13 | 0.1 | 0.1 | 0.09 | 0.11 | 0.06 | 0.07 |
| chr17_17850953_17857768_17857856_17861475 | TOM1L2 | 0.87 | 0.88 | 0.9 | 0.73 | 0.78 | 0.86 | 0.86 | 0.88 | 0.82 |
| chr17_73207257_73207701_73207740_73208313 | COG1 | 0.37 | 0.27 | 0.41 | 0.08 | 0.08 | 0.19 | 0.18 | 0.27 | 0.21 |
| chr17_76089321_76090328_76090398_76091142 | EXOC7 | 0.06 | 0.03 | 0.06 | 0.05 | 0.06 | 0.03 | 0.04 | 0.08 | 0.06 |
| chr17_76089321_76090328_76090482_76091142 | EXOC7 | 0.04 | 0.02 | 0.04 | 0.04 | 0.04 | 0.02 | 0.02 | 0.05 | 0.04 |
| chr18_34882202_34884727_34884777_34890299 | DTNA | 0.98 | 0.98 | 0.98 | 0.98 | 0.98 | 0.98 | 0.98 | 0.99 | 0.98 |
| chr18_5397427_5398020_5398144_5406776 | EPB41L3 | 0.42 | 0.7 | 0.44 | 0.94 | 0.94 | 0.76 | 0.8 | 0.43 | 0.65 |
| chr18_58333893_58341677_58341798_58342905 | NEDD4L | 0.97 | 0.97 | 0.97 | 0.96 | 0.96 | 0.97 | 0.98 | 0.98 | 0.98 |
| chr19_45297955_45298142_45298223_45299810 | MARK4 | 0.68 | 0.8 | 0.74 | 0.89 | 0.88 | 0.84 | 0.84 | 0.73 | 0.72 |
| chr19_49146006_49146165_49146193_49148082 | PPFIA3 | 0.91 | 0.93 | 0.88 | 0.99 | 0.98 | 0.96 | 0.96 | 0.91 | 0.92 |
| chr2_121410970_121418621_121418730_121425138 | CLASP1 | 0.17 | 0.34 | 0.22 | 0.57 | 0.52 | 0.38 | 0.46 | 0.27 | 0.29 |
| chr2_127059156_127060595_127060641_127062114 | BIN1 | 0 | 0.01 | 0.01 | 0.01 | 0.01 | 0.01 | 0.01 | 0 | 0 |
| chr2_171707378_171712766_171712827_171715327 | DYNC112 | 0.13 | 0.34 | 0.19 | 0.82 | 0.75 | 0.42 | 0.47 | 0.14 | 0.31 |
| chr2_178535829_178536333_178536463_178537603 | TTN-AS1 | ND | ND | ND | ND | ND | ND | ND | ND | ND |
| chr2_237749325_237751199_237751272_237753308 | LRRFIP1 | 0.08 | 0.06 | 0.07 | 0.02 | 0.03 | 0.04 | 0.04 | 0.07 | 0.05 |
| chr2_240771105_240772569_240772597_240773113 | KIF1A | 0.55 | 0.68 | 0.4 | 0.63 | 0.61 | 0.69 | 0.71 | 0.49 | 0.38 |
| chr2_27094612_27094799_27094935_27096728 | KHK | 0 | 0 | 0 | 0 | 0 | 0 | 0 | 0 | 0 |
| chr2_40177856_40178386_40178494_40428472 | SLC8A1 | 0.24 | 0.9 | 0.38 | ND | ND | 0.92 | 0.94 | 0.74 | 0.79 |
| chr2_69350184_69354258_69354313_69354488 | GFPT1 | 0.01 | 0.06 | 0.04 | 0.05 | 0.1 | 0.08 | 0.06 | 0.01 | 0.05 |
| chr2_86166645_86170748_86170842_86171207 | IMMT | 0.99 | 0.99 | 0.99 | 1 | 0.99 | 0.99 | 1 | 0.99 | 1 |
| chr2_8737000_8747144_8747202_8747886 | KIDINS220 | 0.47 | 0.56 | 0.52 | 0.41 | 0.73 | 0.57 | 0.55 | 0.3 | 0.28 |
| chr2_96605512_96606969_96607048_96608507 | KANSL3 | 0.6 | 0.66 | 0.52 | 0.94 | 0.92 | 0.71 | 0.74 | 0.66 | 0.68 |
| chr3_111949076_111949701_111949891_111952571 | PHLDB2 | ND | ND | ND | ND | ND | 0.01 | ND | ND | ND |
| chr3_111958296_111960144_111960181_111962107 | PHLDB2 | ND | 0.02 | ND | ND | ND | 0 | ND | ND | ND |
| chr3_121835392_121836644_121836795_121844452 | EAF2 | 0.6 | 0.63 | 0.55 | 0.73 | 0.72 | 0.71 | 0.73 | 0.71 | 0.65 |
| chr3_180970359_180971074_180971156_180975312 | FXR1 | 0.15 | 0.15 | 0.17 | 0.06 | 0.09 | 0.14 | 0.11 | 0.11 | 0.1 |
| chr3_37065943_37066223_37066326_37075023 | LRRFIP2 | 0.67 | 0.71 | 0.63 | 0.94 | 0.93 | 0.78 | 0.79 | 0.72 | 0.74 |
| chr3_37066326_37072789_37072883_37075023 | LRRFIP2 | 0.15 | 0.17 | 0.18 | 0.18 | 0.18 | 0.16 | 0.14 | 0.14 | 0.14 |
| chr3_37121692_37127629_37127681_37129062 | LRRFIP2 | 0 | ND | ND | ND | ND | ND | 0.01 | 0.02 | 0.03 |
| chr3_52679727_52681703_52681821_52682091 | PBRM1 | ND | ND | ND | 1 | ND | ND | ND | ND | ND |
| chr3_62499269_62513637_62513707_62516058 | CADPS | 0.96 | 0.96 | 0.85 | 0.79 | 0.8 | 0.96 | 0.94 | 0.96 | 0.81 |
| chr4_101026062_101029165_101029196_101032266 | PPP3CA | 0.68 | 0.8 | 0.83 | 0.68 | 0.69 | 0.83 | 0.84 | 0.66 | 0.57 |
| chr4_113330471_113331971_113332071_113333053 | ANK2 | 0.17 | 0.35 | 0.19 | 0.71 | 0.58 | 0.34 | 0.46 | 0.21 | 0.34 |
| chr4_113373450_113378089_113378182_113381456 | ANK2 | 0 | 0 | 0 | 0 | 0 | 0 | 0 | 0 | 0 |
| chr4_113500512_113502935_113502978_113509637 | CAMK2D | 0.6 | 0.67 | 0.29 | 0.97 | 0.94 | 0.63 | 0.75 | 0.44 | 0.65 |
| chr4_1802026_1802913_1803065_1803691 | FGFR3 | 0.02 | 0.02 | 0.02 | 0.02 | 0.03 | 0.02 | 0.02 | 0.03 | 0.03 |
| chr4_185514338_185514702_185514891_185523361 | PDLIM3 | 0.83 | 0.82 | 0.8 | 0.9 | 0.9 | 0.82 | 0.83 | 0.8 | 0.85 |
| chr4_42556041_42569160_42569206_42574618 | ATP8A1 | 0.28 | 0.71 | 0.56 | 0.94 | 0.89 | 0.48 | 0.78 | 0.3 | 0.61 |
| chr4_8009103_8019617_8019672_8029655 | ABLIM2 | 0.74 | 0.79 | 0.72 | 0.69 | 0.7 | 0.78 | 0.79 | 0.71 | 0.76 |
| chr4_82829059_82830936_82830976_82842139 | SEC31A | 0.05 | 0.05 | 0.04 | 0.06 | 0.06 | 0.06 | 0.07 | 0.06 | 0.05 |
| chr5_103187377_103189166_103189227_103190841 | PPIP5K2 | 0.33 | 0.25 | 0.37 | 0.21 | 0.21 | 0.23 | 0.22 | 0.09 | 0.32 |
| chr5_88823928_88882954_88883000_88887501 | MEF2C | 0.95 | 0.92 | 0.95 | 1 | ND | 0.93 | 0.94 | 0.96 | 0.99 |
| chr6_138433681_138441982_138442115_138447000 | NHSL1 | 0.05 | 0.16 | 0.12 | ND | ND | ND | ND | ND | 0.18 |
| chr6_160143650_160155974_160156075_160158515 | SLC22A1 | 0.87 | ND | ND | ND | ND | ND | ND | ND | ND |
| chr6_29608734_29609228_29609380_29610923 | GABBR1 | 0.94 | 0.96 | 0.93 | 0.99 | 0.97 | 0.94 | 0.96 | 0.93 | 0.95 |
| chr6_54160433_54160739_54160800_54169527 | MLIP | 0.09 | 0.13 | 0.01 | ND | ND | 0.17 | 0.16 | 0.15 | 0.06 |
| chr6_56463765_56464684_56464757_56466077 | DST | 0.4 | 0.5 | 0.4 | 0.65 | 0.63 | 0.56 | 0.6 | 0.45 | 0.53 |
| chr6_75892691_75894813_75894841_75895230 | MYO6 | 0.03 | 0.01 | 0.01 | ND | ND | 0.02 | 0.01 | 0.02 | 0.01 |
| chr7_128849579_128849975_128850075_128850383 | FLNC | 0.48 | 0.42 | 0.55 | 0.94 | 0.83 | 0.5 | 0.43 | 0.33 | 0.36 |
| chr7_44234677_44239588_44239664_44240706 | CAMK2B | 0.83 | 0.87 | 0.93 | 0.98 | 0.97 | 0.92 | 0.92 | 0.91 | 0.66 |
| chr7_51184200_51187913_51187959_51190849 | COBL | 0 | 0 | 0.01 | 0 | 0 | 0 | 0.01 | 0 | 0 |
| chr7_74743521_74744138_74744251_74744757 | GTF21 | 0.89 | 0.78 | 0.89 | 0.58 | 0.49 | 0.86 | 0.87 | 0.92 | 0.92 |
| chr7_81997251_82001658_82001716_82005422 | CACNA2D1 | ND | 0 | 0 | 0 | 0 | 0.01 | 0 | ND | 0 |
| chr8_11861480_11864375_11864484_11868000 | CTSB | 0.96 | 0.93 | 0.93 | 0.99 | ND | 0.93 | 0.93 | 0.98 | 0.99 |
| chr8_22528578_22531301_22531329_22532224 | PPP3CC | 0 | 0 | 0 | 0 | 0 | 0 | 0 | 0 | 0 |
| chr9_105512937_105513598_105513632_105534495 | FSD1L | ND | 0.01 | ND | 0.04 | ND | 0.08 | 0.06 | ND | 0.57 |
| chrX_103962724_103964227_103964295_103964505 | TMSB15B | ND | ND | ND | 0.96 | 0.97 | ND | ND | ND | 0.87 |
| chrX_15827373_15840496_15840592_15845378 | AP1S2 | 0.08 | 0.13 | 0.05 | 0.08 | 0.01 | 0.08 | 0.14 | 0.05 | 0.02 |

| | | Brain - | | Brain - | | | | | | | | |
|---|---|---|---|---|---|---|---|---|---|---|---|---|
| | | Nucleus | Brain - | Spinal | | | | | | | | |
| | | accumbens | Putamen | cord | Brain - | | | Heart - | Heart - | | | |
| | | (basal | (basal | (cervical | Substantia | Colon - | Colon - | Atrial | Left | | Muscle - | |
| | | ganglia) | ganglia) | c-1) | nigra | Sigmoid | Transverse | Appendage | Ventricle | Liver | Skeletal | DRG |
| Coordinates | Gene | (psi) | (psi) | (psi) | (psi) | (psi) | (psi) | (psi) | (psi) | (psi) | (psi) | (psi) |
| chr1_114739891_114741526_114741674_114749820 | CSDE1 | 0.04 | 0.05 | 0.18 | 0.09 | 0.08 | 0.04 | 0.57 | 0.62 | 0.01 | 0.94 | 0.05 |
| chr1_149010596_149012590_149012777_149016298 | PDE4DIP | 0.47 | 0.46 | 0.13 | 0.13 | 0.17 | 0.07 | 0.93 | 0.97 | 0 | 1 | 0.76 |
| chr1_150625809_150626478_150626527_150627311 | ENSA | 0.91 | ND | ND | ND | 0.96 | 0.91 | ND | ND | 0.91 | 0.07 | ND |
| chr1_154157722_154169304_154169384_154170399 | TPM3 | 0.05 | 0.08 | 0.16 | 0.14 | 0.04 | 0.02 | 0.57 | 0.36 | 0.03 | 0.97 | 0.21 |
| chr1_168004794_168015780_168015952_168019544 | DCAF6 | 0.71 | 0.88 | 0.66 | 0.93 | 0.59 | 0.58 | 0.92 | 0.95 | 0.08 | 0.99 | 0.77 |
| chr1_168015952_168019544_168019668_168022987 | DCAF6 | 0.19 | 0.09 | 0.2 | ND | 0.17 | 0.19 | 0.03 | 0.02 | 0.81 | 0 | 0.15 |
| chr1_19148627_19149762_19149796_19150576 | UBR4 | 0.36 | 0.23 | 0.07 | 0.11 | 0.04 | 0.03 | 0.25 | 0.36 | 0 | 0.85 | 0.19 |
| chr1_19339618_19342751_19342865_19344357 | CAPZB | 0.35 | 0.39 | 0.58 | 0.42 | 0.35 | 0.15 | 0.51 | 0.57 | 0.01 | 0.84 | 0.11 |
| chr1_29053313_29058588_29058646_29060421 | EPB41 | 0.79 | 0.87 | 1 | 1 | 0.09 | 0.44 | 0.04 | 0.01 | 0.35 | 0.01 | 0.84 |
| chr1_46583330_46585873_46585975_46594112 | MKNK1 | 0.41 | 0.36 | 0.14 | 0.12 | 0.34 | 0.2 | 0.39 | 0.58 | 0.06 | 0.89 | 0.19 |
| chr1_51750196_51756319_51756359_51760689 | OSBPL9 | 0.3 | 0.41 | 0.42 | 0.42 | 0.63 | 0.76 | 0.57 | 0.43 | 0.96 | 0.15 | 0.29 |
| chr10_122228009_122229345_122229487_122237394 | TACC2 | 0.78 | 0.74 | 0.52 | 0.49 | 0.03 | 0.02 | 0.76 | 0.76 | 0.01 | 0.94 | 0.32 |
| chr10_13597536_13597644_13597672_13600243 | PRPF18 | 0.41 | 0.33 | 0.04 | 0.05 | 0 | 0 | 0.1 | 0.15 | 0 | 0.82 | 0.4 |
| chr10_24474061_24494499_24494605_24495146 | KIAA1217 | 0.89 | 0.9 | 0.59 | 0.59 | 0.33 | 0.63 | 0.51 | 0.57 | 0.92 | 0.06 | 0.14 |
| chr10_24536894_24542506_24542543_24542692 | KIAA1217 | ND | ND | ND | ND | 0 | 0.01 | 0.33 | 0.42 | 0.01 | 0.83 | ND |
| chr10_29524716_29526960_29527057_29529704 | SVIL | 0.51 | ND | 0.58 | 0.27 | 0.38 | 0.31 | 0.82 | 0.9 | 0.09 | 0.99 | 0.48 |
| chr10_310093_311536_311567_327005 | DIP2C | 0.08 | 0.11 | 0.08 | 0.11 | 0.21 | 0.19 | 0.11 | 0.05 | 0.16 | 0.94 | 0.59 |
| chr10_60042760_60053676_60053728_60055657 | ANK3 | 0.01 | 0.01 | 0.01 | 0.01 | 0.01 | 0.01 | 0.06 | 0.07 | 0.01 | 0.96 | 0.04 |
| chr10_73388415_73396043_73396110_73396518 | ANXA7 | 0.39 | 0.28 | 0.37 | 0.25 | 0.02 | 0.01 | 0.52 | 0.59 | 0.01 | 0.92 | 0.36 |
| chr10_76877891_76884988_76885018_76887290 | KCNMA1 | 0.39 | 0.54 | 0.38 | 0.47 | 0.08 | 0.07 | ND | ND | ND | 0.14 | 0.94 |
| chr10_7802855_7806973_7807011_7807658 | ATP5C1 | 0.95 | 0.93 | 0.86 | 0.91 | 0.45 | 0.72 | 0.04 | 0.04 | 0.97 | 0.02 | 0.82 |
| chr10_95367700_95371325_95371428_95375972 | SORBS1 | 0.3 | 0.22 | 0.02 | 0.02 | 0.07 | 0.08 | 0.33 | 0.46 | 0.08 | 0.96 | 0.13 |
| chr10_95367700_95371983_95372050_95375972 | SORBS1 | 0.16 | 0.1 | 0.05 | 0.06 | 0.06 | 0.05 | 0.32 | 0.46 | 0.13 | 0.96 | 0.06 |
| chr10_95384286_95395000_95395076_95397219 | SORBS1 | 0.67 | 0.67 | 0.72 | 0.69 | 0.04 | 0.04 | 0.65 | 0.73 | 0.01 | 0.87 | 0.25 |
| chr10_95432577_95434621_95434718_95437489 | SORBS1 | 0.47 | 0.54 | 0.67 | 0.68 | 0.1 | 0.1 | 0.74 | 0.87 | 0.04 | 0.9 | 0.17 |
| chr11_113805047_113806488_113806585_113808297 | USP28 | 0.51 | 0.5 | 0.52 | 0.47 | 0.05 | 0.04 | 0.83 | 0.88 | 0.05 | 0.91 | 0.35 |
| chr11_115178776_115198405_115198439_115214607 | CADM1 | 0.46 | 0.73 | 0.65 | 0.65 | ND | ND | 0.06 | 0.06 | 0.98 | 0.4 | 0.68 |
| chr11_12239586_12243986_12244113_12249183 | MICAL2 | 0.98 | 0.96 | 0.88 | 0.83 | 0.11 | 0.06 | 0.85 | 0.92 | 0 | 0.95 | ND |
| chr11_65344070_65344579_65344622_65345665 | DPF2 | 0.48 | 0.38 | 0.56 | 0.37 | 0.14 | 0.1 | 0.54 | 0.63 | 0.05 | 0.88 | 0.53 |
| chr11_70378196_70382087_70382159_70383002 | PPFIA1 | 0.99 | 0.98 | 0.97 | 0.98 | 0.52 | 0.76 | 0.47 | 0.43 | 0.99 | 0.14 | 0.71 |
| chr11_73420000_73425021_73425049_73430689 | FAM168A | 0.91 | 0.88 | 0.89 | 0.87 | 0.18 | 0.15 | 0.6 | 0.61 | 0.02 | 0.53 | 0.71 |
| chr11_7639871_7641032_7641066_7641478 | PPFIBP2 | 0.3 | 0.25 | 0.25 | 0.25 | 0.02 | 0 | 0.41 | 0.51 | 0 | 0.85 | 0.1 |
| chr12_101680530_101684381_101684425_101685581 | MYBPC1 | 0.02 | 0.02 | 0.03 | 0.01 | 0.55 | 0.15 | 0.27 | 0.82 | 0.92 | 0.95 | 0.06 |
| chr12_122341698_122347374_122347480_122352725 | CLIP1 | 0.47 | 0.49 | 0.44 | 0.52 | 0.14 | 0.12 | 0.48 | 0.7 | 0.07 | 0.91 | 0.38 |
| chr12_128899288_128906906_128906955_128912423 | GLT1D1 | 0.1 | 0.13 | 0.22 | 0.07 | ND | ND | ND | 0.86 | 0.01 | 0.99 | 0.79 |
| chr12_39311554_39315931_39315971_39319905 | KIF21A | 0.41 | 0.43 | 0.6 | 0.56 | 0.29 | 0.11 | 0.67 | 0.8 | 0.07 | 0.96 | 0.57 |
| chr12_39315971_39318072_39318202_39319905 | KIF21A | 0.8 | 0.66 | 0.4 | 0.61 | 0.93 | 0.94 | 0.26 | 0.24 | 0.86 | 0.16 | 0.97 |
| chr12_55699990_55700261_55700394_55700898 | ITGA7 | 0.29 | 0.44 | 0.56 | 0.56 | 0.97 | 0.96 | 0.21 | 0.31 | 0.89 | 0.13 | 0.22 |
| chr12_71157788_71277350_71277413_71282715 | TSPAN8 | ND | ND | ND | ND | 0 | 0 | ND | ND | ND | 0.96 | ND |
| chr12_89591296_89598656_89598744_89599116 | ATP2B1 | 0.78 | 0.7 | 0.22 | 0.2 | 0.01 | 0 | 0.21 | 0.44 | 0.01 | 0.83 | 0.03 |
| chr13_75312899_75324236_75324402_75326196 | TBC1D4 | 0.04 | 0.01 | 0.01 | 0.04 | 0 | 0 | 0.74 | 0.87 | 0.01 | 0.96 | ND |
| chr13_75836458_75838139_75838197_75840084 | LMO7 | 0.99 | 0.99 | 0.65 | 0.94 | 0.96 | 0.98 | 0.72 | 0.54 | 0.98 | 0.04 | 0.7 |
| chr13_75838197_75838346_75838389_75840084 | LMO7 | 0.01 | 0.02 | 0.15 | 0.08 | 0.01 | 0.01 | 0.94 | 0.97 | 0 | 0.61 | 0.25 |
| chr14_100831535_100835738_100835880_100836166 | MEG3 | 0.02 | 0.03 | 0.04 | 0.02 | 0.48 | 0.67 | 0.82 | 0.85 | 0.9 | 0.7 | 0.06 |
| chr14_35690004_35700161_35700303_35721687 | RALGAPA1 | 0.84 | 0.8 | 0.8 | 0.8 | 0.05 | 0.03 | 0.63 | 0.78 | ND | 0.91 | 0.93 |
| chr14_90593627_90603252_90603304_90610741 | TTC7B | 0.44 | 0.37 | 0.07 | 0.05 | 0.8 | 0.7 | 0.66 | 0.69 | 0.05 | 0.88 | 0.11 |
| chr15_20458448_20461191_20461343_20462342 | HERC2P3 | 0.02 | 0.03 | 0.07 | 0.05 | 0.03 | 0.04 | 0.03 | 0.04 | 0.01 | 0.08 | 0.96 |
| chr15_33842036_33843487_33843575_33844861 | RYR3 | 0.51 | 0.55 | 0.15 | 0.19 | 0.03 | 0.03 | ND | ND | ND | 0.95 | ND |
| chr15_47766678_47768580_47768749_47770496 | SEMA6D | 0.66 | 0.77 | 0.72 | 0.46 | 0.63 | 0.15 | 0.96 | 0.94 | ND | 0.97 | 0.15 |
| chr16_119256_123506_123546_124936 | NPRL3 | 0.06 | 0.06 | 0.04 | 0.04 | 0.38 | 0.42 | 0.47 | 0.53 | 0.88 | 0.41 | 0.13 |
| chr16_2264761_2266109_22662172267662 | RNPS1 | 0.04 | 0.29 | 0.01 | 0.24 | 0.04 | 0.05 | 0.19 | 0.56 | ND | 0.87 | ND |
| chr16_30201107_30201195_30201294_30202689 | SLX1A- | 0 | 0 | 0 | 0.01 | 0.02 | 0.07 | 0.02 | 0.01 | ND | 0.07 | 0.92 |
| | SULT1A3 | | | | | | | | | | | |
| chr16_58509353_58510644_58510684_58511421 | NDRG4 | 0.11 | 0.11 | 0.14 | 0.08 | 0.52 | 0.48 | 0.01 | 0.01 | ND | 0.18 | 0.98 |
| chr17_17850953_17857768_17857856_17861475 | TOM1L2 | 0.89 | 0.88 | 0.91 | 0.86 | 0.33 | 0.19 | 0.85 | 0.85 | 0.02 | 0.62 | 0.67 |
| chr17_73207257_73207701_73207740_73208313 | COG1 | 0.55 | 0.33 | 0.05 | 0.06 | 0.06 | 0.03 | 0.48 | 0.62 | 0.02 | 0.88 | 0.08 |
| chr17_76089321_76090328_76090398_76091142 | EXOC7 | 0.04 | 0.06 | 0.13 | 0.09 | 0.71 | 0.86 | 0.14 | 0.15 | 0.97 | 0.44 | 0.13 |
| chr17_76089321_76090328_76090482_76091142 | EXOC7 | 0.03 | 0.05 | 0.08 | 0.06 | 0.64 | 0.87 | 0.09 | 0.09 | 0.96 | 0.31 | ND |
| chr18_34882202_34884727_34884777_34890299 | DTNA | 0.98 | 0.98 | 0.98 | 0.98 | 0.05 | 0.07 | 0.21 | 0.19 | 1 | 0.82 | 0.89 |
| chr18_5397427_5398020_5398144_5406776 | EPB41L3 | 0.51 | 0.41 | 0.53 | 0.49 | 0.12 | 0.08 | 0.05 | 0.02 | 0.01 | 0.65 | 0.87 |
| chr18_58333893_58341677_58341798_58342905 | NEDD4L | 0.99 | 0.93 | 0.87 | 0.86 | 0.98 | 0.99 | 0.18 | 0.19 | 1 | 0.24 | ND |
| chr19_45297955_45298142_45298223_45299810 | MARK4 | 0.77 | 0.74 | 0.28 | 0.5 | 0.09 | 0.06 | 0.5 | 0.61 | 0.05 | 0.88 | 0.82 |
| chr19_49146006_49146165_49146193_49148082 | PPFIA3 | 0.91 | 0.89 | 0.76 | 0.85 | 0.59 | 0.11 | 0.23 | 0.07 | ND | 0.03 | 0.93 |
| chr2_121410970_121418621_121418730_121425138 | CLASP1 | 0.21 | 0.18 | 0.1 | 0.35 | 0.02 | 0.02 | 0 | 0 | 0.02 | 0.01 | 0.83 |
| chr2_127059156_127060595_127060641_127062114 | BIN1 | 0.01 | 0.01 | 0 | 0 | 0.07 | 0.04 | 0.06 | 0.15 | 0.02 | 1 | 0.06 |
| chr2_171707378_171712766_171712827_171715327 | DYNC112 | 0.3 | 0.15 | 0.03 | 0.09 | 0.04 | 0.21 | 0 | 0 | 0.06 | 0 | 0.81 |
| chr2_178535829_178536333_178536463_178537603 | TTN-AS1 | ND | ND | ND | ND | 0 | 0.01 | 0.62 | 0.82 | ND | 0.94 | ND |
| chr2_237749325_237751199_237751272_237753308 | LRRFIP1 | 0.05 | 0.08 | 0.11 | 0.1 | 0.6 | 0.35 | 0.63 | 0.67 | 0.04 | 0.91 | 0.09 |
| chr2_240771105_240772569_240772597_240773113 | KIF1A | 0.43 | 0.34 | 0.12 | 0.26 | 0.47 | 0.47 | 0.01 | ND | ND | ND | 0.83 |
| chr2_27094612_27094799_27094935_27096728 | KHK | 0 | 0 | 0 | 0 | 0.01 | 0.09 | 0 | 0 | 0.87 | 0 | ND |
| chr2_40177856_40178386_40178494_40428472 | SLC8A1 | 0.62 | 0.4 | 0.48 | 0.81 | 0.03 | 0.03 | 0.85 | 0.93 | ND | ND | ND |
| chr2_69350184_69354258_69354313_69354488 | GFPT1 | 0.06 | 0.08 | 0.03 | 0.01 | 0.14 | 0.04 | 0.42 | 0.71 | 0.01 | 0.83 | 0.28 |
| chr2_86166645_86170748_86170842_86171207 | IMMT | 1 | 1 | 1 | 0.99 | 1 | 1 | 0.19 | 0.13 | 1 | 0.51 | 0.97 |
| chr2_8737000_8747144_8747202_8747886 | KIDINS220 | 0.55 | 0.41 | 0.14 | 0.21 | 0.03 | 0.02 | 0.31 | 0.64 | 0 | 0.83 | 0.32 |
| chr2_96605512_96606969_96607048_96608507 | KANSL3 | 0.62 | 0.59 | 0.66 | 0.59 | 0.09 | 0.05 | 0.43 | 0.6 | 0.05 | 0.91 | 0.54 |
| chr3_111949076_111949701_111949891_111952571 | PHLDB2 | ND | ND | 0 | ND | 0.03 | 0.02 | 0.55 | 0.9 | 0.01 | 0.84 | 0.08 |
| chr3_111958296_111960144_111960181_111962107 | PHLDB2 | ND | ND | ND | ND | 0 | 0 | 0.67 | 0.8 | ND | 0.92 | ND |
| chr3_121835392_121836644_121836795_121844452 | EAF2 | 0.53 | 0.55 | 0.76 | 0.65 | 0.07 | 0.05 | 0.63 | 0.84 | 0.02 | 0.88 | 0.64 |
| chr3_180970359_180971074_180971156_180975312 | FXR1 | 0.16 | 0.18 | 0.08 | 0.12 | 0.65 | 0.39 | 0.83 | 0.85 | 0.03 | 0.98 | 0.06 |
| chr3_37065943_37066223_37066326_37075023 | LRRFIP2 | 0.69 | 0.66 | 0.75 | 0.64 | 0.01 | 0.01 | 0.8 | 0.85 | 0.04 | 0.92 | 0.52 |
| chr3_37066326_37072789_37072883_37075023 | LRRFIP2 | 0.2 | 0.16 | 0.1 | 0.11 | ND | 0.92 | 0.92 | 0.97 | 0.92 | 0.96 | 0.06 |
| chr3_37121692_37127629_37127681_37129062 | LRRFIP2 | ND | 0 | 0.01 | 0 | 0.01 | 0 | 0.93 | 0.94 | 0 | 0.99 | ND |
| chr3_52679727_52681703_52681821_52682091 | PBRM1 | ND | ND | ND | ND | 0.11 | ND | 0.78 | 0.83 | ND | 0.94 | ND |
| chr3_62499269_62513637_62513707_62516058 | CADPS | 0.92 | 0.87 | 0.74 | 0.43 | 0.61 | 0.23 | 0.92 | 0.97 | ND | ND | 0.09 |
| chr4_101026062_101029165_101029196_101032266 | PPP3CA | 0.73 | 0.83 | 0.45 | 0.59 | 0.07 | 0.04 | 0.19 | 0.49 | 0.01 | 0.86 | 0.16 |
| chr4_113330471_113331971_113332071_113333053 | ANK2 | 0.34 | 0.18 | 0.06 | 0.13 | 0.02 | 0.03 | 0 | 0 | ND | 0 | 0.82 |
| chr4_113373450_113378089_113378182_113381456 | ANK2 | 0 | 0 | 0 | 0 | 0.01 | 0.01 | 0.45 | 0.49 | ND | 0.84 | ND |
| chr4_113500512_113502935_113502978_113509637 | CAMK2D | 0.5 | 0.34 | 0.24 | 0.36 | 0.5 | 0.22 | 0.46 | 0.64 | 0.01 | 0.96 | 0.54 |
| chr4_1802026_1802913_1803065_1803691 | FGFR3 | 0.01 | 0.02 | 0.03 | 0.02 | 0.93 | 0.94 | 0.15 | 0.08 | 0.86 | 0.97 | ND |
| chr4_185514338_185514702_185514891_185523361 | PDLIM3 | 0.79 | 0.82 | 0.84 | 0.85 | 0.98 | 0.97 | 0.74 | 0.57 | 0.87 | 0.01 | ND |
| chr4_42556041_42569160_42569206_42574618 | ATP8A1 | 0.78 | 0.43 | 0.18 | 0.19 | 0.07 | 0.02 | 0.53 | 0.83 | ND | 0.92 | 0.54 |
| chr4_8009103_8019617_8019672_8029655 | ABLIM2 | 0.77 | 0.69 | 0.5 | 0.66 | 0.13 | 0.06 | 0.3 | 0.44 | ND | 0.95 | 0.21 |
| chr4_82829059_82830936_82830976_82842139 | SEC31A | 0.05 | 0.05 | 0.04 | 0.05 | 0.19 | 0.48 | 0.1 | 0.09 | 0.94 | 0.18 | 0.09 |
| chr5_103187377_103189166_103189227_103190841 | PPIP5K2 | 0.38 | 0.1 | 0.03 | 0.16 | 0 | 0 | 0.04 | 0.03 | 0 | 0.97 | 0.07 |
| chr5_88823928_88882954_88883000_88887501 | MEF2C | 0.89 | 1 | 0.93 | 0.92 | 0.96 | 0.96 | 0.97 | 0.98 | ND | 0.1 | ND |
| chr6_138433681_138441982_138442115_138447000 | NHSL1 | 0.03 | 0.06 | ND | 0.16 | 0.96 | 0.94 | 0.05 | 0.04 | 0.54 | ND | ND |
| chr6_160143650_160155974_160156075_160158515 | SLC22A1 | ND | ND | ND | ND | ND | ND | 1 | ND | 0.91 | 0.08 | ND |
| chr6_29608734_29609228_29609380_29610923 | GABBR1 | 0.96 | 0.94 | 0.9 | 0.93 | 0.1 | 0.07 | 0.25 | 0.21 | 0.03 | 0.05 | 0.72 |
| chr6_54160433_54160739_54160800_54169527 | MLIP | 0.02 | ND | 0.01 | ND | ND | 0.02 | 0.34 | 0.51 | 0 | 0.81 | 0.02 |
| chr6_56463765_56464684_56464757_56466077 | DST | 0.38 | 0.43 | 0.59 | 0.53 | 0.16 | 0.09 | 0.66 | 0.73 | 0.01 | 0.91 | 0.46 |
| chr6_75892691_75894813_75894841_75895230 | MYO6 | 0.01 | 0 | 0.02 | 0.01 | 0.97 | 0.98 | ND | ND | 0.98 | ND | ND |
| chr7_128849579_128849975_128850075_128850383 | FLNC | 0.44 | 0.36 | 0.38 | 0.34 | 0.98 | 0.97 | 0.25 | 0.22 | 0.94 | 0.02 | ND |
| chr7_44234677_44239588_44239664_44240706 | CAMK2B | 0.94 | 0.93 | 0.81 | 0.41 | ND | ND | 0.01 | 0.01 | ND | 0.8 | 0.83 |
| chr7_51184200_51187913_51187959_51190849 | COBL | 0.01 | 0 | 0 | 0 | 0.02 | 0.01 | 0.36 | 0.4 | 0.01 | 0.99 | ND |
| chr7_74743521_74744138_74744251_74744757 | GTF21 | 0.88 | 0.9 | 0.93 | 0.92 | 0.93 | 0.92 | 0.93 | 0.87 | 0.95 | 0.95 | 0.02 |
| chr7_81997251_82001658_82001716_82005422 | CACNA2D1 | 0 | ND | 0 | 0 | 0 | 0 | 0.29 | ND | ND | 0.9 | ND |
| chr8_11861480_11864375_11864484_11868000 | CTSB | 0.96 | 0.97 | 0.95 | 0.94 | 0.89 | 0.88 | 0.89 | 0.91 | 0.86 | 0.94 | 0.11 |
| chr8_22528578_22531301_22531329_22532224 | PPP3CC | 0 | 0 | 0.01 | 0.01 | 0 | 0 | 0.12 | 0.19 | 0 | 0.91 | 0.1 |
| chr9_105512937_105513598_105513632_105534495 | FSD1L | 0.06 | ND | 1 | 0.16 | ND | ND | ND | ND | ND | 1 | 0.07 |
| chrX_103962724_103964227_103964295_103964505 | TMSB15B | 1 | ND | ND | ND | 0.98 | 1 | ND | ND | ND | ND | 0.15 |
| chrX_15827373_15840496_15840592_15845378 | AP1S2 | 0.07 | 0.04 | 0.01 | 0.01 | 0 | 0 | 0.41 | 0.6 | 0 | 0.93 | ND |

**The columns for Table 5 are further described as follows:
Coordinates: chromosome and splice site coordinates of the alternatively-spliced exon (from hg38). The 4 coordinates indicate the upstream constitutive 5′ splice site, the 3′ splice site of the alternative exon, the 5′ splice site of the alternative exon, and the downstream constitutive 3′ splice site, but the values are all in ascending order regardless of transcribed strand.
Gene: gene name for the gene that contains the screened exon.
Exon length: length of the exon in number of total nucleotides.
Upstream intron sequence by SEQ ID NO: sequence of selected upstream intronic sequence.
Exon sequence by SEQ ID NO: native sequence of the screened alternative exon.
Native 5′ splice site by SEQ ID NO: native 5′ splice site of the alternative exon.
Native 5′ splice site score: score of the native 5′ splice site of the alternative exon.
Exon sequence (with internal ATGs removed and ATG at the end) by SEQ ID NO: native exon sequence with all internal ATGs mutated, and with an ATG at the end of the alternative exon.
Compensated 5′ splice site sequence by SEQ ID NO: a 5′ splice site that has been mutated to match the native splice site strength.
Compensated 5′ splice site sequence score: score of the compensated 5′ splice site.
Downstream intron sequence by SEQ ID NO: sequence of selected downstream intronic sequence.
Downstream intron sequence (with compensated 5′ splice site): sequence of selected downstream intronic sequence with the compensated 5′ splice site.
Kozak sequence by SEQ ID NO: sequence that surrounds the ATG start codon. The first 2 bases following the ATG are GT in the context of a GFP coding sequence.
Kozak sequence score: a score for the efficiency of the Kozak sequence.
5′ splice site sequence (~1 bit stronger) by SEQ ID NO: a 5′ splice site selected to be approximately 1 bit stronger than the native 5′ splice site.
5′ splice site score (~1 bit stronger): the score of this 5′ splice site.
5′ splice site sequence (~1 bit weaker) by SEQ ID NO: a 5′ splice site selected to be approximately 1 bit weaker than the native 5′ splice site.
5′ splice site score (~1 bit weaker): the score of this 5′ splice site.
Subsequent columns denote psi values of each alternative exon in various tissues of interest.

Applications of this High Throughput Screening Approach to Identification of Alternative Exon Cassettes that can be Regulated by T-Cell Activation

The ability to increase or decrease exon inclusion in response to T-cell activation provides utility for various therapeutic purposes, such as CAR-T therapy or other immunotherapies. A major challenge in the context of CAR-T for solid tumors is T-cell exhaustion, a state in which the engineered T-cells no longer exhibit sufficient potency to eliminate tumor cells expressing the neoantigen due to co-expression of multiple inhibitory receptors. It has been previously shown that transcription factors such as T-bet can repress the expression of these inhibitory receptors and can instead sustain the activity of T-cells during chronic infection (26) and also enhance antitumor activity and limit T-cell exhaustion in CAR-T cells (27). However, constitutive over-expression of T-bet may also lead to undesired or autoimmune-like responses (28,29), and thus the addition of T-bet must be regulated in a context-dependent manner. Thus, an exon that is activated by T-cell activation might be engineered to control translation of T-bet or other cargoes that can modulate the state of the T-cell, thereby preventing or limiting T-cell exhaustion.

Publicly available transcriptome datasets in which T-cells were transcriptionally profiled before and after activation (30) were mined, and a set of 98 alternative exon cassettes were chosen (Table 6) to test for splicing behavior within the context of a lentivirus that can integrate into the T-cell genome. Two intronic regions, along with splice site-proximal exon fragments, were selected and fused together to form a new exon cassette. These alternative exon cassettes will be packaged into a lentivirus capsid and will be used to transduce naïve T-cells (31); T-cells will then be activated, RNA will be harvested, and deep sequencing libraries will be prepared and sequenced to identify alternative exon cassettes that show changes in splicing patterns upon activation.

TABLE 6
Table of T-cell activation-responsive exons.

| | | | Sequence 1 | | Sequence 2 |
|---|---|---|---|---|---|
| | | | (by SEQ | | (by SEQ |
| Gene | Strand | Coordinate 1 | ID NO) | Coordinate 2 | ID NO) |
| TNFAIP8 | + | chr5: 118691618-118691740 | 1884 | chr5: 118691793-118691916 | 1982 |
| HRAS | − | chr11: 533177-533299 | 1885 | chr11: 533335-533458 | 1983 |
| NPRL3 | − | chr16: 136567-136689 | 1886 | chr16: 136846-136969 | 1984 |
| NHP2L1 | − | chr22: 42083626-42083748 | 1887 | chr22: 42084308-42084431 | 1985 |
| NHP2L1 | − | chr22: 42078260-42078382 | 1888 | chr22: 42078568-42078691 | 1986 |
| RBM12 | − | chr20: 34246752-34246874 | 1889 | chr20: 34246913-34247036 | 1987 |
| RBM12 | − | chr20: 34243024-34243146 | 1890 | chr20: 34243243-34243366 | 1988 |
| RPL17 | − | chr18: 47017802-47017924 | 1891 | chr18: 47018180-47018303 | 1989 |
| BC021234, | + | chr22: 46746088-46746210 | 1892 | chr22: 46746337-46746460 | 1990 |
| BC069212 | | | | | |
| LUC7L | − | chr16: 278232-278354 | 1893 | chr16: 278378-278501 | 1991 |
| LUC7L | − | chr16: 277141-277263 | 1894 | chr16: 277312-277435 | 1992 |
| RPL22L1 | − | chr3: 170585702-170585824 | 1895 | chr3: 170585900-170586023 | 1993 |
| ABI1 | − | chr10: 27059904-27060011 | 1896 | chr10: 27060012-27060118 | 1994 |
| NUCB2 | + | chr11: 17308081-17308203 | 1897 | chr11: 17308241-17308364 | 1995 |
| NUCB2 | + | chr11: 17312687-17312809 | 1898 | chr11: 17312889-17313012 | 1996 |
| TM2D2 | − | chr8: 38852737-38852859 | 1899 | chr8: 38853290-38853413 | 1997 |
| OGDH | + | chr7: 44686943-44687065 | 1900 | chr7: 44687110-44687233 | 1998 |
| OGDH | + | chr7: 44687156-44687278 | 1901 | chr7: 44687335-44687458 | 1999 |
| TUBA1A | − | chr12: 49579993-49580115 | 1902 | chr12: 49580218-49580341 | 2000 |
| CDC42 | + | chr1: 22405099-22405221 | 1903 | chr1: 22405351-22405474 | 2001 |
| UBA52 | + | chr19: 18682514-18682636 | 1904 | chr19: 18682696-18682819 | 2002 |
| ANKS6 | − | chr9: 101498184-101498306 | 1905 | chr9: 101498882-101499005 | 2003 |
| STAG3L3, | − | chr7: 72470781-72470903 | 1906 | chr7: 72470981-72471104 | 2004 |
| BC073780 | | | | | |
| SRSF6 | + | chr20: 42087693-42087815 | 1907 | chr20: 42088037-42088160 | 2005 |
| USP15 | + | chr12: 62768071-62768193 | 1908 | chr12: 62768293-62768416 | 2006 |
| ARAP1 | − | chr11: 72403698-72403814 | 1909 | chr11: 72403815-72403930 | 2007 |
| RFC5 | + | chr12: 118455395-118455517 | 1910 | chr12: 118455597-118455720 | 2008 |
| C22orf29 | − | chr22: 19833569-19833691 | 1911 | chr22: 19839986-19840109 | 2009 |
| LUC7L | − | chr16: 258500-258622 | 1912 | chr16: 258640-258763 | 2010 |
| KIAA1704 | + | chr13: 45603274-45603396 | 1913 | chr13: 45606219-45606342 | 2011 |
| ARHGAP17 | − | chr16: 24950585-24950707 | 1914 | chr16: 24950895-24951018 | 2012 |
| TRA2A | − | chr7: 23561226-23561348 | 1915 | chr7: 23562028-23562151 | 2013 |
| PDXK | + | chr21: 45175258-45175380 | 1916 | chr21: 45175622-45175745 | 2014 |
| TRA2A | − | chr7: 23561640-23561762 | 1917 | chr7: 23562028-23562151 | 2015 |
| RPL13A | + | chr19: 49993007-49993129 | 1918 | chr19: 49993324-49993447 | 2016 |
| ALDOA | + | chr16: 30077097-30077219 | 1919 | chr16: 30077225-30077348 | 2017 |
| IRF3, | − | chr19: 50167600-50167722 | 1920 | chr19: 50167907-50168030 | 2018 |
| BC013599 | − | chr16: 3182751-3182873 | 1921 | chr16: 3183172-3183295 | 2019 |
| HRAS | − | chr11: 532142-532264 | 1922 | chr11: 532732-532855 | 2020 |
| FAM192A | − | chr16: 57212314-57212436 | 1923 | chr16: 57212741-57212864 | 2021 |
| SNRNP70 | + | chr19: 49605271-49605393 | 1924 | chr19: 49606821-49606944 | 2022 |
| SRSF2 | − | chr17: 74731754-74731876 | 1925 | chr17: 74731934-74732057 | 2023 |
| RPS24 | + | chr10: 79799862-79799984 | 1926 | chr10: 79799985-79800083 | 2024 |
| TRA2A | − | chr7: 23561651-23561773 | 1927 | chr7: 23562028-23562151 | 2025 |
| EIF4H | + | chr7: 73604477-73604599 | 1928 | chr7: 73604613-73604736 | 2026 |
| SIGIRR | − | chr11: 406743-406865 | 1929 | chr11: 406970-407093 | 2027 |
| CPSF7 | − | chr11: 61187842-61187964 | 1930 | chr11: 61189057-61189180 | 2028 |
| HMGN1 | − | chr21: 40719205-40719327 | 1931 | chr21: 40719386-40719509 | 2029 |
| HMGN1 | − | chr21: 40717656-40717778 | 1932 | chr21: 40717861-40717984 | 2030 |
| CENPV | − | chr17: 16251830-16251952 | 1933 | chr17: 16252624-16252747 | 2031 |
| HMGN1 | − | chr21: 40719205-40719327 | 1934 | chr21: 40719381-40719504 | 2032 |
| HMGN1 | − | chr21: 40717656-40717778 | 1935 | chr21: 40719195-40719318 | 2033 |
| SRRT, | + | chr7: 100480286-100480408 | 1936 | chr7: 100480688-100480811 | 2034 |
| NDUFV1 | + | chr11: 67375771-67375893 | 1937 | chr11: 67376170-67376293 | 2035 |
| NAA25 | − | chr12: 112487187-112487309 | 1938 | chr12: 112487392-112487515 | 2036 |
| SBDSP1 | + | chr7: 72301172-72301294 | 1939 | chr7: 72301370-72301493 | 2037 |
| NUMB | − | chr14: 73745889-73746011 | 1940 | chr14: 73746109-73746232 | 2038 |
| MFSD12 | − | chr19: 3542674-3542796 | 1941 | chr19: 3542952-3543075 | 2039 |
| GIT2 | − | chr12: 110384961-110385083 | 1942 | chr12: 110386628-110386751 | 2040 |
| NDUFV1 | + | chr11: 67374329-67374451 | 1943 | chr11: 67374772-67374895 | 2041 |
| FAM136A | − | chr2: 70527872-70527994 | 1944 | chr2: 70528712-70528835 | 2042 |
| EIF4A2, | + | chr3: 186505999-186506121 | 1945 | chr3: 186506186-186506309 | 2043 |
| LETMD1 | + | chr12: 51445775-51445897 | 1946 | chr12: 51446006-51446129 | 2044 |
| RPL13 | + | chr16: 89627248-89627370 | 1947 | chr16: 89627448-89627571 | 2045 |
| RPL13 | + | chr16: 89627535-89627657 | 1948 | chr16: 89627714-89627837 | 2046 |
| DHFR | − | chr5: 79933602-79933724 | 1949 | chr5: 79933805-79933928 | 2047 |
| DHFR | − | chr5: 79929596-79929718 | 1950 | chr5: 79929788-79929911 | 2048 |
| RPL17 | − | chr18: 47017622-47017744 | 1951 | chr18: 47017792-47017915 | 2049 |
| ATXN1 | − | chr6: 16326525-16326647 | 1952 | chr6: 16328678-16328801 | 2050 |
| CHTOP | + | chr1: 153614619-153614741 | 1953 | chr1: 153614882-153615005 | 2051 |
| NHP2L1 | − | chr22: 42083983-42084105 | 1954 | chr22: 42084308-42084431 | 2052 |
| NHP2L1 | − | chr22: 42078260-42078382 | 1955 | chr22: 42078568-42078691 | 2053 |
| TSEN15 | + | chr1: 184041191-184041304 | 1956 | chr1: 184041305-184041428 | 2054 |
| ATG12 | − | chr5: 115176094-115176216 | 1957 | chr5: 115176286-115176409 | 2055 |
| FAM49B | − | chr8: 130915458-130915572 | 1958 | chr8: 130915573-130915696 | 2056 |
| FAM49B | − | chr8: 130883521-130883643 | 1959 | chr8: 130883719-130883842 | 2057 |
| SRSF7 | − | chr2: 38975940-38976062 | 1960 | chr2: 38976292-38976415 | 2058 |
| FAM107B | − | chr10: 14595221-14595343 | 1961 | chr10: 14595363-14595486 | 2059 |
| PCMTD1 | − | chr8: 52751907-52752029 | 1962 | chr8: 52752175-52752298 | 2060 |
| BC013599 | − | chr16: 3182751-3182873 | 1963 | chr16: 3182923-3183046 | 2061 |
| n/a | − | chr2: 220082292-220082414 | 1964 | chr2: 220082506-220082629 | 2062 |
| USP1 | + | chr1: 62901875-62901997 | 1965 | chr1: 62902210-62902333 | 2063 |
| ANO8 | − | chr19: 17438871-17438993 | 1966 | chr19: 17440327-17440450 | 2064 |
| WDR61 | − | chr15: 78577503-78577625 | 1967 | chr15: 78578043-78578166 | 2065 |
| SLC25A3 | + | chr12: 98989111-98989233 | 1968 | chr12: 98989416-98989539 | 2066 |
| PRKAR1B | − | chr7: 767127-767249 | 1969 | chr7: 767290-767413 | 2067 |
| MIB2 | + | chr1: 1564314-1564436 | 1970 | chr1: 1564668-1564791 | 2068 |
| RIOK3 | + | chr18: 21053293-21053415 | 1971 | chr18: 21053567-21053690 | 2069 |
| LMNA | + | chr1: 156107345-156107467 | 1972 | chr1: 156108525-156108648 | 2070 |
| MALT1 | + | chr18: 56378053-56378169 | 1973 | chr18: 56378170-56378285 | 2071 |
| DNASE1 | + | chr16: 3706003-3706125 | 1974 | chr16: 3706163-3706286 | 2072 |
| MAP4K2 | − | chr11: 64568272-64568394 | 1975 | chr11: 64568480-64568603 | 2073 |
| CUGBP2 | + | chr10: 11308461-11308583 | 1976 | chr10: 11308617-11308740 | 2074 |
| MAP2K7 | + | chr10: 7970593-7970715 | 1977 | chr10: 7970717-7970840 | 2075 |
| PTPRC | + | chr1: 198671416-198671538 | 1978 | chr1: 198671636-198671759 | 2076 |
| ZNF496 | + | chr1: 247485900-247486022 | 1979 | chr1: 247486084-247486207 | 2077 |
| FAM136A | − | chr2: 70528554-70528676 | 1980 | chr2: 70528712-70528835 | 2078 |
| COX20 | + | chr1: 245005146-245005268 | 1981 | chr1: 245005337-245005460 | 2079 |

**The columns for Table 6 are further described as follows:
Gene name: name of the gene.
Strand: transcribed strand for this gene.
Coordinate 1: the coordinates of the upstream intron and 5′ end of the exon, including the 3′ splice site (hg19 coordinates).
Sequence 1 (by SEQ ID NO): the sequence corresponding to coordinate 1.
Coordinate 2: the coordinates of the 3′ end of the exon, including the 5′ splice site, and downstream intron (hg19 coordinates).
Sequence 2 (by SEQ ID NO): the sequence corresponding to coordinate 2.

### References of Example 5

1. Wang, E. T. et al. Alternative isoform regulation in human tissue transcriptomes. Nature 456, 470-476 (2008).
2. Gerstberger, S., Hafner, M., Ascano, M. & Tuschl, T. Evolutionary Conservation and Expression of Human RNA-Binding Proteins and Their Role in Human Genetic Disease. Adv. Exp. Med. Biol. 825, 1-55 (2014).
3. Cooper, T. A. Use of minigene systems to dissect alternative splicing elements. Methods 37, 331-340 (2005).
4. Coulter, L. R., Landree, M. A. & Cooper, T. A. Identification of a new class of exonic splicing enhancers by in vivo selection. Mol. Cell. Biol. 17, 2143-2150 (1997).
5. Lorson, C. L., Hahnen, E., Androphy, E. J. & Wirth, B. A single nucleotide in the SMN gene regulates splicing and is responsible for spinal muscular atrophy. Proc. Natl. Acad. Sci. 96, 6307-6311 (1999).
6. Wong, M. S., Kinney, J. B. & Krainer, A. R. Quantitative Activity Profile and Context Dependence of All Human 5′ Splice Sites. Mol. Cell 71, 1012-1026.e3 (2018).
7. Orengo, J. P., Bundman, D. & Cooper, T. A. A bichromatic fluorescent reporter for cell-based screens of alternative splicing. Nucleic Acids Res. 34, e148 (2006).
8. Boyne, A. R., et al. International Patent App. No. PCT/US2016/016234, entitled “Regulation of gene expression by aptamer-mediated modulation of alternative splicing”, filed Feb. 2, 2016 and published as International Pub. No. WO2016126747A1.
9. Monteys, A. M. et al. Regulated control of gene therapies by drug-induced splicing. Nature 596, 291-295 (2021).
10. Freyermuth, F. et al. Splicing misregulation of SCNSA contributes to cardiac-conduction delay and heart arrhythmia in myotonic dystrophy. Nat. Commun. 7, 11067 (2016).
11. Cheung, R. et al. A Multiplexed Assay for Exon Recognition Reveals that an Unappreciated Fraction of Rare Genetic Variants Cause Large-Effect Splicing Disruptions. Mol. Cell 73, 183-194.e8 (2019).
12. Lawlor, M. W. & Dowling, J. J. X-linked myotubular myopathy. Neuromuscul. Disord. 31, 1004-1012 (2021).
13. Childers, M. K. et al. Gene Therapy Prolongs Survival and Restores Function in Murine and Canine Models of Myotubular Myopathy. Sci. Transl. Med. 6, 220ra10 (2014).
14. Yeo, G. & Burge, C. B. Maximum entropy modeling of short sequence motifs with applications to RNA splicing signals. J. Comput. Biol. J. Comput. Mol. Cell Biol. 11, 377-394 (2004).
15. Noderer, W. L. et al. Quantitative analysis of mammalian translation initiation sites by FACS-seq. Mol. Syst. Biol. 10, 748 (2014).
16. Adamson, S. I., Zhan, L. & Graveley, B. R. Vex-seq: high-throughput identification of the impact of genetic variation on pre-mRNA splicing efficiency. Genome Biol. 19, 71 (2018).
17. Carithers, L. J. et al. A Novel Approach to High-Quality Postmortem Tissue Procurement: The GTEx Project. Biopreservation Biobanking 13, 311-319 (2015).
18. Cook, K. B., Kazan, H., Zuberi, K., Morris, Q. & Hughes, T. R. RBPDB: a database of RNA-binding specificities. Nucleic Acids Res. 39, D301-D308 (2011).
19. Fugier, C. et al. Misregulated alternative splicing of BIN1 is associated with T tubule alterations and muscle weakness in myotonic dystrophy. Nat. Med. 17, 720-725 (2011).
20. Singh, R. K., Kolonin, A. M., Fiorotto, M. L. & Cooper, T. A. Rbfox-Splicing Factors Maintain Skeletal Muscle Mass by Regulating Calpain3 and Proteostasis. Cell Rep. 24, 197-208 (2018).
21. Farazi, T. A. et al. Identification of the RNA recognition element of the RBPMS family of RNA-binding proteins and their transcriptome-wide mRNA targets. RNA 20, 1090-1102 (2014).
22. Nakagaki-Silva, E. E. et al. Identification of RBPMS as a mammalian smooth muscle master splicing regulator via proximity of its gene with super-enhancers. eLife 8, e46327 (2019).
23. Naqvi, S. et al. Conservation, acquisition, and functional impact of sex-biased gene expression in mammals. Science (2019) doi:10.1126/science.aaw7317.
24. Tabebordbar, M. et al. Directed evolution of a family of AAV capsid variants enabling potent muscle-directed gene delivery across species. Cell 184, 4919-4938.e22 (2021).
25. Chan, K. Y. et al. Engineered AAVs for efficient noninvasive gene delivery to the central and peripheral nervous systems. Nat. Neurosci. 20, 1172-1179 (2017).
26. Kao, C. et al. Transcription factor T-bet represses expression of the inhibitory receptor PD-1 and sustains virus-specific CD8+ T cell responses during chronic infection. Nat. Immunol. 12, 663-671 (2011).
27. Gacerez, A. T. & Sentman, C. L. T-bet promotes potent antitumor activity of CD4+ CAR T cells. Cancer Gene Ther. 25, 117-128 (2018).
28. Austin, J. W. et al. Overexpression of T-bet in HIV infection is associated with accumulation of B cells outside germinal centers and poor affinity maturation. Sci. Transl. Med. (2019) doi:10.1126/scitranslmed.aax0904.
29. Shimohata, H. et al. Overexpression of T-bet in T cells accelerates autoimmune glomerulonephritis in mice with a dominant Th1 background. J. Nephrol. 22, 123-129 (2009).
30. Martinez, N. M. et al. Alternative splicing networks regulated by signaling in human T cells. RNA 18(5), 1029-1040 (2012).
31. Bernadin, O. et al. Baboon envelope LVs efficiently transduced human adult, fetal, and progenitor T cells and corrected SCID-X1 T-cell deficiency. Blood Adv. 3, 461-475 (2019).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present disclosure, and without departing from the spirit and scope thereof, can make various changes and modifications of the present disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A transgene comprising:
(i) a constitutive exon and one or more intronic sequences, each from a first gene;
(ii) an alternatively-spliced exon cassette, wherein the alternatively-spliced exon cassette comprises:
(a) an alternatively-spliced exon, and
(b) flanking intronic sequences,
wherein each of (a) and (b) are from a second gene; and
(iii) a coding region of interest from a third gene,
wherein the alternatively-spliced exon comprises an ATG start codon.

2. The transgene of claim 1, wherein the first and second gene are the same gene; the first and third gene are the same gene; or all of the first, second, and third genes are the same gene.

3. The transgene of claim 1, wherein the first gene is survival motor neuron 1 (SMN1).

4. The transgene of claim 1, wherein the constitutive exon comprises exon 6 of SMN1, or a portion thereof.

5. The transgene of claim 1, wherein the constitutive exon comprises a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 102.

6. The transgene of claim 1, wherein the constitutive exon comprises a polynucleotide having a nucleic acid sequence as set forth in SEQ ID NO: 102.

7. The transgene of claim 1, wherein the one or more intronic sequences of (i) are or are derived from intron 6 and/or intron 7 of SMN1.

8. The transgene of claim 1, wherein the one or more intronic sequences of (i) comprise(s) a polynucleotide having at least 70%, at least 75%, at least 80%, at least 90%, at least 92%, at least 95%, at least 98%, or at least 99% sequence identity, relative to a nucleic acid sequence as set forth in SEQ ID NO: 103 and/or SEQ ID NO: 104.

9. The transgene of claim 1, wherein the one or more intronic sequences of (i) comprise(s) a polynucleotide having a nucleic acid sequence as set forth in SEQ ID NO: 103 and/or SEQ ID NO: 104.

10. The transgene of claim 1, wherein the second gene is a gene selected from the group consisting of: CAMK2B, PKP2, LGMN, NRAP, VPS39, KSR1, PDLIM3, BIN1, ARFGAP2, KIF13A, and/or PICALM.

11. The transgene of claim 1, wherein the second gene is bridging integrator 1 (BIN1).

12. The transgene of claim 1, wherein the alternatively-spliced exon comprises exon 11 of BIN1.

13. The transgene of claim 1, wherein the alternatively-spliced exon comprises a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 37 or SEQ ID NO: 38.

14. The transgene of claim 1, wherein the alternatively-spliced exon comprises a polynucleotide having a nucleic acid sequence as set forth in either SEQ ID NO: 37 or SEQ ID NO: 38.

15. The transgene of claim 1, wherein the flanking intronic sequences of (ii) are or are derived from intron 10 and/or intron 11 of BIN1.

16. The transgene of claim 1, wherein the flanking intronic sequences of (ii) each comprise a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 15 or SEQ ID NO: 16.

17. The transgene of claim 1, wherein the flanking intronic sequences of (ii) each comprise a polynucleotide having a nucleic acid sequence as set forth in either SEQ ID NO: 15 or SEQ ID NO: 16.

18. The transgene of claim 1, wherein the alternatively-spliced exon cassette comprises a polynucleotide having at least 70%, at least 75%, at least 80%, at least 90%, at least 92%, at least 95%, at least 98%, or at least 99% sequence identity, relative to a nucleic acid sequence as set forth in any one of SEQ ID NOs: 107-778.

19. The transgene of claim 1, wherein the alternatively-spliced exon cassette comprises a polynucleotide having a nucleic acid sequence as set forth in any one of SEQ ID NOs: 107-778.

20. The transgene of claim 1, wherein the third gene is myotubularin 1 (MTM1) or calpain 3 (CAPN3).

21. The transgene of claim 1, wherein the coding region of interest comprises a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 1881 or SEQ ID NO: 1882.

22. The transgene of claim 1, wherein the coding region of interest comprises a polynucleotide having a nucleic acid sequence as set forth in either SEQ ID NO: 1881 or SEQ ID NO: 1882.

23. The transgene of claim 1, wherein, if the wild-type alternatively-spliced exon does not comprise an ATG start codon, the alternatively-spliced exon comprises 1-3 nucleic acid substitutions, relative to the wild-type alternatively-spliced exon, to form the ATG start codon within the alternatively-spliced exon.

24. The transgene of claim 23, wherein the ATG start codon is formed in the alternatively-spliced exon by 1 nucleic acid substitution.

25. The transgene of claim 23, wherein the ATG start codon is formed in the alternatively-spliced exon by 2 nucleic acid substitutions.

26. The transgene of claim 23, wherein the ATG start codon is formed in the alternatively-spliced exon by 3 nucleic acid substitutions.

27. The transgene of claim 1, wherein the alternatively-spliced exon is retained in the spliced transcript.

28. The transgene of claim 1, wherein all native start codons located 5′ to the ATG start codon located within the alternatively-spliced exon are disrupted or deleted.

29. The transgene of claim 1, wherein the alternatively-spliced exon cassette is located 5′, relative to the coding region of interest.

30. The transgene of claim 1, wherein the constitutive exon is located 5′, relative to the alternatively-spliced exon cassette.

31. The transgene of claim 1, wherein the one or more intronic sequences of (i) flank the alternatively-spliced exon cassette.

32. The transgene of claim 1, wherein the alternatively-spliced exon comprises a heterologous, in-frame stop codon.

33. The transgene of claim 32, wherein the heterologous, in-frame stop codon is at least 50 nucleotides upstream of the next 5′ splice junction.

34. The transgene of claim 32, wherein the heterologous, in-frame stop codon elicits nonsense-mediated decay.

35. The transgene of claim 1, wherein the alternatively-spliced exon is retained in the spliced transcript in distinct tissues.

36. The transgene of claim 35, wherein the alternatively-spliced exon is retained in the spliced transcript in skeletal muscle, and/or wherein the alternatively-spliced exon is not retained in the spliced transcript in heart and/or liver tissue.

37. The transgene of claim 1, wherein the flanking intronic sequences of (ii)(b) are or are derived from native flanking introns of the alternatively-spliced exon.

38. The transgene of claim 1, wherein the flanking intronic sequences of (ii)(b) each comprise at least one modification, relative to a naturally occurring intronic sequence.

39. The transgene of claim 38, wherein the modification is a substitution or deletion of one or more nucleic acids.

40. The transgene of claim 1, wherein the ATG start codon is located at the 3′ end of the alternatively-spliced exon.

41. The transgene of claim 40, wherein, if the wild-type alternatively-spliced exon does not comprise an ATG start codon at its 3′ end, the first 10 nucleotides of the flanking intronic sequence which is immediately 3′ to the alternatively-spliced exon comprise 1-5 nucleotide substitutions, relative to the wild-type flanking intronic sequence which is immediately 3′ to the wild-type alternatively-spliced exon.

42. The transgene of claim 1, wherein the one or more intronic sequences of (i) each comprise at least one modification, relative to a naturally occurring intronic sequence.

43. The transgene of claim 42, wherein the modification is a substitution or deletion of one or more nucleic acids.

44. The transgene of claim 1, wherein the coding region of interest comprises at least one modification, relative to a naturally occurring coding region of the third gene.

45. The transgene of claim 44, wherein the modification is a substitution or deletion of one or more nucleic acids.

46. The transgene of claim 44, wherein the coding region of interest comprises a deletion or disruption of a native start codon.

47. The transgene of claim 44, wherein the coding region of interest comprises at least one heterologous stop codon.

48. The transgene of claim 47, wherein the at least one heterologous stop codon is at least 50 nucleotides upstream of the next 5′ splice junction.

49. The transgene of claim 47, wherein the at least one heterologous stop codon elicits nonsense-mediated decay.

50. The transgene of claim 1, further comprising a 3′ untranslated region (UTR).

51. The transgene of claim 50, wherein the 3′ UTR comprises a polyadenylation (pA) site and a cleavage site.

52. The transgene of claim 51, wherein the polyadenylation site is an SV40 pA site.

53. The transgene of claim 1, further comprising a promoter, wherein the promoter is located 5′, relative to all of (i), (ii), and (iii).

54. The transgene of claim 53, wherein the promoter is a tissue-specific promoter.

55. The transgene of claim 54, wherein the tissue-specific promoter is an MHCK7 promoter.

56. The transgene of claim 1, wherein the alternatively-spliced exon cassette comprises a nucleic acid sequence which is 450 to 650 nucleotides in length.

57. A recombinant viral genome comprising the transgene of claim 1.

58. The recombinant viral genome of claim 57, wherein the recombinant viral genome is a genome from a recombinant adeno-associated virus (rAAV).

59. The recombinant viral genome of claim 58, wherein the transgene is flanked by AAV inverted terminal repeat (ITR) sequences.

60. The recombinant viral genome of claim 59, wherein the AAV ITR sequences are AAV2 ITR sequences.

61. The recombinant viral genome of claim 57, wherein the recombinant viral genome comprises a polynucleotide having at least 70%, at least 75%, at least 80%, at least 90%, at least 92%, at least 95%, at least 98%, or at least 99% sequence identity, relative to a nucleic acid sequence as set forth in either SEQ ID NO: 105 or SEQ ID NO: 106.

62. The recombinant viral genome of claim 57, wherein the recombinant viral genome comprises a polynucleotide having a nucleic acid sequence as set forth in either SEQ ID NO: 105 or SEQ ID NO: 106.

63. An rAAV particle comprising a recombinant viral genome according to claim 57.

64. The rAAV particle of claim 63, wherein the rAAV particle comprises AAV serotype 1, 2, 3, 4, 5, 6, 7, 8, 9, or 10, or AAV derivative or pseudotype AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAV5hH10, AAV2 (Y→F), AAV8 (Y733F), AAV2.15, AAV2.4, AAVM41, and AAVr3.45.

65. The rAAV particle of claim 63, further comprising at least one helper plasmid.

66. The rAAV particle of claim 65, wherein the helper plasmid comprises a rep gene and a cap gene.

67. The rAAV particle of claim 66, wherein the rep gene encodes Rep78, Rep68, Rep52, or Rep40, and/or wherein the cap gene encodes a VP1, VP2, and/or VP3 region of the viral capsid protein.

68. The rAAV particle of claim 65, wherein the rAAV particle comprises two helper plasmids.

69. The rAAV particle of claim 68, wherein the first helper plasmid comprises a rep gene and a cap gene and the second helper plasmid comprises a E1a gene, a E1b gene, a E4 gene, a E2a gene, and a VA gene.

70. A recombinant viral genome comprising a transgene, wherein the transgene comprises:
(i) a constitutive exon and one or more intronic sequences;
(ii) an alternative exon cassette comprising:
(a) an alternatively-spliced exon;
(b) at least a portion of the intron immediately upstream of the alternatively-spliced exon; and
(c) at least a portion of the intron immediately downstream of the alternatively-spliced exon,
wherein, if the wild-type alternatively-spliced exon does not comprise an ATG start codon at its 3′ end:
(1) the 3′ end of the alternatively-spliced exon comprises 1-3 nucleic acid substitutions relative to the wild-type alternatively-spliced exon to form an ATG start codon, and
(2) the first 10 nucleotides of the intron immediately downstream of the alternatively-spliced exon comprise 1-5 nucleic acid substitutions relative to the wild-type intron immediately downstream of the wild-type alternatively-spliced exon; and
(iii) a coding region of interest.

71. The recombinant viral genome of claim 70, wherein the 1-5 nucleic acid substitutions of (2) increase splice site strength.

72. The recombinant viral genome of claim 70, wherein any wild-type start codons within the alternatively-spliced exon located upstream of the ATG start codon at the 3′ end of the alternatively-spliced exon are disrupted or deleted.

73. The recombinant viral genome of claim 70, further comprising a tissue-specific promoter upstream of the alternative exon cassette.

74. The recombinant viral genome of claim 73, wherein the coding region of interest is or is derived from a naturally occurring coding region of MTM1 or CAPN3.

75. The recombinant viral genome of claim 74, wherein the tissue-specific promoter is an MHCK7 promoter.

76. The recombinant viral genome of claim 75, wherein the alternative exon is exon 11 of the BIN1 gene.

77. The recombinant viral genome of claim 76, wherein the constitutive exon is exon 6 of the SMN1 gene.

78. The recombinant viral genome of claim 77, wherein the alternative exon cassette promotes skeletal muscle expression of the coding region of interest and reduces cardiac muscle expression of the coding region of interest.

79. The recombinant viral genome of claim 78, wherein the alternative exon cassette is approximately 600 nucleotides in length.

80. A method of treating a disease or condition in a subject comprising administering a recombinant viral genome according to any one of claim 57-62 or 70-79, or an rAAV particle according to any one of claims 63-69, to the subject.

81. The method of claim 80, wherein the subject is a mammal.

82. The method of claim 81, wherein the mammal is a human.

83. The method of any one of claims 80-82, wherein the recombinant viral genome or rAAV particle is administered to the subject at least one time.

84. The method of claim 83, wherein the viral genome or rAAV particle is administered to the subject 2, 3, 4, 5, 6, 7, 8, 9, or 10 times.

85. The method of any one of claims 80-84, wherein the viral genome or rAAV particle is administered to the subject parenterally, subcutaneously, intraocularly, intravitreally, subretinally, intravenously (IV), intracerebro-ventricularly, intramuscularly, intrathecally (IT), intracisternally, intraperitoneally, enterally, via inhalation, topically, or by direct injection to one or more cells, tissues, or organs.

86. The method of any one of claims 80-85, wherein the viral genome or viral particle is administered to the subject by intravenous injection, intramuscular injection, intrathecal injection, or intravitreal injection.

87. The method of any one of claims 80-86, wherein the disease or condition is a disease or condition selected from the group consisting of Dentatorubral-pallido-luysian atrophy (DRPLA), myotonic dystrophy type 1 (DM1), myotonic dystrophy type 2 (DM2), Fragile X syndrome of mental retardation (FMR1), Fragile X tremor ataxia syndrome (FXTAS), FRAXE mental retardation (FMR2), Friedreichs ataxia (FRDA), Huntington disease (HD), Huntington disease-like 2 (HDL2), Oculopharyngeal muscular dystrophy (OPMD), Myoclonic epilepsy type 1, Alzheimer's disease, ALS/FTD, spinocerebellar ataxia type 1 (SCA1), spinocerebellar ataxia type 2 (SCA2), spinocerebellar ataxia type 3 (SCA3), spinocerebellar ataxia type 6 (SCA6), spinocerebellar ataxia type 7 (SCAT), spinocerebellar ataxia type 8 (SCA8), spinocerebellar ataxia type 10 (SCA10), spinocerebellar ataxia type 12 (SCA12), spinocerebellar ataxia type 17 (SCA17), Syndromic/non-syndromic X-linked mental retardation, Emery-Dreifuss muscular dystrophy type 2, familial partial lipodystrophy, limb girdle muscular dystrophy type 1B, dilated cardiomyopathy, familial partial lipodystrophy, Charcot-Marie-Tooth disorder type 2B1, mandibuloacral dysplasia, childhood progeria syndrome (Hutchinson-Gilford syndrome), Werner syndrome, Dilated cardiomyopathy (DCM), Hypertrophic cardiomyopathy (HCM), Restrictive cardiomyopathy (RCM), Left Ventricular Non-compaction (LVNC), Arrhythmogenic Right Ventricular Dysplasia (ARVD), takotsubo cardiomyopathy, Duchenne muscular dystrophy, Becker muscular dystrophy, Limb-girdle muscular dystrophy, Facioscapulohumeral muscular dystrophy, Congenital muscular dystrophy, Oculopharyngeal muscular dystrophy, Distal muscular dystrophy, Emery-Dreifuss muscular dystrophy, dementia, Parkinson's disease (PD), a PD-related disorder, Prion disease, a motor neuron disease (MND), Progressive bulbar palsy (PBP), Progressive muscular atrophy (PMA), Primary lateral sclerosis (PLS), Spinal muscular atrophy (SMA), a bladder cancer, a breast cancer, a colorectal cancer, a kidney cancer, a lung cancer, a lymphoma, a melanoma, an oral cancer, an ovarian cancer, an oropharyngeal cancer, a pancreatic cancer, a prostate cancer, a thyroid cancer, a uterine cancer, Down syndrome, Prader-Willi Syndrome (PWS), Bloom Syndrome, Cockayne Syndrome Type I-216400, Cockayne Syndrome Type III, Cockayne Syndrome Type I, Hutchinson-Gilford Progeria Syndrome, Mandibuloacral Dysplasia with Type A Lipodystrophy, Progeria, Adult Onset Progeroid Syndrome, Neonatal Rothmund-Thomson Syndrome, Seip Syndrome, Werner Syndrome, Replication Focus-Forming Activity 1, myotubular myopathy, Danon Disease, and/or centronuclear myopathy.

88. The transgene of claim 1, wherein the ATG start codon is in the same reading frame as the coding region of interest.

89. The transgene of claim 1, wherein the ATG start codon is within up to 5, 10, 20, or 30 nucleotides upstream of the 3′ end of the alternative-spliced exon.

90. The transgene of claim 1, wherein the ATG start codon is within up to 5, 10, 20, or 30 nucleotides upstream of the 3′ end of the alternative-spliced exon and is in the same reading frame as the coding region of interest.
